(19) United States
(12) Patent Application Publication
(10) Pub. No.: US 2023/0348928 A1
(43) Pub. Date: Nov. 2, 2023

(54) PLANT REGULATORY ELEMENTS AND METHODS OF USE THEREOF

(71) Applicant: PIONEER HI-BRED INTERNATIONAL, INC., JOHNSTON, IA (US)

(72) Inventors: PRIYANKA BHYRI, JOHNSTON, IA (US); NANDINI DASGUPTA; KRUPA DESHMUKH, JOHNSTON, IA (US); SCOTT DIEHN, WEST DES MOINES, IA (US); KNUT MEYER, WILMINGTON, DE (US); GILDA RAUSCHER, JOHNSTON, IA (US); KEVIN G. RIPP, DES MOINES, IA (US); LYNNE EILEEN SIMS, POLK CITY, IA (US)

(73) Assignee: PIONEER HI-BRED INTERNATIONAL, INC., JOHNSTON, IA (US)

(21) Appl. No.: 18/006,423

(22) PCT Filed: Aug. 4, 2021
(86) PCT No.: PCT/US21/44433

§ 371 Date: Jan. 23, 2023

Related U.S. Application Data
(60) Provisional application No. 63/063,467, filed on Aug. 10, 2020.

(51) Int. Cl. C12N 15/82

(52) U.S. Cl. CPC C12N 15/8279; C12N 15/8274; C12N 15/8262

(57) ABSTRACT

The present disclosure relates to the field of plant molecular biology, more particularly to regulation of gene expression in plants.

### REFERENCE TO SEQUENCE LISTING SUBMITTED ELECTRONICALLY

A sequence listing having the file name “7667_SeqList.txt” created on Aug. 4, 2020 and having a size of 370 kilobytes is filed in computer readable form concurrently with the specification. The sequence listing is part of the specification and is herein incorporated by reference in its entirety.

### FIELD

The present disclosure relates to the field of plant molecular biology, more particularly to the regulation of gene expression in plants.

### BACKGROUND

Expression of heterologous DNA sequences in a plant host is dependent upon the presence of operably linked regulatory elements that are functional within the plant host. Choice of promoter sequence may determine when and where within the organism a heterologous DNA sequence is expressed. Where expression in specific tissues or organs is desired, tissue-preferred promoters may be used. Where gene expression in response to a stimulus is desired, inducible promoters are the regulatory element of choice. In contrast, where continuous expression is desired throughout the cells of a plant, constitutive promoters are utilized. Additional regulatory sequences upstream and/or downstream from the core promoter sequence may be included in the expression constructs of transformation vectors to bring about varying levels of expression of heterologous nucleotide sequences in a transgenic plant.

Frequently it is desirable to express a DNA sequence in particular tissues or organs of a plant. For example, increased resistance of a plant to infection by soil- and air-borne pathogens might be accomplished by genetic manipulation of the plant's genome to comprise a tissue-preferred promoter operably linked to a heterologous pathogen-resistance gene such that pathogen-resistance proteins are produced in the desired plant tissue. Alternatively, it might be desirable to inhibit expression of a native DNA sequence within a plant's tissues to achieve a desired phenotype. In this case, such inhibition might be accomplished with transformation of the plant to comprise a tissue-preferred promoter operably linked to an antisense nucleotide sequence, such that expression of the antisense sequence produces an RNA transcript that interferes with translation of the mRNA of the native DNA sequence.

Genetically altering plants through the use of genetic engineering techniques and thus producing a plant with useful traits may require the availability of a variety of regulatory elements. An accumulation of promoters and other regulatory elements would enable the investigator to express at desired levels and cellular locales recombinant DNA molecules. Therefore, a collection of promoters would allow for a new trait to be expressed at the desired level in the desired tissue. Thus, isolation, characterization, and creation of regulatory elements that may produce an expression pattern that is unique and serve as regulatory regions for expression of heterologous nucleotide sequences of interest are useful for the genetic manipulation of plants.

### SUMMARY

Compositions and methods for regulating expression of a heterologous polynucleotide sequence of interest in a plant or plant cell are provided. DNA molecules comprising novel polynucleotide sequences for regulatory elements that initiate transcription are provided. In some embodiments the regulatory element has promoter activity initiating transcription in a plant cell. Certain embodiments comprise the nucleotide sequences set forth in SEQ ID NOs: 1-206. Also included are functional fragments, segments, or variants of the sequences set forth in SEQ ID NOs: 1-206 wherein said sequences have regulatory activity and/or initiate transcription in a plant cell, or a polynucleotide sequence comprising a sequence having at least 85% sequence identity to any one of the sequences set forth in SEQ ID NOs: 1-206, wherein said sequences have regulatory activity and/or initiate transcription in the plant cell. Embodiments also include DNA constructs comprising a promoter operably linked to a heterologous nucleotide sequence of interest, wherein said promoter is capable of driving expression of said heterologous nucleotide sequence in a plant cell and said promoter comprises one of the nucleotide sequences disclosed herein. Embodiments also include DNA constructs comprising an enhancer and a heterologous promoter operably linked to a heterologous polynucleotide sequence of interest, wherein said enhancer and heterologous promoter are capable of driving expression of said polynucleotide sequence in a plant cell and said heterologous promoter comprises one of the polynucleotide sequences set forth in SEQ ID NOs: 1-206. Embodiments further provide expression vectors, and plants or plant cells having stably incorporated into their genomes a DNA construct as is described above. Additionally, compositions include transgenic seed of such plants.

Embodiments also include DNA constructs comprising a promoter operably linked to a heterologous polynucleotide sequence of interest, wherein said promoter is capable of driving expression of said heterologous polynucleotide sequence in a plant cell and said promoter comprises one of SEQ ID NOs: 1-206, or a functional fragment thereof, as disclosed herein. Embodiments further provide expression vectors, and plants or plant cells having stably incorporated into their genomes a DNA construct as is described above. Additionally, compositions include transgenic seed of such plants.

Downstream from the transcriptional initiation region of the regulatory element will be a sequence of interest that will provide for modification of the phenotype of the plant. Such modification includes modulating the production of an endogenous product as to amount, relative distribution, or the like, or production of an exogenous expression product, to provide for a novel or modulated function or product in the plant. For example, a heterologous polynucleotide sequence that encodes a gene product that confers resistance or tolerance to herbicide, salt, cold, drought, pathogen, nematodes or insects is encompassed.

In a further embodiment, a method for modulating expression of a gene in a stably transformed plant is provided, comprising the steps of (a) transforming a plant cell with a DNA construct comprising a regulatory element disclosed herein, or a functional fragment thereof, operably linked to at least one heterologous polynucleotide sequence; (b) growing the plant cell under plant growing conditions and (c) regenerating a stably transformed plant from the plant cell wherein expression of the linked nucleotide sequence alters the phenotype of the plant. In another embodiment, the DNA construct further comprises a heterologous enhancer element.

Expression cassettes comprising one or more of the regulatory element sequences of SEQ ID NOs: 1-206 operably linked to a heterologous polynucleotide sequence of interest are provided. Additionally provided are transformed plant cells, plant tissues, seeds, and plants comprising said expression cassettes.

Description of Sequences

TABLE 1
Sequence Listing Description

| SEQ ID | |
|---|---|
| NO | Sequence name |
| 1 | GM-CAB AB80 PRO (MOD1) |
| 2 | GM-CAB215 PRO (MOD1) |
| 3 | GM-LTP1B PRO (MOD1) |
| 4 | GM-PSAL PRO (MOD1) |
| 5 | GM-VSP25 PRO (MOD1) |
| 6 | GM-VSPB PRO (MOD1) |
| 7 | CA-MetE PRO (MOD1) |
| 8 | CA-GAPDH PRO (MOD1) |
| 9 | CA-HSP90-1 PRO (MOD1) |
| 10 | CA-LHCB2-1 PRO |
| 11 | CA-LHCA3-1 PRO (MOD1) |
| 12 | CA-WD40 PRO (MOD1) |
| 13 | CA-HSP90-2 PRO (MOD1) |
| 14 | CA-CAB-CP26 PRO (MOD1) |
| 15 | PV-LTP PRO |
| 16 | GM-nsLTP15 PRO |
| 17 | MT-CAMT PRO (MOD1) |
| 18 | CA-SAG PRO (MOD1) |
| 19 | MT-ALP PRO (MOD1) |
| 20 | MT-VSPA PRO (MOD1) |
| 21 | MT-GRP-LG485 PRO (MOD1) |
| 22 | MT-MIP PRO (MOD1) |
| 23 | MT-LOX PRO (MOD1) |
| 24 | MT-MIPS PRO (MOD1) |
| 25 | MT-CP12-1 PRO (MOD1) |
| 26 | CA-UNK PRO (MOD1) |
| 27 | CC-UNK PRO (MOD1) |
| 28 | MT-PEROXIDASE PRO (MOD1) |
| 29 | MT-CSRP PRO (MOD1) |
| 30 | CA-MuDR PRO (MOD1) |
| 31 | MT-LLR PRO (MOD1) |
| 32 | CA-RUBISCO PRO (MOD1) |
| 33 | MT-RUBISCO PRO (MOD1) |
| 34 | CA-UBI PRO (MOD1) |
| 35 | MT-LHCB1 PRO (MOD 1) |
| 36 | CA-CAB PRO (MOD1) |
| 37 | CA-UNK PRO (MOD1)-V1 |
| 38 | GM-SHMT4 PRO (MOD1) |
| 39 | GM-ADF3 PRO (MOD1) |
| 40 | GM-ADF3(2) PRO (MOD1) |
| 41 | GM-TMA7 PRO (MOD1) |
| 42 | GM-CCDC72 PRO (MOD1) |
| 43 | MT-GARP PRO (MOD1) |
| 44 | LJ-AP(HAD IIIB) PRO (MOD1) |
| 45 | LJ-CA2 PRO (MOD1) |
| 46 | MT-CA2 PRO (MOD1) |
| 47 | MT-Beta-amylase PRO |
| 48 | LJ-Beta-amylase PRO |
| 49 | GM-Beta-amylase PRO (MOD1) |
| 50 | GM-ACTIN7 PRO (MOD1) |
| 51 | MT-ACTIN7 PRO (MOD1) |
| 52 | CA-ACTIN7 PRO (MOD1) |
| 53 | CC-ACTIN7 PRO (MOD1) |
| 54 | GM-GAPC2 PRO (MOD1) |
| 55 | GM-GAPC1 PRO (MOD1) |
| 56 | GM-GAPC1-2 PRO (MOD1) |
| 57 | GM-GAPC2-2 PRO (MOD1) |
| 58 | CA-GAPC PRO |
| 59 | CA-TIP1 PRO (MOD1) |
| 60 | CA-CWLP PRO (MOD1) |
| 61 | CA-PSI-LHCI PRO |
| 62 | CA-ASR PRO (MOD1) |
| 63 | CA-THI1-2 PRO (MOD1) |
| 64 | CA-PPI-1 PRO (MOD1) |
| 65 | CA-PPI-2 PRO (MOD1) |
| 66 | CC-TIP1 PRO |
| 67 | MT-TIP1 PRO (MOD1) |
| 68 | CC-UBI PRO |
| 69 | LJ-UBI PRO (MOD1) |
| 70 | GM-PPI(CYP19-1) PRO |
| 71 | GM-PPI(CYP18-3) PRO (MOD1) |
| 72 | LJ-PPI PRO (MOD1) |
| 73 | GM-TUBA2 PRO (MOD1) |
| 74 | PV-TUBA2 PRO |
| 75 | MT-TUBA2 PRO |
| 76 | CC-TUBA2 PRO (MOD1) |
| 77 | GM-SAHASE PRO (MOD1) |
| 78 | PV-SAHASE PRO (MOD1) |
| 79 | MT-SAHASE PRO (MOD1) |
| 80 | LJ-PIP1-4 PRO (MOD1) |
| 81 | PV-PIP1-4 PRO (MOD1) |
| 82 | CC-PIP1-4 PRO (MOD1) |
| 83 | GM-PIP2-4 PRO |
| 84 | CC-PIP2-4 PRO (MOD1) |
| 85 | LJ-PIP2-4 PRO |
| 86 | LJ-GAST-1 PRO (MOD1) |
| 87 | GM-GAST-1 PRO (MOD1) |
| 88 | CC-GAST-1 PRO (MOD1) |
| 89 | GM-SKP1 PRO |
| 90 | CC-SKP1 PRO (MOD1) |
| 91 | LJ-SKP1 PRO |
| 92 | GM-14-3-3 PRO |
| 93 | GM-14-3-3(2) PRO (MOD1) |
| 94 | PV-14-3-3 PRO (MOD1) |
| 95 | CC-14-3-3 PRO (MOD1) |
| 96 | GM-HMG2-2 PRO (MOD1) |
| 97 | PV-HMG2 PRO (MOD1) |
| 98 | CC-HMG2 PRO (MOD1) |
| 99 | GM-SAMD (MOD1) |
| 100 | CA-SAMD (MOD1) |
| 101 | PV-SAMD (MOD1) |
| 102 | GM-HMG2 PRO (MOD1) |
| 103 | MT-UBI2 PRO (MOD1) |
| 104 | MT-UBI3 PRO |
| 105 | CC-UBI2 PRO (MOD1) |
| 106 | LJ-UBI2 PRO |
| 107 | VU-UBI1 PRO (MOD1) |
| 108 | VU-UBI2 PRO |
| 109 | GM-MTH3 PRO (MOD1) |
| 110 | GM-MTH3-2 PRO (MOD1) |
| 111 | CA-MTH3 PRO (MOD1) |
| 112 | MT-MTH3 PRO (MOD1) |
| 113 | GM-RCA2 PRO (MOD1) |
| 114 | PV-RCA2 PRO |
| 115 | MT-RCA2 PRO (MOD1) |
| 116 | VU-RCA2 PRO (MOD1) |
| 117 | GM-LOX PRO |
| 118 | VU-LOX PRO |
| 119 | CC-LOX PRO (MOD1) |
| 120 | PH-LOX PRO |
| 121 | MT-MTH2A PRO (MOD1) |
| 122 | VU-MTH2A PRO |
| 123 | VU-MTH2A-2 PRO (MOD1) |
| 124 | CC-MTH2A PRO (MOD1) |
| 125 | GM-METE PRO |
| 126 | MT-METE PRO |
| 127 | CC-METE PRO |
| 128 | LJ-METE PRO |
| 129 | CA-METE PRO (MOD1)-V1 |
| 130 | CC-HMG2 PRO (MOD1) |
| 131 | GM-CAB2 PRO-V1 |
| 132 | GM-EFTU2 PRO-V1 |
| 133 | GM-HMG2 PRO (MOD1) |
| 134 | GM-HMG2.2 PRO (MOD1) |
| 135 | GM-MTH2 PRO |
| 136 | GM-PSID2 PRO-V2 |
| 137 | GM-SAMS PRO |
| 138 | GM-UBQ PRO |
| 139 | HA-UBI1 PRO |
| 140 | LJ-UBI1 PRO |
| 141 | NT-UBI4 PRO |
| 142 | PP-MTH1 PRO (MOD 1) |
| 143 | PV-HMG2 PRO (MOD1) |
| 144 | VV-UBI6 PRO |
| 145 | VV-UBI7 PRO |
| 146 | At-RBCS1A PRO F |
| 147 | At-RBCS1A PRO Tr368 |
| 148 | At-RBCS1A PRO Tr3748 |
| 149 | CA-LHCB2-1 PRO F |
| 150 | CA-LHCB2-1 PRO Tr336 |
| 151 | CA-LHCB2-1 PRO Tr58 |
| 152 | CA-RUBISCO (M1) PRO F |
| 153 | CA-RUBISCO (M1) Tr300 |
| 154 | CA-RUBISCO (M1) PRO Tr59 |
| 155 | CA-RUBISCO (M1) PRO 5UTR |
| 156 | CA-UBI (M1) PRO F |
| 157 | CA-UBI (M1) PRO Tr344noAP |
| 158 | CA-UBI (M1) PRO Tr42(344)noPM |
| 159 | CA-UBI (M1) PRO Tr344YAP |
| 160 | CA-UBI (M1) PRO Tr42(344)YPM |
| 161 | CA-UBI INTRON1 |
| 162 | CM-RBCS1_PRO Tr327 |
| 163 | CM-RBCS1_PRO Tr62 |
| 164 | LJ-UBI PARTIAL INTRON (TR7) |
| 165 | LJ-UBI 5UTR + INTRON (TR6) |
| 166 | LJ-UBI CORE |
| 167 | LJ-UBI (TR150) |
| 168 | LJ-UBI (TR300) |
| 169 | LJ-UBI (TR500) |
| 170 | LJ-UBI PRO no intron |
| 171 | CC-UBI PARTIAL INTRON (TR7) |
| 172 | CC-UBI 5UTR + INTRON (TR6) |
| 173 | CC-UBI CORE |
| 174 | CC-UBI (TR150) |
| 175 | CC-UBI (TR300) |
| 176 | CC-UBI (TR500) |
| 177 | CC-UBI PRO NO INTRON |
| 178 | CA-ACTIN7 (CORE)(with intron) |
| 179 | CA-ACTIN7 (TR150) |
| 180 | CA-ACTIN7 (TR300) |
| 181 | CA-ACTIN7 (TR500) |
| 182 | GM-PPI(CYP18-3) PRO (MOD1) (TR500) |
| 183 | GM-PPI(CYP18-3) PRO (MOD1) (TR300) |
| 184 | GM-PPI(CYP18-3) PRO (MOD1) (TR150) |
| 185 | GM-PPI(CYP18-3) PRO (MOD1) (CORE) |
| 186 | LJ-PPI PRO (MOD1) (TR500) |
| 187 | LJ-PPI PRO (MOD1) (TR300) |
| 188 | LJ-PPI PRO (MOD1) (TR150) |
| 189 | LJ-PPI PRO (MOD1) (CORE) |
| 190 | CA-TIP1 PRO (MOD1) (TR500) |
| 191 | CA-TIP1 PRO (MOD1) (TR300) |
| 192 | CA-TIP1 PRO (MOD1) (TR150) |
| 193 | CA-TIP1 PRO (MOD1) (CORE) |
| 194 | CA-HSP70 PRO (MOD1) (TR500) |
| 195 | CA-HSP70 PRO (MOD1) (TR300) |
| 196 | CA-HSP70 PRO (MOD1) (TR150) |
| 197 | CA-HSP70 PRO (MOD1) (CORE) |
| 198 | CA-WD40 PRO (TR1) |
| 199 | AT-RBCS1A PRO |
| 200 | CM-RBCS1 |
| 201 | AT-UBQ 10 PRO |
| 202 | CA-ATPASE-B PRO (MOD1) |
| 203 | CA-CWAH PRO (MOD1) |
| 204 | CA-GAPDH PRO (MOD1) |
| 205 | CA-HSP70 PRO (MOD1) |
| 206 | CA-LTP1 PRO (MOD1) |

### DETAILED DESCRIPTION

The article “a” and “an” are used herein to refer to one or more than one (i.e., to at least one) of the grammatical object of the article. By way of example, “an element” means one or more element.

The disclosure relates to compositions and methods drawn to plant regulatory elements and methods of their use. The compositions further comprise DNA constructs comprising at least one polynucleotide sequence for the regulatory region of a promoter operably linked to a heterologous polynucleotide sequence of interest. In particular, isolated nucleic acid molecules comprising any of the polynucleotide sequences set forth in SEQ ID NOs: 1-206, and fragments, variants and complements thereof are provided.

The regulatory element sequences, SEQ ID NOs: 1-206, include polynucleotide constructs that allow initiation of transcription in a plant. In specific embodiments, a regulatory element allows initiation of transcription in a constitutive manner. Such constructs may comprise regulated transcription initiation regions associated with plant developmental regulation. Thus, the compositions disclosed herein may include DNA constructs comprising a nucleotide sequence of interest operably linked to a plant promoter, particularly a constitutive promoter sequence, more particularly a promoter and intron sequence. In another preferred embodiment, the DNA construct further comprises a heterologous enhancer element.

The nucleotide sequences may also find use in the construction of expression vectors for subsequent expression of a heterologous nucleotide sequence in a plant of interest or as probes for the isolation of other regulatory elements. One embodiment is provided for DNA constructs comprising a regulatory element polynucleotide sequence set forth in SEQ ID NOs: 1-206, or a functional fragment or variants thereof, operably linked to a heterologous polynucleotide sequence of interest, and any combinations thereof.

The term “regulatory element” refers to a nucleic acid molecule having gene regulatory activity, i.e. one that has the ability to affect the transcriptional and/or translational expression pattern of an operably linked transcribable polynucleotide. The term “gene regulatory activity” thus refers to the ability to affect the expression of an operably linked transcribable polynucleotide molecule by affecting the transcription and/or translation of that operably linked transcribable polynucleotide molecule. Gene regulatory activity may be positive and/or negative and the effect may be characterized by its temporal, spatial, developmental, tissue, environmental, physiological, pathological, cell cycle, and/or chemically responsive qualities as well as by quantitative or qualitative indications.

Regulatory elements such as promoters, enhancers, leaders, and intron regions are nucleic acid molecules that have gene regulatory activity and play an integral part in the overall expression of genes in living cells. Isolated regulatory elements, such as promoters and leaders that function in plants are therefore useful for modifying plant phenotypes through the methods of genetic engineering. A promoter is useful as a regulatory element for modulating the expression of an operably linked transcribable polynucleotide molecule.

As used herein, a “gene expression pattern” is any pattern of transcription of an operably linked nucleic acid molecule into a transcribed RNA molecule. Expression may be characterized by its temporal, spatial, developmental, tissue, environmental, physiological, pathological, cell cycle, and/or chemically responsive qualities as well as by quantitative or qualitative indications. The transcribed RNA molecule may be translated to produce a protein molecule or may provide an antisense or other regulatory RNA molecule, such as a dsRNA, a tRNA, an rRNA, a miRNA, and the like.

The regulatory element sequences or variants or fragments thereof, when operably linked to a heterologous polynucleotide sequence of interest may drive constitutive expression of the heterologous polynucleotide sequence in the tissue of the plant expressing this construct. The term “constitutive expression,” means that expression of the heterologous nucleotide sequence is found throughout the plant or in a majority of tissues of the plant.

As used herein, the term “protein expression” is any pattern of translation of a transcribed RNA molecule into a protein molecule. Protein expression may be characterized by its temporal, spatial, developmental, or morphological qualities as well as by quantitative or qualitative indications.

As used herein, the term “promoter” refers generally to a nucleic acid molecule that is involved in recognition and binding of RNA polymerase II and other proteins (trans-acting transcription factors) to initiate transcription. A promoter may be initially isolated from the 5′ flanking region of a genomic copy of a gene. Alternately, promoters may be synthetically produced or manipulated DNA molecules. Regulatory elements may comprise promoters and promoter activity. As used herein, “promoter activity” refers to the ability of a regulatory element to initiate transcription. Promoter activity may occur in vivo, such as in a cell, or in vitro.

In one embodiment, fragments are provided of a regulatory element disclosed herein. Regulatory element fragments may exhibit promoter activity, and may be useful alone or in combination with other regulatory elements and regulatory element fragments, such as in constructing hybrid regulatory elements (See International Patent Publication Number WO 2017/222821). In specific embodiments, fragments of a regulatory element are provided comprising, or alternatively consisting of or consisting essentially of, at least about 50, 95, 150, 250, 500, or about 750 or more contiguous nucleotides of a polynucleotide molecule having promoter activity disclosed herein. Such fragments may exhibit at least about 85 percent, about 90 percent, about 95 percent, about 98 percent, or about 99 percent, or greater, identity with a reference sequence disclosed herein when optimally aligned to the reference sequence. As used herein, the term “regulatory element segment” is a fragment of a regulatory element characterized by an abundance of recognizable regulatory element motifs (See Higo, K et al. (1998) Nucleic Acids Research), wherein the regulatory element segment produces a desired or unique expression pattern when combined with at least two other regulatory element segments.

A regulatory element or a regulatory element segment may also be analyzed for the presence of known promoter motifs, i.e. DNA sequence characteristics, such as a TATA-box and other known transcription factor binding site motifs. Identification of such known motifs may be used by one of skill in the art to design hybrid regulatory elements having a desired or unique expression pattern when compared to the source or parent regulatory element. Nucleotide sequence motifs found in regulatory elements have been previously characterized and many are available in the PLACE database (Higo, K et al. (1998) Nucleic Acids Research; dna.affrc.go.jp/htdocs/PLACE/, which can be accessed on the world-wide web using the “www” prefix; See also, PCT Application Number WO 2014/164399). In some embodiments, a regulatory element segment comprises about 15, 20, 25, 30, 35, 40, 45, 50, 75, 100, 125, 150, 175, or 200 motifs per 1000 nucleotides. In some embodiments, a regulatory element comprises at least one motif for about every 5, 10, 15, 20, 25, 30, 35, 40, 45, or 50 nucleotides. In one embodiment, a hybrid regulatory element comprises a segment, fragment, or variant of SEQ ID NOs: 1-206, wherein the segment, fragment, or variant of SEQ ID NOs: 1-206 comprises about 15, 20, 25, 30, 35, 40, 45, 50, 75, 100, 125, 150, 175, or 200 motifs per 1000 nucleotides.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide sequence. Unlike promoters, enhancer elements do not usually include a transcription start site (TSS) or TATA box. A regulatory element may naturally comprise one or more enhancer elements that affect the transcription of an operably linked polynucleotide sequence. An isolated enhancer element may also be fused to a heterologous promoter to produce a heterologous promoter cis-element, which confers an aspect of the overall modulation of gene expression. A regulatory element or regulatory element fragment disclosed herein may comprise one or more enhancer elements that effect the transcription of operably linked genes. Many enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element may function to bind transcription factors that regulate transcription. Some enhancer elements bind more than one transcription factor, and transcription factors may interact with different affinities with more than one enhancer domain Enhancer elements may be identified by a number of techniques, including deletion analysis, i.e., deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis using known cis-element motifs or enhancer elements as a target sequence or target motif with conventional DNA sequence comparison methods, such as BLAST. The fine structure of an enhancer domain may be further studied by mutagenesis (or substitution) of one or more nucleotides or by other conventional methods Enhancer elements may be obtained by chemical synthesis or by isolation from regulatory elements that include such elements, and they may be synthesized with additional flanking nucleotides that contain useful restriction enzyme sites to facilitate subsequence manipulation. Thus, the design, construction, and use of enhancer elements according to the methods disclosed herein for modulating the expression of operably linked transcribable polynucleotide molecules are encompassed.

As used herein, the term “5′ flanking region” refers to a DNA molecule isolated from a genomic copy of a gene and is defined generally as a polynucleotide segment beginning at the protein coding sequence start site and extending 5′ through the 5′ untranslated region and into the promoter region. These sequences, or leaders, may be synthetically produced or manipulated DNA elements. A leader may be used as a 5′ regulatory element for modulating expression of an operably linked transcribable polynucleotide molecule. Leader molecules may be used with heterologous elements or with their native elements.

As used herein, the term “hybrid” refers to a single synthetic DNA molecule produced by fusing a first DNA molecule to a second DNA molecule, where neither first nor second DNA molecule would normally be found in that configuration, i.e. fused to the other. The hybrid DNA molecule is thus a new DNA molecule not normally found in nature. As used herein, the term “hybrid regulatory element” refers to a regulatory element produced through such manipulation of DNA molecules. A hybrid regulatory element may combine three or more DNA fragments. Thus, the design, construction, and use of hybrid regulatory element according to the methods disclosed herein for modulating the expression of operably linked transcribable polynucleotide molecules are encompassed. In one embodiment, a hybrid regulatory element comprises three or more DNA defined segments. In another embodiment, a hybrid regulatory element comprises 4 or more DNA fragments. In one embodiment, a DNA fragment may be a parent fragment. As used herein, a “segment,” and “parent segment” are interchangeable and intended to refer to fragments of native “parent regulatory elements” that have been analyzed for motifs that are predicted to produce a regional tissue expression pattern. A combination of parent segments or variants thereof, may result in a hybrid regulatory element expressing a gene of interest in a ubiquitous tissue expression pattern that is unique from each individual expression pattern of the parent regulatory elements. In one embodiment, a parent segment may be a variant of a parent regulatory element. In one embodiment, parent regulatory elements set forth in SEQ ID NOs: 1-206 may be used as parent regulatory elements to generate parent segments and variants thereof. Also, included as parent regulatory elements are functional fragments, segments, or variants of the polynucleotide sequences set forth in SEQ ID NOs: 1-206 wherein said polynucleotide sequences initiate transcription in a plant cell, and a polynucleotide sequence comprising a sequence having at least 85% sequence identity to the polynucleotide sequences set forth in SEQ ID NOs: 1-206, wherein said polynucleotide sequences initiate transcription in a plant cell.

Hybrid regulatory elements are provided that produce an expression pattern in plants that is unique relative to parent regulatory elements, wherein the hybrid regulatory element contains segments or fragments of more than one parent regulatory element. In one embodiment, the hybrid regulatory element produces a tissue specific expression pattern that is different relative to the regulatory elements. In another embodiment, the hybrid regulatory elements broaden the expression pattern to a ubiquitous expression pattern in a plant tissue relative to regional tissue expression patterns expressed from a given set of parent regulatory elements. In another embodiment, the hybrid regulatory elements express a narrower range of expression relative to a broader range of expression patterns expressed from a given set of parent regulatory elements. In another embodiment, the hybrid root regulatory elements may produce a constitutive expression pattern that differs from a non-constitutive expression pattern of the parent regulatory elements.

In one embodiment, the polynucleotide sequences disclosed herein, located within introns, or 3′ of the coding region sequence may also contribute to the regulation of expression of a coding region of interest. Examples of suitable introns include, but are not limited to, the maize WS6 intron, or the maize actin intron. A regulatory element may also include those elements located downstream (3′) to the site of transcription initiation, or within transcribed regions, or both. A post-transcriptional regulatory element may include elements that are active following transcription initiation, for example translational and transcriptional enhancers, translational and transcriptional repressors, and mRNA stability determinants.

The regulatory elements, or variants or fragments thereof, may be operatively associated with one or more heterologous regulatory elements in order to modulate the activity of the heterologous regulatory element. Such modulation includes enhancing or repressing transcriptional activity of the heterologous regulatory element, modulating post-transcriptional events, or either enhancing or repressing transcriptional activity of the heterologous regulatory element and modulating post-transcriptional events. For example, one or more regulatory elements, or fragments thereof, may be operatively associated with constitutive, inducible, or tissue specific promoters or fragment thereof, to modulate the activity of such promoters within desired tissues in plant cells.

The compositions may encompass isolated or recombinant nucleic acid. An “isolated” or “recombinant” nucleic acid molecule (or DNA) is used herein to refer to a nucleic acid sequence (or DNA) that is no longer in its natural environment, for example in an in vitro or in a heterologous recombinant bacterial or plant host cell. An isolated or recombinant nucleic acid molecule, or biologically active portion thereof, is substantially free of other cellular material or culture medium when produced by recombinant techniques, or substantially free of chemical precursors or other chemicals when chemically synthesized. An isolated or recombinant nucleic acid is free of sequences (optimally protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule may contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. The regulatory element sequences disclosed herein may be isolated from the 5′ untranslated region flanking their respective transcription initiation sites. As used herein, the terms “polynucleotide” and “nucleotide” are both intended to mean one or more nucleotide and may be used interchangeably in the singular or plural.

Fragments and variants of the disclosed regulatory element polynucleotide sequences are also encompassed by the present disclosure. As used herein, the term “fragment” refers to a portion of the nucleic acid sequence. Fragments of regulatory sequences may retain the biological activity of initiating transcription, more particularly driving transcription in a tissue specific or sub-tissue specific manner. Alternatively, fragments of a polynucleotide sequence that are useful as hybridization probes may not necessarily retain biological activity. Fragments of a polynucleotide sequence for the regulatory region may range from at least about 20 nucleotides, about 50 nucleotides, about 100 nucleotides, and up to the full length of SEQ ID NOs: 1-206.

A biologically active portion of a regulatory element may be prepared by isolating a portion of the regulatory sequence and assessing the promoter activity of the portion. Nucleic acid molecules that are fragments of a regulatory polynucleotide sequence comprise at least about 16, 50, 75, 100, 150, 200, 250, 300, 350, 400, 450, 500, 550, 600, 650, 700 or 800 nucleotides or up to the number of nucleotides present in a full-length regulatory sequence disclosed herein.

For polynucleotide sequences, a variant comprises a deletion and/or addition of one or more nucleotides at one or more internal sites within the native polynucleotide sequence and/or a substitution of one or more nucleotides at one or more sites in the native polynucleotide. For polynucleotide sequences, variants may be identified with the use of well-known molecular biology techniques, as, for example, with polymerase chain reaction (PCR) and hybridization techniques as outlined below. Variant polynucleotide sequences may include synthetically derived polynucleotide sequences, such as those generated, for example, by using site-directed mutagenesis. Generally, variants of a particular nucleotide sequence of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular nucleotide sequence as determined by sequence alignment programs and parameters described elsewhere herein. A biologically active variant of a polynucleotide sequence of the disclosure may differ from that sequence by as few as 1-15 nucleic acid residues, as few as 1-10, as few as 6-10, as few as 5, as few as 4, 3, 2, or even 1 nucleic acid residue.

Variant polynucleotide sequences also encompass sequences derived from a mutagenic and recombinogenic procedure such as DNA shuffling. With such a procedure, regulatory element polynucleotide sequences may be manipulated to create new regulatory elements. In this manner, libraries of recombinant polynucleotides are generated from a population of related sequence polynucleotides comprising sequence regions that have substantial sequence identity and may be homologously recombined in vitro or in vivo. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The polynucleotide sequences of the disclosure may be used to isolate corresponding sequences from other organisms, particularly other plants, more particularly other monocots. In this manner, methods such as PCR, hybridization and the like may be used to identify such sequences based on their sequence homology to the sequences set forth herein. Sequences isolated based on their sequence identity to the entire sequences set forth herein or to fragments thereof are encompassed by the present disclosure.

In a PCR approach, oligonucleotide primers may be designed for use in PCR reactions to amplify corresponding DNA sequences from cDNA or genomic DNA extracted from any plant of interest. Methods for designing PCR primers and PCR cloning are generally known in the art and are disclosed in, Sambrook, supra. See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York), herein incorporated by reference in their entirety. Known methods of PCR include, but are not limited to, methods using paired primers, nested primers, single specific primers, degenerate primers, gene-specific primers, vector-specific primers, partially-mismatched primers and the like.

In hybridization techniques, all or part of a known polynucleotide sequence is used as a probe that selectively hybridizes to other corresponding polynucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides and may be labeled with a detectable group such as ³²P or any other detectable marker. Thus, for example, probes for hybridization may be made by labeling synthetic oligonucleotides based on the regulatory element sequences of the disclosure. Methods for preparation of probes for hybridization and for construction of genomic libraries are generally known in the art and are disclosed in Sambrook, supra.

For example, an entire regulatory element sequence disclosed herein, or one or more portions thereof, may be used as a probe capable of specifically hybridizing to corresponding regulatory element sequences and messenger RNAs. To achieve specific hybridization under a variety of conditions, such probes include sequences that are unique among regulatory element sequences and are generally at least about 10 nucleotides in length or at least about 20 nucleotides in length. Such probes may be used to amplify corresponding regulatory element sequences from a chosen plant by PCR. This technique may be used to isolate additional coding sequences from a desired organism or as a diagnostic assay to determine the presence of coding sequences in an organism. Hybridization techniques include hybridization screening of plated DNA libraries (either plaques or colonies, see, for example, Sambrook, supra).

Hybridization of such sequences may be carried out under stringent conditions. The terms “stringent conditions” or “stringent hybridization conditions” are intended to mean conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions may be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C. and a wash in 1 times to 2 times SSC (20 times SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C. and a wash in 0.5 times to 1 times SSC at 55 to 60° C. Exemplary high stringency conditions include hybridization in 50% formamide, 1 M NaCl, 1% SDS at 37° C., and a final wash in 0.1 times SSC at 60 to 65° C. for a duration of at least 30 minutes. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the thermal melting point (Tₘ) can be approximated from the equation of Meinkoth and Wahl, (1984) Anal. Biochem 138:267 284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching, thus, Tₘ, hybridization, and/or wash conditions can be adjusted to hybridize to sequences of the desired identity. For example, if sequences with 90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3 or 4° C. lower than the Tₘ; moderately stringent conditions can utilize a hybridization and/or wash at 6, 7, 8, 9 or 10° C. lower than the Tₘ; low stringency conditions can utilize a hybridization and/or wash at 11, 12, 13, 14, 15 or 20° C. lower than the Tₘ. Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen, (1993) Laboratory Techniques in Biochemistry and Molecular Biology—Hybridization with Nucleic Acid Probes, Part I, Chapter 2 (Elsevier, New York); and Ausubel, et al., eds. (1995) Current Protocols in Molecular Biology, Chapter 2 (Greene Publishing and Wiley-Interscience, New York), herein incorporated by reference in their entirety. See also, Sambrook.

Thus, isolated sequences that have promoter activity and which hybridize under stringent conditions to the regulatory sequences disclosed herein or to fragments thereof, are encompassed by the present disclosure.

In general, sequences that have promoter activity and hybridize to the polynucleotide sequences, and fragments thereof, disclosed herein will be at least 40% to 50% homologous, about 60%, 70%, 80%, 85%, 90%, 95% to 98% homologous or more with the disclosed sequences. That is, the sequence similarity of sequences may range, sharing at least about 40% to 50%, about 60% to 70%, and about 80%, 85%, 90%, 95% to 98% sequence similarity.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Another indication that polynucleotide sequences are substantially identical is if two molecules hybridize to each other under stringent conditions. Generally, stringent conditions are selected to be about 5° C. lower than the Tₘ for the specific sequence at a defined ionic strength and pH. However, stringent conditions encompass temperatures in the range of about 1° C. to about 20° C. lower than the Tₘ, depending upon the desired degree of stringency as otherwise qualified herein.

Modifications of the isolated regulatory element sequences of the present disclosure may provide for a range of expression of the heterologous polynucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

The regulatory elements disclosed herein may be used to increase or decrease expression, thereby resulting in a change in phenotype of the transformed plant. The polynucleotide sequences disclosed herein, as well as variants and fragments thereof, are useful in the genetic manipulation of any plant. The regulatory element sequences are useful in this aspect when operably linked with a heterologous nucleotide sequence whose expression is to be controlled to achieve a desired phenotypic response. The term “operably linked” means that the transcription or translation of the heterologous nucleotide sequence is under the influence of the regulatory element sequence. In this manner, the regulatory element sequences disclosed herein may be provided in expression cassettes along with heterologous polynucleotide sequences of interest for expression in the plant of interest, more particularly for expression in the reproductive tissue of the transformed plant.

The regulatory elements of the embodiments may be provided in DNA constructs for expression in the organism of interest. An “expression cassette” as used herein means a DNA construct comprising a regulatory element of the embodiments operably linked to a heterologous polynucleotide expressing a transcript or gene of interest. Such expression cassettes will comprise a transcriptional initiation region comprising one of the regulatory element polynucleotide sequences of the present disclosure, or variants or fragments thereof, operably linked to the heterologous nucleotide sequence. Such an expression cassette may be provided with a plurality of restriction sites for insertion of the polynucleotide sequence to be under the transcriptional regulation of the regulatory regions. The expression cassette may additionally contain selectable marker genes as well as 3′ termination regions.

The expression cassette may include, in the 5′-3′ direction of transcription, a transcriptional initiation region (i.e., a hybrid promoter, or variant or fragment thereof, of the disclosure), a translational initiation region, a heterologous polynucleotide sequence of interest, a translational termination region and optionally, a transcriptional termination region functional in the host organism. The regulatory regions (i.e., promoters, enhancers, transcriptional regulatory regions, and translational termination regions) and/or the polynucleotide of the embodiments may be native/analogous to the host cell or to each other. Alternatively, the regulatory regions and/or the polynucleotide of the embodiments may be heterologous to the host cell or to each other.

As used herein, “heterologous” in reference to a sequence is a sequence that originates from a foreign species or, if from the same species, is substantially modified from its native form in composition and/or genomic locus by deliberate human intervention. For example, a regulatory element operably linked to a heterologous polynucleotide is from a species different from the species from which the polynucleotide was derived or, if from the same/analogous species, one or both are substantially modified from their original form and/or genomic locus or the regulatory element is not the native regulatory element for the operably linked polynucleotide.

The termination region may be native with the transcriptional initiation region, may be native with the operably linked DNA sequence of interest, may be native with the plant host, or may be derived from another source (i.e., foreign or heterologous to the regulatory element, the DNA sequence being expressed, the plant host, or any combination thereof).

The regulatory elements disclosed herein, as well as variants and fragments thereof, are useful for genetic engineering of plants, e.g. for the production of a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of heterologous nucleic acid, including those transgenics initially so altered as well as those created by sexual crosses or asexual propagation from the initial transgenic.

A transgenic “event” is produced by transformation of plant cells with a heterologous DNA construct, including a nucleic acid expression cassette that comprises a transgene of interest, the regeneration of a population of plants resulting from the insertion of the transgene into the genome of the plant and selection of a particular plant characterized by insertion into a particular genome location. An event is characterized phenotypically by the expression of the transgene. At the genetic level, an event is part of the genetic makeup of a plant. The term “event” also refers to progeny produced by a sexual cross between the transformant and another plant wherein the progeny include the heterologous DNA.

As used herein, the term plant includes whole plants, plant organs (e.g., leaves, stems, roots, etc.), plant cells, plant protoplasts, plant cell tissue cultures from which plants can be regenerated, plant calli, plant clumps and plant cells that are intact in plants or parts of plants such as embryos, pollen, ovules, seeds, leaves, flowers, branches, fruit, kernels, ears, cobs, husks, stalks, roots, root tips, anthers and the like. Grain is intended to mean the mature seed produced by commercial growers for purposes other than growing or reproducing the species. Progeny, variants and mutants of the regenerated plants are also included within the scope of the disclosure, provided that these parts comprise the introduced polynucleotides.

The compositions and methods disclosed herein may be used for transformation of any plant species, including, but not limited to, monocots and dicots. Examples of plant species include corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis) and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed include, for example, pines such as loblolly pine (Pinus taeda), slash pine (Pinus elliotii), ponderosa pine (Pinusponderosa), lodgepole pine (Pinus contorta) and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea) and cedars such as Western red cedar (Thuja plicata) and Alaska yellow-cedar (Chamaecyparis nootkatensis). In specific embodiments, plants of may be crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Heterologous coding sequences expressed by a regulatory element sequence disclosed herein may be used for varying the phenotype of a plant. Various changes in phenotype are of interest including modifying expression of a gene in a plant, altering a plant's pathogen or insect defense mechanism, increasing a plant's tolerance to herbicides, altering plant development to respond to environmental stress, modulating the plant's response to salt, temperature (hot and cold), drought and the like. These results may be achieved by the expression of a heterologous polynucleotide sequence of interest comprising an appropriate gene product. In specific embodiments, the heterologous polynucleotide sequence of interest is an endogenous plant sequence whose expression level is increased in the plant or plant part. Results may be achieved by providing for altered expression of one or more endogenous gene products, particularly hormones, receptors, signaling molecules, enzymes, transporters or cofactors or by affecting nutrient uptake in the plant. These changes result in a change in phenotype of the transformed plant. In certain embodiments the expression patterns of the regulatory elements disclosed herein are useful for many types of screening.

General categories of polynucleotide sequences of interest that may be utilized with the regulatory sequences disclosed herein include, for example, those genes involved in information, such as zinc fingers, those involved in communication, such as kinases and those involved in housekeeping, such as heat shock proteins. More specific categories of genes, for example, include genes that confer resistance to an herbicide; transgenes that confer or contribute to an altered grain characteristic; genes that control male-sterility; genes that create a site for site specific DNA integration; genes that affect abiotic stress resistance; genes that confer increased yield genes that confer plant digestibility; and transgenes that confer resistance to insects or disease. Still other categories of transgenes include genes for inducing expression of exogenous products such as enzymes, cofactors, and hormones from plants and other eukaryotes as well as prokaryotic organisms. It is recognized that any gene of interest can be operably linked to the regulatory element of the disclosure and expressed in the plant.

Genes may encode a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC® Accession Numbers 40098, 67136, 31995 and 31998. Other non-limiting examples of Bacillus thuringiensis transgenes being genetically engineered are given in the following patents and patent applications and hereby are incorporated by reference for this purpose: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO 1991/14778; WO 1999/31248; WO 2001/12731; WO 1999/24581 and WO 1997/40162.

Genes encoding pesticidal proteins may also be stacked including but are not limited to: insecticidal proteins from Pseudomonas sp. such as PSEEN3174 (Monalysin, (2011) PLoS Pathogens, 7:1-13), from Pseudomonas protegens strain CHAO and Pf-5 (previously fluorescens) (Pechy-Tarr, (2008) Environmental Microbiology 10:2368-2386: GenBank Accession No. EU400157); from Pseudomonas taiwanensis (Liu, et al., (2010) J. Agric. Food Chem. 58:12343-12349) and from Pseudomonas pseudoalcaligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxinology Journal 3:101-118 and Morgan, et al., (2001) Applied and Envir. Micro. 67:2062-2069), U.S. Pat. Nos. 6,048,838, and 6,379,946; a PIP-1 polypeptide of U.S. Pat. No. 9,688,730; an AfIP-1A and/or AFP-1B polypeptide of U.S. Pat. No. 9,475,847; a PIP-47 polypeptide of US Publication Number US20160186204; an IPD045 polypeptide, an IPD064 polypeptide, an IPD074 polypeptide, an IPD075 polypeptide, and an IPD077 polypeptide of PCT Publication Number WO 2016/114973; an IPD080 polypeptide of PCT Serial Number PCT/US17/56517; an IPD078 polypeptide, an IPD084 polypeptide, an IPD085 polypeptide, an IPD086 polypeptide, an IPD087 polypeptide, an IPD088 polypeptide, and an IPD089 polypeptide of Serial Number PCT/US17/54160; PIP-72 polypeptide of US Patent Publication Number US20160366891; a PtIP-50 polypeptide and a PtIP-65 polypeptide of US Publication Number US20170166921; an IPD098 polypeptide, an IPD059 polypeptide, an IPD108 polypeptide, an IPD109 polypeptide of U.S. Ser. No. 62/521,084; a PtIP-83 polypeptide of US Publication Number US20160347799; a PtIP-96 polypeptide of US Publication Number US20170233440; an IPD079 polypeptide of PCT Publication Number WO2017/23486; an IPD082 polypeptide of PCT Publication Number WO 2017/105987, an IPD090 polypeptide of Serial Number PCT/US17/30602, an IPD093 polypeptide of U.S. Ser. No. 62/434,020; an IPD103 polypeptide of Serial Number PCT/US17/39376; an IPD101 polypeptide of U.S. Ser. No. 62/438,179; an IPD121 polypeptide of US Serial Number U.S. 62/508,514, and δ-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry50, Cry51, Cry52, Cry53, Cry 54, Cry55, Cry56, Cry57, Cry58, Cry59, Cry60, Cry61, Cry62, Cry63, Cry64, Cry65, Cry66, Cry67, Cry68, Cry69, Cry70, Cry71, and Cry 72 classes of δ-endotoxin genes and the B. thuringiensis cytolytic Cyt1 and Cyt2 genes.

Examples of 6-endotoxins also include but are not limited to Cry1A proteins of U.S. Pat. Nos. 5,880,275 and 7,858,849; a DIG-3 or DIG-11 toxin (N-terminal deletion of α-helix 1 and/or α-helix 2 variants of Cry proteins such as Cry1A) of US Patent Numbers 8,304,604 and 8.304,605, Cry1B of U.S. patent application Ser. No. 10/525,318; Cry1C of U.S. Pat. No. 6,033,874; Cry1F of U.S. Pat. Nos. 5,188,960, 6,218,188; Cry1A/F chimeras of U.S. Pat. Nos. 7,070,982; 6,962,705 and 6,713,063); a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E, and Cry9F families; a Cry15 protein of Naimov, et al., (2008) Applied and Environmental Microbiology 74:7145-7151; a Cry22, a Cry34Ab1 protein of U.S. Pat. Nos. 6,127,180, 6,624,145 and 6,340,593; a CryET33 and CryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US 2008/0295207; ET29, ET37, TIC809, TIC810, TIC812, TIC127, TIC128 of PCT US 2006/033867; AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020, and AXMI-021 of WO 2006/083891; AXMI-010 of WO 2005/038032; AXMI-003 of WO 2005/021585; AXMI-008 of US 2004/0250311; AXMI-006 of US 2004/0216186; AXMI-007 of US 2004/0210965; AXMI-009 of US 2004/0210964; AXMI-014 of US 2004/0197917; AXMI-004 of US 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of US20110023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488; AXMI-R1 and related proteins of US 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230, and AXMI231 of WO11/103247; AXMI-115, AXMI-113, AXMI-005, AXMI-163 and AXMI-184 of U.S. Pat. No. 8,334,431; AXMI-001, AXMI-002, AXMI-030, AXMI-035, and AXMI-045 of US 2010/0298211; AXMI-066 and AXMI-076 of US2009/0144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of US 2010/0005543; and Cry proteins such as Cry1A and Cry3A having modified proteolytic sites of U.S. Pat. No. 8,319,019; and a Cry1Ac, Cry2Aa and Cry1Ca toxin protein from Bacillus thuringiensis strain VBTS 2528 of US Patent Application Publication Number 2011/0064710. Other Cry proteins are well known to one skilled in the art (see, Crickmore, et al., “Bacillus thuringiensis toxin nomenclature” (2011), at lifesci.sussex.ac.uk/home/Neil_Crickmore/Bt/which can be accessed on the world-wide web using the “www” prefix). The insecticidal activity of Cry proteins is well known to one skilled in the art (for review, see, van Frannkenhuyzen, (2009) J. Invert. Path. 101:1-16). The use of Cry proteins as transgenic plant traits is well known to one skilled in the art and Cry-transgenic plants including but not limited to Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval (see, Sanahuja, (2011) Plant Biotech Journal 9:283-300 and the CERA (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one pesticidal proteins well known to one skilled in the art can also be expressed in plants such as Vip3Ab & Cry1Fa (US2012/0317682), Cry1BE & Cry1F (US2012/0311746), Cry1CA & Cry1AB (US2012/0311745), Cry1F & CryCa (US2012/0317681), Cry1DA & Cry1BE (US2012/0331590), Cry1DA & Cry1Fa (US2012/0331589), Cry1AB & Cry1BE (US2012/0324606), and Cry1Fa & Cry2Aa, Cry1I or Cry1E (US2012/0324605). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279, 6,137,033, 7,244,820, 7,615,686, and 8,237,020, and the like. Other VIP proteins are well known to one skilled in the art (see, lifesci.sussex.ac.uk/home/Neil_Crickmore/Bt/vip.html which can be accessed on the world-wide web using the “www” prefix). Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366).

Further transgenes that confer resistance to insects may down-regulation of expression of target genes in insect pest species by interfering ribonucleic acid (RNA) molecules through RNA interference. RNA interference refers to the process of sequence-specific post-transcriptional gene silencing in animals mediated by short interfering RNAs (siRNAs) (Fire, et al., (1998) Nature 391:806). RNAi transgenes may include but are not limited to expression of dsRNA, siRNA, miRNA, iRNA, antisense RNA, or sense RNA molecules that down-regulate expression of target genes in insect pests. PCT Publication WO 2007/074405 describes methods of inhibiting expression of target genes in invertebrate pests including Colorado potato beetle. PCT Publication WO 2005/110068 describes methods of inhibiting expression of target genes in invertebrate pests including in particular Western corn rootworm as a means to control insect infestation. Furthermore, PCT Publication WO 2009/091864 describes compositions and methods for the suppression of target genes from insect pest species including pests from the Lygus genus.

RNAi transgenes are provided for targeting the vacuolar ATPase H subunit, useful for controlling a coleopteran pest population and infestation as described in US Patent Application Publication 2012/0198586. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein S27A; an insect proteasome subunit such as the Rpn6 protein, the Pros 25, the Rpn2 protein, the proteasome beta 1 subunit protein or the Pros beta 2 protein; an insect β-coatomer of the COPI vesicle, the γ-coatomer of the COPI vesicle, the β′-coatomer protein or the ζ-coatomer of the COPI vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+-ATPase G-subunit protein and an insect Tbp-1 such as Tat-binding protein. PCT publication WO 2007/035650 describes ribonucleic acid (RNA or double stranded RNA) that inhibits or down regulates the expression of a target gene that encodes Snf7. US Patent Application publication 2011/0054007 describes polynucleotide silencing elements targeting RPS10. PCT publication WO 2016/205445 describes polynucleotide silencing elements that reduce fecundity, with target polynucleotides, including NCLB, MAEL, BOULE, and VgR. US Patent Application publication 2014/0275208 and US2015/0257389 describes polynucleotide silencing elements targeting RyanR and PAT3. PCT publications WO/2016/138106, WO 2016/060911, WO 2016/060912, WO 2016/060913, and WO 2016/060914 describe polynucleotide silencing elements targeting COPI coatomer subunit nucleic acid molecules that confer resistance to Coleopteran and Hemipteran pests. US Patent Application Publications 2012/029750, US 20120297501, and 2012/0322660 describe interfering ribonucleic acids (RNA or double stranded RNA) that functions upon uptake by an insect pest species to down-regulate expression of a target gene in said insect pest, wherein the RNA comprises at least one silencing element wherein the silencing element is a region of double-stranded RNA comprising annealed complementary strands, one strand of which comprises or consists of a sequence of nucleotides which is at least partially complementary to a target nucleotide sequence within the target gene. US Patent Application Publication 2012/0164205 describe potential targets for interfering double stranded ribonucleic acids for inhibiting invertebrate pests including: a Chd3 Homologous Sequence, a Beta-Tubulin Homologous Sequence, a 40 kDa V-ATPase Homologous Sequence, a EF1α Homologous Sequence, a 26S Proteosome Subunit p28 Homologous Sequence, a Juvenile Hormone Epoxide Hydrolase Homologous Sequence, a Swelling Dependent Chloride Channel Protein Homologous Sequence, a Glucose-6-Phosphate 1-Dehydrogenase Protein Homologous Sequence, an Act42A Protein Homologous Sequence, a ADP-Ribosylation Factor 1 Homologous Sequence, a Transcription Factor IIB Protein Homologous Sequence, a Chitinase Homologous Sequences, a Ubiquitin Conjugating Enzyme Homologous Sequence, a Glyceraldehyde-3-Phosphate Dehydrogenase Homologous Sequence, an Ubiquitin B Homologous Sequence, a Juvenile Hormone Esterase Homolog, and an Alpha Tubuliln Homologous Sequence.

The isolated regulatory element sequences disclosed herein may be modified to provide for a range of expression levels of the heterologous nucleotide sequence. Thus, less than the entire regulatory element region may be utilized and the ability to drive expression of the nucleotide sequence of interest retained. It is recognized that expression levels of the mRNA may be altered in different ways with deletions of portions of the promoter sequences. The mRNA expression levels may be decreased, or alternatively, expression may be increased as a result of regulatory element deletions if, for example, there is a negative regulatory element (for a repressor) that is removed during the truncation process. Generally, at least about 20 nucleotides of an isolated regulatory element sequence will be used to drive expression of a polynucleotide sequence.

Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acid Res. 15:9627-9639.

Expression cassettes comprising sequences disclosed herein may also contain at least one additional nucleotide sequence for a gene to be cotransformed into the organism. Alternatively, the additional sequence(s) can be provided on another expression cassette.

Where appropriate, the polynucleotide sequences whose expression is to be under the control of a regulatory element sequence of the present disclosure and any additional nucleotide sequence(s) may be optimized for increased expression in the transformed plant. That is, these nucleotide sequences can be synthesized using plant preferred codons for improved expression. See, for example, Campbell and Gowri, (1990) Plant Physiol. 92:1-11, for a discussion of host-preferred codon usage. Methods are available in the art for synthesizing plant-preferred genes. See, for example, Murray, et al., (1989) Nucleic Acids Res. 17:477-498.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the heterologous polynucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences may act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385). See, also, Della-Cioppa, et al., (1987) Plant Physiology 84:965-968. Methods known to enhance mRNA stability may also be utilized, for example, introns, such as the maize Ubiquitin intron (Christensen and Quail, (1996) Transgenic Res. 5:213-218; Christensen, et al., (1992) Plant Molecular Biology 18:675-689) or the maize Adhl intron (Kyozuka, et al., (1991) Mol. Gen. Genet. 228:40-48; Kyozuka, et al., (1990) Maydica 35:353-357) and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

Reporter genes or selectable marker genes may also be included in expression cassettes. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330.

Selectable marker genes for selection of transformed cells or tissues may include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518).

Other genes that could serve utility in the recovery of transgenic events would include, but are not limited to, examples such as GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449).

Expression cassette comprising a regulatory element operably linked to a polynucleotide sequence of interest may be used to transform any plant. In another embodiment, an expression cassette comprising the sequences of SEQ ID NOs: 1-206 operably linked to a polynucleotide sequence of interest may be used to transform any plant. In this manner, genetically modified plants, plant cells, plant tissue, seed, root and the like may be obtained.

Certain disclosed methods involve introducing a polynucleotide into a plant. As used herein, “introducing” is intended to mean presenting to the plant the polynucleotide in such a manner that the sequence gains access to the interior of a cell of the plant. The methods of the disclosure do not depend on a particular method for introducing a sequence into a plant, only that the polynucleotide gains access to the interior of at least one cell of the plant. Methods for introducing polynucleotide into plants are known in the art including, but not limited to, stable transformation methods, transient transformation methods and virus-mediated methods.

A “stable transformation” is a transformation in which the polynucleotide construct introduced into a plant integrates into the genome of the plant and is capable of being inherited by the progeny thereof “Transient transformation” means that a polynucleotide is introduced into the plant and does not integrate into the genome of the plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055 and Zhao, et al., U.S. Pat. No. 5,981,840), direct gene transfer (Paszkowski, et al., (1984) EMBO J. 3:2717-2722) and ballistic particle acceleration (see, for example, U.S. Pat. Nos. 4,945,050; 5,879,918; 5,886,244; 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926) and Lecl transformation (WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., and (1996) Nature Biotechnology 14:745-750 (maize via Agrobacterium tumefaciens).

In one embodiment, DNA constructs comprising a regulatory element may be provided to a plant using a variety of transient transformation methods. In another embodiment, DNA constructs comprising the disclosed sequences SEQ ID NOs: 1-206 may be provided to a plant using a variety of transient transformation methods. Such transient transformation methods include, but are not limited to, viral vector systems and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethylimine (PEI; Sigma #P3143).

In other embodiments, a polynucleotide may be introduced into plants by contacting plants with a virus or viral nucleic acids. Generally, such methods involve incorporating a polynucleotide construct of the disclosure within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855 and WO99/25853. Briefly, the polynucleotide of the disclosure can be contained in transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide construct, for example, an expression cassette comprising one of SEQ ID NOs: 1-206, stably incorporated into its genome.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the embodiments containing a desired polynucleotide is cultivated using methods well known to one skilled in the art.

The embodiments provide compositions for screening compounds that modulate expression within plants. The vectors, cells and plants can be used for screening candidate molecules for agonists and antagonists of the regulatory element sequences of SEQ ID NOs: 1-206. For example, a reporter gene can be operably linked to a regulatory element sequence and expressed as a transgene in a plant. Compounds to be tested are added and reporter gene expression is measured to determine the effect on promoter activity.

In one embodiment, a regulatory element, for example sequences SEQ ID NOs: 1-206 may be edited or inserted into a plant by genome editing using a CRISPR/Cas9 system.

In an aspect, the disclosed regulatory elements may be introduced into the genome of a plant using genome editing technologies, or previously introduced regulatory elements in the genome of a plant may be edited using genome editing technologies. For example, the disclosed regulatory elements may be introduced into a desired location in the genome of a plant through the use of double-stranded break technologies such as TALENs, meganucleases, zinc finger nucleases, CRISPR-Cas, and the like. For example, the disclosed regulatory elements may be introduced into a desired location in a genome using a CRISPR-Cas system, for the purpose of site-specific insertion. The desired location in a plant genome can be any desired target site for insertion, such as a genomic region amenable for breeding or may be a target site located in a genomic window with an existing trait of interest. Existing regulatory elements of interest could be either an endogenous regulatory element or a previously introduced regulatory element.

In another aspect, where the disclosed regulatory element has previously been introduced into a genome, genome editing technologies may be used to alter or modify the introduced regulatory element sequence. Site specific modifications that can be introduced into the disclosed regulatory elements compositions include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. Such technologies can be used to modify the previously introduced polynucleotide through the insertion, deletion or substitution of nucleotides within the introduced polynucleotide. Alternatively, double-stranded break technologies can be used to add additional nucleotide sequences to the introduced polynucleotide.

An “altered target site,” “altered target sequence.” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

All publications, patents and patent applications mentioned in the specification are indicative of the level of those skilled in the art to which this disclosure pertains. All publications, patents and patent applications are herein incorporated by reference to the same extent as if each individual publication, patent or patent application was specifically and individually indicated to be incorporated by reference.

The above description of various illustrated embodiments of the disclosure is not intended to be exhaustive or to limit the scope to the precise form disclosed. While specific embodiments of examples are described herein for illustrative purposes, various equivalent modifications are possible within the scope of the disclosure, as those skilled in the relevant art will recognize. The teachings provided herein can be applied to other purposes, other than the examples described above. Numerous modifications and variations are possible in light of the above teachings and, therefore, are within the scope of the appended claims.

These and other changes may be made in light of the above detailed description. In general, in the following claims, the terms used should not be construed to limit the scope to the specific embodiments disclosed in the specification and the claims.

Efforts have been made to ensure accuracy with respect to the numbers used (e.g. amounts, temperature, concentrations, etc.), but some experimental errors and deviations should be allowed for. Unless otherwise indicated, parts are parts by weight; molecular weight is average molecular weight; temperature is in degrees centigrade; and pressure is at or near atmospheric.

### EXPERIMENTAL

### Example 1: Identification and Cloning of Regulatory Element Sequences

Regulatory element sequences were identified using a combination of a proprietary expression database for soybean and the Legume Information System portal (LIS: www.legumeinfo.org; Dash S, Campbell J D, Cannon E K, Cleary A M, Huang W, Kalberer S R, Karingula V, Rice A G, Singh J, Umale P E, Weeks N T, Wilkey A P, Farmer A D, Cannon S B. Nucl. Acids Res. (2016) 44: D1181-D1188). Candidate genes were identified based on their expression profiles across different tissues and developmental time points. The coding and 5′ flanking regions for these genes were extracted from LIS so a BLAST search could be performed using Phytozome to confirm the sequence annotation. Phytozome (phytozome.jgi.doe.gov/pz/portal.html) is the Plant Comparative Genomics portal of the Department of Energy's Joint Genome Institute. The site provides users a place for accessing, visualizing and analyzing Joint Genome Institute sequenced plant genomes and other selected genomes. The 5′ flanking sequences from candidate genes were synthesized for testing and ranged between 700 bp to 3500 bp. The sequences were relieved of open reading frames of 300 bp or greater, restriction sites that would hinder cloning and allergen/toxin hits identified through the COMPARE (comparedatabase.org/, which can be accessed on the world-wide web using the “www” prefix) database and an internal proprietary toxin database. DNA fragments were synthesized and cloned into an expression vector containing a proprietary trait gene as a reporter and a transcription termination sequence.

### Example 2: Agrobacterium-Mediated Transient Expression

A transient expression system under control of the AtUBQ10 promoter (Day, et. al., (1999) Plant Mol. Biol. 40:771-782; Norris S R et al (1993) Plant Mol Biol. 21(5):895-906) was used as a control construct. The agro-infiltration method of introducing an Agrobacterium cell suspension to plant cells of intact tissues so that reproducible infection and subsequent plant derived transgene expression may be measured or studied is well known in the art (Kapila, et. al., (1997) Plant Science 122:101-108). Briefly, excised leaf disks of soybean (Glycine max), were agro-infiltrated with normalized bacterial cell cultures of test and control strains. After 4 days leaf disks were analysed for protein expression. Control leaf discs were generated with Agrobacterium containing only a DsRed2 fluorescence marker (Clontech™, 1290 Terra Bella Ave. Mountain View, CA 94043) expression vector. Leaf discs from non-infiltrated plants were included as a second control. Results are shown in Table 2.

TABLE 2
Expression score of regulatory elements in soy transient assay

| PROMOTER | Score | SEQ ID NO: |
|---|---|---|
| AT-UBQ 10 PRO | 9 | 201 |
| CA-ACTIN7 PRO (MOD1) | 6 | 52 |
| CA-ASR PRO (MOD1) | 4 | 62 |
| CA-ATPASE-B PRO (MOD1) | 0 | 202 |
| CA-CAB PRO (MOD1) | 6 | 14 |
| CA-CAB-CP26 PRO (MOD1) | 3 | 36 |
| CA-CWAH PRO (MOD1) | 0 | 203 |
| CA-CWLP PRO (MOD1) | 2 | 60 |
| CA-GAPC PRO | 0 | 58 |
| CA-GAPDH PRO (MOD1) | 3 | 8 |
| CA-HSP70 PRO (MOD1) | 6 | 205 |
| CA-HSP90-1 PRO (MOD1) | 3 | 9 |
| CA-HSP90-2 PRO (MOD1) | 4 | 13 |
| CA-LHCA3-1 PRO (MOD1) | 0 | 11 |
| CA-LHCB2-1 PRO | 2 | 10 |
| CA-LTP1 PRO (MOD1) | 5 | 206 |
| CA-MetE PRO (MOD1) | 5 | 7 |
| CA-METE PRO (MOD1)-V1 | 5 | 129 |
| CA-MTH3 PRO (MOD1) | 0 | 111 |
| CA-MuDR PRO (MOD1) | 0 | 30 |
| CA-PPI-1 PRO (MOD1) | 4 | 64 |
| CA-PSI-LHCI PRO | 3 | 61 |
| CA-RUBISCO PRO (MOD1) | 6 | 32 |
| CA-SAG PRO (MOD1) | 0 | 18 |
| CA-SAMD (MOD1) | 6 | 100 |
| CA-THI1-2 PRO (MOD1) | 4 | 63 |
| CA-TIP1 PRO (MOD1) | 6 | 59 |
| CA-UBI PRO (MOD1) | 7 | 68 |
| CA-UNK PRO (MOD1) | 0 | 26 |
| CA-UNK PRO (MOD1)-V1 | 0 | 37 |
| CA-WD40 PRO (MOD1) | 0 | 12 |
| CC-14-3-3 PRO (MOD1) | 3 | 95 |
| CC-ACTIN7 PRO (MOD1) | 2 | 53 |
| CC-GAST-1 PRO (MOD1) | 0 | 88 |
| CC-HMG2 PRO (MOD1) | 3 | 98 |
| CC-LOX PRO (MOD1) | 0 | 119 |
| CC-METE PRO | 3 | 127 |
| CC-MTH2A PRO (MOD1) | 7 | 124 |
| CC-PIP1-4 PRO (MOD1) | 3 | 82 |
| CC-PIP2-4 PRO (MOD1) | 3 | 84 |
| CC-SKP1 PRO (MOD1) | 0 | 90 |
| CC-TIP1 PRO | 3 | 66 |
| CC-TUBA2 PRO (MOD1) | 0 | 76 |
| CC-UBI PRO | 6 | 68 |
| CC-UBI2 PRO (MOD1) | 9 | 105 |
| CC-UNK PRO (MOD1) | 0 | 27 |
| GM-14-3-3 PRO | 5 | 92 |
| GM-14-3-3(2) PRO (MOD1) | 4 | 93 |
| GM-ACTIN7 PRO (MOD1) | 3 | 50 |
| GM-ADF3 PRO (MOD1) | 0 | 39 |
| GM-ADF3(2) PRO (MOD1) | 0 | 40 |
| GM-Beta-amylase PRO (MOD1) | 1 | 47 |
| GM-CAB AB80 PRO (MOD1) | 4 | 1 |
| GM-CAB2 PRO-V1 | 5 | 131 |
| GM-CAB215 PRO (MOD1) | 6 | 2 |
| GM-CCDC72 PRO (MOD1) | 1 | 42 |
| GM-EFTU2 PRO-V1 | 6 | 132 |
| GM-GAPC1 PRO (MOD1) | 2 | 55 |
| GM-GAPC1-2 PRO (MOD1) | 1 | 56 |
| GM-GAPC2 PRO (MOD1) | 2 | 54 |
| GM-GAPC2-2 PRO (MOD1) | 3 | 57 |
| GM-GAST-1 PRO (MOD1) | 3 | 87 |
| GM-HMG2 PRO (MOD1) | 3 | 133 |
| GM-HMG2.2 PRO (MOD1) | 3 | 134 |
| GM-LOX PRO | 0 | 117 |
| GM-LTP1B PRO (MOD1) | 4 | 3 |
| GM-METE PRO | 2 | 125 |
| GM-MTH2 PRO | 4 | 135 |
| GM-MTH3 PRO (MOD1) | 2 | 109 |
| GM-MTH3-2 PRO (MOD1) | 0 | 110 |
| GM-nsLTP15 PRO | 1 | 16 |
| GM-PIP2-4 PRO | 4 | 83 |
| GM-PPI(CYP18-3) PRO (MOD1) | 5 | 71 |
| GM-PPI(CYP19-1) PRO (MOD1) | 5 | 70 |
| GM-PSAL PRO (MOD1) | 2 | 4 |
| GM-PSID2 PRO-V2 | 3 | 136 |
| GM-RCA2 PRO (MOD1) | 5 | 113 |
| GM-SAHASE PRO (MOD1) | 0 | 77 |
| GM-SAMD (MOD1) | 6 | 99 |
| GM-SAMS PRO | 6 | 137 |
| GM-SHMT4 PRO (MOD1) | 2 | 38 |
| GM-SKP1 PRO | 4 | 89 |
| GM-TMA7 PRO (MOD1) | 1 | 41 |
| GM-TUBA2 PRO (MOD1) | 0 | 73 |
| GM-UBQ PRO | 9 | 138 |
| GM-VSP25 PRO (MOD1) | 0 | 5 |
| GM-VSPB PRO (MOD1) | 2 | 6 |
| HA-UBI1 PRO | 9 | 139 |
| LJ-AP(HAD IIIB) PRO (MOD1) | 0 | 44 |
| LJ-Beta-amylase PRO | 0 | 48 |
| LJ-CA2 PRO (MOD1) | 3 | 45 |
| LJ-GAST-1 PRO (MOD1) | 2 | 86 |
| LJ-METE PRO | 3 | 128 |
| LJ-PIP1-4 PRO (MOD1) | 4 | 80 |
| LJ-PIP2-4 PRO | 0 | 85 |
| LJ-PPI PRO (MOD1) | 4 | 72 |
| LJ-SKP1 PRO | 3 | 91 |
| LJ-UBI PRO (MOD1) | 6 | 69 |
| LJ-UBI1 PRO | 10 | 140 |
| LJ-UBI2 PRO | 6 | 106 |
| MT-ACTIN7 PRO (MOD1) | 3 | 51 |
| MT-ALP PRO (MOD1) | 0 | 19 |
| MT-Beta-amylase PRO | 0 | 47 |
| MT-CA2 PRO (MOD1) | 2 | 46 |
| MT-CAMT PRO (MOD1) | 0 | 17 |
| MT-CP12-1 PRO (MOD1) | 4 | 25 |
| MT-CSRP PRO (MOD1) | 0 | 29 |
| MT-GARP PRO (MOD1) | 5 | 43 |
| MT-GRP-LG485 PRO (MOD1) | 1 | 21 |
| MT-LHCB1 PRO (MOD 1) | 7 | 35 |
| MT-LLR PRO (MOD1) | 2 | 31 |
| MT-LOX PRO (MOD1) | 3 | 23 |
| MT-METE PRO | 2 | 126 |
| MT-MIP PRO (MOD1) | 0 | 22 |
| MT-MIPS PRO (MOD1) | 3 | 24 |
| MT-MTH2A PRO (MOD1) | 1 | 121 |
| MT-MTH3 PRO (MOD1) | 0 | 112 |
| MT-PEROXIDASE PRO (MOD1) | 0 | 28 |
| MT-RCA2 PRO (MOD1) | 4 | 115 |
| MT-RUBISCO PRO (MOD1) | 3 | 33 |
| MT-SAHASE PRO (MOD1) | 5 | 79 |
| MT-TIP1 PRO (MOD1) | 2 | 67 |
| MT-TUBA2 PRO | 0 | 75 |
| MT-UBI2 PRO (MOD1) | 7 | 103 |
| MT-UBI3 PRO | 8 | 104 |
| MT-VSPA PRO (MOD1) | 1 | 20 |
| NT-UBI4 PRO | 7 | 141 |
| PH-LOX PRO | 0 | 120 |
| PP-MTH1 PRO (MOD 1) | 6 | 142 |
| PV-14-3-3 PRO (MOD1) | 5 | 94 |
| PV-HMG2 PRO (MOD1) | 3 | 97 |
| PV-LTP PRO | 4 | 15 |
| PV-PIP1-4 PRO (MOD1) | 1 | 81 |
| PV-SAHASE PRO (MOD1) | 1 | 78 |
| PV-SAMD (MOD1) | 7 | 101 |
| PV-TUBA2 PRO | 0 | 74 |
| VU-LOX PRO | 3 | 118 |
| VU-MTH2A PRO | 7 | 122 |
| VU-MTH2A-2 PRO (MOD1) | 7 | 123 |
| VU-RCA2 PRO (MOD1) | 1 | 116 |
| VU-UBI1 PRO (MOD1) | 9 | 107 |
| VU-UBI2 PRO | 8 | 108 |
| VV-UBI6 PRO | 7 | 144 |
| VV-UBI7 PRO | 9 | 145 |

*Expression on a scale of 1-10 (low to high) with the AtUBQ10 promoter used as a positive control

### Example 3: Agrobacterium-Mediated Stable Transformation of Arabidopsis Plants

The expression cassettes described for the transient assay were re-cloned into a vector backbone suitable for stable transformation purposes. These vectors were used to transform Arabidopsis thaliana plants using the floral dip procedure described in Clough and Bent, 1998. Briefly, about 4-week old Arabidopsis plants with floral buds were dipped in a bacterial suspension of Agrobacterium strain C58 cultured in YEP medium comprising 5% (w/v) sucrose 10 and 0.05% (v/v) Silwet-77 (Mohanty et al. 2009). The transformed plants were selected by germinating T1 seed on solid media containing the herbicide, BASTA, at a concentration of 10 μg/ml. Single copy events were identified by qPCR and used for promoter characterization. Results are shown in Table 3.

TABLE 3
Expression score of regulatory elements in Arabidopsis
on a scale of 1-10 (low to high)

| Promoter | Score | SEQ ID NO: |
|---|---|---|
| CA-ACTIN7 PRO (MOD1) | 5 | 52 |
| CA-CAB PRO (MOD1) | 9 | 14 |
| CA-CAB-CP26 PRO (MOD1) | 4 | 36 |
| CA-CWLP PRO (MOD1) | 2 | 60 |
| CA-GAPC PRO | 3 | 58 |
| CA-GAPDH PRO (MOD1) | 4 | 8 |
| CA-HSP70 PRO (MOD1) | 2 | 205 |
| CA-HSP90-1 PRO (MOD1) | 2 | 9 |
| CA-HSP90-2 PRO (MOD1) | 1 | 13 |
| CA-LHCA3-1 PRO (MOD1) | 4 | 11 |
| CA-LHCB2-1 PRO | 6 | 10 |
| CA-MTH3 PRO (MOD1) | 5 | 111 |
| CA-PPI-1 PRO (MOD1) | 5 | 64 |
| CA-PSI-LHCI PRO | 5 | 61 |
| CA-RUBISCO PRO (MOD1) | 9 | 32 |
| CA-THI1-2 PRO (MOD1) | 4 | 63 |
| CA-TIP1 PRO (MOD1) | 5 | 59 |
| CA-UBI PRO (MOD1) | 7 | 68 |
| CA-UNK PRO (MOD1) | 3 | 26 |
| CA-WD40 PRO (MOD1) | 7 | 12 |
| CC-ACTIN7 PRO (MOD1) | 5 | 53 |
| CC-METE PRO | 5 | 127 |
| CC-MTH2A PRO (MOD1) | 6 | 124 |
| CC-TIP1 PRO | 2 | 66 |
| CC-UBI PRO | 4 | 68 |
| CC-UBI2 PRO (MOD1) | 8 | 105 |
| GM-ACTIN7 PRO (MOD1) | 4 | 50 |
| GM-Beta-amylase PRO (MOD1) | 2 | 47 |
| GM-CAB215 PRO (MOD1) | 6 | 2 |
| GM-CAB AB80 PRO (MOD1) | 2 | 1 |
| GM-GAPC1 PRO (MOD1) | 4 | 55 |
| GM-GAPC1-2 PRO (MOD1) | 4 | 56 |
| GM-GAPC2 PRO (MOD1) | 4 | 54 |
| GM-GAPC2-2 PRO (MOD1) | 1 | 57 |
| GM-GAST-1 PRO (MOD1) | 3 | 87 |
| GM-HMG2 PRO (MOD1) | 2 | 133 |
| GM-LOX PRO | 3 | 117 |
| GM-LTP1B PRO (MOD1) | 1 | 3 |
| GM-METE PRO | 5 | 2 |
| GM-MTH3 PRO (MOD1) | 2 | 103 |
| GM-MTH3-2 PRO (MOD1) | 5 | 110 |
| GM-PIP2-4 PRO | 3 | 83 |
| GM-PPI(CYP18-3) PRO (MOD1) | 5 | 71 |
| GM-PPI(CYP19-1) PRO (MOD1) | 3 | 70 |
| GM-PSAL PRO (MOD1) | 4 | 4 |
| GM-RCA2 PRO (MOD1) | 8 | 113 |
| GM-TUBA2 PRO (MOD1) | 3 | 73 |
| LJ-AP(HAD IIIB) PRO (MOD1) | 1 | 44 |
| LJ-Beta-amylase PRO | 2 | 48 |
| LJ-CA2 PRO (MOD1) | 4 | 45 |
| LJ-GAST-1 PRO (MOD1) | 5 | 86 |
| LJ-METE PRO | 6 | 128 |
| LJ-PIP1-4 PRO (MOD1) | 5 | 80 |
| LJ-PPI PRO (MOD1) | 2 | 72 |
| LJ-UBI PRO (MOD1) | 1 | 69 |
| LJ-UBI2 PRO | 6 | 106 |
| MT-ACTIN7 PRO (MOD1) | 4 | 51 |
| MT-Beta-amylase PRO | 4 | 47 |
| MT-CA2 PRO (MOD1) | 6 | 46 |
| MT-CAMT PRO (MOD1) | 9 | 17 |
| MT-CP12-1 PRO (MOD1) | 8 | 25 |
| MT-CSRP PRO (MOD1) | 4 | 29 |
| MT-GARP PRO (MOD1) | 3 | 43 |
| MT-GRP-LG485 PRO (MOD1) | 2 | 21 |
| MT-LHCB1 PRO (MOD 1) | 10 | 35 |
| MT-LLR PRO (MOD1) | 6 | 31 |
| MT-LOX PRO (MOD1) | 2 | 23 |
| MT-METE PRO | 4 | 126 |
| MT-MIP PRO (MOD1) | 4 | 22 |
| MT-MTH2A PRO (MOD1) | 4 | 121 |
| MT-MTH3 PRO (MOD1) | 6 | 112 |
| MT-PEROXIDASE PRO (MOD1) | 5 | 28 |
| MT-RCA2 PRO (MOD1) | 8 | 115 |
| MT-RUBISCO PRO (MOD1) | 6 | 33 |
| MT-SAHASE PRO (MOD1) | 3 | 79 |
| MT-TIP1 PRO (MOD1) | 4 | 67 |
| MT-UBI2 PRO (MOD1) | 5 | 103 |
| MT-UBI3 PRO | 7 | 104 |
| PP-MTH1 PRO (MOD 1) | 5 | 142 |
| PV-LTP PRO | 5 | 15 |
| PV-PIP1-4 PRO (MOD1) | 3 | 81 |
| PV-SAHASE PRO (MOD1) | 3 | 78 |
| PV-TUBA2 PRO | 3 | 74 |
| VU-MTH2A-2 PRO (MOD1) | 7 | 122 |
| VU-RCA2 PRO (MOD1) | 9 | 116 |
| VU-UBI1 PRO (MOD1) | 7 | 107 |

### Example 4: Expression Analysis of the Regulatory Elements

Protein quantification of the reporter gene was used to assess promoter performance. Mass spectroscopy/spectroscopy results were converted into an arbitrary expression score for a comparative analysis between expression systems. Expression scores are listed on a scale of 1 to 10 with 1 being the lowest and 10 being the highest expression level.

### Example 5: Comparative Analysis Between Soybean and Arabidopsis Systems

Some of the promoters were evaluated in both the soy transient assay and in stable, transformed Arabidopsis lines as described in Example 2 and 3. Results are shown in Table 4.

TABLE 4
Expression score of regulatory elements in soybean
and Arabidopsis on a scale of 1-10 (low to high)

| | Score in soy | Score | SEQ |
|---|---|---|---|
| | transient | in arabidopsis | ID |
| Promoter | assays | stable lines | NO: |
| CA-ACTIN7 PRO (MOD1) | 6 | 5 | 52 |
| CA-CAB PRO (MOD1) | 6 | 9 | 14 |
| CA-CAB-CP26 PRO (MOD1) | 3 | 4 | 36 |
| CA-CWLP PRO (MOD1) | 2 | 2 | 60 |
| CA-GAPC PRO | 0 | 3 | 58 |
| CA-GAPDH PRO (MOD1) | 3 | 4 | 8 |
| CA-HSP70 PRO (MOD1) | 6 | 2 | 205 |
| CA-HSP90-1 PRO (MOD1) | 3 | 2 | 9 |
| CA-HSP90-2 PRO (MOD1) | 4 | 1 | 13 |
| CA-LHCA3-1 PRO (MOD1) | 0 | 4 | 11 |
| CA-LHCB2-1 PRO | 2 | 6 | 10 |
| CA-MTH3 PRO (MOD1) | 0 | 5 | 111 |
| CA-PPI-1 PRO (MOD1) | 4 | 5 | 64 |
| CA-PSI-LHCI PRO | 3 | 5 | 61 |
| CA-RUBISCO PRO (MOD1) | 6 | 9 | 32 |
| CA-THI1-2 PRO (MOD1) | 4 | 4 | 63 |
| CA-TIP1 PRO (MOD1) | 6 | 5 | 59 |
| CA-UBI PRO (MOD1) | 7 | 7 | 68 |
| CA-UNK PRO (MOD1) | 0 | 3 | 26 |
| CA-WD40 PRO (MOD1) | 0 | 7 | 12 |
| CC-ACTIN7 PRO (MOD1) | 2 | 5 | 53 |
| CC-METE PRO | 3 | 5 | 127 |
| CC-MTH2A PRO (MOD1) | 7 | 6 | 124 |
| CC-TIP1 PRO | 3 | 2 | 66 |
| CC-UBI PRO | 6 | 4 | 68 |
| CC-UBI2 PRO (MOD1) | 9 | 8 | 105 |
| GM-ACTIN7 PRO (MOD1) | 3 | 4 | 50 |
| GM-Beta-amylase PRO (MOD1) | 1 | 2 | 47 |
| GM-CAB AB80 PRO (MOD1) | 4 | 6 | 2 |
| GM-CAB215 PRO (MOD1) | 6 | 2 | 1 |
| GM-GAPC1 PRO (MOD1) | 2 | 4 | 55 |
| GM-GAPC1-2 PRO (MOD1) | 1 | 4 | 56 |
| GM-GAPC2 PRO (MOD1) | 2 | 4 | 54 |
| GM-GAPC2-2 PRO (MOD1) | 3 | 1 | 57 |
| GM-GAST-1 PRO (MOD1) | 3 | 3 | 87 |
| GM-HMG2 PRO (MOD1) | 3 | 2 | 133 |
| GM-LOX PRO | 0 | 3 | 117 |
| GM-LTP1B PRO (MOD1) | 4 | 1 | 3 |
| GM-METE PRO | 2 | 5 | 2 |
| GM-MTH3 PRO (MOD1) | 2 | 2 | 103 |
| GM-MTH3-2 PRO (MOD1) | 0 | 5 | 110 |
| GM-PIP2-4 PRO | 4 | 3 | 83 |
| GM-PPI(CYP18-3) PRO (MOD1) | 5 | 5 | 71 |
| GM-PPI(CYP19-1) PRO (MOD1) | 5 | 3 | 70 |
| GM-PSAL PRO (MOD1) | 2 | 4 | 4 |
| GM-RCA2 PRO (MOD1) | 5 | 8 | 113 |
| GM-TUBA2 PRO (MOD1) | 0 | 3 | 73 |
| LJ-AP(HAD IIIB) PRO (MOD1) | 0 | 1 | 44 |
| LJ-Beta-amylase PRO | 0 | 2 | 48 |
| LJ-CA2 PRO (MOD1) | 3 | 4 | 45 |
| LJ-GAST-1 PRO (MOD1) | 2 | 5 | 86 |
| LJ-METE PRO | 3 | 6 | 128 |
| LJ-PIP1-4 PRO (MOD1) | 4 | 5 | 80 |
| LJ-PPI PRO (MOD1) | 4 | 2 | 72 |
| LJ-UBI PRO (MOD1) | 6 | 1 | 69 |
| LJ-UBI2 PRO | 6 | 6 | 106 |
| MT-ACTIN7 PRO (MOD1) | 3 | 4 | 51 |
| MT-Beta-amylase PRO | 0 | 4 | 47 |
| MT-CA2 PRO (MOD1) | 2 | 6 | 46 |
| MT-CAMT PRO (MOD1) | 0 | 9 | 17 |
| MT-CP12-1 PRO (MOD1) | 4 | 8 | 25 |
| MT-CSRP PRO (MOD1) | 0 | 4 | 29 |
| MT-GARP PRO (MOD1) | 5 | 3 | 43 |
| MT-GRP-LG485 PRO (MOD1) | 1 | 2 | 21 |
| MT-LHCB1 PRO (MOD1) | 7 | 10 | 35 |
| MT-LLR PRO (MOD1) | 2 | 6 | 31 |
| MT-LOX PRO (MOD1) | 3 | 2 | 23 |
| MT-METE PRO | 2 | 4 | 126 |
| MT-MIP PRO (MOD1) | 0 | 4 | 22 |
| MT-MTH2A PRO (MOD1) | 1 | 4 | 121 |
| MT-MTH3 PRO (MOD1) | 0 | 6 | 112 |
| MT-PEROXIDASE PRO | 0 | 5 | 28 |
| (MOD1) | | | |
| MT-RCA2 PRO (MOD1) | 4 | 8 | 115 |
| MT-RUBISCO PRO (MOD1) | 3 | 6 | 33 |
| MT-SAHASE PRO (MOD1) | 5 | 3 | 79 |
| MT-TIP1 PRO (MOD1) | 2 | 4 | 67 |
| MT-UBI2 PRO (MOD1) | 7 | 5 | 103 |
| MT-UBI3 PRO | 8 | 7 | 104 |
| PP-MTH1 PRO (MOD 1) | 6 | 5 | 142 |
| PV-LTP PRO | 4 | 5 | 15 |
| PV-PIP1-4 PRO (MOD1) | 1 | 3 | 81 |
| PV-SAHASE PRO (MOD1) | 1 | 3 | 78 |
| PV-TUBA2 PRO | 0 | 3 | 74 |
| VU-MTH2A-2 PRO (MOD1) | 7 | 7 | 122 |
| VU-RCA2 PRO (MOD1) | 1 | 9 | 116 |
| VU-UBI1 PRO (MOD1) | 9 | 7 | 107 |

### Example 6: Deletion Analysis

Deleting segments of the 5′ end of the full-length regulatory element can alter the expression pattern and provide insight into important sequence markers in the regulatory region. SEQ ID NOs: 147 to 198 are truncated versions of the full-length regulatory elements of At-RBCS1A (SEQ ID NO 199), CA-LHCB2-1 (SEQ ID NO: 10), CA-RUBISCO (SEQ ID NO: 32), CA-UBI (M1) PRO (SEQ ID NO: 68), CM-RBCS1 (SEQ ID NO: 200), LJ-UBI (SEQ ID NO: 69), CC-UBI (SEQ ID NO: 68), CA-ACTIN7 (SEQ ID NO: 52), GM-PPI(CYP18-3) PRO (SEQ ID NO: 71), LJ-PPI PRO (SEQ ID NO: 72), CA-TIP1 PRO (SEQ ID NO 59) CA-HSP70 (SEQ ID NO: 207) and CA-WD40 PRO (SEQ ID NO: 12) (See Table 5).

It has been shown that the intron from the maize UBI promoter/intron combination commonly used for transgene expression (Christensen et al. Plant Mol Biol. 1992, 18(4):675-89; and Christensen and Quail, Transgenic Research 1996, Volume 5, Issue 3, pp 213-218) has promoter activity (internal data, look for external publication). Truncations were made with selected promoters listed in table 5, which includes either 5′UTR, introns, UARs containing the TATA box, or a combination of these, to determine transcriptional activity.
SEQ ID NOs: 147 and 148 are truncated versions of the full-length regulatory element At-RBCS1A (SEQ ID NO 199).
SEQ ID NOs: 150 and 151 are truncated versions of the full-length regulatory element CA-LHCB2-1 (SEQ ID NO: 10).
SEQ ID NOs: 153-156 are truncated versions of the full-length regulatory element CA-RUBISCO (SEQ ID NO: 32).
SEQ ID NOs: 157-161 are truncated versions of the full-length regulatory element CA-UBI (SEQ ID NO: 68).
SEQ ID NOs: 162 and 163 are the truncated versions of the full-length regulatory element CM-RBCS1 (SEQ ID NO: 200).
SEQ ID NO: 164-170 are the truncated versions of the full-length regulatory element LJ-UBI (SEQ ID NO 69).
SEQ ID NOs: 171-177 are the truncated versions of the full-length regulatory element CC-UBI (SEQ ID NO: 68).
SEQ ID NOs: 178-181 are the are truncated versions of the full-length regulatory element CA-ACTIN7 (SEQ ID NO: 52).
SEQ ID NOs: 182-185 are the truncated versions of the full-length regulatory element GM-PPI(CYP18-3) PRO (SEQ ID NO: 71).
SEQ ID NOs: 186-189 are the truncated versions of the full-length regulatory element LJ-PPI PRO (SEQ ID NO: 72).
SEQ ID NOs: 190-193 are the truncated versions of the full-length regulatory element CA-TIP1 PRO (SEQ ID NO: 59).
SEQ ID NOs: 194-197 are the truncated versions of the full-length regulatory element CA-HSP70 (SEQ ID NO: 207).
SEQ ID NOs: 198 is the truncated versions of the full-length regulatory element CA-WD40 PRO (SEQ ID NO: 12).

TABLE 5
Truncations of regulatory elements and Expression
score on a scale of 1-10 (low to high)

| SEQ ID | | LENGTH | |
|---|---|---|---|
| NO: | PROMOTER | (bp) | Score |
| 146 | At-RBCS1A PRO F | 1549 | 4 |
| 147 | At-RBCS1A PRO Tr368 | 569 | 3 |
| 148 | At-RBCS1A PRO Tr3748 | 275 | 2 |
| 149 | CA-LHCB2-1 PRO F | 1506 | 5 |
| 150 | CA-LHCB2-1 PRO Tr336 | 421 | 5 |
| 151 | CA-LHCB2-1 PRO Tr58 | 145 | 1 |
| 152 | CA-RUBISCO (M1) PRO F | 1506 | 7 |
| 153 | CA-RUBISCO (M1) Tr300 | 737 | 6 |
| 154 | CA-RUBISCO (M1) PRO Tr59 | 496 | 6 |
| 155 | CA-RUBISCO (M1) PRO 5UTR | 428 | 6 |
| 156 | CA-UBI (M1) PRO F | 1506 | 7 |
| 157 | CA-UBI (M1) PRO Tr344noAP | 438 | 6 |
| 158 | CA-UBI (M1) PRO Tr344noPM | 137 | 4 |
| 159 | CA-UBI (M1) PRO Tr344YAP | 869 | 7 |
| 160 | CA-UBI (M1) PRO Tr344YPM | 568 | 5 |
| 161 | CA-UBI INTRON1 | 473 | 1 |
| 162 | CM-RBCS1_PRO Tr327 | 402 | 7 |
| 163 | CM-RBCS1_PRO Tr62 | 137 | 1 |
| 164 | LJ-UBI PARTIAL INTRON (TR7) | 235 | NT |
| 165 | LJ-UBI 5UTR + INTRON (TR6) | 517 | NT |
| 166 | LJ-UBI CORE | 569 | NT |
| 167 | LJ-UBI (TR150) | 704 | 4 |
| 168 | LJ-UBI (TR300) | 844 | 4 |
| 169 | LJ-UBI (TR500) | 1049 | NT |
| 170 | LJ-UBI PRO no intron | 949 | NT |
| 171 | CC-UBI PARTIAL INTRON (TR7) | 300 | NT |
| 172 | CC-UBI 5UTR + INTRON (TR6) | 646 | NT |
| 173 | CC-UBI CORE | 703 | NT |
| 174 | CC-UBI (TR150) | 836 | 3 |
| 175 | CC-UBI (TR300) | 1030 | 5 |
| 176 | CC-UBI (TR500) | 1183 | NT |
| 177 | CC-UBI PRO NO INTRON | 903 | NT |
| 178 | CA-ACTIN7 (CORE)(with intron) | 308 | NT |
| 179 | CA-ACTIN7 (TR150) | 444 | NT |
| 180 | CA-ACTIN7 (TR300) | 604 | NT |
| 181 | CA-ACTIN7 (TR500) | 794 | NT |
| 182 | GM-PPI(CYP18-3) PRO (MOD1) | 604 | NT |
| | (TR500) | | |
| 183 | GM-PPI(CYP18-3) PRO (MOD1) | 406 | 4 |
| | (TR300) | | |
| 184 | GM-PPI(CYP18-3) PRO (MOD1) | 266 | 3 |
| | (TR150) | | |
| 185 | GM-PPI(CYP18-3) PRO (MOD1) | 123 | NT |
| | (CORE) | | |
| 186 | LJ-PPI PRO (MOD1) (TR500) | 583 | NT |
| 187 | LJ-PPI PRO (MOD1) (TR300) | 386 | 3 |
| 188 | LJ-PPI PRO (MOD1) (TR150) | 242 | 2 |
| 189 | LJ-PPI PRO (MOD1) (CORE) | 96 | NT |
| 190 | CA-TIP1 PRO (MOD1) (TR500) | 630 | NT |
| 191 | CA-TIP1 PRO (MOD1) (TR300) | 404 | 3 |
| 192 | CA-TIP1 PRO (MOD1) (TR150) | 260 | 3 |
| 193 | CA-TIP1 PRO (MOD1) (CORE) | 120 | NT |
| 194 | CA-HSP70 PRO (MOD1) (TR500) | 620 | NT |
| 195 | CA-HSP70 PRO (MOD1) (TR300) | 455 | 4 |
| 196 | CA-HSP70 PRO (MOD1) (TR150) | 280 | 3 |
| 197 | CA-HSP70 PRO (MOD1) (CORE) | 139 | NT |
| 198 | CA-WD40 PRO (TR1) | 1274 | 3 |
| 200 | CM-RBCS1 PRO F | 1004 | NT |

*NT means not tested.

What is claimed is:

1. A recombinant polynucleotide comprising:
(a) a polynucleotide having at least 95 percent sequence identity to the nucleic acid sequence of any one of SEQ ID NO: 1-206;
(b) a polynucleotide of any one of SEQ ID NO: 1-206; or
(c) a fragment of any one of SEQ ID NO: 1-206;
wherein the recombinant polynucleotide has regulatory activity.

2. The recombinant polynucleotide of claim 1, wherein the recombinant polynucleotide further comprises a heterologous polynucleotide.

3. A DNA construct comprising a heterologous transcribable polynucleotide molecule operably linked to the regulatory element polynucleotide, wherein the regulatory element polynucleotide comprises:
(a) a polynucleotide having at least 95 percent sequence identity to the nucleic acid sequence of any one of SEQ ID NO: 1-206;
(b) a polynucleotide of any one of SEQ ID NO: 1-206; or
(c) a fragment of any one of SEQ ID NO: 1-206,
wherein the regulatory element polynucleotide has regulatory activity.

4. The DNA construct of claim 3, wherein the regulatory element polynucleotide further comprises a heterologous polynucleotide.

5. The DNA construct of claim 3, wherein the heterologous polynucleotide molecule is a gene of agronomic interest.

6. The DNA construct of claim 5, wherein the heterologous polynucleotide molecule is a gene capable of providing herbicide resistance in plants.

7. The DNA construct of claim 5, wherein the heterologous polynucleotide molecule is a gene capable of providing plant pest control in plants.

8. A heterologous cell stably transformed with the nucleic acid molecule of claim 1.

9. A transgenic plant or plant cell stably transformed with the DNA construct of claim 3.

10. The transgenic plant or plant cell of claim 9, wherein the transgenic plant is a dicotyledon plant cell.

11. The transgenic plant or plant cell of claim 9, wherein the transgenic plant is a monocotyledon plant cell.

12. A seed of the transgenic plant of claim 9, wherein the seed comprises the DNA construct.

13. A method for expressing a polynucleotide in a plant comprising introducing into a plant cell a recombinant polynucleotide, said recombinant polynucleotide comprising a regulatory element capable of increasing expression of a heterologous polynucleotide, wherein said regulatory element comprises:
(a) a nucleotide sequence of any one of SEQ ID NO: 1-206;
(b) a sequence that is at least 95% identical to any one of SEQ ID NO: 1-206; or
(c) a nucleotide sequence comprising a fragment or variant of the nucleotide sequence of any one of SEQ ID NO: 1-206, wherein the nucleotide sequence has regulatory activity in a plant cell;

14. The method of claim 13, wherein the heterologous polynucleotide encodes a gene product that is involved in organ development, stem cell development, cell growth stimulation, organogenesis, somatic embryogenesis initiation and development of the apical meristem.

15. The method of claim 13, wherein said a heterologous polynucleotide is an endogenous gene of the plant.

16. The method of claim 13, wherein the heterologous polynucleotide encodes a gene product that confers drought tolerance, cold tolerance, herbicide tolerance, pathogen resistance, or insect resistance.

17. The method of claim 13, wherein said plant is a dicot.

18. The method of claim 13, wherein said plant is a monocot.


SEQUENCE LISTING (206 sequences)

SEQ ID NO: 1 - DNA, 1986 bp, Glycine max
ttgtccaagtgtgactctgaggagatatctattggttagaaacttttgaaaggtttgatctcatatcacttacctttttggattgttttctctctaaagaaatatcacaaatgcctcttccatccaataagagtttgccatttaagattttggataagctatatcagctaaatgaactgtaaaatggaacacggttagaatatcaagtactgcatgcaatgtagggaccaataaaagtggggtgtggccgtagaataaaaccttccaaacaactgtcaagtatatataagatatgtaaaaacttggtatttcatcatgttattttagatttgtcttcttttaaaaagaaaaactaaatcaaacgaaatctataaaaaatgtatttaattttaaccagtaaatttaagaaaaataatgtacttttaaaaaagaaagtttacgagagtttgtcttctttttcagtctttattttaagtttcaaaatctaagaatatattatttaactaattttaatttaaaagtgatttttaaattttaaaaaagacttattttatcaaataacttttactttaaatttaaagttcattatttttatctttaaaataaattttaattaaagaaatgaaaacaaacactatctatgtcttattacttaacgatttttaatctatgctttttatagactttcatttcttcctccttttggctatacatgttcatttatttgtactttactacattttttttttatgttggggaaaataaaagaggctaattagtgcgtaaaagtcgtttatatacgtatatcaaataattaatatcacatataattctcttactttataatttctttaagtacatgtttaataatacgattgtaattttcttttgatgtgctgagccatgtgttcaatagtaatgataataaataattaaattttatctaaaaaatctcaccatatacatacattattttaatagagcttagattcatgagtcttataataggataacaatattatcttgattatctttattattaggccgagggggtacactttacataattgtatttcaaaagtatttcttcaagagtaaacaaaagctagcacagatgaaaaaacattttaaaaaaattatttgaccttcatgtacgagtgctttctaaattaaataattgactgtatagaggtgccttctaaattctcctatattatttcagcttgctttctttcttattttccccagtcgctcacgaaaatctcctattctaatatcttgtgcagttttggcaatcaacatgtattagtgagggtgtgactgtgattactttgatttttgaaactaaaacaataccaaacaaaaaccctctggtaacgtgaagtaatagtttttttggtactgaaagaaaaaagatagccatgtatttatttagtttattacaactaactatatttcaatttgatgtaagtgccccctcattaaaatggacctgtgtaaccatcaacctctagttcgctcttttcaccatttgtctctctgtctctgacttggcaatatttgaaattttgtggttctcatttcccttagtacaacaccagatgtggacccaaaaatatctcagacattgaaactaaggatagccacataattcaagccattttccacgtcacctcctcaatggaatagcataagaaaataagttaaacaacatatctactggaatcccatagtcctacgtggaagacccacattggtcagaaaagcagagaaagaatctaacccatttagtggattataggggtgccaagtgtaccaaacaaaatctgaaagccccaatgagatagtagcaatagataggccaagataagaaccccaaccacttgaagcccatataaataaacccccacacaacttcactgaatcactcacaactccataacacaaggcagaaagcaagctcatcctagagttttaaaaggatcc

SEQ ID NO: 2 - DNA, 1583 bp, Glycine max
gcagatgaagataaagaaaatgaaaagataaactaaattagtctagactttcttggctaaagtagagctttagagtacaaattcaaacaaacttacatcccccgaaagtcttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagaccgtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgagttgtgcatatcctcatccacagtatcatacgatcttctataaacctttgtagtagctttcttcattctccacaacacagaacaaaaacaaagaaaaaaaaaaacccttagccaacacaaccatcggatcc

SEQ ID NO: 3 - DNA, 1651 bp, Glycine max
ccctagcaagttgaatgttgatcacgagaccctacaaattaaaatacgtgtatacatcaaatgtatgatattaagccaggtgtacaaaagatgaaacattttgtttgcagcattaaattgaacccaactaccaactaactcatgccagccacagcacaggacttttgtatcaattcagttgaaaatatacagcacattttttttaaatatattatttctcaaaattgacatattttaaagcatttttatttttaaataaaaagattggaaagatttgaaaaatgagtacacctgtgtaataatgtaaaaaagaagaaaaataataataacattttaagtaatttggattatgaaaagtagaagaatttttttaaaaaaatctactagcttcctttgttagagggacatgagtcctctacattccttctacagcctctctatctcgatctctctaactttctttatatttctactgtatgcattttttcttcttctttctttttccttttattatttttggctacagagacaatagaggaatatctattttgtttattagggtttcagataaaaaaaaatgtcaatttttatttattcgaaaataaatatcaattttgtgaatagtaatataagaaagataaaaaaaattacgcttctctgaacaaaatattttctccatcaagggaaattattctttctagtcttaattatcattttttaatttctttccatctctctttttcctcttctaaagaataaaaaaacgacatattatacgaatacagttatgtgaataattaaaagatttaactgcataacacaaatgaactcattttaacgaattaaataaattgagtagttgactcgcttttgaaatgaaacaaacaaagtttaacataatatgttttatccctttttatgtcacaccacttgacacacaacaaaaacacaaaaaaaaagggtgaaaagaggtttttattatttccataaatgtaattagaagtaattgcagatcgaagtgattaactagagagacattcacattcacattcactccccccacatcattaactcttgtactctttctctcccaaccttcttattaagtatgtttaccattcaccaaaaaaaaaagtatgtttaccactcctcttactttatcctcttttaaataaatttccctcttcctacattgtcgcaaacaaagtaaatgtaaatatacattttattttggggtgtatatattggagcagaagaaagagaaatgaaaaaaaaagaaaaaattaataaatatgctaattgacataatataaaataaaacgaaaaaaatacatatataattataagagttgaaaacaacatccacaaaccacttgagtttaagcctttcactttacactacttttttttgtcatatcacgggacacttaacaaaatcattgaggaaaatagtgaaaagaagtgtttatttatttttccattaatattgaagtgatattaactagagaaaattaatcacattcacgttcacattcacttccccctctatataaaccctacacccagcaccgctcatactcaccaagtgaaccaatatagcccaagccctatacaaccaaaccctaacacacacccatttgattcagtttcc

SEQ ID NO: 4 - DNA, 2160 bp, Glycine max
actaacacataatataggaaaactaataggaagaactaaaattaaaaaaaataaaattcagacattaaaaaaatatatattaaaggactaaaatccaaactatatgtctttaaaggaaaaagattgagagcaactttgagctgtggatgagctaattgcctatgtgtaaagctttatagtcaaaagaattattttggatggtcaatataatgcggcaatcaaacagttgttgtcctctcctccccaagaaagcagccagaataaaaaagaaattcatgaaaaagcaagcatgaacccagaaattcttacgtactctatggaaccaaaattacaaaataaaataaaatagaagtttagtattgggcaatctattgagtatgcaccaagtattagaattttatgacctatatatccacgcttttgattaacttcccttcactataaaactcataattttaccgacattttcctcgtactgatttttttagtttattttttaatttaatagtgatgcatatttaaatacaaattatcgttaatataactttttaaataatttcataaaaattaataaatttaccatacgtacctaactcttgtttcttaccttttttttttattatagaaaatactaatatttttatgatagataatcgtattcaagttattacgaatgataaaaaaaatttctaaatatttaacataattacattcttaaacctttttttaaaactacattctcaaaacttaaaacttaaattaacatgttaattaatctacctacttacttgatatttttaacatgaagagttggagatggattaaaatcactaaaccaatttctttgtctttacgtatatttttatataattaattaattaaaagttattataaaaatatatttaaaaataattattataaaatactaattttaccatacataataatcatataacagtgtggaaatattattttaccgttgacacattttattttattttattgcttttagatataatgtaaaagtagagcaaaaataaggattttttttcctgcaccccataaaaatttatcagcactcctttcggaaaacccaattcaaaatgtaagattatattctagattgaaattttcaaaatataagggcgtgttgataaatttgggaaattgcttcgtgcacttcaatacttctttctacacctacttcaaaatgtgaaattatactaactctttgattttatatataagaaacaaaaaaaaaccatttttatcctatttattaaaaaaatataattactttattgttaatttttttatccccatttaattatgagatctattaatgatatagacttatttttccataaaatagatacatttattgagttatctataaaataagacatactcattgattgaatttttaaaataccacctctaaatccatttaatattatggataatcttaaaatagaattaaaatttataacaacttaaccaatcaaattaattttattggtctgaagaattagataattgtttattacatataagatcaaaatataatattttggattagtctttttggaatatgaagagttgtacattccaaaaagactaatccatcgaaaatataaatcatacatctcaaaaagtttaacccaaaactaagaatacataggaacaccccttatttctaaactagtaaagatggatggaaagaagaaaatcaaagatacaagtgataataatttatatcccttaatcaaatgatgtagtatttgattcttgattaatttttgactatagaataaatcatgttacaagataaatatgcattttgtgcatgatcatagtttctaatgaaaattagtcaatatttctgacaacttcctatcaatattacggctagaaaaaaaagtgggcctgcaagattggcttacccacccaaaaagcccatcacaaagatataaaaatatcaattaaatttggaaaggttggttgtgttatggataagggataaggatttgatacgcaaatcagccaatgagataagagggccttcaacagccacaaccttgatcttgcacttagccagttcggtccctcacttaagccactgtgtggaactaacaacaacacaaggccatcatcggatcc

SEQ ID NO: 5 - DNA, 1962 bp, Glycine max
attcaaataaagagagaaactaaaatttcataaaagagaatacgtgtattaattttatttttggttgacatgactttttcatactaagttggacaccaattgtgtgtgagacttataccatcttgtgagtgttgaacattcttagtgtataacactgataatataaaggggtagacacttttggtcttatagcgtgtgtgatgctgattaataattaacaaaatattttcttttttgagtgtggatgatatatgtgaataacacttaagtcctttaataactttgctcacgtccacttgtcataaacttattaatatatcaaaaatttgtattttttgttaaacatcaaaataggtataaatggtcgtccaaacctaacaatgagcatttaatgttaatatgaattgaagatcatattaacatgtattacatcggacctgagttctttgttgttcctatctcagttgtccccatgctgtttcttgaaaatcacacaattttaaattaaatcataaaattaaatattaacacaaactaaatagataacatcgaatcagctaaaaaaaagacaacaaaaaaaccaatttagtattacatctttcaaacaagacatatgttgcctaaacaattttttggtacatttcttaattgaaaattttacactattctgaataatttattagatacataatatctatgtgcaaatataaaatccatacaacttttcagtgttataggtgaactcatgtatcacgtgttctaataagtctctatatattcagatttaagtgataagagtggaccccgaaaagaaactgtttttttatatatataaacaatactatgctataataaattttatatatatatatatatatatatatatatatatatatatatatatatatatatatatatatattaagaagaagaaaaaaagcgcaagagcatcgatcaaatgaacgaataatgctcaattttcttttattaaaagtataatttaatttatacatattgtatgatgtaatattggtataaaatatttaacatatttgatgataaatatttactaaaaataaaaatttgtatgattatctttatcaacacaattttttttccattcataaaatttgaatgtgactttacttaagagattcaatataattaactatcatcaaacagcttcctggtatatgaacatgcaggctttttctttgtaatctgaggggtatttcttatgcctcatatgaatattattctctctttgccgtttcagaaaaaaaaaaaaaaacttaagaatatacatgcatatatatatatatatatatatatatatacatttatcttaaatatatattttaaagtaattattataaaaataaattattttataataaagaaccatctatgatcaaataaatgatatatataacttgaatttgcctgctgttagataatttaacaactaattaactgtaagtaatctcagttccttaccgctagaaagattaacatttaatgttctgacacattcagaatttgttatgtacgtcctttacattacacccaatactttatctttcaaaatgagtttctcacgttaaaaaaaaaaaaaaaaaagttgcatccaacagactttaatttgtacaaaatgcgttttgattattaaattctaaaaaattaacctacctgattgttttttttaaaaaaaaaatatgttcaaccaaattctattgaagattaaaaaaaaagtagacaaaaaacataaaagaaaataacattctggactagtaaaatgaattattggaccgtaaaaattagcttattacagttcagaaatattttatcctctatgcagtaggcagaggtattgtctatttaaagaagcaaatcctcgatgaggaaagtgcacaagtgagtttgttgatcgagataactagaaacagtgtgggatccggatcc

SEQ ID NO: 6 - DNA, 2081 bp, Glycine max
atattcaaggttatcaaattagaaagtttacgcagactcgtaagagttttatagactcgactcgtagattcaactcgtagactcgtaagagtctacttcatataaaaataataacaaaatatttgtaaaaaacatactaattaaacatttcaaatatataataaagcaaaacagtaaatcataaagttccgaatatttaaaccaagtctagtaataatacattacttgtagaggttatagtagtggtagatcattctcatcgagagtttgatattattagagaacaagagatttgatattattaaatgtgagaattttgtatttgagaataacatgctcaatgaagatatgttgaatgctgaacacacataaaacaataaaaaaatgacttacattttcgtttaatatttttaacttgctaactcgttgactcggtgataaacttgagagtctatcgagtttactcagaatttatagagtttactagaatctactaaaaaaaagtttactcaagagtcaactcggagaggacaagtggactcgtgaactcgtaagaattaacggattaactctagagtttgataagcatgatcatatcttttaagtaattgtatgcaaaattgatgtgttagatttatgtcacaagatttattctttatatttatatctatattctatatttcaatactaggggatcggaaaaaagaatttaattttatatcgttgctgataaagaaaattgtagacgtactgaatagaagattgatatttagaggtgtcaacgcattctaaatagtcggttatgttaaaatccataatatataagatatctatatatcaaatatcttttatttctaaattgctaccttttctgaaagatgctactaagttttataataaaattagcataaaataaaattttcacctcccaattttaaaccgaactaacactaagttagcacttttaaaatataatttgaaataagttctatcaaaaagttatgttaaatatatttaaaaaaaaaatttactatctacaatagtcttactcaagtaaagtgagttatttttaatattaatagagaatatctgtggattatactaaaaaaatattaattttagtttgattcgcattgatccaattccaaccaaggaactgatgactccgtttaacaacgccttgttttttcaaagattaattttattatttatttattatatcacatattatatttaacgttttctctctttattttttattttcttttacaatagtaaacaattgggattgaacataaatttttatacatctatataccccgaactctatttttattaggctgacactaacgagttggatctcaatcacctctattatttatttcttcatattcttttcgtataaactataaagggatatttaggtacaaataaaaaaaaaaccctagaaccttcaagatgttgtagttgaacgcatgtatcacgtgcattaattagtctctatctatccatattttaggtcacgagagtggaccccgaaataaattgcaattattattttaaaaattatgctaaattaagaacatttatttatacattaatgcgtttttattgttaaattctaaaaattagcctacttaatatattttaaaaaataaaattatgtttaatctaatttctgttgtttatactaaaaataatgatttctgttgaagagaaaaaagagacaaaaaaaaaaataacatttaggaatactacaataaattattggaccgttaaaatcagcttacagctcactaaggttttgtcctcaagccaaataattgaggagtaagactatgaatatcaggaatagtttatttaataaaagaatttgtgtttgatttttaatctagtaaagtaagagaaacttgtgatttcttacacaggataacacttttgatttaattttgagataaaagtgatacgtgtgaatctaggaagaggcgttgcctatttaaagaagctaaccctccacaaggaattaaggtgcaagagtttgttgtgagctataagctagtttatcgtgaggagaaggatcc

SEQ ID NO: 7 - DNA, 1506 bp, Cicer arietinum
ttaagattatatgttgattatatatatattttgtatatttaagattagatgaagtaggtgatttaaattttgtaccacatatatttaatttatttaatctatttttgtttatattatgagataagatagaaagaactttgttagtccatcataactattaatgagtacgactttaaaataattatgataaaaataaaaaatctctaataatataataattatgattgaatgacaatacaaaaaacttttatgctgataatgtacatgaattaaattatttattatttaatagaaaaataattgtttttaaaaaactcttaattatttactatcttattttatctactttttttctacaatatattactaaaataataattaatattacagtgaacttttgaatattaatttttctacaaatccgacaattaaaaaagaattgatggaacatattataaaatagaaaattaattaattttattattctatttcactttgcaaagaaaattattctatttcaatatttttccttttaataaaaaagatagttaattccaataaattatataataaaaataaaattttaagaaagaaaagcgacaaaaaatgggggagggtgggattgtgttaaatctgtcactcagtactgagagaaaacggctataaataacacgaaagccattccatattgctcgcactctgttctctgcttcaatcacctcccttaagaagaatcagagaactagtctcctacacttaaccgaaaggtacgagactcatttctatctccctcttttactttcttctatttttcactttatgtatatgttaccgtcatgctgaatttttcattttccgtctttccattttcctccccccctaaataaccgtcaattcaacttcattttccactctagatccactgttaaactcaacgttcaactttcgtcaacacttttagttatagtactgtttcacaacgtgtagatccaccggatctgaaacttctaatctggtagttcatcaaatcgaagttacaaaacccgtgattttcgttaattgacttaattcacacttagatccaatgttttaactgtctttgtgcttattgaactcacttagttacttactttttcgtgcttttagtttatgctaatcattttccacttcaattaatttctcgcttatttttcgcgtgtttttaactgttgagtagcttataattaaatcttgttttatcatcaaatcattgttacaaacacattaatttttgtttaattcacactctattttcgagatcgagtgcttttgaagtaacctagttgcttattctacagtctctaactaattaattaattaatttccacttcaattagttccgtttgatttgtgctttgaacagttaataaatagcttggaatctgaatcttgtaaccgaagaattacaatattattttcctattgttttttaaatctggagttgattcgatttacagaaaaggatcc

SEQ ID NO: 8 - DNA, 1506 bp, Cicer arietinum
tattttaaaatttaaagttgttacatatatttgaagcacaaatactcactttcattaaaccaaaaactctaacacgatcatatagaattataagtttactgcatataataaaactatcacattcaaatatcataatagaactataagtctattcatatttaaataacatgtttgtatctaaaaagtgtttataaaatgacaacataaaatagtggtttatccttgaaactagtcctcctattccacataatttgtaatagattgaaataactgaatgaattgctaattaatccaaattcatagaactataagaagaaacaaaacagtgcaatttggagtccaacttgtgagcaaacatcgaacccaacttggaactccaatgaagagtggccataacatgactctttgtccgttagtatctcctatcacatattaatttgtttgtcatttttcttaatcttattctaataaagtaaacaatttattttaaaaaataaatagaatattatcaattattgattaataaattaaagatagatattatagttacatatgagcaaacaatgaatattgaaaaataatttaaattttttcttcattttatactctatattttctagtcactttagacaaatttgtttttttttgttatccatgttgtccacatatttaatctatattaatttttgttttgtaatttttatactttctacctatgaatctgtaaaagttgaataatttctagaagttattattctacatccaaattatcttatattataaataacattaccagttaaacagatttttcctaataaaaaagaaggttaaaataaatatatcaaactaagaaaaaagtagtcctattcttattaaaaaccaattaaggaaagtgtagaatttcatagaattttattgtccattgatataatttgaaaacccgaagacttaatccatttaagatttcacaaaattaaagtccaacactcaaattacaaaagaaaaaaaaaataaaccttaatttgagttctgaaattgtaacttcgatcaatttagaattttaaatttataaaatattaagttactcttttacattttttaacccattagcaaaaagttccataagatagtttgatagaaaaactattatgagtgattttaaacaatttaagaaaacaacttataatttgaagaacaaaaatagtagtttacttggaaaaatatgagtgagtgagagtgatatcatgattggacttggaagcacaaaaacaaagcatgaattacagccattcattgaatgaaaatgctatccaaaatctaatccttacttggttgggctcatgagtcacaaccacacatccacgttgacacctcttactcgatcacttcactttatagtacataataataacattacatgtaactaagaatttgtttcccttgcaaagctcaatactacaacttcaaaaaaatcactttttctttcttgtagacttaatcggatcc

SEQ ID NO: 9 - DNA, 1506 bp, Cicer arietinum
atactactccaagagacactaatgtctaggtagaacactttttctagacatatttacccttaagatcaaacaacaattttttttctaaatataattttaaatttaaatctactgtaatatgtttttttgactatattatacttttgcctcttaagttatttttaagtttcattttaattctttaagtttttttcattttattttagtatggtaaattacaaacgttaaacaatttgatctcttaaattataatattagatattattgaaaaaaaaactaaagtatataacataacattagtactaaaatatttaatgtttgcaatttaaaagactaaaatataataaaaatatgttgagtgatcaaaattattaatatttacaaattacgagatcaaaattaaacaaaataacttaaaaaatcaaaattgcaatttagtgtacttttgaaaaataaatattagtaaattaaattatattaattttcaaaatcaaatcattacccataattccaagaaaataaaagggactccaaaagtaactacaaatttaggtatgactttttttgtgtacgaatttattgttagacaccttttttttctttctaaatttaaatttatagttaaaacatataccactgatatcctaattgaaatttaaataaattcaaataatatatattataaaattaaacacaaactaaatttggaaccgtttttaaaaccttctaatgacgttaatatgattttatctaaaaaccaaatggttaaaatattttaatataattttatttaaatttaattagaaaaataaaatctaaatttatattgttagaatttttttattaaaattttagtttatctctttctcttttacaaaacaaacatgattttgaagaagaaaatagtatattgttataatttaataaaaaattaaattttatctaaataagttaataaatgataaaaaaaagttatttaaatatcctctatttacttttataaaataaattactatatatatatattatatattttgtaaaacaaaaatatttttttataaattatataatcaatttatcaaatgagacgtaaaaaataaattattgttattttagaaaaaaatcattattgttcttattcagaaaaaaaacttatcaaatcgaataaaataaaaaagaacaaatgaaataaaattaataaaaaagaccaaatcaaaattgaagaaaagcaagccatgctcccgatgcaagtggacagttgagagtcaagtgtactaacaaataacagcaatgatgtaacattattattattattttaatttagtcattatttagtttgtaaatagaaatagaaggatctagaaaagttgaaaccctagctaacacttactatataactcttcgcattctctttcacactccattctcattttcgcttcttctctgcaactctctctctcatttctaaatccctaattcgactcagaaattagatcggcaggatcc

SEQ ID NO: 10 - DNA, 1506 bp, Cicer arietinum
caattctataataaataaaacattatattcgagttttttttgtttattgcacagtaaaaaaaaaaactaaacaataatacattagaacaaaaattattactttgccttcataattttttgttaatactatcggttgttcagtcaaatttgatttatttttatataattggagattgaaatataatttattttcaccatttttaccttgtttagctttaattaaggtcttgtacccaaaaaaactttaattaaggtcttgttaaaaaacatcacttaaggtttttcctccttttaattatcatatttttctttattattaaggctaaaaaaaaaaaccaataacgcactcttcaaatatacttcatttgactttcactctagtatttttaatcattggttatggtgtgtgttggtttgaggagaatgaagaagagacaacgaattattttaaatgtaaattgtttggttcaattatttagatgtgaggggagagagaaaagagaagagagcaaaatccctctaaaaccaattttaactcctcttcaaaagtgaggagatttgaagagaggaaaagtaatcatatatagaataaaataatttattgtttaatgtgatcttagatatgcaattttacattttgccttctttaaaatcctatttaaccaaatacaaaaaaaaaaaaaaaataataataataataataattcaccctctcctccataaaatctctctccttgatagggtcatttacctcttctcatatcctgggatcataattcccacttgagccaattttgtaatttagcagagatgaacttccaccttgtattcttgaggttgagagtctttctttgcctgtttgtcttttgcaccttcatatttatgtaatttttttcctccaacgtatagatgtaataccaattttaaaagataaaatatttaaatatgtttttaattcttgtagtgtgttttagcctcacatttgtttcttttcaaagagtaaaagcacacaatatttgcatttttttagagactaaaatccaataaaatttgtataatgaccaaaattaaattttatcaattttctagaaatcaactgtaaattttatttttaaaaaatgtaatgagcaatggggttgcactatagcaccagccaaaatgtccaaaccactcttccaagtttcagtttttgtttctcaagtttggtggacctaattttccttggctcacattcaccctctccaaaacatctaagcaagcaaaattatgtatccacatggcacaaagagagccacattattagtacaaaatatcaggttgagaatccacatccaaaatcaccaccaatgagatgttgctgactagtgcatatcctaatccacaacatcttatgatttccaccgtgtttactctctacgcttctataaaccattgcagcctttccttattcttcacaacactcactagctagtagcaacaaagggaacaaacaacacaatcggatcc

SEQ ID NO: 11 - DNA, 1506 bp, Cicer arietinum
gttattgtttcactttgcaattaggaatgacaaagtacttacatccgttctcactttcaagtctcgactcccttgctcgttccataacactactgcaatctattggtggaaagctctattttgtgacttgcgtaaaagtttgtggccacgatcgatctgtttgagcggtttatagagagcaatgcgatcaaaatatttaagtaatttactaatttgacacttaatgtgcgaagtaataattacatggttagattgttgacaagataatgactcgattcagcagctgaattgatgaaattggtgtttcaaaaacaaacaaaaagttgaggtatcatgacactaagtttgatatgtggcatcaatgaattttgatacatatacttgtcttataagagactacttttatgagtttttcaagtgtcctctaaaccatttaggccagaaaatatgtgtgattattgcaggttatagtgggtctgaatgcctgcaataacaatttattttattgattaagaaatgggttttttacaaaaattagatacatttttgtaaaagtgcgttaagcaatagatcattttcattttatcgaagttctaaattatagcttcatattttgtttttttaattgttaattcaagttggcatccattttagatttattgaacacgtcaaataattatcaaaatcaattttatagttttaaatttacttttacctcaacacaattaatttaaacatgccctaaatttagttcttgattgagtgtatggataaaaccaagtgtttgggagattaggctgatattctcgtgaactgcttcatttacaaaataaatatactatacttttgggagcaacttatactggaagtgaaaaaggaatttaccactgtcaaaaaaaaaagaaagagaaatctaccacaatttaaacattaataattaacgggtgagaaagtaacaatagttaagattattaactaaaagaagaagaatgacttaaccattaatatatggatgatccttggtcacttatttttctcaatattggtcattaagttcaaattttgtgacgttggggagagttttggttgaaatattccacgtggatgtagaatagagggtaataaaaatattaatggcaaaaattatccacaagtgaggtagaggtaagattacattgatagtcctatattttgtgtaccaacaaaaacgtaacagcaatgtggggaataacataagtactcttatccgattcatcatattacgtgtataaacatggttggagaacaagatcgtaatctcatccttactatattgtgtcatacaatatccaccactacaacttccacgttgcttgtccttcagcttagaggtagtggcttagcttccttcatatcttcctccacctatcattcattcttaaccatctaacatttttttaataagcaaaaaagaagtctattgctgtagttagttgtaaaggtaacaaaggaggggaaaacggatcc

SEQ ID NO: 12 - DNA, 1506 bp, Cicer arietinum
tattaaatttaaaagtaaatattaataattaattttaaaaaatatgtatgatttaatgacaagggtagttttatcattttatataatattataaattattatccaaaattaaaattaaaaatataattattaattttaatgtaattttttaacaagggtagtttttcgatttacatataatatatattatatattcatactttaaaaagtttgaaagtgtcgatgaatatttttaatataaaattattttattatttatttaatattttttgttaatttataatatttaaaaatattttaagttaatttgaaaaaatattatattattgttacacaatagtgattttgtcatttaaatatgatatattttatatcatgtcatgatgaaatatgtactaaatgcatgacatgaaaaaataatgttatataaatataaatataaatagaaaaagaatttgaaattaataaaaataaacatatcaataattaattttataaatatatataatttttattacaaggtaattttttaatttacatattctaaaaaattaaaccaattggtattgaatatttaaaaataaaattattattaaaatttacaaaaatatgaatatgaattaaatttttttaacaataaatataattctttttgtagatatagtttcattatttacttataatatatatcatatctatatttaatttgtctaacatatataatagaattattttaattagtcattactttttaaattgaaattatttgtttatgagagttaatggattttgtcaaactaattaattttctctgtatttatttatttataaaaattaaagatatttccaattaattaatttgaataaaatatatgtaattatatcacaaaagtgattttgttatatatttttaatcttaaaagtgatagtaaatttcaccttaataaacttaaatataataatatccacatttattatttaaatatgatatatataccaaaatataatatatattaataaaaaaatacaatataataaataatgttctacactattattttttctttcttaaaaccacaatttttttggagctaggatgttttttattctatctcttaactctataaattacgtaatttgggttggactttattatattcaatttgtcatagttcttgcttcggctttccctctcttcactgttctctcatctcttccctccgttttttctccttcacttttcttcattcattctcctctaccttcctccaaccatgaacgcatccattatcggtttgttttctatttttcttcttccgatttctctatctcccaatcccctcgccattcctgcaccttcattccgttcacataattgtcacgctaatatccattaaaataaaaaccattgccctaatgtttatacgtcaatttaatttaatttcgtattgtttttattttgattctaagttctgacattatggttatttataatttatcggatcc

SEQ ID NO: 13 - DNA, 1535 bp, Cicer arietinum
ttcagaactttttactaccttgaatttccactccactggctagcttcaaagtcctagatgcttcacataaaaaataaaaatcacatatcgcaatgcactgaaccttttcaattcacaaaataagaatatatatataaaaagaaaaagacagacaaaccgtatgacttctggttcattcatgtctggcataattggtgcagccaaagcaggataagcataacataacatgtggttaatactcaaaaactgaagaatcagccttcttgaataacatcttttcactattaatgtagaaattttgttgttgaagctctttgcatcctccatgcacctggattcatgtggggcttgaagcaaattgttaagagaaacgcttctcaagaagcatgataacttttttttaggagaaactattttgaattgaaacatatcaattcactgttcagaaaaagaaaaatagtagcatattataatcaaaattatccttgcgctacctaaattttccaacaacaaaaaaatggctgaagttttcataaaaatataaaacacggtaaaattaaagtacccttaaagagtaaaaatattatgagggatactacagatttttatttatataatgcaattacaataatctgatagcaaatgctacacgaaaaaataataatgtttgtggaaaataaatgtaaacataagcaagtaaaagaacaatattgaaatattatacaataagcataaagatgaaggcagaaagtgaaataattaattcagcacaaataaatataaaaaaagtgaaataatggacataattgtttaagaaaattgaattgaattaaattaaattagaggggaaacagtttataccttggagaagcatagacggaggcagcacaaattggtggaagtgagagaaatgataaaattcccattctcaatttggaagttcacaattgagggccaacctcaaaatcgaatactacaccattatctctatctatttatctatgtatcaccaaattcatagtcacataagctactcatttatgattttggacccacgcgagaccccactttcccttttaagtctaagaatatttttataaagaaatgataaatttatgttaattaaacaaatttcagagagtattaatttaatagttgtatttataaaaattaaagagaattataaaataatgacgaggaatatacgtaataattgaatgtaataagaaaaagagcgtactcgtataataatgtgaactatgcaatgagattcccattatgatttcggcccaagcctgccttactgtcttaaagtaaattcgggtgtccgtcacagaaggatctagaagtcaagtctttcctaacaagtatacgttttcgttatcgaaggatctagaaccctgcttaacttctttctatataaccactcatttttctaacaactccaaattcatttctaaaccctaactacttttgtagatttcactttcgtctcacgttctctactcttcaattcgatctattctcacggatcc

SEQ ID NO: 14 - DNA, 1041 bp, Cicer arietinum
tggagaagatgatggaattgtccatttattttttttatgtacaattgtattcaactaagaactatatcagaggtgtcaacgtaattgagattccacaatcttatagttaaaaaaacatatatgttttaatatactggatgatttgcattacgaatgagtttgtgtttatgaatacgattttttgtgatcaaagtactggattgaacgcaattaaaatgtcatacatgcaaaacatcttggtcgttggatgctggttcatattccaaatttttatttaaaatataatttggtaatgttgaaatataaactattcgatcttgatcgacggccaagatatctaactgcactattttgtctgcaatatttttctaatggcattgattgtatcatttttaatcatcaagttacttatggcaattgaagttacaagccttgtatctttctaaattaaatttaacaaatcattcatccgtgacttattcaaattcttattccacactacaagtccaccaaatggtcccaacagaatatactaaaaaaagtattttcatcaaaactgtaacatctttcaaccgttcgatttagtcgacaactgatatcatttcactgtaatctgcactgtctaatcttaaatcaacggctgaaattaattacagcgtaaactacatgattgtactgcagcaggcaattcagatcccaaaatgaatggtagaaaaaaacttgaagaaacagataaaacatgttatccaaacacaagcaaccaaccagatcagaatctaaccacatcagcagcatcatcacccatgtatcatattctaaccaatcaaaacaaaacattggatttcacatctttaacataaaccaatcaaaaaccatgatcatgaatctgacgtgtcatgtctacatccactaaaccatgcatcatatacaacctcattttcttcataatttttcaacacttccattgtgtgaactacaacacattcatttcatccaccttagtgtatttttttttgaaggggtttcctaaataacggatcc

SEQ ID NO: 15 - DNA, 1500 bp, Phaseolus vulgaris
taaaaatgtttaataaatttactataacaaaaattgaaaaagtggatacaatattaactgtaaaatattaatttgattttttttagttttactgatcatttaattaaaaattgaataaaatcaaatcacaaaagttgataatagagagacaccattttgaagagaaggagaaagagaaagagaaagagagagatgaaatatcgttttattaagtaaatgtataaaaaaacttttcacacattggaattaagaaatgcatatatctatacccacctatataaagggttcttcgttataattattttttagtagggattcttcattataattattttttagtgtacataattttttttctgattataaattaaacattttgtcattttatatgaattatttaaaaataaaattattttttaattttttaaaattaatattaatgtacattttttatacgcaaattaactcttccacttttttcttaatttcttattttaaagtaacaatagaaagaaagataaagcatactctcacccatttttacacaaaattccacatatcaaaataaaataacctctacttctaatcatctcttcacttcattaaagcagacgtgaacatcatcacactcaaaactgtcaatgaaagaaggaaaaagtttaaaaaagaatataaacttaaactagagaaaaagaaaagaaaaaattgtttaattaatttatcatttttcttttatatattattttttttttcaattttaactcatccaaaattaattatatatgtatttaaatttctatcatcataaaaattatacaaaacacaataacacctattttcgctaatctatatataaagtttacataatctatattaagtatgttaataaaataatatgtctttttgtattgtttatacataacaaattatgaatattttattaaaataataaataaaattattgtttttacttattttttttccaataaaataattatatttttctcctctttttcttcatctgaccaaacgcaccgtcagtgaccgacaaaacatacagtaagttgttgggttggattcatccatttgcgtgccgccgctgacaccgtggattgtgcgaaacagttactgcaacttacttttacttgggaacagtgatgattgacacgtgtagcattactgcagctcttcacattaattagccatgcaagcaatcagagacccagtgcaaatcattgaattatcttccaaacctcatgccaagtacccaacgacaatgtcaccatccagcacagaagcacatggtcattcaaatcagcccacccaccaaaatcaacccacttcaattctcataaagcaccatcctcatcttctctcccactcaactatataaacaccacacctcatcacttctcttcaccacccacatcacaaagcaacaccaagaagatatcgtataccacttctctccccttttagagaaacattctttctgcgact

SEQ ID NO: 16 - DNA, 1569 bp, Glycine max
aattttttggaaatatgtatgttttgagaatttgaatttcggaaatagtatgttataacataatttaagaatttagatacaacatttccaatgaaaattcttaaaaggcttcttaagttaccactttttattggaataaatctatatggtaaaaaaaagtttattaaagataaaatttgtatcttctataacaaactgttttttagtaataaaaaataatatttatttgtcacctaatagcatcataattaagtgaaaacaaaaaatataaaagtgtcaattttttaaagttttttaggattttattattagagtaaaattatgtatgaatttcttataataacatacaatatggaaaccacaacaagtactatgaaaatcataaaaaagtaacttaaaaacatattagatattgtttgaaaattcttcagtttacttaaaaacatctataaaatttccatcattttaaaaaatttggattattcaacatccaaacaaatcatttggcattttaaattcatttaaaaatatcaaattcctcttaaatttttacctaaacaatattaaaattcaattatattatttttttcatttagaagactcttttgcttaaagggactaaatacaatttcacacggactaacactatgttggtggtttgatattcataatgcttaacttaatttattatatatatatatatatatatatatatatatatatatatatatatatatatatatatatatatatattttcaataactttggcccgctaactttgtttgacacaagactaagttagactatcaaatccaattgaagaaagaaaaatgtttgttggacatcccttatgttaatatatacttagagagaaaaaataataaaaactaaataaatacaagagtgattatattttgtcaacaaggaattttaaacagtcaattaacatttttttttattttcctcttttaagcacatgagatcatcaatcaaattaattattttttttctctttaagttatataaaactcaagattgtttaagaaatattttttaaaatataataagtactcaatgtgacaaaagagagataatgacaaatataatgtataagtgagatgtttaaaagatcactactctcgaaataaaattcagtctaattcaatctagcccaaatggactagataaagcaactcaatttgacaactctagccgtgctaaccaagaattggcaattacaaaatatccaaaattgaaaccattttgatatacaaattaaagaaatccgtgcaataaagtggcctacactacaacaagaaagcagaagaagaggtaacgaagtcgcaagtggttgcatgtaatgtaagtaacaccggcctatagctcacccaccaatataataaacccccatcaattttcataaattcatctaacgtcgccactcatttccaatctatccactcatctatataaacaccacactacatcacttgttctcaccacattccaaaacacaaacacatacattgtagtatcacttttgtctcggaatttgttcttttgagtt

SEQ ID NO: 17 - DNA, 1584 bp, Medicago truncatula
ctcaaacggtcttaagctcaaaaagcataggtgtaaaaagacatgtgagaacaagagagtacaaatatagggtccaaacccatatgatgattctatggccctattagtaacaggcctgtggcaattcaaattttttcttttttttgtttgtgaaatggtgttttttattttgaaatgggctgggccgtggtagtttttccgggctagccctatccaatctagactgtagcttttctgacatccaatatgtcttagaaacacaaataaatgtctgaaaaacccaacaattgttagctatcgaggatattgtagtattttatttatatttttaagaagtttatgatgttaaaaaagcaattgtctccaatctagtaattgttcacaaacacgtaaaaagaaatacttgaacggtgtgtaactaacattctctactaaatgatagttacaaaagtttgaaatgttttagtttcttttattgcaatctattatccatatccatcttccctcagaaccaaaacaaatctacatagttattaaccttacatgagtttttggtcgtccccaaacacacacttaactttttttttttttttgaaagggcacacacttaactattaaatttcatgaatgaatctaccacattaatccaaaaaattatactcttctagtaagtgattaaattatcctattgtgcatgaatattcttactagcttagcttagtcatagtttactattcttatattaccgacataccaatcactattttgtggtgaaattattgaaggtctatgtccatatatgacacacatgtgctaacgtgtcaatattcagacggtgaagaaaattgaagatgtgaccacaaggtttgtcattacttatgtcctatagttttgcactatgctaattttttttagtcctatagttttaaactatgctaattttttttaactcgatatccaatcaaaaaatcgattaattcgacaggatcaatctcaccatctatttgtaggggctccgttttaagccaaagtaaagttttgtatggacttgtccatcgaaattgtgaccagaggaaattgaatccgaacctttgagaggagcacactctaagatcccaagctaactactatgccgacccaaataggttatgcactatgctaatgatcttgttcattcaacaaatcataaagttgaaaactccaactttagatttacctcaaattaactctctaatagctttagaaaatgcatttatcgacatcaatgtagtgacattgtagtgcagaagtaccaagctgtagtggtaaaggagaacatatactttaatggttatcatctccaccacacactcatcaatcacattatgatattacttcaataatgcacatggttgtgaatctcaaccacactctatctgccacgtttttgaactttagcaatcaaccacatatttcatcacaccttatcgtctttattaaccattgaatttgcaacttgcaaaccaatccaacacacctcaattggagactgttcttccctcacccctcacttgaaacatctcattctttcctttttacacaaac

SEQ ID NO: 18 - DNA, 1120 bp, Cicer arietinum
gtgactggtgactggtgactggtgactggtgactggtgactggtgactggtgactggtgactggtgactggtgactggtgactggtgactggtgactggtgactggtgactgggggctcgtgagtcgggactggtgactgggtgcaggccagccagccagcctgcttgcctggtgactagggacttgtgactgggtgctggccagccatccaaccagcaggcctgcttgcctggcgactagggacttgtgactgggtgctggccagccagcctgcttgcctatgagccttccctagtcacagactgtcgccgacggagaataagtctaagtcatgcaccgtgactgacacagtttggttcgagtgacccacgagccttccctagtcacggacgttgaataagtttttgccataaaatgtccatgactactaaggcctacgtgcccttgatttgttcgagtgtttccaccgcaagcgtgtatcatcagtacggttatcatgtgcatgacctagtatgcgcataggtcgggacaaattgccttaggacgacacgcgcccaatccctcgatggagggcatagccctgggcctattttggtatttgctaaacccttaatggcacatgtacaagaaggttactcatgggtggggagggacgaatcgaagcgactaaggctgaatctcagtggatcgtggcagcaaagccactatgccacttacaatacctttctacccgccgctcaataggaattgcgtttcaaggtgtcccgcaaggctcatccgccttacgaggtccaccaacgacacgtgcctctggggggccaaggccccctactgctggtcggcaagcgaacgacgggcacacgcatcgcttctagcccggattctgacttagaggcgttcagtcataatccaacgcacggtagtttcgcgccactggcttttcaaccaagcgcgatgaccaattgtgtgaatcaacggttcctctcgtactaggttgaattactatcgcgacactgtcatcagtagggtaaaactaacctgtctcacgacggtctaaacccagctcacgttccctattggtgggtgaacaatccaacacttggtagattctgcttcaca

SEQ ID NO: 19 - DNA, 1652 bp, Medicago truncatula
tgatttataattgactgcgaccctttttgaagaacataaaatatattatccgcatatactaaaagaccaataatgcttcaattattttctttttcattgtaattgatgcttcagttttgtttcaaggcttatatttacacaaaaagtcatgtgataggattaactttaattatagtttatgtaaaaaaatctttttccgatatatagtagaaaagatgttaaaaaacaggcttaaggtaatttcaacatgtagaaagaacaccgttatatgtagtatgaagagtagatcagatgaagaagagtatatgcaggggataatttagaaaaactatgagagaaactattaaaaaagatttagaggttaatcaaaatatggtttaggatagaatattatgatgtaatttgattcatatagccaaccctatttagtaggatacaacttgtctgttcttgtaaacaattttaatttggacgttcatttgattttttttattgaagtggacataaaatgattgattaattcgacgatcaattccactatccactagaggggaccaattgaaacctaattaactctgttaatgaagatttgtcaccatattatatattattatttattggtggaacgttatgcggtacattcataaatagaacgcttttgctacattaacttcttaaaaaggataaatttatgatcatttttatgaaccaaaactatttgtcgataattatgtagttaatgagaggttataagcattatacttttttttatatatttttttttaatatacacttttatgattaataaaaatttattataagaatggtaatataatcgagtaacttcatttcaagaatggtagaatgtgtcaaggttcaaattttgggtggtttcagagtagtttaagacaaagttcgaaaatgaaaaaatgagaaagaatctaaggctgcatgtatataagtgtatttcagattatataagtcgaaactttttttttgtttggattatatatttttataatcaaacggatgtaaaattgaaaaaatggagggttgaaaaaagaaagaaaaaaaaaaactattgctgcgtaaaaagtgggaaaataaattctttataataaacatgtacaataataaaacatgtacaaaataatcattttgtactaactgctacttggaaaagctgtgataaacaacaaaaaaactacaaagttattccaatcttgtttcttttcactgctggccttttatttgttttgcaattggattggattgaactggagagaaaataaattcaaacttttttttaggaagaataaatttaaacttttattattgttaaaaaataatttgttgtgagtactactacaatattaatatataaatctttttttcttttaaattaaatgtgtctaacgataaaactcacacacacaatatgttgagaaatgaagaattacttatgagactataaatatgctttttaattttatcattttaggcaattatctcacaatgtctcaacaaattttaaatacgtccttcgtgaataatctcttgattttttaaagggcctaaagacattgacaggagaaagaaaactgctccactttatcatactacatagtattatatattagtgggaactaaga

SEQ ID NO: 20 - DNA, 1600 bp, Medicago truncatula
ggtcctaccggattatttttaggtgtttatatgcatagacagatgaatgggtgggcaaagagcgacccatgtacctccttctcacatcttagcattcaaaagcagacaatgcatggaacacgcttcaaactaaatcagaataattcttattgaacttttgaaaaatttaacatttttgtttcaacaattttttctttcgtttttaggcaaaattgttaaagttgttcttaaaccttaaaaaaatacgttaaaaataaaggttgtctataacaatgcttaaatgagttatcatttaataaaaaaatatgtgacatgattttaaacattgtagtaccttaatatattattttggcgtctggtaaataaataatattgaaaataatatagtgaagaaaacgtataaaaaattgttgtgtatgtacttttttttaaatattatagaaaatacgaaaatatttttataattgaaaatctttttaaccactaaactatttaaaaaaaaaaaaaaaaaaaaacaacgatcgaaagatgttattaccctgttcgaaaaacaaacaaaacacatggaaagatgcaccttatgaactcccattaaataatatagggttaattaagtaaacggtctttataaatattcacaattttgtttttagtccttataaaatgaaatcatactttttagtctctataaaatttttcatcagcatttttagtccctataaaaaatttcatcagcatttttagtctctatcaaaaatttccatcaacatttttagtccctaaaatgcttaaggaaaatgttacatcgagtaaaaagtgtgattttattttgtagggactaaaaacaaaactatgaatatttatagggcctaaaaacttaattaagccaataatatatactgaattttgtttcgtttatatcatattatccataacattatccatgcataacgtatgtgcatcagataatttttttttcttacaaaatatgggcatcaaataatattgaccacaaaaattgttataaactacccagttttgacataatatcatccataccagtagtagttttgaaatattgaaatccatgaattaatagattctctatgtataatttacgcatgagagttaaaaatgcgatcttatatagcgattcaacctcacttaatttaaattcttcatatgttttactctcttgatgaatgtgaaagattatctctctcatttataaattacatatgagatgaccaatttccattttttatttttttaaaaagttaaattaacatcggagagaatcgaatctgagacctcaataaaggaacacattctcataatcaaagtgggtttcaatactcatatttgatttccattttaaaacgagttgattttttttgttttcaataaaatatattttccaggaattttgtaggaccacatatatctttttgtaaaaaaaagaaaaagtttatcattttcattttcctatttttattttcacaaccttatagtcccaaaactatagagatattgtctatataaagaaccaaaccctcaacaacaataagcaaacaaagttacaagaaacatacacataaga

SEQ ID NO: 21 - DNA, 1542 bp, Medicago truncatula
accgaaggacgataagaatactcataccacctcaaattaaagaccaataactacttttttaaaaatattcaaaaagttacattaattaatgattgcattgaatttgatgttttttcaatatttttttttatgataaaaatcggagagagtactctcttgccaattcttttatttagttgggatctatgtatctccatgagatgagatagcagttatgaaccaccagtagtattctttcaatagtttgtcgctttcagccaattgcaaatataatcctacttcactttcatagcctccgatttttactattccctttcgtttgaaagatatctcatgttagtttgtccactatttttattgtgttaagacattcaaacgacagttatatgaaattttgattgtatttaaaattaaaattatagatattttgtatttttatattgtgataaacttcaattttattttccgatgacacctataatataacaaagggtaaaatatgcttttcatccctgcaattataggccgtttgatttttcatccatgagcttactaatcctcccattttgcccctgtaattactaatcatttgaaagttcgtccctctccccggataatcactgccacgtcatcaaattagcaaaatggcatgggaattgacaaaaatacccctgacttatttttaaaaatttaaaaatctgaaaataattttaaaaaattaaaaaaatataattttttgaaatatgaaaaaaatataaaattatataattttttaaaatctgaatatataatttcgaaaattattaaaaaatctgattttttttaaagatttttttaaaattcagaatattaatttcgaaaaaatctgattatatattttttaaaattatgtgaaaaattatttcattttttttcgaaaaagttattttcaaaatatttttataatttttttattttttaaaattattttcagattttttaatttataaaaacaattttcagaattttaaaaatagatcaggggtatttttgtcaattcccatgccattttgctaatttgatgatgtggcagtgattatctgggaagagggacgaactttcaaatgattagtaattacaagggcaaaatgggaggattagtaagctcagggatgaaaaatcaaacggcctataattgcagggatgaaaagcatattttaccctataacaaatatagtataaattcatatatttttaaatgacgtcaataatataacatttttataggcaattttgtaagagtataattgaaagtgaaaaaatcatcctaaaaccatgcatttttgttgtacatagtatagattatgaatagtatcaaaattaccaaacaatatttcaaaatttatagataagattgaatttatactcactagctaccttcccaactaccaagtaaaggtccttacatctccctctccttccctccattatatatagagcatacttcttactagttttctcatcgttcactaccaaatattacatttgaacagatattgaaca

SEQ ID NO: 22 - DNA, 1600 bp, Medicago truncatula
tccttgaaattgaaagccatatccttaattgctaggttgaatatcttttatatcccaagttcaaatatgaaattttccactttgtatttgtgagtttacaacggtgaacaagagaactatttctgaaaattgaattttgttcaatataggtcacaaaaggtctgtgcatggaccattcaccggccactcggagactctcaactttttagccaatagcaatggaccaattaaaggatcgcatgtagatatccaaaaacacattttttgtgacaacataaaataatcaatagttaataagtgagcaattgtttttatttgaaaggttcaaagagagcaatttattttataacgtaaaaaattacatcccttgataaattcaacatgcataacaaatccgttgctaaaacatcaattgttaatttatcatcaatgattagaatgccttagtatatcttgtagagtgagagcttactctttttctaaaacttttacaaacagaaaagtcaaaaagtccaaaaaagtgaaattggattagcaccaactaaccgaccactttcccataatatatatagaaaactaaacaaaaacaacaaacttatgacatgttaattcattgaaattattatatgacaccaacaaatttttgtactcactaattttcaaattccacaattctttagacactggccggtgctattagtggcccaaacctattcatgttttccacttctacttttttttaatgattatgattatttccttcacattatgccccaccaccaaacatcacctctaccactttcccctgctcacatggaggaatcattcattagcttccagacaattctgataaagcacacaaatgacagctaaggttgccactaaaagacttgattggatatgcatggatctgttgtacacataaccatattctctttatcccttatacataaccatattcaactaattcagagattaattttaacatttgtctaattagtcttgcattatagataagtttgtttttggttgttggcttaataagtctaatcacatctcattcgtttaatggagagatttcttgattcagtaatgaaaataaatttatgcataatgtaagatttatatcacttgtgcatcataaacttgacattaatttgatactggaatagtttacaactatcaacaaaattttcgtcgaaaaatcatttcgctatctttaatcatgaggctacctctcccaaccacattttttcaattacgagagtcgaatccagaatcttgcttaagaaacctaagtctaacttcacttttgaccaatgcaatgttgcttcatttgtaaaactaagaaattcattacgattattttcattgatttaacgattcagattaatcgactgtataatatactaaataataatataactataaccaacttattaaagaagccttataatataatacattagtaatagtatctaacaacttgatcagctataaatagaactcactagataagtcttttgtgttatattttcctcatttagaacatcttccttatcttattctctccactaggttctcctagaaacttctgaag

SEQ ID NO: 23 - DNA, 1544 bp, Medicago truncatula
agattgtataggaaggtgattttacgttcaaaacaaaaaactaaggaaaagtgagatcaaagattcggttgtcggatgcgttgagataaatatatatttagatacccaaagaaaaagttttacaaattacgttaagtcatcaagatttttcaaaagctactatttcaataaaattgtgacaattcaacgtatatctgacaacttatagctcttcgtaaattatttacattcttctatcaaaaactctatacaaattttaacaagctatatactaaaatagaattataaatttaatgcatgatttatcaaagtgagtttatcaaaatcatagtgatccatcataatcttgtaaaagctataaagtggaacttttacataatcgaaaatcgtgatatcttagtttgattccagtttttgaatttatagggttttgacaaacacaatcatcaaagttattaatatttcaaaatctataaataagataagatgattgttgtaccaaaaaaaaaaaaaagataagatgattgatcgaaattactttatttagctattgaaattgaaatgtttcacgcaaactaatgtgattgtttttttttttttttgttattacgccctacaattttttagactaatcagtgattcactcccaatttcaacgtctctttcattggcaaattttaacaatttaaacaagataaaggaaaactaaataatatttgggtttaaccctccaatttttacagaattactaatccgataatataaagtttggtggtgaaggaaatatagcatgactaagaatgtgttttatctaaaaatcaaatcaaattcttccaaataattggttttggaatggtgctcattaactattttaaccaaattatcttgttagaccatctctaataggagagtcttagacatttaaaatccggtatatattaggtatatcctttggagaattttttttttccaagtcttataagaccgatagtctcacttaagacctctaatcctaaacgggacccacactagtttaatattaaaaaattaaaatataatgatataaaattattgatagttgattagtaggtcctatttaaaaaatttgtgtgagatgctagttggaggaattgaatgcttactactattaaaaaatttataaattaatgatatgcgcatgtgtgacccatttaagtatccaaatatggaatgtctcaattgatactattactaatatattaatatattataatgtataaaatacatatagaaaaatgagaaaaatataatgtatagaaaaatagttgaaagagatacatctccaatcaaggtgatagaatgaatatattattattttatataccacgagaggtttggctggagagtgtttagcacgtggtgtataatgagtgactcaaaaagaaaagtctaaataaaaattaattatccaaatatcttgtatagtcctataaatagtgaacacttccatagaagtgagcaagccacaagcatttctctcattgtccaaatcaaaagtagcaaaa

SEQ ID NO: 24 - DNA, 1584 bp, Medicago truncatula
gcagaggagaaacccacattttcaactcaattatttatttgtaaaaaaaaactcacttttattattatttaaagtttatatcaatttttcaaaaaagaaacaactatgaaacacggtttgctgaattttttctcatcggatgatagtgggtccatcagataaacacatttttttaattattatcataagactctagatatcatgttgaataagagagaaaagagagatatgatgaaaatcgtgattgaattgcactacggaaggtttattatataggagttaagtagtttaaagactaaactagaatacatttactaaacggaatattagacataatattttatcttaagtaacttaaagtgtaagttaatatatatttacaacgtaaatatacttatttactatttattttaatatcaacaatattgtacaatgtgacattttctcatattacaataaggcccataaaacgttaagaatgactctggtcatgttactcttcatgtcgtggtccctcctccaaggacaagggattgtgcacctgagacggtctgacaacattactcttgttgccccgaccctaatttaccccttatcacttgcagatgttatcattgtaggtttgtcacctctaatccggtcaatatgttggtccatatctgaaacaatcgaaatgacatatacaaatcattaaaaatatgttttccacacatcccaaacaagtttacataatatagtacgatgaccaatctagtttgagttatataatttgaacatgtccaacacccccaaaaagaaaatcaatttaaggaacaaaatatcgaagaaatgcgaccttacatcatgtttagtggttgaaaaggcttgaaatatgatgattttatcaaaaaagaaagaggaatcacacaatatgtggtggttcagatgttgggtggtgtgagggtttttttgttttgcttgtatatacttatgattcacatcatataatcttaggttgtgttttttaggttcaaattacgggctcgtttgtttcggattatttttgaaaaaaatatttatctttaaaccaaaaaatcccttttattttttatttttgcagtgtttgtttcatatttttgaaaaatccattttgttaatccacgtaggtgctattttgttatcccaagaaattgaactagcgtgagtgttaattcctacgtaagttaatttatgccgcgtgacttaagtcgtgtaaaaaaacgaaattttaaatcatgttaattttacacttgcgttgattaaccagcatatggatatttttgaaatggctgctgggcgcggatgttttgtgccggatgatgagcggaattacttgtattattgttgtcaattgtagtggcccaagtaaaaaaaactgcatggacccaagacatagctccactcattttgtaccacgtgtagtagcactattgcggttttttctataaatgaagccatcttcaacaacgtgaagctacacctcaacaagtaaccacccttcttgagaaaaataactctcttcctttttttctctcgtgatttgttcattttcttcaagtaacaaaa

SEQ ID NO: 25 - DNA, 1500 bp, Medicago truncatula
cccttgctactccctcattcaagtctcaactcccaaagtcttcgaatgtttctttctatcttcaaaaattctctaaggtgttgtttgggagtttggaagggaggggctgactttgaggggtcggaaatataagaaaaatgaagaagtctttcacattttttgaaagagtattttttttaaaaatgataaattactgcttatgattaatatattttttaatcttaattatatgataccagcctaaattgaatttgaataattaatataaaccatccaaaatcatcttttaagccaattgagtttcccccaaattacgaaggttttgtagtccgaagaaaaataaaccccctaattcttcctcttttaaagtcttctatttcttccatttgatcatgtttttttccaaagcacttccctcattttcccgtcaaccttcccgccatagcctagggtgctccttcactcgtatttattgtgacccctctcacttaagtttgatttactgttttgtgaatgggatatttttgctacctctatggcaggaaaggtcagccttatgtctgataacccttcagtttgcttatttatcaaccaatttataacctacatattatttagtagccttttcttgttaggaatacaatgaagtacaaaaattgtttcccattagtttatgtttttacaaaaattgttgccaacttggggacatgactttgttctctacatttacgtataatttaacaaggttacaggagattgagttgaggctttcaatacgtggttcttaatttgaatgtgtagaagacttatgaaaggattgtctgtcctagaagaaccagtgagtagataagaactctcgtttggttatttgatgacccttttcggttatgtaattcaagtgagttttgtggattaattctcaagccgtgagtggtaaactcctgcggcatgcttcttaacattagtacagtctcaggccaataacttttgccttgttgcccaatgtttacagattgaagttaagcaatagatacgcaacaatttgagatgatatattttgtttgtattgtattattctatgacaaatatcagaacatctggttggctaaaataagagaccgtattaatagacggctgatattttcacttccaaacaaacatttttctttaagactaccaaaatatcaagattttattatgactattaaactaatgatacaaaatatacaagaatatttgcatgactaaattcaagtgagctagaaaggtttctaacaaaaatgaaatttctcttgaaataattagaaagtaataaaaataatatcttaagatgggtccaacgtggaagataacaagaggttgtgaaaagataagaagtatgcgtccagaaatattcaccaacattggttatatatcccgccattacattccctaaccccattaaattaaacctcatctttatcacaccaacaaaaaaaaaaaaaaacaatagctgcagtt

SEQ ID NO: 26 - DNA, 755 bp, Cicer arietinum
tgtcgggagggaagcggatgggggccggcgatgtgtctcggtcggatgtggaacagtttgtgcgggtccgccaatcgactcgggactacgaccgacgcagtttgtgatggtggcccaagcctgggctgttgaaatgctcgcggagacgtctacatcacgattgtggacggcagcgcgcgccgtttcggcgtgcttcggcacttgcgcgctcctggcgtcggcctgtgggctcccctatcgacccgtcttgaaacacggaccaaggagtctgacatgtgtgcgagtcaacgggcgagtaaacccgtaaggcgcaaggaagctgattggtgggagcctcttgtgggttgcaccgccgaccgaccccgatcttctgtgaagggttcgagtgagagcatacctgtcgggacccgaaagatggtgaactatgcctgagcggggcgaagccagaggaaactctggtggaggcccgcagcgatactgacgtgcaaatcgttcgtctgacttgggtataggggcgaaagactaatcgaaccgtctagtagctggttccctccgaagtttccctcaggatagctggagcccgagggcgagttctatcgggtaaagccaatgattagaggcatcgggggcgcaacgccctcgacctattctcaaactttaaataggtaggacggtgtggctgctttgttgagccatgcctaggaatcgagagctccaagtgggccatttttggtaagcagaactggcgatgcggg

SEQ ID NO: 27 - DNA, 1500 bp, Cajanus cajan
tatcactttatatgtttatatcaagttttgttaaaattgaacaacaacttgaaggtaatcaaagtgctcatattttaatatattttgaaatgtatatgttacgtcaattttattttacaagaatgaagtatcaaatcatctgtaattcatataaaattttgtatatatgatctaagagtaaaaaactttcaatctaacggttgaatttataaaattcacatttaatataaagttattgagcgatgtaatacttagtaaaagttacaccgatgttattataacccttctcttttaaaatattatttttataaattaaattttaacattttaaataaattattaattaacttaatatatttattcatcttaaatctaagatataaaattcaaaaaatgtatttatccttgttttattttctaattatcaaccattaataaaatactaatttaaaatcttatctagttatatcataatattttcatatcaagtttaaaattagatattagttatacaaaaatattaaaatcatatagtaaattattaataagattctagttaaataatttaaaataatttaaaataatcaattttatcacacaacaataaagaaatattaatagttaagaaaatacatttgtactcttcctattagaaaaagagaggatgttataattcttttttttttcttaagatatcacatctattctatcaataaaatttaaatataaaaatttatttaaaaataaagttatcataagttagaaatatatattataaatatggatcaaactaaataagaaattttaaaaaattcatatgctctaattaaaaagatgtctaaaaatccaatcgtaaaaaaaactctaaatattttttcacataaataactattggtattaattatgtaggaatgcatataaatatttttttattataaattaatgtcttgctcttacaaaaaaaaaatgggattgatccaaaacttcaatatttttttatttttttttattagctacaagtcatgtaagtgatataacccgataatgaaagctagaacagtatttgatatgaaaatttctacgaatttttttgaacatacgaatttgggttaattaattaattgtaattagcaaaatgtcaagttaaatagtatatacaattataaccaatagaatgcaagttcccctaatcatgacaatctaactcgccccgtgggtattcatttgtccgaacccactccgatttggacgggaaaaactcgtgtttatcaggttcgggttcgagtttttcccgatagataaaatcggggacgagggcgaggacggggatgtcaattctttcctgtacacgccctgcccacgcctcgcttaataatataattaaaaaattatttatatatatttaaataggtagtacggtgtggctgctttgttgagccactcactggaatcgatagctccaagtgggccatttttggtaagcagaactggcgatgcggg

SEQ ID NO: 28 - DNA, 1500 bp, Medicago truncatula
ttcaataattagattccttttaactagaaacctacatcaacagaaaaacaaacaactgcaataattaaagtcttgatataacatgataaaaagacttggactgcaagcaatggcagcaacaaaacaaaattacgatggctcggtcttaaccacctcatacattcctcctacatatttgaggagcgttattctttgctactccctccgtttaaataagtatcgctttaacaaaaaaaaattgttttaaaataaatgtcacttatattttttaagatagaattattattttacttcaaaatatattttaattaattgagtatacaatacttccaaaacattaaataaggttagtttaaaaaccttaaacaaaatcattttgaaacggagagggtggtatctagtaaaagcaatgagtatatgcataatatattgcttgatttggttaaatagtagggtgctcttttcccatctctcaacaatgaggggaacgactttgtgaaaaatgaaggagagaaggaaaaaaaaaattaacagtagtctcgtcaataggaggttaatttttctttgtaatacaaaatttctttaatatgttggaactcgaaaaaaatgcactagagtaaaaatttttaaggttttagagagcctgattgctttcatttatcatactcttaaacttaaaaatatattaatcattaacatttttttcattctctacacaaaaaaacactctcaaataaggtggaatattttcctctctttttctctcctcctcttctctcaaagcattcccctcccactccattcaaaatgtttgcaaaaatttaaataataaatataagtcttggaaaaactaaagttaaataacaataatactttcctgcaccatgtaaaaagtagaataaatagaacgtggccactaacctcaatttcggtcaaaactaaatgttatcaccaaactttaaaaataaaaataaaaacttacagtaaactaaaacaattcactcctcacactaggacactgccaaaaatatctgtaataatcaaagaatcctaataataatatatcatggggaatgaccttttcttattgtactcatgaagaccatgcaatcatagcttaacactagatttagttgttgttttactaaaacattagattcagttagagtatcatctaatctaatctaattaatttattctgaatgagtgtaaaaactctacgccgtcaaattccacctcgcttaaaaaaaaaatagtaacattttacaccagaacaattgaaccttccttagtttggagcataaagtgaattgtctgtttgtgatgagaatgcttaaacattactacttctttgacaatactgattaacgaactagtcttctagggccacattatctcatctaataattaagcttttacccctttaaaaacccattgaactcatctttgatttccacagatagcaacagctaaaaaataatcaaagtactcttaattgac

SEQ ID NO: 29 - DNA, 2311 bp, Medicago truncatula
ttaaggagaataagattgcagtatattgactaatatgtttcttcaaaaaaagtatattgacatatcttgtagatgtagacgagttgctgaaatcaacatttccaaaacctgcctaaatatgaatttcctcttctacacttgtatgaataagatatgaagtttttttgactggtgtgagcttgctgacaaattatagttctgaggttgagttgaacggcgatggaatgcgacatagactcaactaaaaaagatggaaatgatgaagttgatgttgttggcatgttcattataccacttactaatttttttccaagcaaattaggtattagctatcacactctaaagagaccagtgtgttctcattgtaacccgaaatgaaatttatgctcctttaatctagttttttttttttttttttatttagaagaatctgtgtttatgtgcacttttgattttcaagaatctgtgttcattttgatgttttaacaaaaaaatgtatttttttttttaagtcaaaggtaatttcaatgcctttatatttcctttgatttacttatctttcaacattacctgaatttttcaaaatttaataatcaaacccagactgtttcagccacaataataacattttatctgcaatcggagtgaccagctaataatagagtttaaaaattgtttgaaatgccacaccacctcaacgtgaaagcaacttatatagagaatcaggtttcccaaaccattacaacatgttattgcataaaattgtagataaattgcaaaatatacaaaaaataaaaaacaaaaaaggggcattcggagaatcgaactccggacctctcgcacccaaagcgagaatcataccactagaccaaatgccctgtttatgttaagtctttgtataaaacataatatatattaaaactcatatgtaccaacaaggcatagctgcatattagatttgttgtagtgggacataatataagctgtcatcataatggaatggttgggataggtaagactaagatagcaatgatgaatttttacgttcatgaatctattaatataaattttgtctattgaataattaagtttatataggctcattttgttgagttatattggtaaagttatacaaatgttgtgatatgctgacttcaatgatggtagagaatctagatcagacaacaaccattgcacgtaaaagttacaccacacccaactcatcattttatgttgttgcctaattacattgtttgtcacctttattttcttctcaactttatttaatcatgccatagttgttcatatttgtaatacaaaatatataaaaataagaaaaagaacagcatatagatgtttatacaattaccaaaaggttgttaagtttacaaaagatgttttagtttggtctgtcagtaacatgcatgacaacttcatccattcttgtaattcaagaaatgaatgtattctttgtttgaaaaagttagatccaagaatgaaagctagccagctggatgttgaatcataattcctaaggtgctggctggcagttgtgattttcaacctatgggtccccataattaacaaagtagctttctaataaaatttataatttattagaccaaaatatatgcaatttatattaataaacattttatgcactaatttaggacttgattagagaccaactattggtataaagagaaatggttgaatcatttttatttttgacaatttagttttttttataaataaaatggtaacatcccactaaaatcacatttctatgtaaaagttacgaatcatatcttcaagttataaacaaaaatcattctaagcgagaacaatgaaatatatcaaaatcaattttaagtcttcagaatcacttttgattcctttaaaagtgaatccaaacatgcaatcgaagcgatgctgccgatgcaacgatgatatattaaagctgctagcacaaaattactgtaccagcctaaacataggtacttactttaacagcataatcttgcaaagtgcaagttacacttattatttttccttagttttaaagcatttatttatttatattcacaacaaaaaataaaaataaaaagcgaaaagtatagttaggtgctgaaaatttgatcaaaatgattgtcactgacaatataattgttggcaaaactttactttagtgtccacaattttctttatgccacatttctcctccactttttatttttatttctctgctcttcataatctttttgttcatagacttgttgcaagttggtgaggtaagaagataaggaaaaga

SEQ ID NO: 30 - DNA, 1446 bp, Cicer arietinum
aaattaacaaatgcttgaatttatcgactttcagctctatgatttttggataattccatgagtaaagttgaagaaaatgtgtttgagaaatcggagaaagtgtgtttgagaagttggtaatagtgatttttagattaagatggagaaagtgtgtttgatgatgttggtatgaagtgtttatataggaaatcgcagggccaagtagcgatcctcacattcagccaacgtagtaggagatcgcatccccatattgcgaacccttaaggaggtcgcagggctaggaaacgacttctttgggaagtcgtagggacaggaaacgatctctttgggaggtcgtagggccaggtagagaccaattgatgaatcccagccgctcatttcttttaatcgatggctggggatgcttgatgatgaatcctcctaattttagttggaccaatcaaattgatctatttaatctttaatcttgaccatccaaattcatttatcaatggttgtggtttagtagaaaaatatcaatcctcatacttttagttggaccaatcaaattgatctattcaatctttaatcttgaccatccaatttcatttaccaatggttgtggatggttttgtaaaaaaatatgaatcctactaacttctataaatagaaaactagatcatactatatcaccaaaccttctatctcatcatacattctatcaattacataatctaggcttctcaacaagtcatttgtattgtttattacaatggttcaattgtcgatggagttgaagggaaaacatttgtaagtgattacaaaaaggcgttcaaagtacattctagcagcaaccttgctcgtctaaagaatttcattaagaaaaagttacagttcgacgacaacgtgacgataactgatatagtttatcgacaccttgtcttctttggtgaaaatacaaccagatttgaattgatgaaggtttgtgatgacaaagatgttcaactcatgtttgatgtttatgcacaatggtcacaacttggcaccattgagttatacattacatttgaaagtggtcctacttcaacaaatcaaccatcaacctcaaacatgcctaatcccaactacatctcatcacaacactatgaaccatactctagcccttattaacccggcgtatattctccatcacaacacttagaaccatactctagccaaagtgaacaaactcaacatgtccacaatgtctcccaacaacttcatcacaaatctcacccaacaagtccacaatgtgatcttccacttagtgtccattataatatcaatgaggaagaattaggtgattatagtgaagatgatgaaatatacttcgatgaatcatctggtgattctgacgatgatgatgttgtagaagattaggaagcagaagatcaacttatcccacctgtattcgatccaccattccac

SEQ ID NO: 31 - DNA, 1645 bp, Medicago truncatula
acattagaccagagactcctgattcagtcgggacgattatgatattcaattctttgagaacattccacgagcttccattgtttgatcttttgagagaagtattttaaaattttaatagttaatatcttatttgtgatttaatctattataaggtaatttaaatattgttattttaatttttgtttaaaataatttttcaataatcttaattgtgaatttcgtgaaaaaacccttaattatgagtaattttttattttttgaagaagtcagatgcaatatattaaagacggaaagctcatatcatgccacaatgaatcgagttatattttgttggaatttccttgtttggagttcttcatgctagtaacgagactttgagggtacacctttattccaagtaatgtctggatgaaaatcacgatcatgctaattactatgagttagaatatgataacacctttgatcgagtaaccaatatcaggattatatttccaaatccatttgtcagtaacattatcctgcacaacaaagctatctaacagtgaaatacattcaactaccaactccttccccaagcaaaaagtatcctccctccactttcaatatagaccctctgacccccaaccttccctccttatctcatctacataaatactattactaaaacttaactaaaagagacgattaaacttgaagcgcaaagaacctcattctatgcgaggatcattccaaatataagtgctttcaccattccccacttaatataatattctatatactacattacatgaactaaaaaagtgattggtatattatttttaaataatttagtggctagaaataacttcgttaaagaaataatatattaatcttgagtgataacataataacaattaaaaattacatttgaacagagtaattaatactaaataattaaaaagttaaaagtgacattcatttgagatagttttttttatcacttttgtaggacggaaaaataattaaatttaataatattgattttacaaatttatcctcaagtatttgtttactagaatttaccttcgaccatagataattagtttatatcctttttataatgtttattattctttttttaacaaatatacttattattcttgtttatagaaaaatatgtagtaactataagtatattgataaataaataaataaataattgacgaacctaaaatttttaaaaatatcttatgaaaaagaacaaaaatacattactcaaaggtgtcatgtatgtatgcatgtatgtaagcacttagatattaaaaattcacgtggtaggccacataaaatgtctttgtccacatccctaaaaccaagttttttagtcttgattagcctccaagagaccacactaccattaataataatagaatgaaaattttggtcctcagaaaaagaaaaagaaagcgcaacagcttatcacaagtgtgtacaatttttgtattttttagacatatcaacctcaaactcacgagtcttgacttttctactaccaaaagtcatgcaataaagttacacaatactagtaaggaaataccatatatactatagtcccaaaccaaaatttaatctcactcatctcattaagctaagtttagaaatcaaa

SEQ ID NO: 32 - DNA, 1500 bp, Cicer arietinum
attaaggtttatccaaagaaaattttgccaatatgtgaacaaattgtacaaacttcataataccatatctttgaattacgatcacaatgaaaaactaactacttgaagatgacaaaaggcaactaactacatttatattttgtatcagcaaatgccctaattcttacatcatgaatcacaggccttaaagttacactgctagaaaaactgagaacaacattactgcatcaaagttgcaaaataatgccgaagataaacagaaaaaaataagtcagaacctcatcagtaatacaacaaaatttcaaacatgtcatcaacatcaacatcatcattgagtaatacaagctaaagatgaagtttcaaacatctttcagttttttacaggccagttatcctttcataatctcaactattcaatattttagttatagatcctattgtttacatagttatccatgtaatatgctctttaagaatcagctgtctctaactttaatttttgtcttgacattttcaacatattataagggtatgtttagtacttgtgtgtctttttttgttaatcaatgtaacataaatgtctactttgcgaccgtcttcgaggggattttgaattttgtaccatgaggttacaagactaaactcttaactcgtggcgtgattttatttagtcacgcatgacgtatcatagactaaaaacaacattgtttaaaacctaaattacatgattaaaatcaaaaaatgttataatcacagatacttaaatcatatttaaatctaaattaaatattagagaacacattataaagtttttaaatatgtatcttaaaaatagattgattttatgcaattttacttggctgagaggaaaatgtagaaataaatttctcatgtaactatgaagacagatgtacattctcaactaaccaacaaaacggataagagagtgtcagctgagaagtttcattccattaattttttttcttcttattctaatttttattttcctccctaaggagccaaaagacttggacaggaacaaatgttacccattccaatccattcatgataaatttttgtgaagtaacagtcacataattccattatgaggccaatacactagagtagaatttacaaggatacaattaaattattaaaaaaaatatcaacataaccaccatcacatatttacactctactcatgaggagataagattagtggggccataatccacgtggcactatcatagtggtgcctaatgataaggctaagatacaaaaagtttcaaacttgtgtggttaatatggctgcaatgtttatcattttcacaatccaacaaattggtattagacagtagctaattaccacatattttaagatcatattggaaatattgaaaatagcaaaaataaaacattatatatagcaaggtatagtagaagctttgcaaattcagtacaagtgtttctgattaaagagttaagtaagggagaaaaaca

SEQ ID NO: 33 - DNA, 1645 bp, Medicago truncatula
atgttgttttgtttccaaataatattgctacaaagaattaacccattattattctttaattttgtaatagtgggttgaaaactttactaaaatgaaattattgacaagatttcaaattgtgcttatgttgcgatttttaatattgtgaaaacttgcggttaaatatggatgcatttcacaaatcctagctaagagacaaacgccaaaattgataagttagacattttatcatagatttgaacttgggattccacacttgtgtgtacgatattttaagtggttattgacattccgcttataaaaaaatttggtcgatgtaacaacgattaatgtaattgtgacatagtcaccacgatgacatcaaaaacctttatgttgcggtcacaatctctttcgcgtttaaagacttgtttattagtgatttgttactaaatgagggtaaacaaagcaacttagatgaataatatatcttatacgtttggttagtcataagagaaaagaagtccataatttaatttgtcctaagccaaaatgactaggatgcacacaatttcttgttattctatttgagtaatattttgcactcatgttgtggggaagcaatgaacatccccgaccaagtatgaatgaatggtgtgaaactttaaacagaatagtttaataaaacaagaaaattaatgttaatattgtcgtacactcataacctatgaagcacagacacagacacgaccctgacacatacatgtcaacaccaataaaatgacataattcaatgtaatcaagtgttggtgttctgtcagtgtcaaacaatgggacacaccttcaatcaaaagtgttgagcaactcatcatgcacgtgcgtacaatgcttgcctgctcgagcacttttctcgcttatgagtaaactatgagtcatactcaacaacaattcacgtgtatatttatatgcaaagagttcgaccacataattttaaagcataaagtttaaatcttctcgaatatggttttaaaacgactaccacttttaattgataataatttctctttccccaatttatgaaaaacaaaagtttatatatgttggttttttaaacattcctcttaaagcaacaaaactaattggaatcataatatttttcttctgccattctcaatcaagtcactgcaaattataaatcataattttgaaattgtacaaatttaagcacaattggattagaccaagtgtagagctaccaagtacataattttttcagaatttccaaaatccaaaggtatatagacaagaatcttaatatcaacatgattcacttgattattttttgctagtaacttgctgtatatagtcatggatttcccattggtaaattacatttaaccgttatgaggtatgattaattataggtacacttatagattgcatcaatatttagagtggaccctttagataggtacttttgtgaacaaggttactcaaagataaagaaatagataagaagtttcacctcctctactcaaactatacaacatcacacattttttaatacatgaatagtcatatatgtatttcaccattggatatatagcaagtttgagctgaagcatccatcaatcagtagaagcaaaagctgagtacttgaggaaaa

SEQ ID NO: 34 - DNA, 1500 bp, Cicer arietinum
atttgtctccatcgaagagtcatatcagatatctctcaacattgttatttttatgatttaaatatttatttgattatatagatatattattttttatattagtctatataaattttattgtttaaatttaattattataaaattaaagataattaatttttagtttaaaaaaagagttaataaattgcaggataagtgaatttataaatacacataaatatattttttatgaaatattaaaaattaaaacattttataatattaattaattatttaataattagtattattttataaaaaaatacaaataaaaattaaaagaatattttaaaagtgttttaaaacttcgttattcttttatattaatttgatattaagaactaaaaccatttataaaatttaattcaaaattttttatagaaactaaaataaaaaaattatattcataaaaattaaatatatattttttaaatatattttactataaatacaaattttgtttttttactcataaaactcgaacttaataccatatcgaaccaatagtatgatagtagaaccaaaaaattaatttatttaacattgtaaaaaaaaatacaatttatttaaccattctaatattctctttgttgaatttaatataattttcatagcagtcggccttccaacggtggagtggagttcatattgtaaaacagacgacgtgggcccatacgaaataagtgacccaataaagtaacgccgtggtaacgattaatccagattccagtagaaggttgcattcgactccgtcaagattaaacgccaactaccatttcccaattgcaattaataatttccgagaaagaccgccctgaacgcattggataccaaaactaagccagatacaacaagcaacccaacaactagcaccgaatgacatataatcaagacacgtgtcacaattctagtggttacgtttttgacccttttgctataaattaaccccaatgctcccatacaaatcgcagtttcatttctattgaattactttctttgagccctatctacttctcaacaaatcctcaaggtattttccatttcttttctgattttcgattcgggttttcgtctttcctttcgtagatcgtttagaaatcgattagacagagtttatataagctatatttattagaaatgatctcagatcgttttctaatatatattctcaataaatctgtttgaattgcatgatcgtcaattaggtttgttgtatctggtttatgaacaggttttgcatgaacggatctgataacgtttaatttgaaatgcttcatattcgtttgttatacactgtttttaatatgtattagagtcaattcaatttctggtgtttgattttagtttatttgttgttttgatctcaagctgttttggtctgttgttatagattcataatccagtttctaattcgatttctggcgtttgattctgatttatttgttgctgctacttccacag

SEQ ID NO: 35 - DNA, 1500 bp, Medicago truncatula
gattcctattttgttttgtagaaggccgaatatattgagaaaaactttctaatagactatatgtaagacttgaaataaatttctaaagaacgatacttttattatacaatcaaattttgtaaagatgaattaaaccgtaaaaaagcctaaaatggtattagaggggagtgaattaaagccacaataaaaacctaatacaaaaggatgctattactttttactatttagtgttttatgtttggtagactaataattataacagattgaacaaaatgattcacatatcaacttagattgaacatgagtaatcacttagctatggtcttcaatctccatccatttgtcacagatccacatcatagcataatattcaaatatccaataataataggcaagacaaacaattatttgtctaagcatgactgccggcaactacttttgagacacctttgatataattggtcctcaaatacatatcaattatccttttcttttttaattatattcactcacactatgtaaactgtaaagagatatagaaatatcacaaaagccacttcatccagtaagaaagtgccatcttagatttggttggataaggcatttatgctaaatgaactgtaaaatgagaaacatggtaaaaacaccaagtacggcaaaaaaacatggaccatttttaccttggctcatttcctacatgcacaaatttaagatagataaaaatttaagacacgctgcagtgcttactggtttttattgtaaatcaaatgaaacatcttctgctgtttttcaaaccaattatccttaggtattgcattgtaacttaaaaaatcgtgagggtgtgaactgtttcatttttctgaaaataatggcctaaaaaatactgccatgtatgactcaaactttgtgtcttacaaacaattacaattaacccatatagtgttatatttcaatagattcctcatgaattaaaattgacccgtgtaaccatcaaccctgacttggttcttttcataatttgtctacctttatcttctttattatacgttgaaataagaacatgcacctgtccataacaggtttttcaaattgtgtggttctcaagcaactatttacaatatgacagcatgtggacccaaaaaaaatcttacactgctgagtcagtttaaagcatagcctcataaattaagccaatttgatggaaaaataaaattttcagtgatatatctataggaatcccatagttacacatggaacaccaacattggttacaaatcatatcatatgaagaatctaacccatttgtggattttagaggagtgccaagtgtaacatcaaaatcctcaacttccaatgagattgtagctttggataacacaagataagagaacaacaacttcataactaatataaatacacacccatattcaccttaccaaatcaccattcatacgagtacatttatcaaaagcacaacaagcattttaatttcattgcaaa

SEQ ID NO: 36 - DNA, 1500 bp, Cicer arietinum
ggtagacttataacagattgaacaaaatgattttatacatcaaattaattgaacatgagtaatcacatttaatttcaagccatgttcttcaatatcctttttatttgtcacacattcacatcataatatattcaaatatttcctaataggcaagacaaacaatctcatttgtctaagcatgttgagacaccttgatataattggtcctcatacatactaacagatcatgcaatctaaagagatatcataaacacatacttcatccaataagaaattgccatcatatattttggataagtcatgaacattaaaatgggaattaaacttggtaaaaatatcttgtacgacaaaaagcagggaccattaacttgcctcacttcctctatgcacatgtaaacctatggaggaatatccttagacgcccttgtgctacataaaatgatgtggagatataagtgcttaattggtcgtttgatgaaattccgaattattaaagttttttaaaaaaaaaatcagagagttaagtcaaaaaatgacgtggagatataataaaccttttctatttattcttgcaattatctgttttacagtttttgttgtctcaagtctgaacccttcaacatttttaacatatataagatagttagaaattcaaagcacactgcttactgattatttttttgtcaaaatgcatgtgatcgtatgtatacttaatatactatttattgatctttaattgacaactaatttaagataaacacatataaatcggcacaatgagtgaaaactatccttaagtacctcttgcatgataacttaataataaatcatgaggttgtgaactgtttaatttttgttgttgaaaaatgaaaacaatggcctaaataataccagccatgtaagagtaagatccaactcagtgtgttacaattaacaatagtttcttcatgaataaaaattgacccttgtatgtgtaacatcatcaaccctgacttggttgtttgttttcacaatttgtctgcctttatcctgtattccttactacacgttgctctaataattcttgaaatagaaattatccactacagtcacaaatcagtgttttcaaattttgtggttctgagtgcacaagatgtggacccaaaaatttatcttacagatcgtagtcagtgaaagcatagcctcaaaaattaagccaattttatgaattaataataaaatgttaatttatatctacgggaatcccacaggtacacatgtaacaccaacattggttgcaaatcatatgaagaatctaacccatatgtggattagaaaggtgacaagtgtagcaacaaaaatctcaaacctccaatcagattttacctttagataacataagataagagcacaagaacttaatctacaatataaatacacatccagtttaacatggaaactcaccattcatagtttacaaaagcataacaataagaaagtattttctttattattgcaaat

SEQ ID NO: 37 - DNA, 1083 bp, Cicer arietinum
cttagtaacggcgagcgaaccgggataagcccaccatgagaatcggtcgccttcggcgttcgaattgtagtctggagaagcgtcctcagcggcggaccgggccacagtcccctggagaggggcgccggagagggtgagagccccgttgtgctcggaccctgtcgcaccacgaggcgctgtcggcgagtcgggttgtttgggaatgcagccccaatcgggcggtaaattccgtccaaggctaaatattggcgagagaccgatagcgaacaagtaccgcgagggaaagatgaaaaggactttgaaaagagagtcaaagagtgcttgaaattgtcgggagggaagcggatgggggccggcgatgtgtctcggtcggatgtggaacggtttgtgccggtccgccaatcgactcgggactacgaccgacgcggattgtgatggtggcccaagcctgggctgttgaaatgctcgcggagacgtctacatcacgattgtggacggcagcgcgcgccgtttcggcgtgcttcggcacttgcgcgctcctggcgtcggcctgtgggctccccattcgacccgtcttgaaacacggaccaaggagtctgacatgtgtgcgagtcaacgggcgagtaaacccgtaaggcgcaaggaagctgattggtgggatctccttgtgggttgcaccgccgaccgaccccgatcttctgtgaagggttcgagtgagagcatacctgtcgggacccgaaagatggtgaactatgcctgagcggggcgaagccagaggaaactctggtggaggcccgcagcgatactgacgtgcaaatcgttcgtctgacttgggtataggggcgaaagactaatcgaaccgtctagtagctggttccctccgaagtttccctcaggatagctggagcccgagggcgagttctatcgggtaaagccaatgattagaggcatcgggggcgcaacgccctcgacctattctcaaactttaaataggtaggacggtgtggctgctttgttgagccatgcctaggaatcgagagctccaagtgggccatttttggtaagcagaactggcgatgcggg

SEQ ID NO: 38 - DNA, 1643 bp, Glycine max
ccacgacgaacgggaggagttaagcgtgaggagcaagatcctgtcgaccttgggtttgaacacggagatttgagagttcgctatagagagcgagagcctcgtattgcacagcgacaattgaatgttgttgttgttgtagaagaagaacttgtcctttggagccgagtgacgccgctgagttcgctggttgggtttctagacgacacttggctatgggtttgagtgggattgtgattggggttgttgtggttttgggggtggtggtgatgactttgttggtgacggtgaagaagaagaatggtggtggaagaaaggggttggaggggtggtgatgactttgtgtccgagtacagcctgtccgtctcagagcaaaaaacagcgtcgtcgagtttcgtgacgtttgagcgtgggtgaggaagtggtcgagggaggccagaaacaaggtttgtgcattgggaacatggaaatgggtggtggaaggaggagaaaaggtgcagaggggagagagagaagaggaactagagtagagagaaagccagaaggaagaggatcatcacccttcatcttttctacctataatccttcggacaaatattacttgtgacaccataggtgggatatctatgcctccgtaacacagataatgaatttcgattaataacaagaatcttaaataaaaaattttaaatattaaaaatccaatttaaagaaaaaatctattaaatatcagatgccaaaaacatattatatatatatatatatatatatatatatataaatataaaatatcaaaaacatatttaacccttcataatttaataagttttgttgttgtcttagttaaaactgctctattgtttcatgtgttgttattaaattaaatacaagaacattacgaataatgatatgattttattcacatctacaagcaaatttacctaacagatgtgaatgccgtggaagcaagtattttatgctttgaaattgacacacattggagtctctaaagcccaaccaaacgcacgaatatcctgtttggaacaagctaggacataaatttttacaccaaaataaaatttaataatttttgcttttacgttttccacattaaaaacgtgatttattggttgtgaagcatgttttcaaacatactataaattaatctaaatattataaggcaattaagtaataagtgggaaaaaagttaccaaaaatgcacagcaaaagtttaggaagaggaagaagctactctgtttgtaagaccgtgtttatggtgttggcttgtcataatagattcaaattgtattttaaaagaaaattatgtgtatttctatcgaagacaattctacttcaatcaataaaactattataaataataaaatttaggctctgaccaattgatataattatatatttgaaagttggactaagcagaaactagttattaatctgacatgttgaatagggtatttataaatgtaaaaagaggaaagaattgaagaggtggttggtagaaggaagcaaaggctacaagggtcatttgacaggcggtggcaagacttttaaagacgcacacataggcactgggcactcacattagcattccacctcaaagcaacacaagaacactctcttgaacattc

SEQ ID NO: 39 - DNA, 1500 bp, Glycine max
tgattaatttaatctatataataatttgtacggaaaaaatatactatttgggaccctgagattattatgtttggtagaatttgggaggtgggaagattggatgttaggtggaacgaggtcaggagacgacacatctggtcggggaggggaggtagaaaaattatacattgacacaataggataattggtcactagtgagataaaattttaaaaaaatatagatattcatattataggtattatgatttggtatgaaagagaaaaaaagaagatgaaaagtgtttaaaaaataagtttaatatttatgcattgaaatcatgatgaattatgagtagatgtgtgtttaatgtccatctatcatcttcacagtatgggacaacacaaaagcaatatgaatgtgtaggaccacaatgtggccctcaagccccaattcggcccattgggcaatcgaaaagaaaaagcatgcgctgcggatattaataattttgtgacgctccaccacaattccccattcccaaaatttctcattctccctttcctctccgaaccctcgatcactctcacgcgctcctatattcgctcctccaccgtcgctctctcgaacaaccacaacaccatcttcatcacatggtcagtttttttctttctaattctctctcttccttttttcgtttatccgatttgttctcccgcccgcaccaataactaaatccgcgaatttcgagttttttttttgttggattttgttgcatcggaatttctgagctgggttcgtatttgatctgatcgtttaacttgaacggtgccttttttaattttctgaaaaagaataaatcgtggcgttttcagacttgatttcggtgtttcgattgagtttttcccgaattggtagcttaatatgatagttatattgcggatttcagtttacaacagcttgtgatgcgtgatgtgtttgatctgcggagaaatcggttttgtgatctgacatgtggatttaacttcttttatttgataattatgcgtggaattttcaaataaggcatatattcttgttctgtttttgtttttggttgcatatattcttattcttttattagatttattttggtgatgtttcttatattaaatttattaatgagtaaatatgattttattttgggatttaagtgacgacaaacataacgcggctttttatttttttattgatgtttacttattttggcactaatcactgtctgcttctatcccctttatttggacgatactgggtttgttgaattattattatttacttcgctagttggcgacatattcctataacatttcattgttcccaatctacaaatatagtttctttttttgtaagcatttgattttctgtggcaagtttcaactctattatgttgacaatccttttttttttctctttcctggagtaacaatttaaatggaaaaaatattagcaacatatttgtgtacaattacctgggattgatgacc

SEQ ID NO: 40 - DNA, 1500 bp, Glycine max
aggagtgattagcccttaaataaatcttgatatcttgaagaattaatattttacttttgactgaagaatgtgttggataaatttctatttattaatatgatatggcgtggttgtaaagtaaatttctactagaaatttgtgtaaaaactgaagtctttttgtgtaagaatgtgtaaatagttgattaatttaatccatatagtaatgtgtatggacaaagtatagtatctgggaccctgagattataatgtttggtaaaatttgggaggtggaacgaggtcaggggacgacacatttggtcgggagaccgtgaaatttacggtacgggacaacacaatgtggccctcaagccccaattcagcccaatgggctatcgaaaagaaagaaagaaagtttgtgcgctgcggatattaataattttgtgacgctccaccacatttccccattcccaaatttctcattctcccatttcctctcagaaccctcgatcactcccacgcgctcctatatcctctccttcaccgtcgctctctccaacgatcacaacaacatcgtcatcccatgtgcggtttttttctctaatttctctcttccttttttcgttatccgatttgttctcacgcatcaataactaaatccgcgaatttctagcgtttttttttttgttgaatttagtggcgtcgaaatttctgagctggattcgtatttgatctgatcgtttaacttgaacggtgctttttttattttttgtttaaaataaaggaataaatcgtggcgatttcagatcgtatttcggtgcttcggttgagtttttcccaaattcatagcttattatgatatttttattgcggatttcagtttacaacagcttgtgatgtgtgatgtgtttgatctgcgcagaaatcggttgtgatctgacatgtggattgattccattttatttattttattctaattttaattttatgagcatgttgatttaacttcttttatgtgataattatgcgtggaaatttcaattaaagcatatattcttgtctttttttttgtttttggttgcatatattcttattctttcattagatttattttaatgatgtttctatattagatttattaatgaataaatatgattttattttgggactgaagacacgacaaacgtaacacggttttcttaatttttattgatgtttacttgttttggcactaatcactgtctgcttctatccccttgatttggaagataccgtgtttgtggaagtattatttacttatttagttggtcgcatattcctataatatttcattgttatcaatctacgaatttagtcttttttttttggtaagcaatttgatttactttatggcatatttcaacccaattatgttaaacagttaaacaccctttgttttttttctttcccggagtaacaatttaaatgggaaaaaaaaaagattaacaacatatttgtgcacaattacttggtattgatgacc

SEQ ID NO: 41 - DNA, 1188 bp, Glycine max
tgaatattccatcatcgatacatttagacgagtgggctaaaacaaatgtatgtacacttcaatttctgtaaactaattcatgagatttctgttcctacaactataaacttacacttttggtaaagttgtagtatactactgggattctcgatgttagattgttttttgaatgatgcgttgtaaactatacttcattgtatttctattattgtcatttttcaaatttgcttcacgaattgcatcttcactagtttaatcatgcaattttaatttctctctttacttgacatctaatattcaatggaaatacaaacacaacactataatactataatgcacatccacattagtacttggctctcacataaacatttttgtgaaatattatatatcaagttaatcaatgaattaaaattgaacaattgaaataatggagattaaatagatgaaaaaaaaataaatatttaatgaaactaacagttgaaataactaaaaatataaaataatagaaaaaaaataaaattataatttatttaaaaagagtagtcagaaaaattacataaatatattaaaaaataaaataaaaaatataaaaaaattaaaaattaaaaattcatgttctaaaaaatactatttcaaataatattttaaaaaacacttgaagctagcattttttttaccaaataagtaaaataaatatttttaactaataaaaataacttaaaactagctgcaataacttacataacccaaactcaataaatattattcttatgataacaagtcttttcttcaaaatattattcttatagttatactattatcatcaaattcaaattcaaagtaaattaaatatttttataataatttaattcagtaacaatataataaaattattttattttaaaaatcaaataattacagttaaaatacaaaaaataagagtattagaaaaagaatttaaattacacttatcgtactttattcagttacgaataattttacgaaatccgaaaattcagtgacgcctatcctatatatatatattcgtactatataaggaatcgaatgggaggaagaataattgtgaggcgagggtaaggtcgcgttgtgttgcgtcgtgtcgtgcgctgttcctctgtttctcgtttttgtgttcagaccgaaccctagctccgcatcgcaacc

SEQ ID NO: 42 - DNA, 1618 bp, Glycine max
gactgatatttccattgtgaatttttttattcacaccctcctacacataaaggaaacatgattttattgtacaattgtacaatggaatcgtgcagttgtataattgaattgtattttcatgaaatgacatttttgagaaaaaaaaaggtgtcaaccccttgctagaccattagcaatggaagtggggccaatagcaactctcataatggatcgatggacttagtttggtctgatattgttaaagagctaaaggctccataggtttaactagtgtcacttttgctattggcactactcatgtgagtgtcttttaacaatatcccaaaaatacctttttcacaacaacacaacataatcataatattgttgcttatatgggatgaaataaacaaagcacaacaaaaacacaatttcattgtacaagtgtacaacataatcgtgatttcgttatacaagtgtacaacatactcgtgattttgttatgtaacttttttcaccccaaataacatactggaatcatgatttcgttgtataacttttttcacttcaaataacacaataaaatattaattccgttatatatttttttacaattttttttattaattaagtatacaagttacattttttaagcaatcaaataaattatgatataatttaacatttttaataataattaatattttttcaattaaaattaaattaattagataaaagtatcatttataaacataagcaaattaattattatatgattatatttattaacaataatcaaattaatttatgattttaatttgtatcataattaatttgattataattaaaaatgaaggcgaagaaagtagtgagagaagaaaaatgatggagtgctatgatgcaggaattagagaagaaggaagaagggttggaggtgtaagaaaaaaagagaaggctagaatgatcaaattgcaaaatatttttttaattaatgcttttaattttttacaagattatataaagtaaacaatatatattttctatagtaatttttttttatcgtaatacatattattcttacatatggaaacataaagaagttagaatatatttaaagttaaaacacaacaaaaataaattaaaacaaaagaataaaattcagattaaattgatgaaacaaaaaataggatgaccaaattcaataaatattattcttataataacaagtcttttcctaaagctgtgcaaaaaataagacgatattttcttcaaaatattattcttatatttatgctattatcatcaaattcaaattaaataaaatatttttatattaatttaactcactaacaatataataaaataattatattttaaaaaataattatagttaaataaaaatagagtatatgaaaaagaatttaaattacacttatcgtactttattcagttacgattaattttacgaaatccgaaaattcagtgtattctaatatatttgcactatataatgaatcgaatgggaggaagaataattgtgagacgagggtaaggtcgcgttgtgttgcgtcgtgtcgtgcgctgttcctctgtctgtcgtttttgttttcagaccaaaccctagctccgcatcgcaacc

SEQ ID NO: 43 - DNA, 1632 bp, Medicago truncatula
agttctgttttctttaaggatatgatacttcaatcaattttaaaccaaactatagaatcatttataatttttgttgtcaatcagctaaatgtgttgagaacaactatgagaaaaatatcttgtgctaaaaatagtaagaaaataatctttcgaaatatttgtacagactgttaaaatagtaaggaggttaaaggaaattttgctttgcttttgatgcacgtttcggtgcaggacctagtacggaaggtactttaccctaatagaatcattgtgattctttcttttctccactctcttgaagtgctgacattgatcttagtggagtgaggtctgttttgtttgcagtacacgtcgagtaatgagataaggatggagggagcagagtagattattacagtgtttttacagccttcaaccagctgtctacaactttgatcaatgctcttattccaacaactttgccttatttttttcttttcatttggatttggatgtatgcagccaagttggactggtttgatattgatcccatccatccaatccataaagaaaaaagaaacaagacaagaaagtaaattagactatggaccacagtcttgtccttttcaacaaaaattgaacaactattgtagttgaaatgtaagataactcattttggattcaaatgtgaatggttaatctcatatcgtctataaatgagtatatatgtctgatctaatatgtgtagtttccctctcttgtgatcttaaattggatgggaatgtggtttcactctcttatacttttggactctcaatgatccctttgactcctaagcactcgtgagttttagaggatggatcattgtaggtaacgacgttccatcgaactcctaaacaaatagtatcaaaatcatgattcagtttggtagagagtgagagggggatcttggggtttgaattaaatatatgacgtgaaactcacatttaagagagattgttgggttaaatgtgactggttaatctcatatcgtttatgaataagcaaaatattggataaataccatatatcaaatgttataatattttgagtgaatatttattgtttccctctcttgtgatcttaaattggatggggaatgtagtttcactctcttataatttaggactctcgacgatccttttgactctaagcagtcatgagtttttacatttgatatttaattaaaacaaataccactatgtagtaactaatgttctaggtgaaatgcaagataagtactaaatttccatattcgatagatgatctgctatatgttaatacttaagaaattaataaagagaagtgaatgagaacattaaaaatgttatttactcttatttttgttaaatctataccaatcaatcaagtcacaatccttgttaaatcttcctcaaaaaaatccttgttaaatctattttgtaataagaagggaaaaaataaataaacgataatgtgccaaatgttgggttatgtaggtaatttcagctggttgcaaattgagttgtgggaccattttcacctataaatagtagcatgtagaatatcaataaactatactctactcattaacttaaggtgcagtctagctagattttaaaattcttctctgta

SEQ ID NO: 44 - DNA, 1443 bp, Lotus japonicus
ttttgcttatgacctcttctacaattgataataatggtcaacttgttagtactttgaactctcttcacacttctctaattttcaatatgttcaatgatgtctgctatttgggcaactctacttattttaagcagtttttccttctgacaaatggacgtgtgaagctcctccttactttctcaatcctttcctgtccctagagatatataaatatatatatatatatatacatatatatatatatatattatatgctcactcatttttgtggaaaattatttataaaaaaacatttgtctctgctgctagctacatattacaaaatcttgcagttcaaattgacgggtgtgagacgtttgtagtctcacttatcgtgggttttgatctatacatatgtatggtgattggtgagcttgtatctttccttttcattacccaaggtatagttagtatcaactatcaaccatcaattcgacaaacatgtttttaagaatgtttttaactaccacaaactatcgtttaaaaaagagacaaaaacaaaacaatgtttaacgagggttttaaaccttcgtaaaacaaattctttttcactcaaattttattgtgaaaatatttttccctgtggtaaactttgaaagaaaatttacaggtaaaatttgttagtaaattcgcagaaatttttgatgctaaatttgcaagcaaccatttgtcgcttgtaagttctatggcacatatatttgaatattggaaccgtaattaaaatactttaaaaatgaggggccaaaataaatcattttattaatgaggattaaaaaccaatttttataaatatttttccccgcagcattagataaaggggttatttagattacccaagagccaagattaaggtcaagttactcttgcttatacaaaaaaatagtaaaattactatcggtgacccaataatatttgaacataggctatctaaactagaatgattttatccaatattttttttaggtaagactggcaagtcgatccttacaaatttaaaagccaaacaagttctagaaataaaattaataacccattgcaaccaaattaaaaaaactaaaaaaaaatcaaaatgaaatgtcaatcatgtaaaacaagagtcttatatttttttacaaattctaaaatttgtgctcatcatataataattttcaaagtttatgaagatggggagagaggcgtgaggaaaagaaaagttattttcctttaaatttctatgaagtttccaacagtggccccaaaagtgcaagagagattataagtgactctgtccctcgtcattctcttccatcatcccaattatgccaaccaaccaccatacacattttcttgttcatcttccatattctctcatcattattaattctctttaatgctccaaattaaaagatgatgctgaaaataaag

SEQ ID NO: 45 - DNA, 1500 bp, Lotus japonicus
atgactagaagactgtaattaaaaaatatataattatgccagtgttatttttcttctttcagtaatgctatttggtatggatgatttaggcaagaaggggcaaataactcttctattttttctgaaaaataaatattaataacaaaaagtgtacccaaacatccactttttgacgataatttttaactgccgctaatatttataatgattaaaaatcattactaaaacgtatgtcaaagtgaatgttaagctataattactcaacttataaaaatgcaaaataacaataaaaggatgggttggaatacaagccaggttcatattcttcttgcatttggttcagatgtccttggtaagttggtatcaaaaagcatttcctttttctttactcatgtcatgctcaagtttatttttcaacattagtacttcatttttattttctttatttttttctcccttttctttgtccaaaggatgcatatatctaatttcagccatataatgaagcaactaatatatttgctattagttaaagatataactttctataacttatggtccgcatatccttaatatattacgcttaaattagaatttgctttatatattggtcgcttagagatatatttagagattgtccattaatacaacgctctcattaataatggaaattatcaatggtggaaattgaacatataatctactaagagaaagtaagatagccaaaatgtgttggccacactattgctttatacctgacatgagcatatacaaatgttatgaatggatgaaccctccaaatttgtagcaaaagcagtgaatagaacatacagttactgcaagagtgcaatataccctgcaatgttttgtccttttcaattggggggtaagtgattgtatttaattactaattttcaaatacgtggcaaaccagcattgcgtgcaattgtatgtcatggacacacaagttgaaaggaaaggaaacaaccataccctacagcaataataaaccaaagctcttaactcccttggaatactcactaccacaatttctttcttattttatatctaaattcacataagaattaacgtgaatctcatagataatatccacacaaacaagggaatggtccatattcaacaatgtatcaagaattcaaagatcaaatttaatgtgcgcagagtttgattatatgtttgtgtgaaggtaaataatgggggatagaaaaacaaagggtacaagacaagatatgggttgtgatcctattcctgatccaaattgacatttgagttatgatcattctctcgtagttaatctccaacatccaaacctcatctaaaaggataggagtgaagctctccttggcttgactttcattttcattatctaccttgtattcacataaaaatgggggatagaatagccatttgatcatgtcacacacatagctactacatacaattgctatatagcgtagacatttgagtcttcattgcaca

SEQ ID NO: 46 - DNA, 1562 bp, Medicago truncatula
aaagaaataaaaaaatttattctcccaattttcaagtttacatatcagaatatcaaccaagcaaaaatactagatatttccttttacttggtgaattgttggatcattctgtatcaatcaaccacattgaagcttcatgtataagacaagatcaacgatattcaaagaagaaacacagaacgatctaattattcaccaagtaaaaggatacgtctgttaatattgcatcaaagtaaaaacatcacaattgatatatgataaaacagtaaatatgtgatgaaatatgattttttttttttttgtaaaataagatatagttagtaacattcttgttcataaaaagatatagttagtaacataattgaacatagattaatactatgttataaatggaattagtcctcctaagttgtaggaaaaacatagaatattgaacttacagttactgcaatagaacaatataccatatgttttgtccctttcagttgggagaaaagtggttgcattttcacctacgtggcaaaccggcattgagtgcattatatctcaatacacaaattcaaagggacaagacaaccttataccctacatcatcaataacctcttaaccaccttggaattcactaccacatattcttttttcagatattgaaattttcataagaaataacgtgaatctcatagataatatctacacagtaacacaacggtatggcccatatacattgtaacacaacgaaatggtcaatttacattgtagcacaatggtatggtccatatacaccaatgtatgttaagcactcaaaaatataatttttaatgaattgaaaaatatagcaattaaatcttgtatgagaatttagtgttcgacacgtccggaatagaattaccttatgtggagagaacatttgagtaaaagaatataagaaaagtttagattaaggaaatagctcgatgctaatgtaggaaaccattgataaactcaatgagttcgagaattatattcttaccccgcctcaaaaggactaatgagaaatcaaaccattaagataagaagtcatatacaagcaatgtcatacgttaatgagaaacgttgttttgatacgtacaattttgatatatttgtaactcgtatgttgcagaagataaaactaggagaaatacagataaaacatacgatgtcagatgtaatatctatattttaatgttcaaataacataattgtactttaatatttcaaccacttgggggttataacaatttacattttgtgtgtgtaaataagatgatagatttgtgaaggtaaattgagaaaacatagaacaaaaggtaaatgacaagaagaaggcaatccaaagggaccttaaggttatggtcattctcttgtagttaatctccaacattcaaacctcatctaaaaagataggaatgaagctctccttagcttaactttcgttttcattatctacctttggttacattaaaatggggatacccaattggtcatggaacacatcatcatatagctatatagcattgtgatttgagtcttcattgtcaca

SEQ ID NO: 47 - DNA, 1627 bp, Medicago truncatula
ttaatgtagttggaaatagcaaaagagtcttataaaaatgaagaaaaaaaattctaaaaaaatcttataattaaggattataattaatctacttataattaaattgctttgatttatattaagttctgtggagtatatcatactagtagataacatgtcattatcacgttttcctcgtgtgtgccgagaggcatatggggcggtgttactatttaatgtttagtgaacactaactttttttgaaggatagtgaacactaattatattctagagttttcttgaatatttattgattagaatttagaagttgaattatatacacgtttaactcccacattcaatgtgagatggtttcttgatatgaagagggacattctcttttcaacatataagtctcacgattaaaataggaagtaaattaaataaaagttgatcccatcatctttcaagtttatgcgattctttctgttttaattattattatttatttatttttcaaatgcacatgttaaaatagtacttcatctgttttaaaataagtgttattttagccaattgcacatagattaatgaaataatcattagacataacatttttactaaactgcccttactttataagaaaaatacaaacttaaagttgcattaaaaattgtgtgagagaaaagttgaagagagtaatgttggaagaaaaaaattaaagttgtattggaaatgtaaagtgacattcattttcaaacaattttttttttgctaaagtaaaactcattttgaaacggaaggagtacatatttgtggacatatttaaggactctttagtttaagggaaatgctaataagtgccccaagaatacttgtttagaagtcaaaagaaaatactttatcttataaattgtgtattcatcacattgaaacttaaaaaaaaataactttatttacataaaatttataaattatgtcatattttgaatctttagaacacttgttattacaaccaaggtttaatatttttggagtcattgattcttaatagatatttttgtttaaaaattataaataaatgacgtgtacatatgaaatccatttaaagatttttttttttagtttactaccattgtgaatactctctaagttagagtttggttttttgtcgagcaaataccccttatacttttcctgaattgtggattaagtacccctccctcctttccaataaaattggccgttaaaaaaaacattcaacataaaaacctttttaataaataatttgtaagtgaccttattaacatttgttttgactcaaattctattctcactctggtgatctcctagaaccaactcatctaaactttataacacccaaatttcaaggaatcaaaatttttttttctttttttttacatcaaagccccacttcgtgacttcaaagcaatcaataatttatacgtaccacccctttatacatttaactttttatttgtgtccttcaaaacaaaaacaaaaaaactttttatttgtggcgaatatcaatctctgtatctttcataatcaatagctatgtgataactttgatattcactcaagttataaatagagcaacatcaacggcaaaattgcaagcagcaat

SEQ ID NO: 48 - DNA, 1500 bp, Lotus japonicus
agtagttggtgagatgtcaccctcatccagatgtccaaccttgatgtgaggcactaaaaatatagttaaaatagcttgttggaagttttttggtttggtgtagtattgaaactgaaaaaagcaaagagtaaactagactccttcctccttctgaagcaagcagtgacaatcgatcttgaggagggaaatgaccacaggaggaaccaaccgcattgtttgcgcaaaaccaaaatagctaagctatccgcataagagtttgtctcaagacgaccatacttgaaagagatattctaagaacgaaccgtaatgaaaactatctcatccaaaacattatacaatggagaaggcatgatagggcctcaagcaaacagatgacatccgagatggatcaactgtttaatgggcctgaagtgtgtacaacgtcccactagcccgtcttgggcttcagttctggttggacccggtctggttcataaaaaaaaagacaaaaaaagttggccaccgacaaacccagcttcagattggaacaatttcaggttggattatggtaatgaagttttccttttacaataatgaaaccagtggcagtggatgcctggtgacagtggaaaccatatcacctgtcaaacagaaaagcaagttttccttttactgagaacttatttaccgtttgttctgtttcttttttgaggaagaccaccaacacttttaatattctctttttgatgggttaaatcccatatatggtctactaaatttatggtggggcctacatggactttaactaatcaaagagagtgtgagtgttagaaagagtgtaaaaagaatgtgttagcacttaccgtcttgtctactgctaattgctagaacctctcacattgtgccacagctacaaatggtccaatggagctcatgtgatatttttttcaaaaaataaacataaaaataaaaattgaccgagtaaccacttttatactcaaagataaagatacaagatcattcaccttaaagacaaaaaagaaataagcaaaatagcctgtaaaagacaaaaaagaaaaatcaatttagaccctaagatgaagattatgatcaatttagtccttaaagataaaaagataatcaattttagtccttggtaatactaaactgaacatcttttcatcttaaggaactatttttcctaatcctaaatcttgtaagaaaaatgttattccttgattccttgccccttttttaatgcatttaagacatggataaaacaactgaagtttaactgataaaagaaggctctggcatcttaataaaatattttcagcatacattaaaacctagtcaactttcttttccttttttctgtaccataattcttctctctttattttcctccttcttaaaaagccccccccttgatcgtgacttcaaagcaagcagcaacttctacttaacatctcccttttcatcttcacagatatcatatcagagagcttcacaagcggaaag

SEQ ID NO: 49 - DNA, 1669 bp, Glycine max
ggatgaatagaataatgtaattaagaaataataactgatattagacctaatagaataaattataattttgattctcttatatgatttcaatctcctatttttttatgtttttaatttttttatttaaaaaatttataatttttattcaatctttaattatatatatatatatataaatttaagatttaattagatcaaaataaaaaatatataaaattaagaataaaatcaaaattatgaattttttaaataaaaaactaaaattatgaaaaaaaagaaaaaccaaatcaaaaattaaaaattataaaataactaaaattataatttagtctagagttaatgatgtagccataatcttagaatcccccaatcaagtaaaccatttccccacttgctactgatcagtgattgtaaggtgcatgaaaggtttgtacgattgcacaatggccacttgatcttgtgtgacccaagtagtaaattttatgaatattatctcttcatgattgcataatggctgcttgatcttgatgtgtcctacttaataagttttctggatttgatatcaccataatagaattattgaattttgctactttgaacattaaaaaaaaaataataaaactgtatgtgaaattttatacaattattatctttagacttaaatatttttttcttgcaatttaacaattttttaatttttattcctgtaatttttttttgttttaatttttataaaatatgtttgttttatttttttttcttaaaatactttgaacaatgaagaattattgtctaaatcgttttaagaataaaaaataaaataaacatattttgtaagaattaaaatagaaaaaaatataagaataaaaatataaaaaatataaaattacatgaatgaaaaaaatatgtaaatctttatcttttcatttacttacattttcttttatagatacaattacataaaatatatacacaaaaatcaaaggtaacagtgtttttttctcagtttggtgtgtagcataacgcgcctcacggacccacagtgtgatatttgttgttgtgcaaggggccaaaaatgcagctagcagcggcaagttccctattattactattacgttatgagtgcggctgcaactacactaaccacgaaaagacagaaatatcccttatggatcaaattgattcgtaagttaatttttaaaacttactgatcaacttgatccgtaatcttttacggatcaagttgatccgtaaattgatttttaagacttacggatcaagttgatccgtaaattttttacagatcaagttgatccgtaagtcttttacggatcaagctgatccgtaagttgatttttaagatttacggatcaaattgatccgtaaggggagaaattaacagggacaattttgctattttcaaagaatgttgagtgcaccaacaataatgctgggtgtacctagcaacaccctcacgttattattctcttctcttttgttaggtgtacaattattgtttctttttcttctataaaaagcccttgtatcgtgacttcaaacaagcatcaacttctaccgttctacatagcatctcctttaccaccattctagatatcagagaactttgcagctctcttaggtttttaaaaaacaccacgcaaacagcaag

SEQ ID NO: 50 - DNA, 2587 bp, Glycine max
cttttgaatctattttatccaactagtaggtgcatatatgcgttcattcgctgagttatgaacaaaagaaatgaaaataaagaataaaaaaatcaattcatcgatttgttaaaagctacttctatatcaaaaagcttttttttttctaaatgaagatggtatgtttgacaaaattatgaaaatattttctaaaagcaatgcaaaaatatgaaattaaaaaataaagaatcaattcatcaattattaaaggataaaaacttgtttctaaaagattgtatgtttgacagaattattaaagtgataatttttttagcataaataatattaaaaaaattcaaatatttaaaaattgatttaaaacaatataagtttatctaaacaaattatattttttatgaaaaaaagaaaacgtataatgaaaaaaagttatgactgttttttattatataaaattatcaaatctttatgattcttttataaaaaaattaaaaatgattgtcctaatttttaagatgaaagttattagaagtaaaattatgtgtttgtgtgtaatccaataattaaatatttcattttatatttatcaactagttaattacacaaattttaattcaactagtaaatttatgtgtttgtgtgtaagttatttcaattagcttaagctatttttttcatttttatcttactaatttacttgaaatctatttttatcttttttatttagaaactctttttgtctaccatgccactctcaatgatatcacgtctttcacttcttccactttcacattcacacaacgtcagcatggtttaatacaattgataaatagttagatatcctaaacatgtgttctatgaatttgattcttgactatatgcatgaaaaagattattaaaataaataaataaccaaagtcactctaattataaattttcagctatcttttaagaaaaaaaaatctcaagcacattctaaataaatttttaactaattttcagctatctatacacattcacaatgaaggaatgtgagaattgttagaatatttaagaatatgaaaatattaaaaaaaaaaactaagaagccagagtttccactttccaatatgcacttaaataaatagttcaattaagggtatgtttgatataaattaaattttatttgtaactttagcaataaaataaaactgattaaaacatgttaaaaaaattaataagtgttataaaatattttttttaaatgaatatttccataaatttaaataatagtatctttcaaatattgacccaatagaaactagccagcttataaataaataaataaataaatatgaattaatgttgatttaataagggaagaaaaaaaggtagtagaagggtgtgaaatgaaaaagaaaaggctgtcatccaggtcaccctaaccctcttttctcgtggtcgaaatgattccttctatttcaatttttggtcccaaccccaattttaatttcaaatgcattacattacactctctcactctcacaccaaccacacactctctctctctctccgcatatatatacccctcttcttcattctcctctttctctgttctcatttctctttctgtagccctcaatcaccggagagagagtgagtgcaaagccatcgtcttcttctcagacaaggtattcatcttaatcgcttgcatcatagctttgatgcaaagtacgtttccttttgctctttgattcagcgctttttttttttttttttgcaatctcgcatgtttattcttagatttgttcaacagtactactctttattttttaatctgtgtgacattgctacctacctcatcaaaatcatatctggatgtttttgtatctgattatttatattcgtactgcctgatctatactgaatgagatgtagtggattgtggcgatttgtagttttagtgatgcggatggcatatcatagttccgatcttctagacctaggatcttcgatctttttaaactgtacttttacttatttattttaaaatggggatagacatcgaatctataatcttgtgttggatggctttatggtctgatctgttttttatgtgggaatctctcactttttcttttttatttatcattgatctttctttgttgaagatcatctagtgtttgggaacttaagatatcctgatgaatgttgagattttagtagatatcctagtaagtttgagattactagctgcacatactcccttcctatgtttattttattcgccattttctggacgttttccattcatttataaattttggcagttttactttaaaaaaatgatcaggatcggctcctttgtttgtcaatttatgtaccttttctttctaagcaatcataaagtagattatataatactgaattatttcatttgcttgctttcttcccattgatttttgtgtgttatatacaatgtactgttgctgtctcatatagaaaataaacgtaatcttcaaagtctgtaagtggctgggttctgcagattgtctattcaaacttgttaatactggcaatttgttctatgcatacagtagtttggtaaaag

SEQ ID NO: 51 - DNA, 2351 bp, Medicago truncatula
tcacttgtgtgtatgagatgataatggttttgccatcttgtttatctaaaaaaaaaaatgaataaagtttaaatgaatttaatattactttttttttctaaaaaattaaatattacttttacattaattacacatgcgatcctttaagtttgaagtttgtaataaattgatcctttaagttattctggttgaacataaatactttaagtttgagatatgtaatagatgagtcctttaagttattttggtaacacataagtcctttaagatttgtaaatcttttcaacttagtccttctgttacaaaaacattacaaaaacgatgatgtggcagaaagactaaatcgaaaatgtttacaaacttaaaagatttatgtgtgatcaaaataacttaaatgaccaatctcttacaatcctcaaacttaaaggatttatgtgtgacgaaaataacttaaaagaccaatttattacaaatctcaaacttgtcgacctttggtgtaatttaacctaacttctttttttaataagtattgcctacaaactgaccttaatattttctctaggaattaatattctctttaggattttcgtcagtgaattttgtcgttgtatactattaatttgaatactgcgagtttgtttgtagatttatccattatatttttacaaatattaaatatatcattgtactcccacatcccaaattatatgacgttttgaacatttcacatgtattaacaaattttatatgaaaaaagatattataagttgttttacaaaattttctttaataaatgatatgtgaaatataagttaaaaaattgaaagaagatatcataataaataatcaatgctataatacaaaagtaacgattatgtttcattgatattgtaaaattatttataatttaggactgagaaagtatacattatcaatttaaatgaataaatataattttattttatttaaaaagaaaaatactttttgaaattttgcgttttttgtagttacattttttaaaataaaattgaaaaagagaaaacaacgaggtgtgagagtaaggtagaaggaaaggctgtcaaccaggtcacccaatctatcctttttctctttctcgtggttgaaatgattccctctttctctctcctctctctagtatcttccatttcaaaattttggtcttaacccaaatttccaaattcaattttcaaattaaccattttattacggtcactcatcacaagtcacgcccacaaccacacctaaacccacgccactctctcccctaatataaatacccctctcccattttcaatcctcttctctctcatttctcaccacacttctcgccgagaaagaaagacagacttcaaacaagaagcttctctttctttctcctcttcgatccattttctctctactctcacggtatgaaacttccttttctctttatcaacttcattctttttcccgatcattatttaggtttaggtttattcttcttgcttcgttgatttgtatagatttactgtttaatacacctattgttcatgagtttttagtaatcttaactgttttatgctcagatttgtgttaatcatgtatttattttgtgttattgataatttcgattgctattacttgatctatataactgatttagtagatcgtggatactagatacctagttactgatctgtgatctggttgatgcttacggtgcatcgctgtatctgattctgatctgttacatactggatctggaatctatttcaactgatcttgtatcgtctttagattccattttttggttttacttcgaaaatgtttcggttttgttgatttgttgactggatttagggtctgatctgtgttttcatgtgtgaattgtttcgttttttattaggaaattttagtaagccgagtcatagcaaattgaagatttaattacacatttgcaatgagaaactttagtttacatcttgcacatgttttcatcaggtttattctatgacattttgaatatgttaaattttctgattcgttgcactatgtacatgctcttctgttttttttttttttttttgaaaagtcggagttatctaatctaagttcttaatgaagaaaattagacattactgtgttatttcatttctctgttttatcgctattgataattgtgattcttatatacaatgtgagctttttatggggaaccgttttatgtagaacaggatagtattttcagtgaaactaatcttttaaatttgtaactggttgaactttgtttgtggtccatttgaattgttattaacgataatcttattgtgcaaactgtagtttagcaaaag

SEQ ID NO: 52 - DNA, 1500 bp, Cicer arietinum
aaaatattaaatcaattatagtaactaaaataacctgaatttttaaatttaaaaaaaaaactcaaattaagaaaatgcacttcattgttataatcagcaaaaaaaaatataacgaaataaaatagattaatttatcttaaagaatactaaagagaaacaaattattaataacaatttaaaaatataaagtttttatttattatttggctgataactcaatttagttgatttattagttaagaaagacctaaatttggggtgttaaaaaacgttgaaaaaggagaaaagacgacagaggaaaagggtgtgagccaggtcaccctatctatcttttttctgactctcgtggtcgaaatgattccctctctctcgtttcttaaaattcaatctttgttcctaaattaacccaaattccaaattcaattttcaaattacactcatcacgagtcacgcccacaaccacatctacaccactctctccccccatataaatacccctcctcctctttcatttcccttctctctctactcaccgaacacagaaagagagagactccaaccttctctttctttctcctcgattcttcttcacggtattttcatttccctttctcgttctacttcatttccctcgattatttagggttttttctgcctgcttattatacgccgattttttatttcattttttgcaatctttactattttatactcagattcgtctaactttgctcttttacctacttcatcagaatcatctatgtgttttttgtacttattcattattgcgattttcgtattacttgatctatctatgttactcggatgtagtagatcgcggataactaatgagtgatctgtgatctggtatatgcttatgatgcgtcactctatctgaatctggtcttctacactctggatctaggatcttttataactattgtgtgtttgtaccggtcatagattccattcgctagcgttagtttcaaaataattccggttttgttgatttgttgactggatttactgtctgatctgttgttttaagtgggaatctcgccgtttttttttatagaacttcatttggtgtttggaaatttgagatttgagataccctgactcatagttaattgaagattttagtgcatagcttaatgtaaagcttaagattacaggttgaacatgtggatatgttctattttattattgctctactttctttctttgaagacctcaaattgtctaacctttttctttttgttgagctcaaattatctaatataagtacttggtaaatgaaattggttaatgctgtgttatttcatttctttttgttgtgcttgctacacaaatgtgaactttttctgttttagctagaaaaaaatagtgttcgagtaaaactaatcttctaaatttgtaattggctgaattctgtgggtggtccatttgaactttttagcattgacaattttattactgcatactgtagctttgtaaaag

SEQ ID NO: 53 - DNA, 1500 bp, Cajanus cajan
aaagataaaaattgaaaataaaaaaataaattaaagaatgaacaagaacaacatattaattttgcctaaggcttataattattttactaaaacaaaatataaaaaatataaattattttcataagtataaataatatctttaaaaaaatatattaagggaagaaaaagaaaagaaattgatggagggtgtgcggaggaaaaggctgtcagccaggtcaccctatccatcttttctcgtggtcgaaatgattccctctctcccttccttccttccttccttccttccttgtatttcaatttcaattttcctcctaatttcaaatgcattacttacactcacaccaaccacactctctctctctctctctcctcacatatatatatactctctctttctctcttatcaaccccctttctgtagccctcaaccacactgagcctgcacacccattcttctcttctcccacctcgcctctgctcattcgcctccttcctatctctgcttcaaagtaagtctccttttcccctttcattcgccgctttttccactcttctttctccttagatttgttcaacgctatttttgttgcatcttctatgtgagaatgctacctgccgcatctgaatcctatcttgatgtttatgtatttcatgatttatctccatatttctttatctatgtttatgaaacgtactagattgtggcgatctgtccttctacttccgatcttcttcggctaggatctcccatctttctataatctaatctatgcttttatttgtcaaatgtggatagttgtcggttccgtattccagcgttcgatgcctttatgctctgatctggttttcctttctttattatactattcatctctccgtgtaatacgccatttgatccatttggtatttaggaacttaagatatcctgtgtcagaggaatgttgagatttaactaggagctgcacatgcttacttcctatgtgtattctacgatattttccgctcattttttaagttttcctagtttcacttaaaaaaaatgcaaatgatcgcctcctttgtttgtcaattatttatttgcatttgctattgtgttctttctttataaacctcaatttatgtaccttatgtttctatggaattataaagtagatcatataatatactgaattgtttaatttaccaaatttcttcatgcttgaaatgtccgctttctgacatgaaggtagtgtgttgaggatctcacatctaactagtgatatagcttatatagtgtatataactcggaagcaatattcatcttacaaactggttttgtaaagttgagttaggttcaaactcacattcttaagataaacaaagaaccggtgtctcattttgaaaatgagagtttgaaatgaacctaatcctaaagtctgtgataggctggctctgtaggttgttttattcaatttgttggtattgacaatttgtgttatgcttactgtagtttggtaaaaa

SEQ ID NO: 54 - DNA, 1704 bp, Glycine max
aaaataactattttttaccgtatttctttatcttgatgatttagtcctatggttgttatatatatgatcagagagagagttgctatgattttgtcatatttgtgcaacatttatttgtttcattcagcattttttgtttttttattttaaatattaacttatggaaaaggccatatgcaagaagcagccttatccaagaataattaaaaatagacctaaacgctctatgattgtgttgcctgaattacaacaagttcattgcttgttatgctcacaaaatattggatataattaaaatttcatgtttttatcctattcttatgttctattatatctaggaatatgtgtatactttgaggccttggccgcccctaattttttttataatatacatgatattttaaattttagatatttttttaaaatttaattaaaaataatgatatttaaatgttttttattttttaaataatcaattatctttatatgattttattgtaaagaaaaggaagttatttttttaatacacatattttatttccttttttataatttttttgtggagacattacaatttttctcttttttgataaaaatatttgggtatcatttttttttcttaataaattttgctttgataatcctttattgctattttaaaagttgtaagttattgtatgtgattaaatgatatcgtcgctcttacttatatgttgttaatcacgattttgggacatgcaaagaagtattctttcttctatcattaattttcatattaaaattttaatattttttgttcttgtattttctttgcttaaccatttttagtctctactatatcaagtaagaacgtgtgacatgataaccacgtgatctaacaaacatatattattaacattttgtataaaattaatactaatatatatgtttgtttgataaattttaattttgaaaattacctcccctaatgttcaattatgcctttgtccctatcttatatcttattacatcatcattaaacaaaaatgtaaaagcatagttattaaacttgatttgaaatggttggatcgacgtgttaggtggtcatattaaacttgatttgaaatggttgaatctatccgttagatggtcatacttaatggtcatacttaaatagaggagtatgtgttgaaagtctcacatagcaactagccatgagaaagatgacacatgttaaaatcccacatcaaatcaaaaataatattgagataaactatataaataaaaaataattttcaccttataagctgattttgtataattgagttaaattcaaattcatttttttacatgtatccgagtatgattgcatcccaaggaagactaaccttatttaattttgaaaaaaaataccaaaataaaatagaaaaaagaacccacacaattaaacaaatagaacgaaagaaagaaaagcgaatggaatattcgcatatctgttgggcgtgaagcagaaaccacagaaaaaaaaaaaaaaaaaccatacagcgtagtagtccttggaagttggcacagcatcacgagtcacaagccgtaggaatcacgcagttcacttggcctatttacctatcactgcggtcttttactcttctactgccttcttaaaaccctatctctcaccaacttccgctacttcctactccaaccactttcttttttcccgcagtc

SEQ ID NO: 55 - DNA, 1713 bp, Glycine max
tttattataaagaaagtttatataaaaaaaacttgaaaacaactacggttattaataaaaaccgatgttaatatagtaaacaacatcaattatttagaaaaatcgatgtcaacatacaccttaacatcggtttttcaataaatcgatgttggttatttctaataacatcggttatttatacataaccgatgttaatatacaaactgtaacatcggttatttataaataactgatgttatatataactaactacaacaaataagtgtatagatgatggacgttcacattggttatatataaaaaaacgatgttaatctattgtgtaacatcgattttatatcaaaaccgatgtcaacgttcatcatgtatacacttattttgctatagttctttatatataacattgcttatttagaaaaccgatattaacatttttattgtaacatcagtttttaaaaatcgatattaacaataatacattcagcatcagtactttcaacatcggttttaaaatcgatgtagaatgctgtaaataaccgatgttgaaagtatattttctaatagtgtagaggtattcgagtatttgaccaagctgagtacttgaatacttactgtgttgaaagatgcatttaagaaataaggcaagataggggagtatctaagtattcggcatgtccaaatatccgaatattagtggacactttgaagagttcttgaatgtcaaggttgttagcgtgcaagtattcagcatgactaaatacccaaacattggtgaaagctttgaagagttcttgaatgacaaggttgtcagtgtgcggatattcgacatggctaaatatttgaacactgatggaagttttgaagagttcttgaatgtcaaggttattaatgtgtaggtattcgacggcatggtcgaatacggggatatgaaatctaaggagcacttaagtgttcactcaagccgaatgtctaagtatcttttatagaatatacttcaatgtcctaaagattttgttagagatattttaaatgtttgattagaccaaacacttatctaatatactttaaaattacattaaaagttatttgatttgtaacaaaaacattttacattaataatcatactcattaaactcatcttaacatcattttcaacaaatttatccgtacaaaagttaattttgatcataaataattatcaattataaataacaattgatgattgaaataatctaatttttatatattgcaataaaattttacacgcttaattaattaaaattataataaatatgattttcaagtcattattataaaaatgtagatatcagtttattaagtaaatatttaatgaatctttataatattatctaattataaattatctcagattaaaaaaaattaatcataaaaaataagagtaagctcactttttttcaaaaaattaaataaaatagaatcgcattgacgcgaggcagaaatcgcaaaaaatggtatcacgggtcatacgccggtcccgtaggtgtcacccgacccacctacttcacttacctttacctgtcatttttcgctcttctttttttacccttctttcaacctttattaaaaccctatctcactcactctcacccacacaccgtttgttctattcttaactcaacttctgctacccactctcttcttccaagtccaaccacgcttttctctacagtc

SEQ ID NO: 56 - DNA, 1656 bp, Glycine max
attatcaccgtttttggctatttcattgatttattaaattaaatatcatgccaccctccaaccattgccacaagtttcgaaccccagaaagaaagtaaaaggctagtgagtggtttgaaaattgaaagccacgtgtatgcgaacattggcttccacttcaaaaacgtgatgatgtgcccactgaatccgatcctcctcgctcctttgtgatttctcattagaaaatagaatctagaaactataggatagcgttacacacttacaaaatataagtatttcactcaatttttgacaagttgttattttttcggtaaattatgataatgacattttaattttagtacatgaatgagttaatgttaaaaatataaggaataagaagttagcttttataattttatgataatattaataataataataataatagtgattttttaagatatgaaaaactaaatttatgttttttttcccaaataactgctaattagtatgaataggataggattagtacaatctattgcaggaaagtatgtgttcatgttttattagacaaaaattaaacaaaattttaaaataaaaaacagaggaaatcatgccttggcttggtaacttactatcttctggtccttcatatgataaacaaacagtgtttttttcccctaatcataagaatcatataattatttttaaatgtattaataactatttttttatatctttaatttgttgtgaagtcttttaatgatcactcattattcatgaaagtatatacagttaatgaactattaataatataacttattctcatcgtgtaacaagtatttttcatgtattatgagtagtgatattatatgtaaccacttcttatatccattgattttatggatatttttaaaataaaatttgaatttatattagtattaattaaaagtaactactttaatcatttttatttgtcttgattatttaatcttatggttttcatttgtgatgatgatcaaagatagtatgatagtatgattttgttatatttgtgcaacacttagttatgtttaataattttttttaaaaaaatataaatatattgaaaaggtcatatgcaagcggtagcctcacccaagaataattaaaatagacccaaattctctgaataaatagacctaaatactccatgaatgtgtttcattgtttgttatttgatgttcatcaaatatcaaatataattaaagctcatcatattctcgtacagtatagtattagtattatatcctgctcactaaaccaaacatctaagaataaccttatttcatttagaaaaaaaaaaacccaagtaaaattgaaaaaagaatcaaaacaataaaaagagagaaaagcgaatggaatattcgcatatctgttggcgtgaaacagaaaccacaaaaaaaaaaaaaaaaaaaacggtacagcgtagtagtccttggcaaagcatcacgagtcacaaggcggtcccgtaggagtcacgcacttcacttggcccatttacctgtcattgcggtcttttactcttctcaataccttattaaaaccctatctcactcactcactcacaccgttccatttctcaacaacttctgctacttcctactccaaccgcacttctgctccgcaattatc

SEQ ID NO: 57 - DNA, 1883 bp, Glycine max
ctttaggcctccaacaacccattactgaaccaaacttgccaaagtcttttatttccgtaatttggaaatatctatgtagttggtttcttaatttaattaaaggtggaatgaaaactctccctcacacgtcacaactttgtatgatagctcaaggtttgtatcttctggctcataaatttcaaggttgtctgcttcagcatgataagggaacaaggaaacacactgtacatggcttagcgttctacgctggtattcatccggaagaagtttggatagaacaagttcccatccacttcgtatcgatctcttctattttttttttggtaatcaattttttttccatactctcgatatcatttcattagaaattaatctcatttgtgatgtatgattattattaacctaaaaaattttatacgcgatgagaatataatatttttattaaataaattattctcacttatatgaatataatgatattttagaagttttatctttatctcaaccctcttgattaatgttagtgaccattttatatattgttgtttcgtgatttcttattattcgaacatgtgtgcttggaatatttgaatcggtcctaaaaaaagcaattacctatgccttattcttgtaaataaattttaaaggcatatataaagtgaatattcagtaaataatttaatgatttttcagaatattatctaattataagttatctcaaaaaaatctaatcataaaaaaataaaaaaaatgtgtatttggataacattcaaaatcatgataaatcataataagttgaatcaaccttgattttgataaaaaaaattatatgtttggattatcttcaaaattgatagaatcaattttaatggaaaaaaaaactaggtagagttaaagcaaatagaagtaaattttacttcttccaaaattaattctacttgaattttaactaatttactttcacatcaattcactcatgcatatgtttattatttatttcttaattttaaaatataattaaatatgtcaaaataaaaatataattaaaagtattgctagatttatttaattttaaatataatttatttaatttatcctcacacttatatctttactggtgtgataaatcttattttcttatctattttatccaatttcattgttttacaagaaatattattctatttcaaaagttaaactattatacacatattttcttaatttgaattatctctatttaattttaatttttatatataaaactaattataataaaaagtgtacataaatattaaaacccaattttgatcattataccttcaaaattaattttgattaaaagcattcaaataatatattttttaaaaatcacttatttataaccttttatccgaataaaaatcacgttatttcatgattaattctgtgaaattaattttatgaggctaatcaaaacgcacacttaagctcatttaataaaaataatagaatcacattgacgtgaggcagaaatcgcaaaaaagggtatagcgtaactcatggcacagtatcacaggtcatacgccggtcatgtaggtgtcacccacccgacccacctacttcacttacctttacgtgccattttgctcttttttacccttctttcaaccctttattaaaaccctatctcgctcgcactcacccacacaccatccgttctagtctcaacttctgctcttccaactcctacaacgcttttctctgcactcagatctaggtaagcaaataaccaccacactctcacgcttttctttccattttcaactctaccgcgtttcgtttctctcgatctgacatttttcttctttctgtttctaaatgaatggcatggatcaggcggatcc

SEQ ID NO: 58 - DNA, 1500 bp, Cicer arietinum
tgaaatgatctttaatatatataaataaaattaataacaaaaataagttaatgagagggccacattatcaaaaatagtgagggaccaaaaaaaatctcaataattataagtatcataaataatatgattaaaaattaatttttgataaaatagaggataaaaaacacatttaagccttaaatataagactttattgaaatattttattttctccttttacaagatatttttcttaaattttttaagtttattcattatttacttattagtctacctttccctatgttacattgttttcttcttctttaatatgtcataaatactataataaaaatataaatacttaacgcttatatattatttaaaattaactttcagtccatttatttaaaaaaattagtttttggttttgtattttatatattgtgaaattttttcgatcacactcgattttttatgatgttatatgttcaattatgacctgactgtgatgaatcattacaattaaattgaatctatctcattaatcaatatcgatttccttattgtttattagacaaaactctaagagcctttgttaataacatggtaagcaaataattatgaagaaatagaagaatgtttaacgagattctattttatgcgattcagttgagcaataacagtgttgtctcgttaaggaagatactacatcatgaaaaacaaaagggacaaactaaattctacaaattgtaaaataggaaattaaaaatccaattttaaaaataaaagaccaaaaactaattttgaataaagtaactaaaaatagccttaagtatttatcttctagagtaaatcagtaaaacaatttaaattgtttaccaatttaataatacagtagattttattaatctccagagtcttatatatataaagatgattataacaagtttttttatacgtaaaagaaataatcaatattattataagttcacgtaatatataattaaatattcgactattaaattaagatgttgaccataacaatatcaacctatatcaattgaattaatacctatatcaattgaattaatacgtaaattaacaaatattcttctagagtactttggaatggagggatttaaaatcgtgtaatgtaatacgtagttacgacatttactattcaactcaaacataaacggcttcgaatattccacaaaattccgtgcgattccttctagtaatcttctcctacggtcaaataataagactacaagcgtttaactctttttgataaatcaggggataccctgggttttcactttccacctagcagtaaacaaaatcagcggctactaccttaccccttctccaagcacaaatgtcacgtgacatgcacacattctcttcttacacaacccttttaaatagcattgcttagcctttgttttctccaccaaaccacactctaatctcatttctctattatttttcaactctattattctcactctcattcta

SEQ ID NO: 59 - DNA, 1500 bp, Cicer arietinum
cattctatatattaagattaatcatgtcatttgagttgttattataattcatttgttgtactacgctaagcggctacttcattttattttatgcgcataagatcaccgttaatcgttacaaattctattttatatttgtcttaaaaagaaaaaaacaattctattaatgctataatctatgatcattcacgtcttcttcttaacaaaaaagtaaatgaaatgtgtagtaggtgttattgctcttcgaacaatctagacacaccaaaaaggccagagataattaccacttattatttgcgtgacgtggctcttatccatctgtgacacgtgtcactgcaagaaagggcatccccatattgcttcgctgtatgagagcaactccaacgacttgtggtgttgggtgatatatatccaaaaataattataacaaaatacttcgtttatccttaaatttgtaaaaaaaagtttcatcaaccaaaatttaatatttgatatatttattttaaattttaaattaaataaattaaatttttatatttagttttggtggagtacaaaatcaacacaattaatgaatatctttattgttgatgttaaataattttttgttaatattttcaaaaaattcattattttacatttatataatgattttgaaattgataatggacatgaaaatatgatattttttaaatacgaatataaaattatttaatacaagtagtatatgtgtatgaccattgaccattatctttaaaagcttatagttttattcttttagaaaaaaatttagatataactgttaaatttaaaatatttaattgcagttttgatttatttattattattattaatttacaaatttgatctttttattgtaaaagttaataattttaatatattttagatttttgaattaaaaaaataatgatttgacatattattatgagatacaaataaatgacatagaattatttatatgcatcatcgcatttatagaatcatctatatgcattaattatttatatatatacttttatttaaagtatcttacaacattattttttatctaaaaaatcatagaaatgatgaaaactattgatttttaaaataacatataaattttgtaaaatagtacatatagtacaaaccctttaattagacaaattcaaaactaaaaatacttttttttacaaaaaataaaataaaaaattagaacttagaaatgaataacattaacaggctgtcacgaaatacgatttgtttattttctgggttggtattttataaaattttgattcctcacaaatttggaagtattctaaacgaatttgtaatattgaagtattttaaccggtaaccggttacagcccgtgaaagtggaggccggtcgttctccatataagaagacacaagcgaaggaagcgaaaacaagaagaagcgaaagaaagaaagaaagcgctttcagtgactgagagaggtataaaacgctattccaac

SEQ ID NO: 60 - DNA, 1500 bp, Cicer arietinum
attttttacgtagtattttagctcttcaaattatatattggttttacatattgaataacattgttattttagtatttaatttgacaagtttttatatttgtcgatcgtgatgtagtatgaggttatccaagttaatatgtaaatattttattaattttgacaaaaattactaataaaatattttaatggaggaattaaattgatttatgttctttaatttaaaaaatcaatctttacatttcatggaataaaatgaagcaagttttaaagtgaggttttttcttttaatattccaatttttttaaaaacttatcaatttattactttttgaaaattatatatcaaactatctcttttatttaaaaatcgtcatttgcaaaagcgacttccttaaaaaagtctgagtttgcaataacgatttatttaaaaaaaatttatttattttttttagacttttatgtatctttgaaaaatcgtcattctcaaatgtcatttgatgctatcaaagaaaaaaaaaacaaaataatctttaagcaaattgtcattgtaaactcagatttatttgaggaagtcatatttacaaaatacaccttctaactaagtaaaaaaaatgcagtttgatatataactttctaaaaggagtccattgatatttttataaaaaaattaggttttgaaaaaaaaaactctaaagtgaggttactttgacaagtgatttactataagttgatattgaaaattattcaaattattaataaaaataaatattatttccttattttaaagttgattgatatttagataataaagcaaatgataaagaaaaggcttaattgcagttttggtcctcctattttagctgaatcgcgaaagtagtcaatccattttgtttctccccagttttggttcccaaacagaattttagtccaaaatttgatgaaattttattttttaaagctgtactacaccatttgtggtcatatatttcaggtataattgttgcaaatgagatttgagccactgtatgacttaaataaatgcaaaaaaagtgaaatttcatcaaattttggaccaaaattctgcttggaggaccaaaattggggagaaataaaatgggagactactttcgcgattcagttaaaatagggagatcaaaactacaattaagcctaaagaaaacgaattcctgaaaacaatcatgattagagtatggattattttataaatattggtgagcagtaaattatatgttatgttagttgatcgaaaaagttaccttgaaacgatgaatttgtccatgtaaaagtatttggaagtcaatgattaagaaaaacattgaataaaataaattcccatttgcagtattgtgagttagattgattgattgctgcaacccggtccgtgaatatgactcacgagaaagatgttagaaagttggaagatttgttgagcatataaggatctccactcctccaattattttgagaagcacaacacact

SEQ ID NO: 61 - DNA, 1500 bp, Cicer arietinum
atatattatattagtttaatttttctaaggctaataaaattatgaggcttattaatttacagttcacttattttcatcaaggcataagttaataaccttcgccatttaattttggactatacaacattttgagataattttgacttattataactttgtagtttgtacttttaattttgatgatttgatgggtcaaatttaatcaatgagacatactatcactttaaaattattactttgataatttaaaaggttcactcttaactaatttaataaactatgattctaaaattttaattctaatgatccaatggtttcatttctttctgcaactaataattattatataatatataaataaaaaataaatacttaaattatatattaatttcgacgattaaacggtttaattttaattaattagactaactatgatttaaaaattataatttcaacaatataacggttcaatcttagctaagcatactaaatatgactcaaaaatttcaatttcaaagatcaaatattcaatcttaactaattagtcatattatgtcttataaattttaattttaacgatctaatggttaaatcttaattaattagactaaatattatttagaaattataattttgacaatcgagcagtccaatcataactaattagattaactatgattaagaagttctaattatgatgatctaaaggttcaatcttaactaattagactaaacatgacttagaaattataatatcgacaatcaaacaatccaatcttaacaaattaataaactatcacttataatatatcgcatctattacatgattctctattttttttttgtctatattagatatttagatgtacaattttttcgagttttttttttatcaatatatccttttttttttatttccctaaatgccctaatttaaaaactatttctctaaatgtcttatttttttttaaagcaggaagtcgttctttgaggaagcgaccacttgaagacgataaataaataaaaagggatatattgataaaaaaaaaacctccaatttttcatatcctaagtgcaaatgataccattaattatttcttgataattggtgttatttaagtttgttagcaataatttatatccagctaagatttcccacaaaattgttccttgaattgcacattagaatcttcccttttgtggtgccagtgtgaatgccaatgtaagccacaaacacttggtgtgtcttaattggtccaacaaatatgaaccacaatattctgatttttaatacaaacctatccaatgagattctacatgccacatggcaacactccatctaataccaaattcacattcctatcctttaagataagaaacatttgcatacattaataaaaatttgcacattttctttctcattcaccatccaaataattcacatcaacacaaactcactaagcttagtcactttatcccttgagggcaaaggcagctatc

SEQ ID NO: 62 - DNA, 1500 bp, Cicer arietinum
aaagtatgttaatttaaatatatttatgtaaaaaataaatcggtcaataaacttaattgttaaaattgtatttgaagtcagaagtcaccaatctaatttttaatttgaattgattttaaatgcaacattaattataattgagaacgattgaacttgtcaaaattaattttatttatttaaaatcaattgaatcacctaaacctaaacaccacaaattctctctaattttaaatattaacaattttagaataacaaaaattgatgtatataatcagaaatttaaatacaataaataatttttttattgatataaagttatttatattaaagtgggagggaatatctaataaagatgataagtaataataatatgatatggatacccgacatgttaatagactttatgtatctctcgtggagctttacgcctttaccatcgagcttctgtctttaatgacaaaacacggataaaattttcatccattcttatcaacaattaattcaaaactaaaaatgaacaagaaaataagtcatttaatcaaattaggtctatatatactttattttaagcattgagcaaactaaaaacatgctttaataattttttagatataaaagatttatccatgtgaacacgtatttttagtctaattcatgtattggtactgcaattaaaaaagttaataaaaattatagtaataatggaaaattaaaattaaaaaagaatgagaaataacagaatagtaataatcataatataaaaaatggagggggtattttttaaaataccgtgaattaaatctagttaaacaaaaataaaacccaacccattcatcttaatttctagtagcaagtgagactagggtgggtcaagggtggggaggccttaagtagtggaggtgactggtgagatagatagataatgtggttatttcctattgaccaaaaaaatatatgtggtcatttccttaaaaaaaatcagtttcactatcaatgccattccattttaacaaattaaattaataaaattgtgtcaaaagaaaaatattattttaattaatattacatacattattttaaaaaaattatattgtattttattttgcaataggctaaaatacattaattattaataaaattaatttgataaaattattaccgcaaaaatgaaacggtaatatacaaagaaaaatttaatgcagaagagaaattgtacaatcttcaaggtcaagcaaccatgagaaaatgaatgaaaaaagcagcatgaatggtaattcactaattaaaataaaaagaaaaggaaatatcttaaagcaagatgggccactattttttaatctttcataagatcatttatggcctaccaagaaataaaaaataaaaacttatcatttaacatgtaacattatcatattcactttataaatagggaactaatcctttccaaaaatcatatcacttcatcattataatttcatccatttctctatatcaattacc

SEQ ID NO: 63 - DNA, 1500 bp, Cicer arietinum
acaagatggaacggtcatcagtgtaccatttcacttggtaaaattggtcatagtaagatgaggatgagatgcatagaaaataaggatttatcttttacaagaaagacaagaaaagggtatattagtgaatgaaaaatgacaataatgcagagtcaaaatagtgaaatagtagtattatagtaatgtagaaaaaaatgtgagaatgggaccacagacaggctgctttttgcaaattgcatatggtttgtcaagacaagactatatgcacaataatgaaaatggagtttggccattgcatgtaagaatgtgatcattctgagctggtcttttaataatagaaattgacaccattcatgctcgcagcatgttgaggattactgaatcaattttgcggttattaagctcttgttgctaaatcatgccaaaaatatttatcgttaaagttaatttcattagatttatctccctaaaagaatcagagtcgttaattaaaattaataattataggaaaactacacgcacattttcaatcaaagggtattgttggattgatggcataagatgaaatcaaagggtatattgttagattgatggcataagatgaaatgtaattgaaatagaataaaatacattagttaatattaaaaatttgatgctactggtcctatgtcattccgctatatttcaatgatataaatccatcgagaagaatgttataagtgaaacaaatctaataaaatattcttttctcatcttagatatgatgatagtatataaagacattttgacttttaaatttaacaatcgtttacatcaacaaaagaaaattatttccttttttttaattttttactaacaaataaaatttaagaaataaaaatagatttttttttttaaacaaaccaactcatgatttatatatataaataaagaatgatgtttttttgtttatggattatctagtatatttgtattaataaggctatatcaacatttaattgagagaagatatcaatcagacagtttggccgagtggtctaaggcgccagatttaggctctggtccgaaagggcgtgggttcaaatcccacagctgtcactattttttcacttccatataaaatagggccgttttgtaaagtgacttagcattcactctatattatatatgtaaatatttattgacgtcatatctgcactttacatatagtatattagataatattaaaatagtaataataactaaaatttgagtttaaaaatgataaataaataaataaaagacacttaaaaagaagggagggagtatcttttaatttgtgatgtggatgtacaaaatagtaactgacagccgttcaacacccacttgaattaattgtgagcctttcaaattgtatcctcttcacctctttccttctttataaaactcatctgctactcttctctcacaaatatcaattacctttctattcctaaaatactcttctttctcgcagccaca

SEQ ID NO: 64 - DNA, 1500 bp, Cicer arietinum
taatataatttaactaatataattcaattattcaatttataatataaaataataataaaaatttaaaaaaccataaattatattcataacaaaatatacaaataataatatattaaaaaaattattaattttactcttaatgaaataataaaagaaaaaagtgttcaatagtactgtatgaatactagtactaggtaagtgtcttatacaagtattctactattttagaagtattcatgtttcaaagatttatgatttaccataattaattgcataaaagatattcaattttaaaatgattagttatactcctataaagtataatttgacggtgtctgtcatcattagaattttgaacaattttttagtaccattgatcaaattatgtgacttttttaagtgatctttatttaaatagcttttaatcataatcaatcatgcaaggttgaattgtctagatttagtcatctcactctctcatattaaaattactctcacttgatacatcccgataataagtgaaatgaaatgatgtgttagaatgagatgtgaaaaaaatgaatcttaacgatcgatcaaattatgaaaagttgaaattaaaataaaaaattatatgacttttaaagtaattatatactaaattctcttatctctcattagaacacatcatctcacatcatttaaatagaaaaaaaaaacatttttttttgaatggtgagagatgacactttacctttttatgagagggttgatttccatatttacacacacgaaatgagagggtagaattttggtctcctaaatttttttgagatgttttatttctacaatttttcaattcttacaattaaatatctagttccttttaatctattaaaaatatttatattaataaattgtttaacattattttacttcaaattcaataattaaaatatcaaatttaactcacatcagaaaaaattaaaattctttgatttaaaatcctataaaattctttgattttatattatattgatcttctaactaaatattttaataaaaaaatatttagtaaaataaaattgacagtaaaatcgtatttgaaagcagatgatgtaaaatagctaaaaagaaaagtttgaagaccaaaattccacattattcaaattagaggttacaaatataaatctaatggcaatttttaattttttaagcacgaagcggcgtcgtttgtttgggcctgaaaaaaaacgtatacacccataagacgaaaaaaagcatatcccatccgtgtacttggatcgcccccattaaccatagtcaaatccaaactcaaccatagttacatctcttcctcagaaccccccacgtgtcaagatccaacggtcacttctgaagttactcagaaccacgcgctattataaatatctatgcttgtctttgcttaacccccaacccaaaccctaatccctcttccaattgtacaattagattcaatcatttctattaactgtagcc

SEQ ID NO: 65 - DNA, 1500 bp, Cicer arietinum
gaaaattcagacagtgatccggattagtaacgatatcatacaactttgaacaggcagagttttgttagatgatgttaattttatctggattttgataaattttctgagattcctcattgccattaattaatctcttattttacagttgtggtgttctgtatatgcatataccatgtaaactgtcttctttacccacaatcccacatgatggtgcatgtatccttgcttattttttccttcactcaaacttcgatatagcgaatcttgttcacttgattagttgatattgcactgcacatatggcaacagagcgcacgatttactaaattacaagtaaagcatttgctttaggcccataggatattgttaatcgctaatattctcaatatggattgtggcattttttgtggacggttggatttcttcaagcagagatttatcggacattgcattttgccacatacaaaaaagtgaagatcagttatgcaagaattgcatagtgccgaccgagagcatattgtgcatataaaatgggctcctttatgtgattctgcatatttgaagcagaatttctttttgagaaaaggatgttatcggtcagagaggttttggaatataattaaataattagtagtgactactcttgatatgaaggaaataaattccttccgtaggcaagccgaagaacgtgaggaagatgcccagtgtttgatttggcccaaaacatcaaaatataaaagtttgaggtgcggagtttttacgcaaaggacgacaagttaatgaatcgaatttttatctactagattttaaaacatttaaaattctaacaagctctcacaacacttgacataccaaagaacaattaaaatcaattatattcactcgacataccaaagaacaagaatcgacttagactgttttttaataaataaattgattatagaacaattaaaatcaattatatgtactttactaattttgttttagaattttaaatttaacataattaacaaagtatatgtatctaattttaattgattatgaatgacacaaaactttaaaataccaaaaaatttagtaacgcactaaaagttatgcttaaatgtctaaacttttcaatattttgtattcctatgacacttgttaatctgacatgcacatcagaagctaaataaagaaaatactagtataatacttggttagttactgaggtgttaatttgtgtaagcaagttaaaaatcttgatgcggtcttattgattaggcaataatagtatatattgattccttagaatcagatccgcattgaatcactactactaatggcagtcaaattgcaaagtaaactatagttagtatatcacgactattacgtgtcccctatttatactccaacaaacaaactaactaactaactcatgactgagtaatgcaaactcaattgccatcatcttcttattcttctccaataggtaactttcagttctccgcgccttcttaa

SEQ ID NO: 66 - DNA, 1500 bp, Cajanus cajan
tagtgattgtggctacgttggcagatcatatgcgtaaaatatagtccaaattcgtttgtatattttctttgcaaaagggaaactcatgagtggtgatacacacaatataatgcatccaatgctcatcttttactataattagttccaatctaactctttcataactcttaacttacctcttaagtttagggatcatatattcatttataactagaaccattgaagttgctagctgtcaaaagagagagagaaaaaaaccactgaagttattattactacagtaattagattgaatagagaaagcctgacagaaacatgagcagaatacgccaaaattgaatgaacttttcacttttgttgaaggaagtagtaagcttataaacctgttacatacatacattattgaagtgttgaagttgttgggggtagatcaagtcgatgcctcgaaacagaatattgcagtgattccagatgcacggttccacctgtttgctaaaatatacagaataattgaggggtccagctaagctaatcccatcatgccgcgcgggagattaattttaattctcttaatcaaatttctttccatttaaagcagagctttatgcaaaatctcaaatttacctttctggaaaaagagggtacatcctacatgcaacatttatgtttaataataaacaactaaagtttgccgacgtgtcagtcgcgctctgctaattatacctgctttttccggtgggtcactgacagagtcacagtgcttatcctcttatctcattcattcattatattatcatcaattatgccttataattatagattaagtaagtactaccactttgctaaattttgtagttataacttataattcactagtcccatccattatgttttttacacacgcgatacataatataattctctagccaacgtcactcacaattgctctcttcgaactatccagaaataaaaaaaggccaagccatttccacattccacttatcagcggcctacgtggctcttatccatctcggacacgtgtcagtcgtggaacgggtaacgccgaagcgcttcgctgtcttaagcgcaacgactcgtggtggggatatataccgaaataaaaaaaggaaataatatttatataaaaggataaaataaaaatagaaatttaaaaatagaaaaagggaaagcaattaataatttgaaatgaagagcatgtcacaggctggcacgaaataggattgtttcttttccggcttggtgtttttgaaaaacttgattcccgtcatttttggaagtactgcaaataatttcgaaacctgaaagaaaaaaaaaaaaggaaacaaccgggaaccgggtagagccggtggagtgttgttgagagtggatatataagaggcgaggaagcgaaaagcagtggcaccgaaacagagagttgagtgagaagtgagaagtgagaagtgagaagtgagaagtgagaagcgatcagcgtagtggaag

SEQ ID NO: 67 - DNA, 2306 bp, Medicago truncatula
agacaacatatctagttaatttaggatgcaagtgttgcgttggattagtttcaaccctcccaagtataattcctaaaaaaaaaaaaaaaaaagactccaattataaataagaaataaaactgagcagtactttggcatggtttataaaatacttcgatgataacgtatttaaatagatgataatgcaatgattaacaaccagtgattgcgactgagttggcaaatcatgttcataatatttaatccaaaacaaacacattgaattaatccatcgaatacacaatcgaattaatccattgaatactcattaggagttctctttatttgtaaatgtaactctcactccaaatttgggacggtatacttttctcacccgttacacatttatttgtcgctgtttggtccattatttttggcaaaataatgataataggtacagcacattcttaaaattaaaacaaaggcacaccacattcatttgaactacttggtgaaggaaatatgcttaaagaacttgataaaacggttgggattattgaagttgtcatgccaagtgaggaaacatttatcaatattttttttttgcggcattttaatacttttaaaatattacagttttccattttagaaataaaaaccaaacatattaagtatcataagtttattttttaacaaaagaaacataaaatatggaaaaaactaaaataaatttttgattaagtaaaatgcctcgaaacagaatatttacgtgattcaaggggcacagggttcacctgtttgcttgtacaaataattgaggggaccaacttagctaatagctttgaggaaaaaaaaaaaactgagccactccaatcgtaggacatggattggagggaaatcaaattcaattaccgaaaggtactttttttttttttcctataaaggtaacgttacacaaaaaataccatattataagaaaagtcacatacctacatatcacattttattttattttttaataataaaataacaaatataattcatcaacgtgtcagtcaccgtctgctaataataccttatttttccggtaggcaatgctaatctcctaataatcttatccgatttattatattagtgattagtgattaattaagtcattggagctatagaagattattaaatttgttgcattatgcactacaacaattcacaattactacattttattttatgctcgcaagataatcataacaattctattgctataatcatgtactacgtcttaaaagtaaatgaaattccttaaaaaagaaagtaaatgaaatgtgtagtaggtgctattgctcttggaacaatctagacataaaaaagactagaaataattaccactaataagctgtatgacgtggctcttatccatctcagacacgtgtcagttcaaggatggtggactcccgtattgcttcgctgtatttgagagcaacacaacataggtgatgaggatatatccaaatccaaaatatttgaaataaataaataacacaatcaagtgaaagtggaatcagtcccgtgaatgtagctcggttgttatttatcaagggacattatatgtacgggttggagtatgaaccttgcatttttatttcttcacttataacaaaatcaacttgtaagtgtagatcaatcgaatgatagttagcagaggtatttctaacttctccacatatgatgtgtgtgaatttatctactatattacctaatataaataaatggaattaaatgttcttttaatcctaactcaacaaacaatatcgaaattgttagttgaacatcttcactaatagacttcaaaaacctcacatttgaatgcgagttttagtggttcttaccagattatatgcaaacaaataattgtagtctttttcaaattaatcaaagatattgtgattcaattcaacctcaaaaatcgatcacattcaatcttttagtgagttaaaaaataatattgattcacacaaaaaagaaggaaaagaataacatacatgtcacacaggctgtcatcaaataacattgtttcttttctggcttggtttttattaaaatgttgattcctcccaattttggaggtatcatgaaaaaaaattctaatattgaaatatttcaaccggtaacccgttactaccggtgaaccaaaaggccggtcgttctccatataagtaacacacaagataggaaacgaaaacaagcagaagcgaaaaccagaaagcgctttcacagtaactaaaaaaaaagagtgagtgtttgttaaacaaagcgattatttcaac

SEQ ID NO: 68 - DNA, 1500 bp, Cajanus cajan
gcatgcccaactgaagcttaagcctcatcaccaacccataaaaggctcaacatgccaaatatatatatatataatgccaacaaaatgctactccaaggtagcctacaaaaaaaaatcatctactcaagctcctaccaccaagtggcatccaacagctcacaataacctatagaaaattaaaataaattattttttatttttaaattttgaaagaaaaaaaaaaccaagtgaaaaattagttgctccaataaccgatttctcaataaaattttaaattttttttcaaaaatcttattgaaaaatcggttatccaataagatttatcatctaggctacaaaataggttattcaagtaaataaaaattaacccagattatttaaatattaattaaaaaaataatatatttataaataaaaaaaagtatagaattggagcactagtttaacttattgacttgtgacgacatttaacaattaacatttaagcatcggatatttacgaaaaataagatcaaatctatcactttggatatttcctttttacaccaaggatggatattttttttaatcgttcataattccaagagtaatagagaaaaattaaatatgtaaaaaaattgggatttattaaaaatggatgaattctaaaaatggccaacagattccaaatatagttgcaacgtttatcggttcccggaaaagactcgtacaccaaactttgccgtgtcaatagcgtttacggtcctaattcttccacgcgtcacaacatgattggaagtggtaccggtgccgtgacgaagcaacgcatcagttttgctataaaagcgaagccccggacgccaacttttcttatcaaagcataatagataatcgtattgttgcagagaaagaaaccctatcaaggtatcttcttctcctcttctccttcttattaagatttcgattattagggttttatgtttagatcataggatatgtcgagccctttcattttgatctgggtcgtttcgggtttgtgaaattcttttgaatctaattgtcgtgcatgattttaccttctcaataatcatggatgaggagatctttgcaagcttttattgaataataggctagtttgtttgtaagagatcttaagatctgttgtaatatgtttaatttgtcagatcgtgtacatgtactctgtattgtgtaattgtaaaacctttttttttctctagattggactgaattgtatttctcgtcccgtatctggttgatttaatgattccttttcgtaagggttttacttatgtcgatgttttatacaaatttacagtaatttgtgtatctatgactgtttcctcggtatgttgattgtttaggatgactttgataatcaacgactttgattacttactttcagtcctgttgttttgtataatattttcatcatctattttcattcactgcaatgcgcgtgttgaagaattggcgattgattttttactttttgttttgtag

SEQ ID NO: 69 - DNA, 1400 bp, Lotus japonicus
cgtgggcgtgggcgtgtgttgtctatctccttattatccaaaaaaattcaatatagggttgatagttgagttgcttaaggtggcaaagagttcctatagttaaagaatgaaaagggctagcaacacattcttttacagtttcttttcaacactatatcattgattgattgaaatttatgtaggttcaataaaaatatgggtcacactttcaatttaatgagactcacatgaattttaatttcttaattttgaaaaaatttggatctcctcaaactaaatttctctcgatctttcttaatccatgaccatgaacggttgattttaaatctaacttattaaaaaaaatataatttaaattttacctcatgatgtgaactaagtgaaacttttttttattcatctgaaaattaacaaacaaactaaaaaaactcaaggaactaacacaatatctctcattaccagggtctcaacctccactggaggctcctccaacacacaaaaatcgacgaatgcagaattagctccacacatggtaagaaaatctgcaggggcatcacaatcacttggcaagaaacttttgtagactgtttgatttaaaatttactcattagaaaaaaaaaaaattattagggctcaaatcatgattgaattgagagagatttggatcaaaattcattgagagagacggcttaatatgcaagcaatgacataattattttaaacagaatataatagcatcttctagagcattcaagaagagtgacctgggtcagcttatgtacacgtggcaagtctttactcgtcaaacacaaggatagtttcgtcattgggtcaaaaaataaaactataaataagctcctccttgccctctctattcatcgacattctcattcgaattgaatcagttcatcaaaacctaattcctcttctctctatttctcctaccaaggtgcgtcttcgtctattttgaattcatcaaattgcgactctggtttcattatcttgaattctgattcatcccaaatttatggtttaatatttgtagttctaataggttacaatccgattagggtttgatggattagggatttgtttgttataatctgattagggtttgataattgatcctgcttatgttttcaatgtcatgagagattcactagtttctgattttgtgatctattcattatgtgtgaattcgttccccaaatttgtggttttaatgtttatggtctcaataggtgcttatgattttgatggatttagggtttatattttgatttctatggtgtagttgctgtgttttttatgattttgatggatttagggtttatattttgatttctatgatgtggttgctgttttttgatgttttagtggtttgatgttatcagggatcc

SEQ ID NO: 70 - DNA, 1572 bp, Glycine max
agttcgtgtctcattatttacaacacaattaaggttcgtgtgataatcagacacttaaagtatgttgttcaccttttgagagtgataatatttttttttgtttcttttcctgatagatagcaatgaaaaaaaatagtaaaatataaattaaaaataataaatatgtacttcaaatacggtaattgttattataatggttatagatatagataaattggaaactacatctataccccacacaccacaaaatatcacttcagcttcttcataatctttgccttttccatttgtttttcgagtagtttttatttgtttgattttttttcctaggatcaaagatagatttgaataaaaaaagatatatgtggttatttatttgatttgactatgaataaacttttattcttatcattttttttataatcatcgtactgcgatttgaacgcaagacttcatatatatattatttcaaattttttagcactaggcctactctcatggctattttactctaccctttttgctttgaggcaataagtatttttttttttgttttgtatatcaattttttattttaatatttaattttatacattttcgagtagtattgtttattttgatcataaaaaattgagctaaaacatttttttaaaattaaaatagaataaattaaaagattgaataacttaaattacagtattattattgttgtcgtcctctgttagactgtaatcttatcttatacttatagttttattcacaaattaaagtggaattgattgacttgacacaataatgcatgtgcctgaccagagctactcctaagttctaagaatgttaatttaatgtaacatttttttattaattaaactttctctagatataaaataatgcgtgtggtgtttcacttttcaattgataaattaattaatataaataaaataatagtaaaattcaacaaattttccataattttatttgatataaaaatctaagaagttccctggttcggaactgacagagaccaattttgaaagaatcgacaacgaagatggtccctccattctctagtccataagatcaatccttgtccacatctattcttcatgttttctcgcatcaactaattctctttcacgcaatctcaaccaaaattaatactaaataaaaaactctcacgcaatcacaacggatatataaacagttttctctttttctcttttcttttcttcaaaaagagaattttaatttaaaaatgaaaaaaagcacgaagcggcgtcgtttgttgcggcctgacaaaagtccacacacatgaaagaaagtcattggcataatgacaagagcatatccgtgacctgcgatccgctccaataaccctagtcaactccaaactcaaccatagttacttcacttcactcacaccccgccacgtgtcccaatcgaacggtcactttagcatcacgcgcctctataaatatatatctctatgtctttctcaatcctgagcaaaaccctaatccctatttttcttcctcagtagtgtcatttacgcttctcttctctgcagcaact

SEQ ID NO: 71 - DNA, 1666 bp, Glycine max
ataggttttaagaaaaggtgtacaatcctatattgttattaattattttcattgattttttttttctcaccatctagttatagtctatgtgataatggcataagtattattgaggaggcatccgtgaaatgaaatacttttcacacctctacttgtactcacacaccacaaaatatcactttaccttcttcataatctttgccttttccatttattttcatgtagtttttatttatttgtttttttttcttctaggatcaaagatatatctaaagaacgaaaaaataagatatatctggctatttatttgatttgacaacgaataaacttttactctatcctttattctttgggtttacaagtattttttatattctaagattttttactttaatatttatttttatttcaatcaactactttataaagtcatttatttttatgataaaattaaggtaaataacattttggctaaattagaaattaaagtagaataaattaaaagattgaacaacttaaattattattattagtattattattcaacttaaatgcccagatccctctatcttatactttatcatttcaggaattgattgacttgacacaataatgcatgtgcctgaccagctacgtctaagatgttaataagatagtactttttaatgtaatattttttattattattgattaagctttttctagatataaaataatgcgggtttcagttttcaattgataaattaatagttaatatttttataaaaattaaaatatacaaaaataaagtgataaaaattaataaattttctatcctttttgagtttttgctataaaaatctaaggagaagttccctggttcggaactgacgtagaccaattttgtaagaatcgacaatgacgggtcttttccgatccaaatggtccctccacagtccttagatcaatccttgtccacattcacttggccccatctccatgttttctcacatcaactaattctcaagcaaaaaaataaaataggttctttgaaggaatgatacagtgaccaatttaatttttaaatatgtaaaaattatgataaattaattctattaaatttgtgaatttattttattattaagttataatatttaatgactaatttgataatatattatatttttaagattaatttgatattaaaggataaaatttatgatcaatttttttattaaattatatgttaaaattaatttattgtattttttatatatttggagattaaatttttttttctgttcacactttgtcagcacttttgttgttttttttttcaaaaagagaaaaagagaatataaatttaaatttaaagcagaagagaacgaagcggcgtcgtttgttgcggcctgaaaaaagtccacactcgtgaaagtcattggcataatgacgagcatatccgtgagtgacctctgatccgctccactaaccctagtcaactccaaactcaaccatagttactttacttcactcacaccccgccacgtgttccaatcgaacggtcacttctgcatcacgcgccactataaatatctctctctcgtcatccgcaaccccaagcaaaaccctaatccctctttcttcctcttcctcagtagtgcgattttcgattctcttctctgcaact

SEQ ID NO: 72 - DNA, 1642 bp, Lotus japonicus
tatttaatagcactttgatgcatcaaataagctgagctccatcttgtatgactcaacgcactacttttcaaagtcaccaaatcgcataaaaggtcgtgtaataataaagtaataaactatcaatattttaaattcatcttccatgacccaaataagtggagtttgctaatttactaatttgattttatgaagaagtgatagggatgagtctattttactcaaaatcttacaaagaccaaagttccctctcaccaagttccaccaggaccaattttttgaagaatcgacaccattgagtcttgttcagtctaggtggtcctaatttaacaaaaaaaaaattatgaagaagtatttttttttacaaaatatttatgaaaaagtaagtttttttttatcaaaaaagtaagttagtataaaaaaagttaacggttaacacttttagttttgtaaaataaactcaacatattcttgttaaaggcaactagggatggcaacggaacccgaacccgtgggtccgcccgaacccaaacccgatatgattaggtttgggtttgggtttggtattggtgaaactcgcacccgaacccgaacattatatataaaattgcaaaatgactcttacatatcaggttaacaacttaatttgttaacaatttgatcaaatttgaacattaaggtatcatattttttgacaaaaaaagatataaaatgttttttcacaacaaaataatacaagttgcattttattttgcaaaaaaatttatacattaatttaggttatgacgtgaatttgagggttacaacgggtttgggtaaacccaaaacctgacgggtttgggtttgggtttgggtttatatgagagtttgagtttgggtttgggattggtaaaacccgagcccgaccagactcgttgccatccctaaaggcaacttaaattcaaggttattcctgtaatttcatttttaaagtttcaaataaaattccaatgcgtgttttcattggcgttgggaagagctatcatagtaaacgatgatgggtagaacctcatggtacttcttctagatgaacatggggttatgcttttttttttttgtcaataatggatttcattgataaaaaaggcaagaagcccaatacacggcctgctaaaagtgaggggccacacatacaacaggggaaaaggctgatgatgatcagcaccagaggaataaccttgttaggagtaaatttgtgataaccccacatttcatcgagaattgatcatcgatatattaatcctcgtcgacattcatttggcccacatgaacccccaaaaaagtgattccaaaatacaaaatttttaaattaaaaaaaaaaacgaaaaatacacagtgaattaaagcacgaagcggcgtcgtttgtttgtgcatgaaaagtccacacccataagcatatccgcctccttcgatccgctccactaaccctagttaattcctaactcaaccatagttacatcactccatcacaacacaccacgtgtcctgatccaacggtgaattcacaacgtcttcagatgcacgcgccactataaataaccttcgtcctcacgttcattcgccaaaccctaatcccttttcactcttcatcactctctctgcatcc

SEQ ID NO: 73 - DNA, 1672 bp, Glycine max
cattgtttatcctttgctaagcaaagttatcttgtatttgagctttactttatccattcattgagtgttatcgaatacttgtattcattctaactactgtttatgaaagccaggagtagcttagtgctaaataacacttgggttttcttagatacatgggagtctaagacaatgctagaagtgatataaagaatatttgtatagctaggagtgacaggaccaaatacttttattagttttagtcaaatcaagatttcaagacaaaatcagatttagcacgtgttatgatgtatgttggcttcacttggacctgcatttttagtggtcaaacgtgaaaaattcatttggtgttttgcttgtacgtgattcactccaaggaaattaattttcaggtgctactcaaggttgcttttgcatcaaagtaaaattgatttttagattttgggtgttagattctcatgtttgtccttcatccctctcccattttgtttgccaattcctttaaccattgattatatttcaaggtatttcatagagtggatgaaacagagaggcacaacaccaacaccactgtcggtgaaatatttagctccaaggactcagaggcacctctagccatgactatggtcgtgtggttgttgaagtcaagaaccaaaactgagaggtactactttggtcatattaccaagatgaataatgatgaaaacacagaaaaacatgaaagaggaggtgaaaaatataaagtgaaaaagaaatcatcatcattgaaaaataaatattttacttaaatgattaatataacattattattgtagtggagtaacgttattttttattaaaatgatcaaataaataaatcatcgctaaaaaataaacattttctctaataatcttaattattaattctcttataaaaaatatttttttaacgaggacgttatcaacatcaatataataaattaactaacggtggcaattattttcataatatcaatattaatgagtaattatatgcaaatattaaaacgcattttcataataatttaaacaaacttacttaaaatcaagttgattcaaaatcaattcttaaaaactacatattcaaacacatgtactttcctaacaataattaaacatatttgattatactcaaaatacattctcattaatcaattaaacactctctaaactttacatgagaatcaaactcattctctaattttctttaattaatagggttttttttttatttgaatccaattaaaaaaaaggaatttgaaaagtcgaagcaaaagaatataaaaatttagactgtaactgcccgttggcagagttggacgtgagtggaggggtgcctcgtggaccccgggaaaggtgcacaaacaaaaacaagttcaaaatttgaaaggtaaataaaagggctccaccgctggcaaaaaaaaaaaaaaaaaatcaataattaaccgcaattaaaccctcacacaccatctcgcttccataaatccacgaaaaaaaagcgaagcacagcacagcactgcactcttcgcaacacaacaaaaaaagacaacccagagagcgcgtcttaataaacgctcttctctgaaaacccccatttcttttcttctttcttcttctctttcgcttcttcgatcttcgatcttcgtcgctgaaa

SEQ ID NO: 74 - DNA, 1723 bp, Phaseolus vulgaris
ttttatctttttaaaataataattattttattttgtataccaacttttgggtattttttttctctatatatatttctattttattaatgagactcatatatgatactttgatgtgtgactcttcttctctttaatgtgaaggtatatttctccttctcaaccacttctataccctctttacttcaatctcatgtttgtaacaaccatgtctgtaccttttacgtgatagaagcactgtgaaaattatacaaaagagtataacccaacaaaactccctaacatttataatttaagagggagagagatcatttaataagtaatatttttacatcaaaagtgtgacttttatttattttaaatacaaaactgtaaacatttaaaaactcaaatacaaaatttatttctatttattaataatatattctatgctaaattgtaactttcatcctctatttttttaagaattaaattaaattttaatagttttagtccctgtctctaaaagtcggcaatttagtacttcgttaaatttccaatgttgataatgccaatatttgtacagtaaagattaaagtagacttatgttttttacatgataattcttgttgtgatttttataaaattgtttacaaaattaatatattacaaaaaaattaaatttacctacacatggataataccattaattttgaattatatccatttttattattgtaatattctgtgttattatgatatttagcattaatttagtataatttaaaaataatctcttccaaaagagtcccaccagagaggttattcatgaactatattttaagttagttaagctaaattaataaatttaaagataagtattaatataatatttatatatctaacgttttctatcatatcttggtcttaaaatataatatttatatacgtaaagagatattgaatttatagtaattaattttattttattaatatacttgtatatagactgaaatcaaaagtcacatattaaaatacttatattttaataactaaatttaataaatatacaatattattaaatgtaaggtttaatattaatagtatgtaaattaaaatactgataaaatttgacttaaaaacttaagtgttaaatcaatatctaagtaataaatacattaatgtgggcaatttaattaatataattaaaagttttaaatttgattggatctatttaccgaaaacattgattcgaagatataataaatatttgtaaagttgaccattcgataaatggtgaatgttagttcaaagttgaaggtgattctttaattagtaggagaaaaatgagttgaatgttacttaaaaaaaagaatatgaaaagttgaggaagtaaggaatgaataaaatttggacataatggtgcgttgcagagtgggacgttggaggcgcttcgtggccagaaaggcacccgggaaaggtgcataaacgaaacaagtttaaaatttgaaggtaaaaaaaaggaaccaataattaacagcaattaaactcttaccctccgcttccataaatacaccgaaaccaaccgcacaacgccacagcacttctctcattgcaacacaaacctcaaccagagaaccagagagccagagaactagagaaccagagaggcgtcttaataaacgctcatctctgaaaccctttccctctcttcttccatttcgatcttccttcttcgcccgtcaga

SEQ ID NO: 75 - DNA, 1500 bp, Medicago truncatula
cgcttcatatcaatttcagcatatgctgaaaaagttatttagaaaatccttcttgctagcaatacaccatcattgttctttttttcgtatgaagcgagatcgacattagaatctgccaaatgaacaaagacaacaatgtaggaagtatcattagcccttaatcaacgatcacattgaagtttttattgtaagttctcactttaaaaatgttcaaccagaagacttaacaaaagaagacagcatccaaatgaggataaaaactaaggacagtcttagatcaataatgttctatcatgcttgagatcaaccaaccaatccactattatgtaacaacacaattattagcaacattgtttcgagaaacagctgcatccaatgtcgtcaccatcaaaccaaagaacgcgaaatctatgagcccgtcacaatgttcgttctaattcttgtatgtccccaaaagcaactgttacttgtataagttcatcacaatcttgattttaaatcctcatctgtgattccaaagcagtcttgtgttgttcatgaacagagatgctaatcctagaatgtcatgaatgttcatgttctctggatgcacgagatgaaattttgaaagataaagtttagcatagtaagtcataatttgagtttgagcgttgtaagaggatttggtaatcagtaaagtgaagagtaaaaaaagtgagattatattgtgataattcaataaagagatgaatatgattcaatttaagaaataaattcatattttttttcaagtattttatatgcatacaagtttaattacaaacattctactaatatattttaattgtatcgtgtcctaaatttttaaaaaatttgtgtatccacatacatgtaccgtacctacactcttactatgcatccataattctaccgttagataaataaaaagtctttcaaaaaattgttctacctagaaattaaaatcagatttttttgaacaattctttcttgacgaagtttattaaccacttgttaaaaaaatgagttgatttatagatttttttattattaatttttaataaaagtcgcactagttttgaaaatagaacaattccaaagttttcgtagtggtaaaaatacttgccttagactcgacgggacggattcgatccccatacatttgaataaatataaatatttttgtaaacacaaagttatcataaactcacacacataaagtggaggtattgagagtcgaaccccggtcatgacttatcattcatactaaaaaaaaaggaatgtggcatcaataaataaataatgtgatcgaaaacaaattaaacggttaccctttcttccattcattacggttacaaatacagtggaaaccaaccaaccaagccaacgcaaacacaaaccaccctattattttcttcttaccaatcatcacatcgtaacaagagaggcgtcttaataaacgttcatctctcaatctcttcttcatttcttcaatcttcaaaa

SEQ ID NO: 76 - DNA, 1500 bp, Cajanus cajan
cacctttgtatctaaaaaagaaatggagaatgccttatgttaattcaccttcaaaaagatggtttgtaaaacaccaattgaaacgattaagcaaataaaaactcattgtgtgaactctaatcgtagaatgttttctaataattatccaactcaatgtgttatcttgctcatttaaaacctctaattttatcttctttaaatcatctattcaaatcttaaattcaagttcccctatttatctaattttaattaattttgatgattacacaagtcaaaacgcaacccatatggattctataatctaattacttaatgatatctatgcgcttgcagattttgctcatcaatacgcttccaaggatgtcccttaatgatatcttagtggtcttattacttgggccttgaggacaacaaagcttgggagccctagtctcatctataaggtgcatgaggtgtaagttgttgtagctttgttggcctaacacgcactggtgtatctccccccttgcgagagtgctaggtgtctagcatctttgttcgggtgtctgtcattgacatgtattactagactactttctttgagctttcgggcgacaattataattttgaatgataccaagtgcatataaaactactgagttattacattttatatttatcttgtgaatattaatgtgaaatgcttattgtgatttatatgtatatatgttcttatgatttgttgtggctgtctgactttatctcacaccaagagtatgagtgagatgtatctcactccttttttttattgtttatttttttgttttgtttgggttggacactacttgcatgagagaatcatccataggttggtgttggtgataggatctatatttcttaaacttgtgtggggattacattttgatagagtgtgacattggaactaaaacttgttttcttatatttatgttatgatttaattgtatagatttcatggattttgtatggcatttggagtttcaagcattatttatcttatttggttcatttatgaactatattattaaaggactttgactataattagcttcaattgaatagcttaagaatgcgaattctctaccatacaaaaattaactaattgattttgtttttcaacttaaattaaataacaaaaatccttttataaactaaatatttaaaaaaaataagaatatttaaattttttttaattagaatgttattattggcatatttaaaaaatgagtttggttgcatcgtaatatcccttgagaaaaaagttacaagtatagagagaaaagaagaagccgggaaaggtgcggaaatgaaaaaggtttaaactgtggcataaaagaaaaaaataaccctgaggctcccgcttccataaatacaccacaaacatagcgaaaccccacacaacacaacgcaacactctcttccaaaatacccagagaggcgtcttaataaacgcatctctgcctccgatcccatcgcccaaac

SEQ ID NO: 77 - DNA, 1582 bp, Glycine max
taatttatctccatcaaataaacattttataacatatattttgatacctatatgttgctagctatgatgtccttgaaatatttataatgtgaaataaaaacattgaagattgaaatgagtcttttgtgttttataattttagagaaacttggttgatatcacaaaaaaatgcaagacaaaaatagcgtcaaatgagttttaaaagactaaatgagtaatttaaactaaaaataaataaattcaatggaatataacccatcatccagtgccaaaagacatgggtactacatatgctcactttagtggctatggaacaaatgaacaactatcagttcttcctttttccgttgtaggcatagagacaaagtttacaaattgattgaaatccctataatcatttcctctactctttcttttatttgtggccacagtcacacggtaaacattagaatgaggaaataaaatctcaataaaaaaatctgtcaatgatcgatttcgtgaacttgacccaccccatcaatgattaatttaatactaaatacagttaaacagcaaattagtaagatttttaccagccttccccttccttcgtacctgtctacgcaaaattacctgacttagaggaaaatgaattttctaatgaattacgtatcacaagttctgtctttaacaaatctcttttcatgtgtgctaacctaccttacaaatctccaattatattctctacagatactaatacatgaaaacttttagaaccttttgtaataaaatagattccatatacataaagaggaattaaactttgttcaagacatggctatttggtttggtggttttttatttttattaaaaataaaaaataataatgaaaatgtgtttgattgaatttttattaaaaatattttttcaaacgaatcaaaaattaaaaacaataaagttttgttttcagttgaaatcagaaacttcatttttggtaaaataaaaacacggtcagtgataatgaatataattttaagcaaatctacaaatatatttttttttaaaaatacatttttagtgtttttattttttgacaatagaaaataaaaaatcaaatcaaacatatttttagaattctaatattttaaaaatgaaaataattttcaaaaaataaaaacaaaaaataataaaaaataaaaatacaaatcaaacacacccttacttatttattaaatgtgttaaattatattaattattactattttttatcatataggtaaaaaattacggtgtttttcaatccatcggattaaaaattaatttcactcatgggtgattgtgattgtgaattatataaaaaaaatttacgcacagaaatgaaatataaattctgattgaaaggaggtaagtaaaaggcaaataagaggggtaccctaagttagtattagcaatagcaacaacaacaaaaaaacaagtctagtccacctcaacaactccctctccctcccaaacccttatttatacacacacacactctcattctttcctcaccactccactctctttttctactctttctatctttctttctttcttcttcccc

SEQ ID NO: 78 - DNA, 1576 bp, Phaseolus vulgaris
taaattgaccaaactagcaccaatcaaaacaatgaaatctaataatgtatccactaatttttttactcatctccatgcatctacttataaggtttttaattcttttacctcttgaatcatgcatctgagagattatttagaataacgcaaactaaacaagagtttcaattacaaatgtaattaaatcatcaacatacatccaaaaataattttcaaaatagaaattaaaactaaaaaataagactttttagtacattcacaaaatttaaaataaaaattataacataactttcttcacaaaaatatatgtatatattagagtgaaaatatatacataatcaattatatttttaaattcgaaatcttagagaaatcaacacaatcaattatatcaaatactgattatattgttatatataacatatttataaagatacaattaattatgtcttcaatcaattatatctttaatttttattaatattaatatttttatttattataataaaataaataatataatattattaaaaataaaatataatttttaatcatatttattataaacttttcacttaaatctacccatttcctttaaaaaacaataacattttcaactcttttcccccctcttttttaatactctttccaccccacccccaatcaaaacaaacccaaagtattttttattttgtgttaaaaaggtaatgttacaccaagaagagtcataggaaggctaaaaagatcacaatcaaaatctagtaggttatgcacagtgcactttggatcatttacctgaaaaatttgggtcatcaacttagtgagactaaatttggtgatccttaacctagcaaacctcaaaaccatcctttaacacaccaaaattaagcatgtgtttcttctatccaaacattgacttaatttggtataacaataaaaagaacgaggcatacacatagatgtaattcaaaacttcatataaaaaaattattattttttctatcccacctctaaaattataattttatattatcataataagatttattaaaataaaaaaataatcaaattctttatattttaatatattttgaaaagtatgtattttaatgaattgataaaaaatttatcgaaaagttttgaaaacattgtccgattgaataattaaggatttgataaaacttatagttaggtagtttgacatctctgcatatatattatgaaactgaattaatttactataaatatgtttgacgcgtctccttaatcgtcatttagtttaattaaaagacctttttctttcagctttgaagaaaatattaataatataaatagtgtaaaataaaatataaataggaaaagaaaagaaaagaaaagaaaagaaaatgaaaggtagaaagagtaacctaagttagaataggaggaagaaaaaaaagcagatcgggtagtccactcaccaactcccacactgaacccttatttatacggactctctccttcattctccttcgtcactcactctccttctctttctctctaacccacatttctctcttcttcctctcgctgcgcagcc

SEQ ID NO: 79 - DNA, 1672 bp, Medicago truncatula
aatttaaaatacaagttagagtatattgtacgattagtgggacctattttagagtttacgaaaaatggtatgaatatttagaaaatgtagttgagtggtttaaataattgggaagtgtccagtaatataatatattaagtcagactcatttataccacccatatgagtgatatggatgtggatgctctaacatcttaagccataagcaatggcgggaggtatgcctttgtcttttaatttaagtttcccttgtttaagcataatttcttttatggttttaaagaaatatcttacacattattcttccttttgttaagaaaaaaatgactatactagttgtttacaaatgacactactagaacatccgctaacatggtaaagtaaaggcaatagagagactatcagataatggccaaaggatcaatatcattatctcccaattaggctagaacatcaacacagtagttcccttcgtgaagagtgtgatgcaacccaacatttcaatcttccagcaaataatattgaatggtcgtcagaaagttaaaatggtcgtggagtctataagtactagtggtcactgaccagatagtataattaaaccccacatgtaaacaaagttcaatatcagttagaaagacttttatataaacatgaagcgcatttgaaattctcacactatcatagaaggcagactaaaaggactcctcatgattcctaaccaaacctcaaaaattgttttcttgtgtttctaagagtgccaccatctatattaagaatcatagtctcgacattaccatgatgccaagaaatctcacgagatggtgtcataacatcaaatgcttgacgatctaccgccaattgtgcaaagactgtggctaagctcgaactcactacaagctgttgactttcgaatacaagtttgtttttggaacattaaatattccacatgatccttcactaagttgcgcaccactattaaaagattatctaggccattattcaagtataaagagtgaacacgtatgaggccaacaaccacgccaagtagaatacggatacatgcaacatattgtgcaatgaataatggactcaactacatcactacaacataggtaagacgtatccaactggactcccatgcgatccaaaactattttcgcagggttaaagttatgacaaacttgtcagataaaaatttgaacgccggatcaaaactttcacctttcaaatcaatcctcatctattttcataaattattttgattatgtaaataatttgttttaaacaatactaatttaattttattaagtcaaataacttgcgtagaaaataatccctccacctcatttaacaatttttttgtcaaacacaaatgaagagaaaagaaatgaaaaggacatggagttgaatagtggcaaattgattgaaggaagattcacatttgaaatcatttaatatatttttgggtccataataataataaatcggaaagaataaaaaccggatcaagtccggtccagtacaattccactccaccaacaactccaatcacttaaacctcttctatttatacccactctcatcctcactctcccttcacctctcacactctctcttctctctctctcaaaacccttacacaaacgctgcgtttagtgtaagaaaatcaatc

SEQ ID NO: 80 - DNA, 1500 bp, Lotus japonicus
cgtgtaggattttaactccccagtccactattgctgcaacctgacacgtgtcaattcacaggcgcgagttcaacgtgatattgcgagcatttgttgacaaaaagcaccgcccagccatagaatttggtccccgtatagtcgcatacatcaccacgtttattaccgccccatttcccctattaatcaacccatattcagacaaaaaaaatatataaaaactaaataatattgttactatttaaattttatttcttctataacatgattggttttttccctattttttagtccactccaactataaaaagctagcttacgagtaaaaaatagctctatttttatatttgttaattatttgtatcacatctttaaccaataaaatcttgagcacttactaaatcttgcatgtgattctttaaattatttgatactttaaaactaacagaaaagaagttgagcaattgagctatgttcgagaaacacaagttgatgtaactcaaaaagacatattacgcacactacttaaggtttgatgttccaggaatttttggatttgtaaatttgtggatttaaggagacatgactcatcacatgcgacctgacttgatcacgagtgatattttcattatgagcgatcatagagctcctcatcatacgagttgctagtgattttccagatttaaaaaatagaagtttcgtacccttttatcttaatgtatcattttaacttttgaaataattttttataaattacaatcgaatcctaaatagtgtaaataataagttgtattaaacatttttattttaaaagagcaccggaacaacaaccttaactctctcctccaatgtagtagggagtagcacttacatatgattatattcacttgaataaaaaaaaactaattttgaattttctcctctctaagtataaacatataactaaaaatgtataataataagtttatcataataatagattctatttaaatcactattaagaaacaacagtgtaaataccctaatttaaatgactctaagtagtatcttgacaggtgtttttgaaaatttgagctgatgtctatttagacaattagacaattagataaatccaaaacaagaaatgaaacacaaaaaatgaatgcatttttataattggaggtgaggtaatacatgttccatttttcctgaaaactacagtaatacaacgttccagtgatgcgcagaacatggacggtaatgataggagtggttagaatcacaccagctaggacacaacgttcatcagtgaccagtccctatcatcacagcattaatatattcaacatattacgcctagtgggtcctaccacgctcacctccagttccattaaccacaacatattctcattataaatccgcaccttacccactctctcacaccactccactgacacacactactacacaccaatcattcatcaccaccatcatcagaagaaccagaacaaccactaaccatcg

SEQ ID NO: 81 - DNA, 2063 bp, Phaseolus vulgaris
agaaggaaaaatattccatcttacttgattagaaggaatatgaaatgttagaataggttttcctttttcttcttttttcttgtccacataaattttctgaaaacaaaagagacatatatatgaaaccaaaatttttaaagaattgtgatccctcactgaaacagaaagctaaatggaaattggtaaaaggggtccagaagtgcctagctaaattttatgtgtgtttctaaattagaagccacgagacaaaagatataaagaagagtgtaggaccctgccacccaaaatctccctggagctcatgggtcctaaccttgtgccttttgccagactagaatgaccaactaatcaattttcaaaagattccaatgtttttccccacaattcaaaactcaatatcatcaacaacccttccttttattaatcttatccttacaagtcactcacactaatcaacccactaaactaatgcatattagctacctaatacgtataaataacaacgctacaacctaattcaaattctttatttttcctttccttaaccacctttctatatctctacgtattaccagaggaaaacctgcaattattattattatccaacaaaacaaattgaaagccacagttacaagacatggcctattaacatatctagactttatcgttgtctcatccaccagaaagaaacaaaatatattaaagagaggaaaatgagtgcatatatatgtagaatgaacatttaataatggacgtgagataagtttattataaatgaacattgaacatccatcatccatgtgccgaccatgttttataaatcaaaaggaagaaatgtttgtatataataattagcggacacaaaaaccttgtttcttcatcattatcgttacattttttttgtttctgaaaaaccttgttcatctattaacgttggtaattactatataaacagctttgagtgaaatatttttaaaacaatgatgacatggatttgcagccattagcaaatgcattaatttttgtttgtacttttttcttggtttagatttattattctacaataatcaatatatattagagaatgacttgaattcatattcaacttaaatctattatattaaattgtattaccttacaattttgtgttttaaaagtaggaataaacagtaaattttattctaaaatactgggtatcttaaatagtctttgaaataaacagatcatataaataatctttcaaatattaaaatgatactatatattttttaaatagtattttttttttcgaactaaaaaaaatgaataagtatttaaaagtatgtatttaagagagaagaaaatacctgagacactaaattatatatataaaaaagaatgaaaattgaaaagtataattacttgcatgttggtttgatatgtaggtttacgtgtggttcaatggagccaaattctaaaaatgtaaaactgaaacaaaaagagaaacaaactgtagacacctgacaggagtaacagcagcgaacaacagtagggggagtatttgcgtgttggtttcaaattctcgaatacaccatgcaatgcatgagttggtgacacgtgtcctagtgcaggcgcgagttcaacgtgatacttgtccggtctcgacaatagcaccgcccaggagcaagcgcagtgaaggaccacgtagaattgggtccctgcaatgcaactctctccgtcaaaaacgaaacgttttacccttcttcctgtaacttttttcccagttccgtttattcaaaactacagtaatccacgttccagtgctgcgcggaacatggtcggtctttctaggagtggttggaatcacgccagctaggacgaaccccatcaatcattggtctctatcaaagaaaacaataaaaaattcaacatattacgctggcgggacccactcccactactctcaccctcacttctattaactctaactcattaggattataaatccgcaccatactccttaccctatcacactcactcactcacaactcacaggaatccgaggcaagaagaaggaaaaaaac

SEQ ID NO: 82 - DNA, 1500 bp, Cajanus cajan
atgatcagatttaagttatttatcttccgttgcacattgagtcacaattagaagtactaataaattcaaaaagcaccaataaagatattattcattactaattgaaccaagttttgtaagccgtgtcatttgttttgtcttttctgttcaaagaaaatgtctgaaaatcaatgagaggtaagaaacgaaacttttatggggaagtgaacaaggtgttgatgaatatgagtagacttgtgaagcttcactgaaacagaaagcataatgcgagttgcgaaaaggggtccagaagtgcctagctattttttatgtgtgcttcttaattaggagtcaccagagaaagatataaaggagagacgtaggaccctgccacccaaattctgcctggagctcatgcgtccctaacctgttccttttgccagagtagaatgacaaagaaaattttccaaaagattccaaaacttttgcctcacaactcaaaactcaatatcatccacaaccgttccttttatttatttatttatttattaatcttatccttacaagtcactcaggctaatcaagttactaaactaatgcatattaggtaatccaaataaactagaaaacgctccaagaatattcaaattgtttcttttgcttttttgcttaacgacttctgtatctctctacgtattccgtattcctgagaaaaagctgctattctcatccaactaaaaaaatgaaagctacagttaaggacaaggcctcttcacatacttaatcattgtctcatccacgagaaagaaacaaaatatttaaaagggctcattatgcttaattcaacaagagaggaaattgagtgcgtagaaatgaacattttaataatggacgtgagatagattaacattttaattattgtttcatattttggtctagacaaaactaatacccaaagctaactattaatggagcatgaaattgaattgagggaaactctaaaaaagtgtagagaggtaacagtaacagtaggagtggaggagtatttgcgtgttggtttgtaagtgtggtttctggaatcgaccacgggctatgcaacgcataatgcaagaggaagcgacacgtggcgtataagtgtcgcgagttgaacgtgatatttagaagtgttgacaatggcaccgcccggccgcaaggaccacgtggaattgggtccctgtccctgcttgggttataagttaaaagttaaaaggaagttgtattggagtggtgcgcggagcatggtcggcttgggtaggagtggttggagtgacgccaggtaggagaggggcagtgaatgattggtccctagggcataaaacattaggcggatgggagtgggacccgccacccaccttagatgaattaactggaactcattccgcttataaatccgcaccttcctccttcctccttaccctctctcactctttctttcactcactcactcactcactcactcactcgctagaacaaaggaatccg

SEQ ID NO: 83 - DNA, 1613 bp, Glycine max
gaggataaggtgaatcggccttttgttttgaacctatttggttttaggtgcgtccagataagttcatctggcaagccttcgtcacccaacaaaagtgacaactacttttctccttattttaatcacatcaacaattacactaattaatgattgatagctttcttctatttcccagtacaagatatcttaatatattgctattttctatgctaatatttcttggccactaacttgtgctcgaaagttctacaaaggtatactcaaattttaagaaatggcaaagttcaggcaaattaaccataacttcgcatccaatacattatcctcaaaaataaaaaagataacttcacatcctttagcaaaataaaataatcaacagtttaaaaaactccaattttctatgtgccacgagatcgatcgtatgagattggagttgtattaaattcattttatgtgatgaagttctattaatttacgtaagcaatattcgaaacacacaactgtacaaactaatatgaaaatttcgtggctaatttttatttgtgtatctatttaaattcattattaataatttgtgtacgtttgagaagtatttttagttagttacttagtttttagttccttttacaagtttgtttgaatttattaaccatttattataaataagtttatttagtaatttgtaatattttttataattttctttaattaatgttgatggttaaacatttttggtgataatttatattaaaatttcgattagcaacgttctttagttgaaattaatgctcagtagatgtaatttatccaaaaatctcccaccagcatatcggtggacagaaaagcatatcagaccgttgaattggctattgcttgctgcttaacttgcgtatctaatttaaatctcatggatttaagtaacctatattatttatttgtctcttttgttattttttagttaagaaaatataaaaaattataagcatatatcttatagttagttttgcctttttttttttctatttctcataacatcatatattatatctattttttttcatcctttctttatctctttttctttcccaccctagtgctctcaaaatgaaattcatgtatacaatttttcaaaataaaaaagacaataattattagttaatttactatttacaagacttggccttagcccttagccacatgtaaccgttgaattaaagaacgttattacttattaggcgatgttggcccaccgaaagataaatgtaatactaaaaaaatgcagagttaaatatagaaaagataatggcagaaagagagaacattgagaaaaaaaaaataaggagcacaaggttgcagtgtagggtaaggcgcagcatgggaccatgcaagcgaaatcaagctaatagggcggcaaaaaaatacgctaatcatgtcatccttcccacataggcacatacgtacaaaaccttgacaaattaattattaattccactcattacagcactcttccttaaataccatcatctttcttcacaccacacaccacacaccacacacatagtagctattatactctctagagtaattgcggaatctgctagaattttctaata

SEQ ID NO: 84 - DNA, 1500 bp, Cajanus cajan
ctaaatcccagttcaaaatcgttatttaatttaaaactttgctaaatgaattgatttaattgcaaatatagtggcctcataattgtgaggaggataaggtgaatgacccttttgttctgaacctatttgattttaggtgcgtccagataagttcatctggcaagccttcgtcacccaacaaaagtgacaactacttttctccttattttaatcacatcaacaattacacaatattgaaaatcatcaatgattgatacctttcttttatttcccggtataagatatcttaatctattgctattttttatgctaatatttcttagccactaacttgaggtgctcgaaaattctacaaggttatagtcaaattttataaatgccatagttcaggcaaattaatcataacttcacatcccatacattatcctaaaacgaataaataaaaaagataacttcacatccttcaccacaataaaataaaataataatccacggtataaaaaactccagttttctatgtgccatgagatcgtacaagattggagttgtataaaattcgttttatgcgatgaagttcaatgaattcacgtaagcaaagtattggttttgagacaattatacaaattaatatggaaattacgtcgtcaaattttatttttctatctatttaaatcactaataatgttttggatgtttgataaataagtctattttaattaaactattttttgagtttttatattattagtatgaagttagctagaaactttgattaattatatttaccaagacctcttcttttaattgtaatttttttatttgtaaagtttaaactcaaaatcgtatttcagaaattggaatctaacttgactcgtacatcaatgacataggatattagttttgttaagcttgtgttttctattctgattggttaaacatattttgttataagtcatattaaaatttcttacggcaatgcttttcaattataggtgaaatttatggtgagtaggtttaatttatccaaaaatctgctctaaattggtggacagaaaatcaagagaccgttgtattggaattgggtattggttgctgctaaaaggcgtgtctaagttatatatctgatggaattaaatgacctatattatttatttgtgtctttaaaataatttatttattcaattatgatatttaaagggtaatactaataattgattcagcatttacatgagatggggtgggcttaggcagatgtagcggttgaagtaaggacgatgggccagaaggaaaagataattgagaaaatagaagcagggtgggaccatgcaagctaatagggcggcaaaacggtaattaattagggtaatcatgtcattgttgccacataggcaattaattaataattggattcattaggagagtgttccataaataccatctttctgcgactccatccccactccacagagaaagacagagtgctagtagaatttagtagaa

SEQ ID NO: 85 - DNA, 1756 bp, Lotus japonicus
attaggtgcgtccagataagttcatcttgcttctttctgtcacctaacaaaagtgacaactacttttcatcttattttaatcacatcaacaattacataattctaaaactaatgattgttggctctcttttattggccaacaataacctctcactatttttatgcaaacatttctcagccactaacttttcgagtgcttgaaagttcaaccagaggacatagtaaaattttaagaaatggcaaaagtccaggtgaagtaaagacatcatacccttttagcaattattttttgaaaaaacacacctcaattaactcagagtactattttctagtacagtccttgaaactaatttatatggtttgggtctatttaatgggattatgttcaagatatcatgtaaaaaataatgtttgatatattaatctaagagtaatcagtggagtggacagagaagcaagggttcgaccgttgtattattgcttcttgcttaacatgcttatctatattatatttcccgaccaatttatttcaattctctacacttgccttgagtccttgaccatagccacatgtaaccgttgagttccagaacattagaatcttttaggtcatgttataattaagtgatctttaaaaaccagatgtggagcgtgatcaaaatctcttgctcttaattggtgtttcatttcatgcctcattcatgagaaagtgttatttcaattaaaagtcactcataagagattgtagatatcatattcatattattttaattaacacgacataacttcattatacgagaccgataataagataccaatacgagaacatagaattttgatctagagtatgacaattgttcatctttggtataatcatatcctagtccataggaattgagtacattttgtgtttgtagtttaccgagacatggcaacgagatgggttgggtcgatttaactctcccaaccccgagctagttacgaatggaacggattcgagtgcaggtttgaaaatgtctagataaaaatacaaaaatcaaactcatccctgtcaggtttttaaatattttaattaatcttattaatttaaatctaatacacaattcatatagcaatttttttaattaaaataatttatgaaaatagttgttcataatttgtgcccataatcttaaatataagaagtttaagaattaactcatatttgagttttatttggctactgttataattcaaattatgtaacaagtaagccgggttcaggtattagcaatcttatacccgtcctcatctccgcttcttcgagttgttcaggttcagcttttccaaacgttcaatcaaaacggatttttcttgtcaaaatccggcttaagtttggagaagaagataataataataataataataattattattattattattattgcagaaggatcatccagtaaattgatgattagtggttaagaatcattggggaggagggtagggtagccgcatcgtggggccatgcaagcgaaattaagctaataggggcggcaaaaaaataacgttaatcatgtcatgtcattgttcatctatacgtacaaaccaaaaccatgtcattactccattaattattgattcaattcaatcgaatactctgtcttttctcatcactttcactccttaaatactatccttctctacactcaactctcaatccacacaaaaaatcaacactaccaagagcacacaacacactcttctaggatcc

SEQ ID NO: 86 - DNA, 1500 bp, Lotus japonicus
acaccaatcttactacaaagtaattgaacaaagacacttcaatgaagcatttctacgaacagcttaagggcatcataaaaatcagtttgtacaaatgtagaaatgcaacaccacaattgcagaaaagaaactagactcagatgaacacaatcaagagctgaaataaaaacagtatagaaaaattgaacttgtaaacaactgaattcaagaaaaacgaaccaaatcaacaaggacacaaggagaagaggaaaacgacttcagcgtgctggttggagaagcgggctgggctaccggagatccaccaccgcaccacgtcgattagttcgctgttgccgccgacgaaaggaaaggaagaacgatttcttgtttatgaagaagaacgtgaatgggggaatggggatatatttaaataaaaggattactaagctattaactaaattggtagggattattaaggaattaattaataataaaatatatattttacgaaaataaaatattagaaatgatgtggataataagatgacacatgtcagattataactggtacaacaaagtcatgggtgatataacccaaaattgagcatgggtcgtttagacaacccatattaaaaatatgaatatacaccacagttttaattgaatcattcaatgtacagaacaaatgcgggagcagtacttcacccaggtggtgcccggcccactacacaactggtaccagctatgaactccagaattgcgtcagatccgtttgcatggtactgtaattaaccacaaagtagctggaagtggtccattttgatatatacaatgacacacattcaatttcatcaaaatttcacatcatttttattactccctcctgtcttttttttttataagaaactctttgataaaatcacataaattaaggaaactaataatttttataaaatagatgtctttttcttttcacaagaacaatcatgttaaataccacaaatgttgacaatacatattgggtgtttacaatttctctccaaatttttgcatgtggaataggtctctttacattgtaattgattatttgacataatttaataagagtgtttcttataaaaaggataaaaaaaaattccaaagtattccttataaaaaaaaccagaggggttattaaatttttttgaaaggaaatttcattattcaaatgtaaaaacttgggaacatcattctcaagtttgtagattatttttgaaaagataaattcaaaaatttcacttttatcattgtcataaaaaaacaaaaaacatgtgattaagtgagaccctttctaatttcacatgccagctgtgtcccacttgagcaattaacacttgaacagagtagtaatgtcaccctttcacccgccacccagattcgtcactataaatgacacctctagcaattcacacacactacaacctagtaagcttcaaaggtccttccaatagttctctattctgctaacgcttgttaatcaag

SEQ ID NO: 87 - DNA, 1659 bp, Glycine max
tctttgtttctttacttatatatatatatatatatatatatatatatatatatatatatataatatttgtacatgtacaatgagggcatagttggtaagagaataaaaactagtggaaagtgaggtgaaatatcagagatttactacgaatattaactagatagtacgtgagacaaaaaatactatttgaatcatttatgtactctcggtgtaaaaaaatacatgatcgatatatattctatttccccataatttattagttatgatgtataatttggaatattttgtaattaactagagtaataaaaagattaagaggctcacaaattaaagaattaaaagcagtggcctttggcattaggcaatgatatttacacaccaaaagatttatactttgttttctatttctttatcttgagaacgaaaatataaaatagtaaattcaaacatttttataccctaatttttatattcatgttacatttaattctgtttactgttctttttatgcaatcatatatcgaaattattattttctttctatttagtcttcacctctctctcgatgaatatataaacaccttaaaggtgtgaaccactatttatcctaaattatttaataaacaattgtacctagtatatccacattatcatcactttaagctttatccattagtcagtgattattaactggatcaaacagtgctaattagaagggaaaaaatggttttaaaagcttattgcttgcgaattttttgtagataaaccatttaaaattcttcaagcctttgctgccacaatctgacattcaaataggacgaatgacatttaaatagctccacctattaatattcccttaagattttttaaagaaaaaaaagagagagagagagagaattgaggagggagggaccaggtatagaagccacgtaaacaaagctttagtgctgtttcttggaaggaagaacaactcatagaatgcaatgaatatggcaaattgccaatatcaatatatgcaatgctacgtccatgtccatcatacatataggggacaaataaccattaaaatctaagaattaataaagccaaaaggtacgcggcttcctcacaataataccaaaaccaaaaccactaccttagccagctgtctccccgcttgcaattttgatgtcagacaattttttttaatcatcatcatattgagggtagagtattattaattttattaataattaatttaagttaaagaatctaatttattatttttaatgaaattttctcttttgattattttttttattcacatttacaaatctaaaatcttaaaaaaaattaattttgtttcacttagatcaacaactaatagttattagtggtgacaagcgatttaacttgatcatggtagtaatagctaataaattttgattttaacttcttccttctctttcacctactcttaacttttgttactataaataacccccactcactggctcactctcctacaacgcatcctaaataagcatcttaattcatagtctcttggtccttccttccttcttctgctcaataagtgtgtgccactctaattaattaccacccccttctagacatagttcttctccctctgttctctattctctacactgtgaaaccaag

SEQ ID NO: 88 - DNA, 1252 bp, Cajanus cajan
gaaacagaaagatagagcacaaaaaattgatgatagaggatgacggacccctatgtcacagcctcacagagagatatttgggatatgcctgttgctgtaacccagattccaggggagactgggtccgctcaaaccctaaacgtaaaaaaatgttagcaaatagacagtttcatcttatagatatcactaaacatttttttactagacagtattaccgagactaagattattatggatgataaaaaaaaattataatatttaatatattcacatttaaactcgaaattactaattaaattagaataattttgtatcaattaattcattaaaattgttagtaaataataccattgtgtatagatactcacggataatttgaggaaaatgttattatctatatatttagagtatctttgtttttgcatttcaaaatttttaaaagataattcagactttctaatgcatgaaatacataaaccagaagaaggattcaaattaagttgagattaactttttttttttccaaacacacacttggtcataatcttggaaatgtcgctatatactaagatgataggttaaaagaaattcgttaatctattcaccaagacaggttatcacaaaacatcctaaattaagttgaaatttgttcaagattaccagttgcctcctgaacaaaattcgtatatgcaggggaatggccatagacatggtttcatgcccacgatccgacttgcgacaaacgtcaatctacatctagaaatcaatagagtccaaaggaaaaaaaaacgcggctttagccgaagtaataaaacataatactagcaacatatttcgaatatgttattgaaattgtatagtaattttattaaattaataatttagttggatgcccttttgttcttgaataagacgtaatgtttttttttaaaagaattataaaacgcaatttgttttttgcaggtaattaattagatatattccttgtgtctctaataaaagcatgcattgtgcagtgaaggatgatgttggtacttggcggtactaggaaatggccagctgtctcctgcttgcaactttgatgaaaaagcaattaaaagaaacgaaggagtaataaatatgataagtaagattttcctaatcttgcacctaccacccttcatttgttactataaataacacccttaactggctcacacagcagcacaacctaagctagctagtaagctttcagaactcctctttcttgaca

SEQ ID NO: 89 - DNA, 1581 bp, Glycine max
tattagattcaatttggtcaatttatttttaaatgttctaattatgtccttaattttttgaattgattcaagttggtgtcatctaccatcaattttgatcttctgagataacaaaagaaaattgggatcaaactgaataaattttaaaaaataaaaaacctaattgaatcttttagaaaataaaggaaccaaattaaacataataaataaattaaaggatcaattaaacctaaaatataaattagataattaaaatagtaattaaacctaaaaactaaaaggataaagtaataaatttagtatcaatttattttctgtttttatttttttatattttcttacatttattttaaaaaattggggaaaatatgtattttcttcgccattaactaatttatttaaaagtttattgtatcaaaatatatttgaatttttttaccaaattaattatacgaaaaaaaaatcaaactaaattcatatttgcatgtttatcttctccatatacatttgaattttttcttcatgttttggataaattattattaggctatattttactttttcaatctaatttgttcatattttatcattttcttaaatgatttaaacatttagctttatttctaaacattttttttgttcccgttaaatttcaattttaaatttatttaatattagtagtatttccactctcacatgtctacaatttattttttaaccatatagttctatcaaagatgaaagagatgagtaacaaatcaagtattggataaagataaaaatgaaagtaatttgaatgagatgattgagaaagaaattttttggactgaacttattaaattaaaattgagaagtatatttgaaaggaattaaacaatttaaaataaaaaataaatactcaatatactggataattaaataagatacctgacttgatgaaattataaataaatcctaatacgtcctaatcaataataactcttgaaaatctttcttgttcatgaaccactcttaaaaatattctcaaataagaagagtggttgaatagattatatatttatttttcttgatccttaattaatcttttagtagtaattaataagtatatcgtttatttgttaatgaatctctcatcttaatagaattaaatactaaaataataacatatttttgagtaaaataataatttcttcaaatccatgtccatatctctcaataaaagaaaccatatcaatatccttttttagataacaaaatcatatcaatatcagatcgatagaaaagaaatctaatctaatttaacgtagaaaaaaaagtgtatctccaaaacgactcattaaaaaaaaaaaaaaaactgtgacgcaggagagacacgaggacttaacgttgtaatgatctaatggataaaccaaacaaacgatacattttttaaaataaaaaaaaagaagaaaatgtcccggtagccctataaataaagcccaagcgttgtatttagcttttcatcccaatcagaaaaagttagggtttgtaactttgtcttttgcttgcgaatctgaatcgaataataatcaatcaatcatc

SEQ ID NO: 90 - DNA, 1500 bp, Cajanus cajan
aatttaggtcttatatataagagtagagacaatatttactaaaaataaaatgacaatgagtcattggctggttgatataacatgttagttgtcatattgtgtttttagttaatctgtttggtgacatgcatattgatatagaacaatataagcattttagtgaccaaaacaataataacaaaatattttacttttgttgaggatcaaaaagaacttaaaataaaatattttgaaaaaaaacaatgtgtatatatatatatttgttttttttcttttgtctctatttgactttaaaatatctagatttctatcaataattattttttaatattcaaatatttatacttaaaattttaagttaagttggataatttgagataaccattaacttcatattagagtatcattcaactatttttttaaaatacttttttaagctaaaaaaaatgtctttaatgccatccaactaactaaaaccccgttgttactactcctaataggtctaaattcagttttcaatagaaagttgtttttaattattaagaatacaattttaatttttaaatataaaatatatctcaagtaatatttaaagaaatttatagattaatcccaagcgtgtggttcagtggtaagggttgatccacgtttaagcaatgtcagagattcgagtcctgccttatttatgcagccacgttaaatcttgggaccagctatcccactttgaggtgcctcccttcaatgatggatactccagggacgaatcagaaaaaaaaaaaaaaatttatagatttttaatcttttattttttcattttttcttgagttaaatttatttattcctttcatttacgattttatggagactttctataacttcttagacataaattattcttatcttttttaattatttttatctctgttgatatatgtatataacattttaaattattttatctcttatttgattcagctatctacatatctatgattcaataagaacgtaaattatatttagattaaatctaataattatatccttctattttatggccaatctaccattttaacaaaaataaatatgaaaagaataacatataatctagaaaaataatacatttattacttgaatccatatccatatctctcagtaaaaagtgtaatatttatacttatggttatggtggttaaaaaaaaagagaaaataaatgtatctagtaaaaaaaaaaaaaaaggcatcagaaaaccaaattcgctattaaacaacagtgatttatttaggcacgtgggagagagaggagaatggataaacaaaggaaacgaaacaaaacatttaaaagagaaagaaaaggaaaagagggcccaacccaacccaatagcggtataaataaggcaatccctttattatttacacctttttcaccccaatcagagaaaagctagggtttgcgactttcccttttccgaatccaaatcagaatccaatcaatcggcgatg

SEQ ID NO: 91 - DNA, 1500 bp, Lotus japonicus
aagaatatttcaaaaactgtttaatgtaaaactactcacataaaatttaaaaaattatatatcaaaattacatttgattggattgtacttcacaactactaaaattttatttctcaatataacattcattttttttttatacatcagaaaataactacaagaacagcgaaactaaggaactacacataagctcacaataaaagagtttcaagctcatagttaagaacaacaaggtccaacctacaaacaagctggccaacaccagatttagccaaccaaacaacaacattattgtactctctcaaaatcgactgaatgaaggtcgatcaatcccgcctaaaaaggggctttgatatcctccaaaatgactaaaaaaacactcaaaattcatatattcaaacaataataataatattttcacaattttaagttcttttacaataaaaatttgatatatgcccaagtttcatttgacaatcataatattttaaaagcaatgagtatctcattcatatattcaaacagggttgttaatagcgcgctatagtggcctaatagcggtgaagcgtggcagatcgataattggccacaacgttggagaccttacttgtcgtagtagtgcccaaaatcgcgtatggagcggcgcaactgcgatgcgaggaagaaagaaaatataaaccctaaattcaaaacaaaaaccccaaattgctcccaaatttatacattaggttaagctttttaatggtattttggttttttttttctatttaatgaaacactacgtttcattccctaaagcctctgcaatcaaaaaccctactcattcgagtgaaaatccccaaatttaaacattgggttaaactttataagggcattttagggttttttttttcatttataacgaaacgttgtgtttcgtccttcttcaacctgaagcctctgcaatctctttctttttcctcgatgaaacactgagtttcctccttcttcctcctcctttcttcttcttcctccttctctagcataacgaccatagcgctacacgttgctatagcgttttttgaacttgctgctacgcaacgctatccgtcattaacaacactaattcaaactattttttgctccatcgaaaaactaacactaattcaaactatatgagtaatgatatttacacaatgttaaattcttttaacgacaaaaattgtcattacaccccagctttatttgacaatcataatcttttaaaagcaatgaatatctgccacataggaaacccaaaagacaattcaattttaatttttgttgcatcgggaagaaacccaccaagaggcattgtttggcagtatatataaacccatctcactcgtgcttatcgttcacatcacacaattatccaaccaacaaaaagttagggttagggatttcactttttctatctgcgaaaagggggaagaagaaagaacgaaattctcattctcatccaaaatccatccaaccacc

SEQ ID NO: 92 - DNA, 1500 bp, Glycine max
atatatcattacactactgattcattcattgactttaatattgaaatgtttttataactacttctactttcagctcatcttgagtcagcttcctctacatcatctcctatcaaaagtaaatctaaagcgtagaccattgaaggctcaaccactcgattaaatccattttttttggtcggcgctcaagtaaacaacttgtaagcatgacaattgtacaatcaacataatgtttaattagcactctgctccatccattttcaccacataattgcagaaactagaattgttttagcactagctataatgctatatatagatataagaattttgacatgactttgacagcttttgtgactgtgatcctcaaagcaatttcataccgaagtaagaagtttattattagtattactatatgcttggaatcatagagtctctagggacttctgtttaaatttattgtgatcatatatatttagtaataaccataacttgatgaattaaaataatgatactactatatgctttaatacatgcctataaaaacctgatgtagtaataaaggaaaagaaaaaaataaagtaccggacgggtaaattttcggtgggggttgagatttgagaagtttaagggtgttgctaggtacatctagcactattgttggtgcacctagcattttttattaatgtccaaaatactcctgactcgttgttcctttttaagttgttttttctggttaagttgtgatccgtaagcaaatttttcacatacaaatcaacttcattcgtataaattatacagatgaagttaatccataaatatgaagtttatttgtataaagcatacggattaagtttatcgatttatacagatgaagttcatttgtataaaacatacggatgaactttatctgcatgttttagacggatgaagctcatctgtatgatttatacggatgaagttcatccgtattggttgattttttttttaattccttgaagttttattttttaattattaataggttaaattactcatttgtgcattaaatttttattattattacaaaatttaatatatattaaattttgtaataataaatattttttatttataaaaaaatatatggattaaataattcgtatgatttatatcaaaattaattattacaaattttattatttaaatttacatgcataatatataacaacattatttattttataatataattaactcattagcttgacaaatttgtagagttgtagaggatttatgttgatagatgaataactaaatttactaaatgaagtttaaggatcacaatgtagcataattttatttaacatcaatttttttaaagagatagataaaatttgtttcggaaaaaaaaatagaaaattacttattttaaataaaaataatttaaataaacatattaaaaaaatatttaaggaaaaattacttattttattaaaataaacttgaacaaattgagcttgc

SEQ ID NO: 93 - DNA, 1689 bp, Glycine max
tagaggattattattattattattattattattattattattattattattattattattattattaataagcttttgactagatgtcacctaaatcttaagaagattttcgtgcctctgtgtttatttttaaaaaagaaaaataaaaaaatagtaaaaaaatgtagtgataacaatttttgtagggaaaggattattattattattattattattattattattattattattattattattattattattaataagcttttgactagatgtcacctaaatcttaagaagattttcgtgcctctgtgtttatttttaaaaaagaaaaataaaaaaatagtaaaaaaatgtagtgataacaatttttgtagggaaagtaacatcagttatattttaaaaatttatgaacaaaataaaaaacatttatatatattaaaagataagttaggaataagaaatatgtacaaagggataattgtcattaatgatagttatagataaagaaaaaattaagtagtatatttttacaatcttaaattattcattttttacttaattagttagtaagtttaatattaaattaaattttatactaaatgacacttaaatatttctactttataggttacaatatcaataatgaaaaggtatattaaattacatatacatagaaataattttaaaaggattaaaattaaaaaaatacattacaagaacaaaagtaaatgggagaaaaaataattaaataaagtattacacggtataatcaatcaattatactttttaaaaaacttttacttaacgtgatacttactttgagttttctattttgattttcaaagaaaataagagtaaaaacaagttcgacaaaacaacatatcaaccttctctaaaaaaatgaaaaggcaatattactttagagtatttttcttcttgataaatattttctaaattttaaaaatttaaaaatgaaaattattttcaaaaaataaaaaataaacacttaaatcaaacgtaactttagtataaattgagttaagttaaaatccataaataaatttgacacataataactcaacataaaaaataccttaataaacatttgaataaataagtactactagttgctccctctggtataattataagaaaaactttcattaaaacttcatattaagaataaaaaagaattatttaaaaatataatctcagttaaataaaaaatattttaaaaataattttattaaatataataaagataattaagatttatttatataaaattttacattgtgaattattatattcaaaatcgaaaaccactagggtcaaccaagtaatattaggttggggtaatacacattggatctcaaattcaaatcttagtgtcattattaaacaaaaaaaacttatattcaaaactaagagagtaactttttttaattttccatgcaaaacagaatctgaaataataaataatactccatttgtccctagctgtaagggtatgcagaaaaaataattaatataggaagaaattaaaaaaaatgatcatataaaaattttaaaaataattaggtatgatgaaataaaatagtaagccggttttatgaggcgtagggtccggtgaaaacaaaaagaaaggcagaaaaagcaaagcagacatgaaaagcatgtgaccgtgaagtg

SEQ ID NO: 94 - DNA, 1500 bp, Phaseolus vulgaris
catatatttcaaccataccaacttatttacatttaacttaactatcctcaaccgatataaacaatgtcgcattataagttaaaaaaaaaaagaaaaaaaaatagagagagagaattacaaagtttctactacaaaattcttacttttgttcttgttcattgactataatgtttactgaataaacaactggaaaatcaatttagcgagaacaatattgtctcattttagaatcactcttgttattttactctacaacaactcctaagaaatatttcattaaattatcatttttatgtaaatcagaatccaaattacggatcaagactcagagaactattatctcctatcttgatttggtcgctttactatcttaatggtatctgtaaagatattctgatgttcaagtaagatttttatattcaatgtttgatacaattaatatatgataatttatttttttcttaaaaagtgagtgactatttatatattattatagatagaatttaacgtttaaatcataattaaaaacatatttaattactattttaaactgattaatcttttgctaagttttaacaatttagtcatctcgggtactaaaacctctaaccttcgttcctataataataataataaataatatttggcatcgtgtagagatatgcatcggttttataagcagtaactagtggcttgtttggggtccagtgaaagcacggaacacagattaaaagttgacgaagaaaaaacaaaacatgatgaatgctcaatgttaccgtgagttgcgaggccaagatgggcccacttcaatctaaataacaatatttcatcactaattttaatcaaaatcttaataaaaatattaaatcttaatgtactggggttgacccggtcaactcacgcaaccatcacaccttaaggtacgaggtcgaccaagtggcatatgtggtcgaccgacataagatgttcaagtcaaaggttgacccgattagtaaaggctaacccagtgttaggaaacgacctaattcaatcctaatcgctaaacaaccctctaatccggcccaacagcaagggtccatcaaggtaatataaatgtcacgcattccaagaaagaagttacgtcattatctacagtattctaaccagccgaatacgacatctcactgacttgagcgccgaatacgacatcccattgacttgagtgtcggagtaccatttgcaggtaccccaccagtctgaggagccgttcgacgaagaagatagaaagaacgtgagacacgatcgaccgagtgacacctggagacaatagttctcccagtccccaaacccaaaacccagttcgagtacacttaataaaaattgtataatcaaattataaaaaataaaaaataatttttcattcataaactaaaacgtataatttttttaaaatatatatttttttataaaattgttgtataaaagcacagagacaaccctactgacccgactgctacacta

SEQ ID NO: 95 - DNA, 2343 bp, Cajanus cajan
attttatttacgtcttacaaaaatggtttctttgcaagtagagtaaaaaataaaatattttttaacatgcttaaaaaatatataattacgaacatgtttatatacaaatttaggtatttctctaaaaaaactatttttttttaaatagataagatacttttaacttatatctaaataagttttaaaagctgattttatatgtgtattcatatatataaaaaaaagatatttttttaaaaaaatatttatatataatattacatcttgtgttatctttgtcttgtctctgattcagtcaagcgggcgagagaaattccttgacagaaccaaaagataatattcatgtcttatatttaaataatcaaacctttttaattctagtataaaaaatattgtataaattataataataacaacagcatttgaattttaaatcgtacatatattacactaatctttagttatttgacggaatctagtgattataattgcagcaaatatgtaccatcctaaccaacgtttttttttgttttttgtttttttaccatttatgatttaggtgaagttaaaatccatttggttcatagtttctcaacatgaaaaatactcctgttcaacaatgagttgtctaccaagatgtatatatcactttaatgagctcgtcgtaagtaattcaaattaatttaaataaaaatatcatattttaacccataataatattttctaaattcttttttataagtcacatgtcatatcttgtctttttaaaattgatttacaaaaatttattattcttttttacaaacctcacatattattcaagaaataagaaaaatgcaaacacgagtccacgcatacattgtcaaaaaatattaaattaaaagtagtatataaatatatattttttaatcagagtatcctttaatcaaaatcaataactaatctttgagatactaaaatcaatttaaataattgatctccgtaaaaaaaatatattttcctatactcataaactaggacaaaccaccaattacatgcttgtaaaacagttgtattttgtaaccatttcatactatatcaataaaattaatgtttaattatctttttaatctcgatagtcttataaattttacattttaatgattataattaaaaattttcattttagttatacacgctttttttcttctctttttaattcatataatttatttttataaaaattaaaaaaaatgtttatataattatagagactaaaaaaagttatataattataagaactaaaaaaaattatataattataaagataaaaaaatgatattcctaaaataattttttaattataaaactaattttttaagaagttagaaaaataaaaaataattaaaatcaaagttaatatacacaccaatgcataataaaataaattatagtatattaaatattacggaaaaatgacacattaacccaatagaaatagaataagaaacagaaacagaaggattttgatagtaaaaaataaaaataaaaatgaataatttaaaaataaagtaattttgcgggtcgagtgaaaacgcgggaattgagaaagacagaaaagcaaagagacacgaaaagcatgacgtatgtcagcgtgaagtgcgaggcaatgggtcccacacaccacagcacccaactcacgctactacaaacggtcaacgcttcccactttccacggaatctccgccacccaccacgtgtcacactcctattggacctcacctctcctatatcttagcctccctttccccatcttttccccatttccatttttttctaaaattttctctctccttcaggttcgctctctctgtctctcttacatcaccatttctttttagggtttccattgcgccttttcctttctctattttgttcctcctaggtttcatttcattcatccagacttgcatctgtttctatgttttcattttcgcctttttttttttttttatttccgatttcttctgcttgatttacctcatttgcatctcgattacgctgatttcaatttcatcatctctttaaataaaacgagaaacagatcaagattcgctaaatgctgaattgagtaaaaataaataaaatgattagtcaatactttaacacatagtcagagaaataactagaagagaagtgtagaaataacacgtgatgtggaaagactaaagagaaattgagatgaaaaaatgacaggtgttaataaagtatatatatatagaagaagaagagaagtgttcacgaatcacttggagattgcctaatattgggaaatttacttgtaggattaggcgaatctgagaa

SEQ ID NO: 96 - DNA, 2173 bp, Glycine max
ggaaaacattgaattggagaatgaggtttagggttagtgtgagttttttttccaaagaggtgatagtgatgttgggtggttctttttcatcattgttgttttgtttgtttgagttgacgatagtcaatgtggtgatggttgtgtaacttggtcggtggttgcgtagtgtaatcaatagtggtggcacgatggcagtggtttctattgaagaaggggagaaatggaagaaggagaatgagaaaaaggtggtgaggtggcgttgaagtgtcagtgacaaagcacgatgttgtgatatttcatctgtcacattatatttaatgaaagaaaattaatgatgagtaataaaatgaaacctaaaattttgtagatacttaattgaaaaaaataaatgttaagtaataattttaaaatgagttatattgcaagtacgaaaaacttaattaaacctttatttcttatataaattttcactaaacacatgaaattcattgtctccttttcatttcactctctggtttaaatccttaccaattattaccaaacaaagcactagttagtggaaattgaagaagtgttttgttaatgcatatgagcagtgcatctttgtttttttgttagccagaatacgcgattaagaatatagcaagaaggtggcgttttgttttgtgtggcggataaaaaaaaaaaaatcagcgaagaataagtgttccattggaatgggctttggtagagacaatagttgtccaataaacaagaaatatacttattgggcttattggagctgaaccggaccaaaatcacaatagagaacaccagcaaaaaggggcgttggtttgggcagccagcacaatgataatacacccagcaaaatagtgaaatttcatttttacccttccataaagctgatatggactggcgaatccataaacctaatacggatttaaaaatatattttatgaattaatttaaaaataatggttcatcttggaagcaaaccttattattaataggatgctctaatcaattaagttaggcaaattttgttataaaataattaatgtcgctatatataataataagattttcaatgtatatttaatatacatgtaaatttaaataataaattttgtgataattaattttgatctaataattaatttgtttacatatataaattataaataaaatcataaatttcataaaaaattatatacataaacaaattaattattagatcaaaattaatttataaaatttattatctaaatttatatgtaaacaaatatatatttaaatttttattattatacatagcaatattaattattttataacataattaatttattaattatgttgatgagaacatcatattaataatatgaaaatcacacgtgttgaataattatatgaaccattagtctaattcgtatcatgtagattgattgatatcaattttatacaaagcaaatgcccgtaaaagaggagaatgaagaccgcgggaacgctgaaggaaacacacagaaaacacaatatgatggaaacggaggcatacgcttaggaataacgcagccatcgcaaaataagcgcacggatcaatgacatggcaataaatttaaattcccgcctaaacccctgacctccccttgatttcatttcgtttgcaattgaatttcaatttccctaggggcttaatatctcccctcacttgtgcctagccttatgtcatagtattatcattcattctcatctttctctcttctctctccacccaaaccctaactcccgaccctgttcccctctcggtacgtatttccgctcaatttttcgtcgtttttctgcaattctcttcgttttcaagcgattttcttcgtttttctacgaattcacgccgtaaaactatcctgattgtgtgattgcagtatccgtctgatcaagagagccgatcccaagtaagtttcactcgcttttgatccaaccatgaattgtttgtttctcttacgattcgacgcttcttactgtgaaataatcaaaaaagctaaggttcacgaatttccagatctgagatctgagatctctgtttggtacggcgattatttcagatgtgtgcgttttactatattgtggttttttattctatttattttgctatgaatttcagactttaggatcc

SEQ ID NO: 97 - DNA, 2069 bp, Phaseolus vulgaris
aaaatattgttttaacgagataaaatgttaaatctagataaaagggtgtgattttagttatttatagtttcgtagaccaaaataagtgcttaaaaacttaagttttaaaggatttacacgtgataacttatatatacatgttgttgtcttcctttttttttttttctataacataatatttgactatatataactttaatttcatcctgactaagattgggttatggaatattgttataaagttagtcaactttaatgattagttggtaaatatctttgtcaaatttattcatggaataaacactgactacatacttagtaaatttggtatatataatttatatagtgaaagtttggaaatgtattaaaatcaaaataatatatttaagtgtaattaatttatataataaaatcacaaccatattctatctttacttcaaccttttccctctttttattttaacaattttgtaatcatgaccaccgtaataatgattcacaattggtttatatatgaaaatggtttcttataaaaaaaatgtaaaaaaaggtgtaaagaaagaaactattttttaattcaaaatttagtaatcaattttataattatattctcaatatgaatattttaatcgatattattatttaaaattaatttaattaatttattattatgcttttaaaattttcttataattaatatgttttttaacacataagtaacaacaatcctataattaatgttgagcaaacatccattcacaaaatttcgcaatgaaatattttactatcatattcatttgatttaagaaaattaagtaaatataaattattttttaatgaaataaagaaagttaaatttaacaaattaagttaaaatggagacagactaaataatttaacctaattattttatactaatgtttcctcttttactcattttttagatgactattttttacgatgatagaaatgtaacatacatatataaataattttagatgagtcaaaattgaaaacacaaaaaatggaatgcaaataatgattgaaaatggaagttattttattttataggtaagatttggtatgaaaatgtttaaaggtatgctctatttgttgccttagaataagaaagagtgagaaatcatataacaattatacattaatattaattttaaaaaattaattaattttttaaataattcatgtgaaatgagaaaagggtagatagtggaactgttttgataaaattagttttttttttaatataatgtatgagttatggaaagtgggtgtgtttttgtttggtggatgaaggaagaaggaatgggcttgaaagaaggcaataaaactagaaatgggtttgatagagtggcccaaatcacagtcggaaaatagaagtaaaagcaatgaagaagatcggcgattgcagaagaaagcacagagaaaacacaaaagggtaacggaggcataagcttaggaataacgcacccaacacaaaataaagacacggatcaatgacctggcaaataaatttaaattcccgcctaaacccctgacccctttgatttcatttcatttgcaattgaatttcaatttctgaatgagcttaatatctccccgcatttgtgcctagccttatgtcatagtataacatatcgttcccttatctttctctctcttcttctctccacccaaaccctaactcccaaccctattcccctctctccgtatgtaatcccttcaatttttctctatttttcatcaatttttcatcgttttcatcgatcttctgtgatttccaccgaataaaactaatctcctcgtgttactgcagcatccgtctgatcaaagtaagtttcctgtgcttttttttgtccttccttcatttgtttatttcgcgcacgattcaaggtttcctgcagtgaaatatacgagaaagttgtattttcgactttctggatctcagatctgagatctctgttatgtatcgtgtttaatatatcagatttggtcgttctattatttgattttggattccattgaatttgctttgaatttcagacggatcc

SEQ ID NO: 98 - DNA, 1752 bp, Cajanus cajan
ttattttatctttaatgagtttctagccatgataaatgacacataactcaggctgaaccatctagcatggacccaacttacaacagtagacaagtatgaagttgcttacatgactatgaataatacctccacacaaacaaactgcactttgatataatggtacaatccatcacaattgagcttaagacattcttttaagagtttaacctaattgatataaaaactataaaattatgacagttcaaaagtccacggtcgttcaatccatgaaattctaaaattatgagcttgtttctcaagagccatttgttaacattcacacctacgttctggaacaggaaataattttgcgtcttctgaatcgagccaggagacaaatattaagttgtccagatgactatgaataatacctcccacaaatcgcactttgatataatgatataatctcatcactgttgagtttaacacatccttccgaaagtttaacctaactgatataacaaccttaaagattatggcagtttaaaagtctgcaatcataaatttcaatccatgaaaatttcaaagtatgaccctgtttttatgagccatttgttaaacattcacacttacattctgaacacatgaaacaccacaaaataaataataaaaactcttacatgcatattatacttatattaaaattagtagaaatagacttaaaaatagtagaaaaatggataaataatctaattattacggttaacaaattttctaatgtaatttaataattttaatctaacttctcttccatgtgttttttaaataacaactacatgatttttttccataatttctcttacgtttcttgcactacaaccttaatttttatgtttctaattttaaatataataaaaaagtaatattttattttgttttatgtcataaataaaaataaaataaaataaaattcaatttgagcaaatattccatagaagtgggctatgggtgattcattcgataaatgttacaagtgtccaatacgcatgaaacatgcaatgggctttggcaaattggactggaccgaagtgatggccgaaaagagaaacgagaaagagtaagaagatcggcgatgaagtagaaagcacacagaaaacacaaaatgggaacggaggcataatccaaggaataacgcagccaacacaagagaagggcacggatcaatgacatggcaataaatttaaattcccgcctaaacccctgacctccccttgatttcatttcgtttgcaattgaatttcaatttccctaggggcttaatatctcccctcatttgtgcctagccttatgtcatagtatcatttcccttatctttctctctctccccctctccacccaaaccctaactcccacctcttcgtacgtattttccttcgctttttcatcctttttttttcgttctccacgaattcacgctcagaaatctaactctaactcgttcctctgattgcagcatcagcctgatcaaagtaagtttcatttgtttttgatccaactctgaatttttcatttctctgatggttcgattcttcgtacggtgaaacacaagagaactccaggtttcagatatttacagatctgagatctgaggttttcaaactgcgtctcgatctgtgcttggttccttatgcagattattttagatttgttcgtcataatttttcttggcttttggttctatcggttttgctttgaattgcagacataggatcc

SEQ ID NO: 99 - DNA, 1500 bp, Glycine max
ataaaaatgtcaaagacggggcggggaaaatccaacccccgtcgaggacaagaatgatgtgtaaatatacaccgtgtcgatttcgaggggcgggagtggggtggggtaaacaaaactcagtcccaacctgtcccgttgcctagcctactaacaatatacatatactctcttaaaataaaacatacatatgattatagtatgttgttattattaatatcttgaattactattattctaattataatttaataaaaaatatattataatattattataagaacaagagtaacataaaagtaatacaactaacatcttttcattcatttgtcaatgacaaaattttattaatttttttatgaaatatgcaaacaatgaaataaataaattcattcaaatttctttcaagatatttgtttgacagaacattttacgatatgtgaacataattgtacacccaaccctaacacacaaagaatgggtttagggagggacaagtacgaattttggcctccccagcctaagatcctttgtatacatgagacgaaaaaaaaaattgatagaaaaaaattgataaaaaaaaagtagaataaatgaattgatgtttgtttgattttcttctaataataattttcatctcttgttttacatatacatttaataataaatatttttgtggaactttttatttataaaaaaatatttatttaaaattagataattaaataattgtgtaattttttttactcctttaatatattcctgcatatagtttttggattagctaatttgctttcttaatcttaaaagtatatatatatatatatatatatatatatatatatatatatatatatatatatatataatttagacctgatttataattctacaaattcaaatacacgttttcttcctatagtttacaatcacaatttattaaaaagaaaaaaaaaatcaagggagacttattattttcaccagatttctttttaactaaaggaattcttattttcagtagaccttcttttgcggcggctaaataaaataatactacaacagttcagtccaaaaaaactcactttatttttctccattttgttatagttgaaataaaagagaaaaataatacgaattttttttaattattttttacaaaattcacatcttttatactctagtataattttattttattttcatcctttttatttttatcatcaaaatcaaaacacacgctccttgtacgctttacaagcgcaagaaatccccgagcatttttttgtttaagactagaaccctggccccacccgaaaggatattatagtaaaagccgccgccagctacaccaattactataataatgatgaaacgtcccggaaactaggcaagggtaagaccgttatttcctatcccatcccacctcgttagtcacggacgccctaacataccctccccacacacaccgccaccctataaattcgcaccttcaccgcatcattc

SEQ ID NO: 100 - DNA, 1627 bp, Cicer arietinum
tggaggccagttttctctgttaaatagtcttattttatttaggattgaggcaccataagctgcctaattatgatgtctatgcagatttttgctttaaaaattggacaaaataaaatataaataatttgattttagattagacttgattattgaaaactaattaaataagacaaaatataagctattttaacataatagaataaactattgtttcatatttttaatctaaaattaaattattaatatattttgaattaatagtatcagttatatttttaatgtgaataatatccaattaatatttaaaagagaagttgaaatgagacaaaataaaatttataaattatattttatatataagttgtatgttgaaatttaattaaataagacaatatataagttattttaattatgcgaaagattagaataaaaagaagaaatccatatttttaatccaaagtcatatatttaatggttgtttttgaatgtgattaaggatcatatttcaccttctctaaaaaataattaaataaaaagataaattgaaattagatttgaattctatttaatgtttaagatttgacttgtttgctgaaaactaattatatgagacaaaatgtaggttattttaattatgttaattataaatcacaacaaaatgaagtagttgttctgtcaataaacattaattattaaataatagttataaaagttcaaactattttaattaattgtgttcttatttttactatttttttactccattttattatgatcataattattttattctattagatgattaaaattacttatatttgtcttattcatttaagtagtttttaatatacaagtcaaatcttaactaaaatttgtatattttattttgtctaatttcaaactatcattttttccccttttttgtcttatttttgtggatattttacttttatattctacttttattaaaaaaaattcattataacctgcattaaaatttatatcttatttttattaacaattttcttttttgcagaaaagttagccactttatatattaggttgaattcatttttttttgggtcttatatattgaattaactttaataagatattattcttattttgatacattttaaaattacttgaaaccgcttaatacgtcaaagaatcaaatctactatttactaatatactttttatagtattaaatgaatattttattttagtacgtgacacaatgataatgacgaaaactttaagaaaaaaaatgataatgacaaaaatatccaggcaggtttactttgtgaagctgattttgtcaagtagcagcacaaatcaaaagaaacatgcttaatggatgaagggttaagaatccatctactttaaaaaaaccataaaccatgacccacccctaaatcatggaacaaaaaggaggatattattgtaaaaacagccgtcaatcaatagaataatgatgaagagtcccggaaacgtagtaagggcaagaccgtaatttaaaatataaaacttctttgcgcttctcacggtcacggacgtctcttaaaacaaaccctcgccccacacgctaaccctataaatacactatcacctcacaaatcccaaa

SEQ ID NO: 101 - DNA, 3202 bp, Phaseolus vulgaris
ccaaccatgttttatagaaagtgaaattttttcactccagctaaaatattctcgctcaagcttaaactattttttctttttttcttgttgtatctacaatggattgaattatatatgaatgtttagtttaaacatgaaatgaatattaagatgtatatttaattagttttgtattttttgtttaaataagttatattatatgacgatgctaactgatattgatggaatttgtatgaatatgtggttacatattgagataatatgtgaactattgtttgatttgtgtattaagtattgacacatatgttgtcagagagatcaccttgcttcactaatatcctaagtccatagattaatatacgaggaggtggaaaattgaaggagaagttcttacacattatgatcatatgtagatatatgacttgagttgtaatgaacttatctaattcattttcttaggttgtaaatataggtatatatgtcttgtatattgaatcaaatgttagtttttgataagttatatttaatataagtctttcctgaaaggtcaattaataatttttttattgaatattttttatatataaatctatgtgtgatatatattattgttatacatggacatttgaaagaaattaaatgaatatgatatctcaattaaaatattttcttttatatacttttttttattttaataatttattgttatatgttttttaatgaaatggcaatgacataaaagttatgaaataatttaagaaatattttttcaatgatctgaagatattcacttgatttaagagaaaattcaaggtatatttttattaattaattttcgttcttttatttttttatttcttaacataacacttcttggaggatttcatctgcaaactcaaagtttatgactaaaccaacattacacactatcatttgactattgcatttatttttctagttgccgtaatgagatattataataattattctattataataatatattataataatatctgattatgttaagaaataaaaaaataaaagaacgaatatattccctaaacgaatatattattattctatttaatatattataataatatcccaagaagaagagtaatataaatataaataataaacttataaatcacataatttcattcaacatgtcaaacaacaaactacaaaaattattttataatatgttgtaaaaatttattcaaattcattttctttcatttactatatataaatacaagttttcctcacatttcgtatcataaaagaaaaaatatagcgtggcagatttagttgttattttaacacgatcttatcttgctgaatagaataaaataaaacaactcataaaaaaaagaaactttgtggggaagaagaatcccgaaaaagaggatatagtaaaaacggccacaccaatcaccagaacaatgatgaaacgacccggaaactgggtaagggtaagaccgtcttttcacatccctcacactagtcacggacggccctaaaaaaactcataacaaaccctcctcgcatacacctccaccctataaattcacctgcgcccccctcattccaatccaccaaccatgttttatagaaagtgaaattttttcactccagctaaaatattctcgctcaagcttaaactattttttctttttttcttgttgtatctacaatggattgaattatatatgaatgtttagtttaaacatgaaatgaatattaagatgtatatttaattagttttgtattttttgtttaaataagttatattatatgacgatgctaactgatattgatggaatttgtatgaatatgtggttacatattgagataatatgtgaactattgtttgatttgtgtattaagtattgacacatatgttgtcagagagatcaccttgcttcactaatatcctaagtccatagattaatatacgaggaggtggaaaattgaaggagaagttcttacacattatgatcatatgtagatatatgacttgagttgtaatgaacttatctaattcattttcttaggttgtaaatataggtatatatgtcttgtatattgaatcaaatgttagtttttgataagttatatttaatataagtctttcctgaaaggtcaattaataatttttttattgaatattttttatatataaatctatgtgtgatatatattattgttatacatggacatttgaaagaaattaaatgaatatgatatctcaattaaaatattttcttttatatacttttttttattttaataatttattgttatatgttttttaatgaaatggcaatgacataaaagttatgaaataatttaagaaatattttttcaatgatctgaagatattcacttgatttaagagaaaattcaaggtatatttttattaattaattttcgttcttttatttttttatttcttaacataacacttcttggaggatttcatctgcaaactcaaagtttatgactaaaccaacattacacactatcatttgactattgcatttatttttctagttgccgtaatgagatattataataattattctattataataatatattataataatatctgattatgttaagaaataaaaaaataaaagaacgaatatattccctaaacgaatatattattattctatttaatatattataataatatcccaagaagaagagtaatataaatataaataataaacttataaatcacataatttcattcaacatgtcaaacaacaaactacaaaaattattttataatatgttgtaaaaatttattcaaattcattttctttcatttactatatataaatacaagttttcctcacatttcgtatcataaaagaaaaaatatagcgtggcagatttagttgttattttaacacgatcttatcttgctgaatagaataaaataaaacaactcataaaaaaaagaaactttgtggggaagaagaatcccgaaaaagaggatatagtaaaaacggccacaccaatcaccagaacaatgatgaaacgacccggaaactgggtaagggtaagaccgtcttttcacatccctcacactagtcacggacggccctaaaaaaactcataacaaaccctcctcgcatacacctccaccctataaattcacctgcgcccccctcattccaatcca

SEQ ID NO: 102 - DNA, 2145 bp, Glycine max
ttatttattgattattttatgtgtcaaatttatagtttgtaaagccagtggcatgaggttcaaatccaaattgatgtatctcatattattattgatatttttatttgttttttatactaattactaaatgttttagtaaagagtaatataataagtaacttttttggtatttgtgagttgcaactttgcgagcctcaagattaatcccaaatcacgagttttgctgccttaaccctaatcctaaatcgcgagccccagcctatgttagtcgtcggaccttggaatgttgttaacaaccactcctcattcctcaaggagtgctctgttttttgttttaacatttttttttcgtttcaacctctattcactaacccgtatctttttctcatattggcgactacaataggtcctttgagaatagagttactttgtgtatttgtgtgtttcaccaatagaaccgttgcttggatattccgttaagaaatcatcatggcttgatttgtcgctttgtcttggtcatacaaagtaagagtttgcttctttttttatattgttaccgtctcatgaaattgtttttctttgtgttgcttcttaaattagtaaattataatgcattgaagttttgtccataagagtttgtgctttatcaagtttactttatgtgtatttcattacttgagaaaacttttgcactcaggcgcaaaagaaatcatgattagagtgtaataacatatatgatttgctatggtacaaactgtatatatagatcaactattatgaaattattttaattttaacaacgaacttaagttcaagttttaaatgtaataacatcttttgagcgtggttaagtaattagacatgcttaaaagttggatttgaaactcgtgaaatcaaaatataaatgcaaatttcattagtggaggattttagacatcacctttagctatcggttgaaataaaccgaagtgttgtctaatgtgacaagaattggtgaggtaaacaccaacaacggagacaagattttaggccacaaccaaacgaggaaaagttaacttgaacaactgagttgattagcctaaccaataaaaggttaatgatgggtttagaaagggagttacaagagtgataaaatcctaacaaacaataaaatattttcaaagcaaacaataaaatattttcaaagcgaaaataagtaatatgaaactaaaaaaattgaattaaatggtggaaaggaaagaaagggagaatcacctagaaaatatatacaatttatttatttttatatgaattttgactaaacacgtgaaattcatcatctccttttcatttcatcattaccaaataaagtactagtgtaatggaagaagtatcttgttaatgtatgagtagtgcgtctttgattctcactacacaaaaaatgggcttattagagctggaccgggcccaagtgagtagaaaagaaaggcaacagcgaaaataaacataggcggcgacacagaagaaagcacacagaaaacacaaaatgggaacagagacatacgctcaggaaaagcgcacggatcaatgacatggcaataaatttaaattcccgcctaaacccctgaccttcccttgatttcatttcgtttgcaattgaatttcaatttccctaggggcttaatatctccccgcatttgtgcctagcctcattattatcatagtagatattcattccccttatctttctctcttctctctccccgcccaaaccctaactccctaccctattccgctttctctgtacgtatttcccctcaattttcttcgtctttccggaattctcttcgttttccggctattttcttcagttttctacgaattcctgacctaaaaactaacctgattgtgtgattgcagcatccatctgatcaagagagccgatgccaagtaagtttcacgcgttttttttatcaaccttgaattattcgtctcttacgattcgatgctttgtacggtgaaataatcgaaaaagctagggttcaggaaactccggatctgagatctgagatctctatttcgtacaacgattatttcagatttgtgcgttctactatttcgtggtttttgattctatttatttttctacgaatctcagacggatcc

SEQ ID NO: 103 - DNA, 1606 bp, Medicago truncatula
agcatataatattttatccaaagcaaaggatactcacggtcatccttacctcaaatgagatataataaaatttattgattaaatattatatgtataaagaaaacaatccctttcagctcttttgtgttacaattttctttttaaaaatatcatcaattttgaatacaagtaacacatgtaacaaatagatacgaataacattaaatattaaaaaaatgtaacaaacacgatatgaatatatgtctgtttttttttctttatcatttaaagtgtaacaaactagatacgtatatttatacttacttttttattattccaattatacccttcaacaactttttctataataaagatcattgttggtttcaactaaaaaaaatagattatatattttttgttatattattggtgtcattgaaataaataatcgtgattattttgatttgttatgacttaaaaacaacaacctttatatttttagttttaacccaaatcaaaatttcatacaaaaaacactgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacgtactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttacagtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagagctgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaag

SEQ ID NO: 104 - DNA, 1580 bp, Medicago truncatula
gacgggctctggctaaaaaggtccgattttaaacgggcctaaattttagtgtccaagtcatacattttttcgggtttttgcgggctggcccgacccgttttgacagctataactgacggaagggttaggctttacgaaagtgaaaattgttctggaaacaacaaaattgatgtgcaaatctgctctcaattttagggttatttttacgccgggtttgaatattgagttagaaaataataatatacggcgtgacaatttatataactaattttttttttttgatgaaactaaatctcttttttttttgttacaatggtgaacaaagtagaaaaaatacaagataaaacagaagggaaaaaacataagtatctaggctaacgccttaagaaaagcgttcctactgcatcctccagcagcagagggaggaggccctccagaggggaagagtgaatctccaatttttcattttttgaggcttcaatctttgctaagaaaatctgcacacttgttaccttctctgagagaatgttgcagagtgaaacttcttgagctgagaatatctttgatgttctgaataagcatagcatagacatggtaatgatttaaatcttccttaatgagattaactgtgagaagggaatccgagtagcaggccaattcatcatagtttcggttgactgctaaatctctttctttacagggttttgatttttgtgttcaaaagaagatttgaggtgaatataacattaattcatgatatttggagatatatttgtgtatatttttttttattcaatgaagatgatgaagattcaaaggaagaagatgaagatgatgaacatcttcatcatatttcttgatttttttcatttttaataaaaatatatttaaaaaaaaattatgtatttttttaataaaaaaatgagaaatatgtgttttataaaggaccaaagtgtaacaaataaataagataaatgacctctaatgtaatcatgtctataatattaaaaaattatgattataattattattttttaatgaaaaattaactcatttatgacatttaatgtagccaatgtatattacattttaattggttgatatcatgagaggataaataccttctaaataagatagagtaatctaaaaaaaccatgtttagagacttaaaaaacaacgtctctctccgaaattgttgatttattaacttacttaaaatcaacgcaagataattcacgttggagtcaaatcgtgagttaatttgaggagttttaaaaagtttgaggtgagaatgagaaaaaacaaaacaattttcaaaaagttgtgtagaatctcctagagccttacagaaacgtaatcgcatacgtagtaccaaaattgtcgtagttgatcaacttgattttacacgtggcttaaaattaccggtagataggaggctattttcgtaattgagtcgacacaaccttcatataaatacagcaaccttcatccattgttttcatccttacctatttgcttgcaaagcaaaatttccaaacgctcttttctttcttcttcgacaattctacgattcattcaag

SEQ ID NO: 105 - DNA, 1464 bp, Cajanus cajan
taattttgggcccaaaaaaaaatctctttttcaaaaaaataattaaaatattaaaatattcttaaatttttgcttttttttttaattttacattcacaacatttaattttgggccgaaaaaaaaatctttcaagataggttttgaatagaatttaggtttcatttttttataaatacaccccctttttaaaaagaaaattattaaattcttaaattaccttaatattatttgaaataaataaaaaatattctggaaaagtgcttttcaaaggttgatgatatatacacctcttttttttttctataagtacacctctttttcaaaaaaataattaaaattttaaaaaattcttaaattttttctttttttttttttaattttacattcacaacatttagtttagtatttttttttaaaagtctttctttttttttctttttttaatttttacatgcaccccgttatctttccttcaccctacctattttggtaacatgtaaaaattgaaagagaaaaaaaaagtgtgtgaaaaaaaagaaaaaagtagaatatcatgagccttccgaaaagaaggtgtacttataaaaaaagtataatattttatagtttttttaaaaaaataaagaaggtgtacttataaaaaaaaacaggaggtatatatatcatgagccttccgaaaagaactttccgtcagaataattatagaaagtactttccaaaatactaaaattactattccggaaataactttccttcaaaataattatgaaaaatacttttcgtaacactagaattactattgcgaaaaattctttccggaatattaaccgtgtgtgtatttgttgccataattctggaaagaactttctgcaatagtaatttagtgttacggaaagtactttccagaattattccgacggaaagttattttcggaatagtaattttagtattccggaaagaactttctataattatgtacaaatacacctccgaaaaaaactttccagaataatttttttttattttaaataatattaaggataatttagaaatttaatattttttttttaaaaaaagggtgtatgtataaaaaaatggaggtgtatatatcatcggcctagaatttaataatgtaaaataacattcgagcgtttacattattgacaatgggcccgtataaagctgaggcgctccacttgcaatagtgaggacccaaattgtgacgtggggcccaatataataacgccgtcgtagcagataaagccaacgtcaagaatacgaaattgccaactactccgcaaattgcaattctcaatttcctagaaagactccctcgaaaggcatccgataccaaccatcatcacacgcgtcgcaatatgactggacaagggttccaccgctcttgctataaaatgccacaacctctcctcatttcttcgcaattcattttcaatcaaattag

SEQ ID NO: 106 - DNA, 2305 bp, Lotus japonicus
cgttagttttctaacgtcaaactgacggaggggtactcgctacactgttttgtttcattgagggtacaaacatagacgaaaatagatgaggggtgtcttttacacaaacatgaaacatcaggggcaacaaatgcttttaagcctaaaattaataaaatcatatctaagatcaacaatatctcagcaaagattaaatgggttgactctattatctattggaaaagaaactttaaaataatcgattatgataatttaacaaacgattttgaatctcaaaaagcccgaaaataaattttatttaaattatttatattaaaaaattaattttataaaggcctaattcttagagttcgcttttggcctcaatattggttgagacggccctgcttttctgcaaaaggattttttttgtgttcaataatattttatgcatacttctaatatacttggattaaaaagaaaattgagagataagaagagaaataagtggaaatgagataaagggagatgataagtgcatgggttgggatggtcttattgggatgggcatgacaagtagatttatggcttccaagcgtttgaagttggcaaaaattcgttcatttcagaggctagcgtgttatgtgaaggtctctctcttgtttggaactgcaagtatagaaatgccatttgtgaaggggttgtgcaaagttactttcttccattgagacctaaagagcttgagcttgatctggtattcaccttccacgactactaccaccaccaccaccatcccttccaccttccaccaccgcctccaccactacaaccattgttaccatcacttccaacaccacttttgccgctgcaacctcctaacccaccactaccactacagccgccgcatccatccaccatcgccaccacctccttctaccgcagtcacgtcactaccatcatcatcatcatcactttcaacaccactgctgctgccaccctaaccaccactcagcatcaccattgtcacctatgcagccaccatcacaaccacatcctccatcaccatatcgccattatcgcagctacatcggctgccaccgtcattttatcgctcactaacaccatcttcatttttattatacatatcattattcaatatctcacaaatcctctgaattgatttttttttattatttaatcattttttatgtgtttaaatattaaaaagattgaattcaatgattaaatttgagagtatcttttatttttaacaataatctgagaggatatcgtcctttcttgatacccaataaaaaaaagttgccgaaacggactccaataccaacgttggcgtgcaaatagagtttgaccgtctaccatcttgtccacgcgtctcaacatgattggctgagctggcgttgccgtaagaaatcctcatatcgttctgctataaaaaccaagcaccatatacaacattcttcattcacaaatcacaatagcaaagcaacaaacatctctctgagtctttgcagagaaagaaagaaagaaagaaagaaaaatccctcatctctcaccgtttcaaggtatccacactttctactctttctctctttcatgataagcttcgattcgatttttagggttttgtttgttcgtcgttaatcgaatcgaatttaattggcgttgggcatgattttactgttttattattcaatcaagattagtactttgaaacttttgtttatgaattagcactgggtgttagacttttctatctttggtattgattcaattgttatcgtaaacgagtttaatcgataatcagggtttgaattcgatcgttagggttttgtatttattattaatcgatcatcagaaaaaccacttttattttgatttggattcttaggtgtccctaaaattcttttgaattgaattagtttacatgattttactgtcttattaatgaatcaagatgatggctttctgaaacttttcttgatttacttagccaatttccttagaataattccatgattgaaaattatgttgagatttcagatttaggatatgtattcagtatccaataattgtttagtgtcttaacctaggtttatatttgttttgggtttttacttctttctttctgggctttttgcttctgtatgtgttgttatttccctgtaatttagcatctagcattgtcaccggatagagtttttgtaaaattgatgtacttgctgatccttttcagttctgttgaacataatgaataattggtgattgacacatttgccttttttgttttgatgatgcagatggatcc

SEQ ID NO: 107 - DNA, 1922 bp, Vigna unguiculata
ctcataggctatcacaaaagtttcacacctcaaaaaagacaataccttatatgaagttgagactactaagaatactagagcgagcctctgatcaaagtctaaagaaaatttcaattttcaataaatacaagactcctttgataataaagaatacaaaaaagaacacagaccaattatacaagaaattctctcaaaattcaaaataagacataagtagattgatcgttgcagtagcagcacaaaactcttaacctatcttttagctaggtgtgaaatcaacattctagtacaattaacttattatttttctataataataataatattattataattataattgttttaataaatatgtgtataataaaaaataaattttgtaattatataaatacaaaatttggaaattaacttagaagattatttttatttaaaattttatttttacatttacttaatctgtattttatttatgaataaatatctattaatattacattttttaattcaacattaaattaaataatcacctctttaaatatttatttatctaacaaattgaattatcacatttgtatttttcaatcaattacttcttagaataattacttctctaaataattattttttaaattataatatattttagataattattttttaaataattagtttcatcaattatttttttttaatatatttatttttctaaatattatttcctcagtacatttatatttctgaaattttatttcttgaaatatattttttttttacaattttatttttcaaataacttttttttttaattttacaattctttacctttcatttttaaataagcaaaattaaaatctattttgataattagattttcctattttttttttttgaaaaattacattaatcatttagaacttaattattgagaaactaaatcctgaatttctataataaactgtattatttgaataattattttgcaaagtatgatgttctcgtaggaaagaaaagtgtaagggtatatgtattgttttagaaacaaatttttgtatgaaagtttttctagaatggatattgtttactttattttattcttgatgtcaaagtcctctacatccagttataataataatatttacgagcctttttctttaaacagaggaaaaaaaagtgaaaagaggggtgaaacaaaaaatgttcataccaataaaaaataatagatggcctttgaaaattatctaaaatgtcaaattgataattatgtctattatgaattaaaaaaaaaaggagtaaaaaaaatctctcctttctaattcaattcagcatttaccttaaaaagtatagcacggaatacaagtaatttaatttcttttcttgcaagataacttttccaaagccgagctcaggcaagggactcatctctaaacttgcaaaatggtgaacccagagagaaaataaatgaattatttagtcaatatttatgatgtaatatataaaatatcaatttagataactgtaattgagagaaaatgagatatataaaaacaaatcgtgatatgtttgacaaaaaatattaactgatttttaatttatttttagattaaattcaatataccttttctaaaatcggaggcggtgatagccaacagattttggaagttgttggaaatctcattagttgccgaaatagattcatgccaaacgttgccgtgtcaacacgggttgcttcaattcgcggtctcaattcttccacgtgtcacaatgtgattgaacaagataccgttgcgtgacgaaacgaatgcttcattttgttataaaaagccaagcccacaccacgcttgaattatcctattcccatcccaacacaactctcgttcgaattgtagcagaagaagaaaaccctagctcaccgtttcaag

SEQ ID NO: 108 - DNA, 1365 bp, Vigna unguiculata
aaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaag

SEQ ID NO: 109 - DNA, 1584 bp, Glycine max
gagtgaaaaagatgacaaagaaagaacaaattcaaagtagaataaagtactatacgagttttaataataatcaagcaaaaataatggtaacgtaggtattatcagggttatcaaactcgagattttacgcagattcatgagagttctataaattcgacttgtagactcaactcgtagactcattagagtccacttcatataaaaataataataaaatatatataaataatatattaattaaacatttcaacgatacaataaagtaaaataataaatcataaatttcacaatacttagggggtatttgatttggttgttttctattttcatttttactggaaatagaaaatggtgatagaaatgtgtttggttggatttctgttttcattttcaataaaaatatttttttcaaacgaacaaaaaattgaaaataataaaatctcgttttcagtgttttcaattgaaaccaggaacctcattttgggtaaaataaaaacacggtggcaaggaatgtaattttaagcaaatctaaaaatacattttcttttgaaaacacattttcagtgtttttattttttgaaagcagaaaataagaagtcaaaccaaacatgttttcagaattctaatcttttgaaaatgaacagttttcagaaaataaaaacaggaaacaaaaacagaaaatgaaaatgcaaactaaacacacctttaaataaccaaatctagtaacacttcactactagataataatctgcagatgttatagtagtagtagatcatttctatcaagagtttgatgttattagagaacaagagtttgatgttattaaaggtgaaattttttttattcgagaacaacactaaatgaaagtatgttgaacactaaacaaacagaaaacaaccccaaatgacttacattttggtctaatttttttaacttgctgactcgttgactcagtggtaaactcgagagtctactgagtttacttagagtttattgagtctacccaaagtatactaaaaagagagtttacacacgaatcaactcacagaaaataagtgaactcataaaatcgtaagagttagcgagtaaaccaaaagtttgataacactgggtagtatcaattgtcattatatttgaaaagagaaacaaataaattgagtcaaccatcaccaacatcaataaaatcttatcctatcatgtgagatgggctacatgatacactggttaaaatcaaagttttaaaattgagccaaccgaaaggacattatttaatgtgattgtctcccccaagaatctaattaagttgacatgaggggagaagaggaggccaagcaagccaccatgatttagattccttcagataaaaattctccaaagttcactagccccacacaagctttgtgaggtccacaataaaagtggagctccaatcaatgctacctctcaagtaagatttgctctttcttcctctataaatactggatcatggttcagtgcttggaagcatagcagcatcatacaaagcattgaattcaaaaactaagttcatttcttgtgctttgctttattcctcaaaaccaaac

SEQ ID NO: 110 - DNA, 1616 bp, Glycine max
tttagttttattcattttttataatttcttttgtataaaaagggttttatttttgtaactattcaaaattccaaatattgattaatcgtaaagaaataaaacccattttatatgaaagaaaattaccaaaaaaaaaaaccaaactcttcattgaaatgacgatttctttaaaatattataaattaactttaaaatcgtgattgtacaaacgataagagtcttctaaaaaaatatataaaaaatcaaagtcatatttacaaacacaacttctttacaagtaaaaatgtgtttcaaaacacaagttattcatttccgtagtttattcaatatctacccatataggtaattacttgaaaaattacactcatttgataaataagctgatattcaatactatattctaccataaattattaaaattaaaatatgaaagatgtgattttgttaaaagagtcattttacttcttaagtagttaagactttttaatcttgaccatcgattaacaaatttacttctttttttactttcatataaagaaaaaatcctttcacctaatacatccttacgtgtgactggtgtgagagtgcgtctctccctcacaaagacatcgcatttcacatattatctatgagggcacataattctataaaaactacaaagagctattcactctattcatgttgaaattgtacccaaaattactatatatagttctattacatcaagaaatagatatgaaattacaatcatcttaattccttccttagcaggcacatctatacacagcaaaatttaccctttcactcctagaaactttcagagacaaatctactttattttgaaatgacatccacttgacccgaatatattgatagtgacacaaaattcttccatatcttgatctttaaatccaaagagaagagcccatattttccaaacgaactgagctcatgataggaaatcaaaaggaccatttcacaaaaatatcctacttattgacacatacctccagcagaataagtatcattgaaaaaggagattgaaggattgaaaaagatgataaagaaagaacaaaaacttcaaagtagaataaagtagtttatgataataatcaagcaaaaataatggtaacgtagtatcaattgccattatatttttgaaaatataaaccaagaaattcagtcaaataacaacaaagtcttatcctactagatggggtcaattacataaattatacacaccatttggtacggttaaaagcaaagttttagaaattgagctaaccaaaagggcattatttgatatgtgattgtcttccccaagaatctaattaagttgacatgagaggagaagaggaggccaagcaagcctccatgatttagattccttcagataacaattctccaaagttcactagccccacacatgccttgtgaggtccacaataaaagaggaactccaatcaatgctacctctcaagtaagatttgctcattcccccctataaatactggatcatggttcagggcttggaagcatagcagcatcatacaaagcattgaattcaaaaacttaagttcattccttgtgttttgctttcttcctcaaaaccaaac

SEQ ID NO: 111 - DNA, 1500 bp, Cicer arietinum
tttggtggtaattccattaaattcgagaaatacttgaaatattagactatgacaatcgtaacatgaccaaaagtgattgtagtagtgtcggtggcgggagatggtggtcggtgtttgttgtgacaggtagtagagatggttttcgacgattgtcaaaggtgaggtcggtttgggtggcaggtgggtggatgatgggagatggtggatggaggtcagtggttaatggtggtggaaggtggatggttggtggtggttgtagcgttggtcattgatggtgattgtaagtgatatgtgtttggagatggtgatggcaatggtcatagatggtggttgtggcgatggttggaggtgagtagtcggtgatggcgatcggtaatcaatgattgttggagtggtgcttacgtctactaatggtgacacaaccatcttaccgaaaaaaattataaaacctttggtagtcaaaaattataaaaccaacacaacaccattttatcgaaaaaaattttaattttgaaattgacttttaaaaataaaaaccataaactcacaaccatctagaacgattttgaatctcccaaccaacatttacgattgccaaaataaacctaacaagaaatagaaataaactgaaagtctcaaagctctcagcttcacacgctctttcacaaaaattaacacttttacttttttttaacaaaaaattagagtgagtcaatgatgcccttatttcagatagaaaatactttatgactcatttatcacttacaaataaataaaacacgcaaaataccgaatccaatacatgtgcatttactactagaccaaaacattggaattaaattaatctttttattactattaattaaatttatgaaaacaaatccaactttcattttgaaaaatcatcaactttgtccgaatatattgatagtgacaaaattctaaggcatctaaacttttgaacagaatacaagaacccatatcaccacgacataaaaacttgtgaagaataattagtattgaaaggaaggaaagtggaggagcaaaaatgattagatagaaaaaatactaatttcaaagtagaataaagtagtttactgaatttcagtctagcattcataagggaaacgagttttcagtagtctttatatttgaaaagggaatgaaaaaaaggagctaaagagtaatggaatctaagaatatgaatacgtgactccctcaaccaagaaccaaattaaaattgacatgagaggagcagaggacgctaagcctcaatgattttgattccttcagataaataaatatattctcacatcaaatgctctgtgaggtcttcaatagaaggattaactcaaatctattgacacctccctagtcatatttgcttttcgtcaactataaatacaggacaacggttcacagcttgaaggcacagtatcaacattcaattctacagttaatttcatttcaagagcttttctttcttcaaccgaaccaaac

SEQ ID NO: 112 - DNA, 1564 bp, Medicago truncatula
tagagacttctatattattctttagtgttctagagacttccacattattctctagtattctagagtcttcctaattattcttcaccattctagagacttccatattattcttcatcagttgatatgagctagatcgtgctgccttgacaatagaaatatctcagttggtaagacagcaggatctaactcaaaacatcctcttgaaacactttgtctatcatcgttatgtgtagatttgattccaatttttttttttatgaactcgattcagttggcctcccgatagaaatatgttcgttggcaagccagctggatcgagttcaagatcacctcttcgaataccattctctcatattatgttgttttaagctgcttgcttgtttttttctttatgcattttttaattattttctatgtcatattctatgaaaatcctccaaaaaatacattctcgtgttttcggattttataatccgaattgtcttagtgatttttggattatataatccgaaaatccttagtttttttttttacggtgacgtttcactaaaggttttgcagattagtggagtgtttaatgcatttttagacggcggaacacgttttatttgactgtaattatgatctaggacaggtttaagattatatgtttggatcttcaacaatttccttcactcttcacttcacctctttccttacatattcttaaaaacaaattaattttttaggagtgtttgtggtgtcatggtcattgttgaccgttcatataacccctgcaattgatttgcagcttatgcgggggttatataacggtcaacagaaatatttgacatcaaagaaaacacataaaatcacagatcgtaatatatcggaattcgtagtcctaaaactgctaacaatctggttcggattacataatccgaactagagatatttttgtattttcttctgggcacgcgagaacaatggagggtggaaaaagtgatagtcttttaaaaattcattaactttgtccagacagtgacaaaaaatacttgaacatctaaacttttgaaccgaatacaagaacccatattttctagacactttattccaaggaccatttccgcagtattgacacactaacttatgtaaaatattataactgaaaataatgaaattggaggagtaaaaaagaacatgaagaaagaaaactaactttaaagtggaataaactagtaatgtgttttcaattgtcaataaatttgaaaggagaatgaatagaagaaaagctaaaaagtaatggaatctcaaaatatgaatacgtgactccttctctttaatcaagaatcaaattaaaattgacatgagtggagcataagaaattccttcagataaatattctcacatcacacactctgtgaggtctgaaataaaagatgaagcactaatatatgatacctccctactaagattttccctttcttccactataaatactggacaatggttaacagcttggaagcacagcagcaacattcaattctacagctaatttcattttcatcaacaaaaccaaacaaac

SEQ ID NO: 113 - DNA, 1500 bp, Glycine max
tgagctatagaacggcatggtgcaatctttatataaaagcgccaccaattagatgttgtctcacttgtgtggacttcgattatctgtggtcataatccaaggactcaatttaattctttttttatatttcattaatgttgtaaggttaatttggaaaataaaacaaagggaagacattcaacttcccctctctctagctgcagttaaaccacatgtgagccttaagatgtacacgtgtcaagctataagtaagacatttctttataatgaaaaatacaaaaataaaaaaaaaataaaaattctaagcaatattgagtttctttgattagaatctctggatggatcagatagcaaacttggttttgactggtttggcaaaatgaagaaaaacaacgaaagaaagatgaatttatgtttattttaaatataaagtttttggacgcaaaagtgaaaattttaaacattcttatatacagatatataaataccaaactgaattgatgaagtaatctctttttttttttgggttattctgaagcttaacactgaagaaaactggtgaagttgagaatttcctttaattgcagtatggagcatgtttgtgttcctagagccacaagcaacacgtagaagagcaaaggagagagccagaggaagaggcagagagagtaaaagaacaaggagcttgttatcatcgtagggtccatgaatgtaattaataggactcaatcaccttcatcaaattataatccgactcaccaaaattaatttcttgtatctacccaataataattgagaaacaaatcttatattaataatataaaataattttacatattattgaatcataaattatcttaaaattaaataatgttataaaattattttacgttctaagtggcattaaactcataataattcatgttaaatttatcttcatctgaaaatactctaagattctaagttaaactaatttcatgaaaattgaaaattggatatgttggctactatgtggggtgaacagtaccatctttacgtaaataaaacaccacctcaaatattatggtagttgttctttaaaaaatactgataatattatcattggttatcaagattgaagagcactgaaatgacaataaccaatgctttgaatttgaataaacgacaaaagttctcctcacaggcttgtgtgtgtggcaataacatctccttaatactggtggagatagaaaaagcttgaataccaaccgcaaagagtaggactaagagtgacaaaaaaccttgcaggcacattatgttgccacgtgtcggctgatcaatcttctatgtgtggatgtaaggcttctatgaccaatccttttgtgtacactcactccaccaagcaactactataagtcacagcctcattcatggtcacaagccacttagcactgagtcttgcaattgaagttgaagtcttgcccttctctcttcaactccaaacacaagacatcatcatcc

SEQ ID NO: 114 - DNA, 1834 bp, Phaseolus vulgaris
tgaatgatgtaattaggtgataaaagcacactatattcttatgatataaaaagataaagtagatttgtagaagtctaatgtcttgttatttttcctcttctttcccaaagataaggcatgcaaatactctcatgcaaacaacacttttctccatgcacaatcaaactaattttccagacattttactcttatattgcaatgtcatatctcaggttgtcaacttttacaaacaattcgagttactgtaaatttgtatcactattggagatttttctaccaactttttacaaaacaactatagtgaataaatgtgttattttttatataatgtattataaaaaaactgagataacaaaagaaaaaaaataaagaatatgaaaaaaaaaatatttcttctattttcttttctttacatcaatcaaaatagagagccttaatagattcaagctgtacattcattttagtctagataaaagataaaatataaaacaaagagacaataaaaaataaaattaaaaaagtaaaataaaataaagactagacatatatacaaagagaaacaactaagaattataaaagataaaattaatattaattttcttataacaaccttacatccatattctttgttacaatgttatcaataaaaaaaataaaaaaaataattaaattaaatcaaaataataagtaaaattgaatatgttttagacacattcatttatataatataatatgaaatttattttaactttaaaccatctaaatacttagtacgaaaagaattggaataggttattatgtaggtttaggttttaaaggttaggatttagtataaaaataattgaaataggttattctatgtgtttaggtttttttttatcatcaaagaatgaaattaaattaagagggatacaaaaggggtatccccacccttttacataaaccagaccacattaagaatctccaagaataaaaagatgaacaaacatcccagaaacaatagaccgataatgttttctgcataagctagacattctatatgtttaggttttcaaagttaggatatatgattgaaatgagaaaatgaaaatattaaacaagcttatattttagataattaaacatcaaataatcaattttttgtagttttaaatttgtttcttgttatttggaagcttaacagtgaagaaaaagcagtgaagttgtgaatttcctttagttgcaagtgaagaatgttggccacacagaacagatagaagaaagaaggagagagacatggtagggtcaagggatgtaatagggctcaatccccttcattcatgaaattatctgagactcaccaattaaatttcttgtttctaccaacaataattcatcttcaaatgaacccattggctccaatgtggggtcattagtaccatctctacctctgtaaaaagacaacctcaaaaattatgccacttaccaattctaatattattactcttatgattggttatcaagattgaagatgcttgaatttgaataaacgacacaagttcttcaaggcttgtgtggcaaacaaatctccttaattctggtggagatagaaaaagctgcaatgacaacctcaaaatcctcgactaagagggacaaaaaaccttggaggcacagtatcttgccacgtggctgctgttcaatcttctatgtgtggatcttaggcctctgtgaccaatccttttgagcacaggcaactactataagtcagacgtacccttcgcttatactaagccatccaccactgaatttgcaactcaacaacaaccttaccctttctctccacaacagcacttcc

SEQ ID NO: 115 - DNA, 1785 bp, Medicago truncatula
gttctcccaagaagattagtcgttgttaaacggtgatggaaatttcgtatcaatatctagataacaaaaaaaaaaaatccaactcgaagaaaaagtcaatgacatgttaagtttttgtgctatgatgatttcaaaatttccgttaaatgaaagaattttaaatcgtacattgaaatttgggttaatagttattttgatctttgaatgtgcaacgagtagtcataatagtttctgaatgtatcaaaattccaaagtcgtccctgattatgcacttatataatcaaaatagtctttcgtgttaaagtcttatattaatattgtcatattagtatacttacttattgtcataccaacttacttaatgtcatacaagtccctatagaaagagttatagtcaacattgagggacaattttaacatcatggactaatatattgcacaatcagggataattttggaattttaatacattgagatactattgtgactactccttacacatttagggatcaaaatgactattacctcttgaaattttgataaggtatgtgatgctcttagttcttactgcccctctgttttaaaattaatgacgtttaagaaatattataaaatgtcaaaagatatgacagtttacaaactaaccttattaacaatagagtgtttccatcgttataaattaaagtggaatacattgttttaagagtaatatacataggtttatttggataaacaacttatttgtagttgataacacaattgtttatcataattaatgtttatgtataagctatttttataacaaaagataaaataatgttgaatttttttcttataaactataatctattttcaaaaaaattcttgaagaatttacaaaaataaactgataatagtttaagcaccaatgtcataagttgttttcataaatttttccaaacaatttcacaaaatttatgtcaatagataattcaaataagttaatccaatccgttaattagaaggcttagttggggggaaaataattctaatgaaaatgacatttactttaaaacaattttttttttcctaataaagtgacattattgtgaagcatatcagtgaagaaagtaggaaagttctgaatttcctttaattgcagtatgatgaaagttatgagttcctaggccaacatgcaaaatgtaacagtgacattaactgatgggtccaatccctttaaattatgaaattatcttgaattttccaaattaatttacagaaataaaataattgggtgactaatgcgaaataaaatgataggtttgtatgtaataataatgtaatctaaattgaaagaccaatatgataatgatcagtttaaaatagcaatattatgattggtcactcaccaataaaagacattgatgttttaatttttagtgtaaaatgagagtaaccaatgcttgaatatttagaataaagatgagatgagatgagtcctatccaagtcttgtgtggtaagaaactctccttaatacttgtggagatggaaataccttcaataccaacctcaaggtcattgcttaagagtgaatgagaaatgtagcaactctatgattgccacgtggcagcttggtaatcttcattgtgtggacgttatggtttcaatgaccaatcctattgttacaccctatcaattaatcagctaatataaatccctcaatttgatcacaaatttgcatagaataatttgcttcaatttcaactatctttctcttttgtgcaataccaatcagtgcagtatcc

SEQ ID NO: 116 - DNA, 1787 bp, Vigna unguiculata
taattaaacctataaaatataactacttttcaaatacataacaactacatataaattatttttaataggttaaaagaaaattatattttgagtgcagatagaataaacagccactttacaattgtattttaatgatataataatatatatgtatattttaatttaagaaataaaaggtatgtattaaagtattagcatgataagttattgtcgtttatttattttgagaagtcaattatcaatactcttatttaagaatgcaatttgtttgacgaaaaagtagaaatactaaacaagcttaaattttagataattaaacatcaattatttgaagtgatgtgataatctatttttttttgtgtttttttaatttcttttctgttattccgaagcttatcagtggagaaaaactagtgaagttgcgaatttcctttaattgcaaaaatgaagaatgttagccacacaccacacttagaagagacaaggagagagacagaaagatgttatcatcacagagtccatagatgtaatagggctcaatatccccttcatcaaattatcacagactcaccaatttcatttctgttccgaccaataaaattttatctttaatttttctccacctgaaaataccaacgtagataattccaccaaaactgaaaattgaactcattggccaccatgcggcatggcccccaatttctatttttaacttttagatgtcaaatattaaatttttttaaagtttgttaagatttttaaatcatatacatatacactacgataaaaatcaaattaagtataaatatctctctttttctcatttaatataaatattagttagcattttttttatttcaagaatctttgtatatttttttataaatataaaaggaaaaatattaaattttatatattttttttcataactaatttttaattatgatttggttattggagtttttttagtaattatttatctaagttttcaattagaatatgataaactatttttaaaatttaaattaaaaaatgtatattagttcagaataaatgaataaatttatcttttaaaaaaaaaagttacactcaaaataaagacaagagactcaacttccttatctattccaaagcataataccgaagatttaattagaaaaaatgagacctgagaggttttatatattaataatttggaatgtgatacaaataacattctcaatatatgaaaatattcgatgaatgaaatagatcaaataaagtattcaaaaataatttaaaatagaaaccatatcaaatacaacttacaagttataaattttgaacatattatttttacacccatataattattggtcaagatcatgtgggatcatcagtaccatctttacgtaagtaaaagaccacctcaaatattctggcacttaaaaattctgatattattaccattatgattggttattaagattcaagagcactgaaatgacagtaaccaatgcttgaatttgaataaacgacatgagtctccttcaagacttgtgtggcaaacatatctccttaattctggtggagataggaaaagctgcaataacaacctcaaaatctaagactgagagtaacaacctcaatcttctatgtgtggatgtcaggcttctgtgaccaatccttttgcgcacaagcaactactataagtcagacgtagcctttgatcataagccagctaacactgaatctgcaatcgaagaacaaccttaccctttctctccacaactccaaacaacacttca

SEQ ID NO: 117 - DNA, 1623 bp, Glycine max
ttaccacaaatagtgtatcactctataaacctgagtaccatacctaatgattacaagaaataataacatttttacacaaaaatacttttgtgatcttgtttcacaatctctcttctcacattttctcttcatgtattgtttattcactaaatctcttctttatttagatgaaatattaccaccaattataataaataagttttctccgaatttgaaacaaaaataatttttaatatatctattcaactaagttttatctagtaaaatgtaatcttttactttatatttaaactacttaaatcttaatgataaaaattcaattctcaatatatttacactttatcttttgacttgaaattctacaattttaaacattttatcaatattttttgatttcttttttttatctcattatatcatatattatacttatattttttttctctttttctctaactcactaggtatataggatagcattcgggtgtctaaaaatatttatccttaataatttagcacccaacgctgacactgtcaggagataggagattcattggcaatcaaacacaatcggcagagcaaatgaattaaaataaactacattgataggaagcacgtgaaatgaaaatctctgccaaaatcccgttgtaaaattccaaaccacgtgctaatttactaatgtatcctaaaaatgtttagttatttatattttctttttatatttttaacctttcaagaaagaaaaaactgttctcagtcaccctacctgaggtaactaatcgtttgtattttatttatttatttttataggaagctagtcactctatttcttgtctgagtcagcatagtagttgattcatatatttaatgtgtttgaaaaaacattaataaattgattttgattaaaattgattttataaaattgattttaattaaaattaattttaaagtattataatttatgtttgaatgtttttattataaaattaaattattagaaataaaatttattataaattttatgatacaatataaaatttatttaaaattgttttaacttaaaattaattttaaatttttttgtcacataaaactaaaaatgtaaattttaatttaaaattaatttaaaattctgattcaattcttagatcgtgaaatcaaatatataattaatatagtcattctgaatttatttatgattgttatatttattttctctttcattttaaaatttttcaaatcatagtacatcaattattttatctatctatctctctcttatttcactatctatctctctcttatttcactttctttccacgtcttctatcctaaaaaacagtgtaggtttgtcatcttcctttcgcccgtttatgcatttttatgaaattctcaacaaattatgagcatatttgcaatttactcggagcagaaaattaaatgtctgtgaatgaggtgatccagcattggctcatttattcttggaacatacatgccacatttatctataaataccctcttctagacaaggcacaaggcacaaggcacaggcttccctccctcctttgaagaaacaaattagaagagtgtgttgtgttgtgttgaagtagcaagctagcaaag

SEQ ID NO: 118 - DNA, 1582 bp, Vigna unguiculata
atattatattttattgagaagtggaaaaagtaatcaaggtgtgatcttagggttaggttgttagggaaacaaagcgcacgttcaaacaaagcatgctgcacgcgggtgatgaaaataaattatattttattagagaaaaaaaagtcagttttactagtatcattttattttggaatcgtccaaaatcaattcttaaatttcatgttaataaattatttaaaaacattcaaaattaatttataaaattaaacatacataaaaaatttacatcaaagaaaaatattttcttttaactttttcatcaataaaattaattttttcatataaaataaaattaattttatcaaattgtttcaaacacattctaagtcaattaattcttttttttataggttataatttatcaattttttaaaagatcacgtatttaaaaattaaatacaaattactaattaaaaaaagtaaaagaaaaatatttctttgagataagtatcttaaatgaacacttgaaaaaatcgaaatctgtttattttattaaaatataagtgcaaataaagtcatggattcatccaataatttatcaattttcagttggctgagatttaaaaaaacaaaaacagaaaatttagttctttatagaaaaagtaagttttgaatgatgatcacataaaaaaatattaactatataatacaaaaaaatgtagtcaaatttatcgtaataagattttaagaaagataactaattatttttgtacaagtcagtcaccgacaacctttacaagaataaaatctaaaaatttcacattttttaaggttaatttaaaaatattttttttgattaaaacattaaaaattaatattataaacgaatttaaattctaattcataaattaattaattataatttttatttataatagagattattttaaatacttgataatttataaattaatttattttttgtttttaaaattagttattatttaataatattttatttaaatatttaatggttgaaattagatactaaaattgtacaaattctgattctttagtacatttgtaaggaagaaactcatatttaatttctataatattaaagtttctttgagaaatgaaaatgaaattatcaggtatcctactcaggagaaggcacatacacaaggcaaagaccaatagtttcagttcacgtacaataaatttatagggtttcttctttcttcatcttgtccatttctatataatataacattgaaatttacacttaaaattaaaaaaaatcttttctttttcaatgttcttttcttttcacctcatcatccatgaaaaaacactgtagaaagtttatcaccgtcgggccctacaagaaactacgaagcaatggcgagtgaccgcacaaggagcagaaaaatatcagtaaataaattaattcagcattggctcacatcacatcacttttgtttttggaacagctcatattgatctataaataggatcacgttagaaggcagaaacaaggaacacaaacaagggacgaggattttaagaaagaagatagtgttgtgtttttgtggcaaag

SEQ ID NO: 119 - DNA, 1500 bp, Cajanus cajan
cttttttactctatgttatgataaaattgacaaacgtatgttttacatttataaatttctcatttaacgcatctcttattaaataaattattaaaattatttcagcccacacacacatatatatatatatatatatatatatatatatatatatatatatatataatttttttctttccttcctatttcatccgtgtttctatcataacaaaggtgtttacgaagttttaacaattttcaaaaccttctaagcgtaccgcaatttattcatgagaatgtcgtcaacacattttttaatttgaattcgtatcttacgtaacattattttttatgacagaaattttttaataaatttgaattgaggaagattcttaacaatataatgatattttttaatacacactcttttgattttatgtcaagaaaagtactacaattgaaatgtcatatattaaagtaggaatattttggagaatatatcattaaatgatgagttaaatttgcttatatttaagaacaaaaaatatatgttttttcttcttcttataatagagaacaaaaggagtatgtatgtaaccaattaactatagagtaaaacttaagataatgaactttgatatgtgatatagttttagtaataactaacttagaatatcatctcttatcaaagaaaaaaaaaacctaagatatcttcaaccttaatgaacttgtaaataaaaatcttaaagtataagtagtagtacaatttatattaattttagttatcattttataaagtttataaaaaatttaacattattaaaatgttgcagaatatcactttaatttagtaaaatctttaaaaatgttgtcattaatatataaaatgttacttaaagtcttttaacatatgctttaatatcttatatttgataaaatatggcaaaaatatattagtaacatttacttataatttagtagcatttttaaatattactgaaattacattttgttgtagtgatttcacattttaaaaaatatgagaattaaatatctatattttaatataaaattaattacatattcacaaaagtaaatggactaaaagttataattaattttttcttcttcttatttttacctcataagaatgttttcgaagaaaagtttattctttgatacccgtataaaaatgttacacaaatgaatatgtttggatgttaatctaaaaagacttttaaaacttgtctattaacaaatctttgggaccatatccaaaaatacttgattaatgtatttgtaatgtttgctgtgctagcctgttaaaagaagcacctatggtgagcactccttttggaaaatatatatgagtgatatgagtttgaggtaatggaacattgggtcaatttattcttggaacgtttaggtgacgacattcatctataaatacccacttcctagaggacacaaggcacaaacgaaagaagacttaagagtagtgtgttgttagcaatagcaaag

SEQ ID NO: 120 - DNA, 1670 bp, Phaseolus vulgaris
ttacctcgatgcaacgtacctaataatttaagctttttccttttaatacttaaaaaaaaaatcctaaagaacttttagatgggttttccaaagattaaatcctacaaaaataacatccccttatcaccaattactcaagatgaatagaaaataataatatattattgatagaaattgagtcacttgtttaattagtctatattattccctatttttccgctttctcaagtctgaaaaatgataccacaccataatagtcataattttttctcatcttagagatagtttagaaaaaaagttttttattaaaataaatatttaacttttatttatacactacctatttcttgttaatttaaaattgcatcttaaaataaaattaaaggcttatacaacacactataactaatatattgaacgagttttgtacactccaaagttcaatgtttttagtgtttcaaaatattgcacgtgttataataatacggtagcagaccatgtttatcatggcattcatccacacactttgtagtcactcgaaatttatttctaattattatatttattttctctaggatatcattatttgagtgattagatattttatgtttacatagaattatataataactcatggttgactccagtcattagtaaaaattaattgaattaaagaaaagtatattattttgaaagaatatttaaataaaaagttttaaaaacattgaatttagtaaaagaattacgaaattctctttttataaattttggtgtttgatttgttaatttttcctcactgctagcaaggttgagcttttgtgataaagaataactctaaattttttttttttggagtttgtaataaattgggttgtaaattttttttaaaagataaaaataattaataaaatagagatatttcatgtataccacaacttttatagaaaacaactttattgcagagtgaattttaaacctaactcaactccataaaatcagtttaatgaaatgaggtttgcatccacttatatactatgaaagactctaatctctagtcgatgtgggatctccaacaaacttacatcttagattcttgcactatgagaaacatgcaacttagacaaacaaaaaagtcatttttacaaatctctataaaattgggatgtgagatattatcttatctctcttcttaattctaatataagggttgagtcatctttaaaatatctatatttatttattatttttaataaaaaaaatctgattttatactgtagttgtacgtactatagctgattgatatatatgtgttttttttatatactatctatttgatgattcataactcaaactctattatttttccatattctttccacctcatcatcatccacgagggaaaacagtgcaggtaaggtaatcatgtatcactatcacccttacaaatgatcaaggaatggtgagaggccatattgcaatttagcagaaaaatatcagaaaattacttcagtagagagaggtaattcagcattggttgaaatgactttgttcttggaacacgtgatattgatctataaatagggtgttgtggagagggtagaaggcacaaacaaggcacaaggctttgaagaaagaagaagagtgtgtggtgttgtgttttattggcaaag

SEQ ID NO: 121 - DNA, 1581 bp, Medicago truncatula
gtttgtgtggtttttagcaacatgccgtgtttgttgtatgcttttttacagtaggttattttgtttaataaaatctaatttcgtttatagtgtaaatggtgaacaacaactcattggtggtaaacggtgagttgttagtggtagacgataaaatctaaacagtgaagcaaagaggaagatgctgactgttgcagaacgataatgacagtttttttttcaattgtaaacaatggacacgatttgttggtggtaaatagtgagttgttggtggtagacggcaatatggtaagaggaagaagacaaagagacaaagggtttttttgtgaaaaatgaggtgagattaggtgtgaatttggggaaaaggaaaaaattataataatcacaaacatgctgaaagactaaaatcattctcatatcaacaataagagaaattctagcgcccatattttaacacacacatttcaagtgagttcaaaccaatcaaagatagtgttgaaaagtatgtgttaaattgtgtgttgctagcatttttctcacaataattgtcacaaaaatttaatctttcacttgtaaatccattcaaacttaacatattaaacccaatatttagattctttagatataatggttgtttttagcactaaaaccaagtgttataaatacagagaaagtgagaattgcaccttttataatggatgattacgatgaggcaaggaggaagatgatgacggttgcagaaccatgaagatagtttcatttcaattgcaaacggtgaacaacaacttattggtggtaaacggtgagttgttgttggtagacgacaaaatataaacattgaaacaaggaggaagatgatgacgtttgcagaacgatgatgacagttttgtttcaattataaacgacggacaacaatttgttggtggtagacgtcgaaagggtaacgggaagaagacaataagacaaaggtttttattgtgaaaaatgagatgagactaggagtgaatttgggaagaaaaaaagaattataacaaagattcactcttttaattccatccttttatcccctcaatgatgtggcatcattgatatgcgaacaagcctggacacatcaatgccaaacataaatgtcattagttttaacggagtcacttgatataaagaccgatatgagagacatctcacatgattttttagtccaatgacacgcaaatttttgtaagaaagacaatataataggtaacttctatcattatctaatctaattaatataacttctaatccaacggtagataagatgaaaacgtagcacaccctataaaacacaacacaaaaacccattcacaaatgtgtgagagacgtgtttacgaaaacgacgtggacgtggtgttagatccgcaaatagattaaaatcaccgtaagatcatgacacgtgtccttagatatttcacactctctctcctcatttttcctataaatgcgggttcaacccatgcttctatcattcagaagtaaattgttttgttgtcttaaatcattcatcttttgttgtttgtttgtgtttgatatcatcatcatctgaaa

SEQ ID NO: 122 - DNA, 1540 bp, Vigna unguiculata
tcaaacctaataaaattgtgatggatttttccaccttatttaatgtatttcaacaaaaataaacaaaaagtaaaattgataaacgagattttaattaaaaaaattcaattaaaaataaatgatgtttttctcacctttatcttcttaattgaattttcatttaaataaaatttaattaaaataaaacattatttctactacactttcaatcaaaataaaatctagtcgatacgattatagatagggatggcaaaacgggtcagtccggcccgttttggcccgacccgtttgtggtccggcgaaaacgggtcgggttggcccgccacccataaatgggctgaatattgcgacccgattcattttaggacgggtcagcgagcgaacctgttttctttttaatttttttagttttttataaatttttgtttataaatctattatatatatatataaatttatttatataatatataaaaaatattattttttaattatttttaattttaaatataaaaaagaaacggattggcgggctgaaccgtttttgccagctaaattgacggactaggcggaccgaattacaacgaattaacggactaaaaatttcaatccgacttaccattttcttgacggattggtgggccgacccaatggacctgacccattttgacatccgtgattatgtatagaaatttcttcttcatttcttctcttcacctattttcttcaagaaatccaaaacaacacaatacatacttgagtttgataaactattgataattaaggattttttatcctcaaatatcatgagtataaaatcataatgactattattaattagttaataccaaataatgtctatttgaaatatgttataatttttttttctcacgttaccattactaaaggaacaaaataagaatattacacaatcataattaacagtcacattgtctaataaaaaatataaaaaatttaaattatgaatataaaaagcaattttgatagaaactgatattcgagaaacttcattaaaaatacaatttttaaaataactcaaaatttaatgaagttacaaattgaaaacttaaacttaattatgaaaaggataagaatgattgaaccagtgccacgtcgtcacaattcccatagatattttcacgtttttcatcactaaactctcacccttgattctcccttgttgaccgctatctaacgtcccatccaatggttctcagtttgtcaagcctacattttagaacacacgagaaaaccccacaaaactccactgcaaataatagcgatgatgatgtgtcgaaaacgacgtcgacgtgtgtggagatccgaagaggattaaaagcgccgtcagatatagacacgtggacggtgggaattccattctctctccactcaattgtgttataaattgggatcatggctcctccttgaaactcgaacctgtgtgtgataattgttcttgtgacttgttcttcgtcgttatcttcttttcttctgtgtttgttttttgttacccgagaaa

SEQ ID NO: 123 - DNA, 1582 bp, Vigna unguiculata
tttctacattctcgggtttgtcacaaaactaatttcatcacgtccttataaaaataatatagtttttgtaacttttatttaaattgactcaacatgattgatctctattaattaacaacagtttctaatgagaatttacaattgtcatcactaattaacgtcacattttgtcactttagattcaaggaaataaatcgaatcaattttaaagataaggcatcaaattgaatcttagattaatattacgaataaaataaaaatacattaaaaattaaagtaattaagccaaatatgtattaccttataaaaaatgtggaagacgagacgaatatgtatcggttcaattcgcttttttgtattttgctgaagatacgaatgggaggtttagagaaggaagaaaaataacaattgaaattttcgatattattaatatctcagaagaatctagaaatcctctcagtgtcatattaattacgtactttgtctcgtagccatttattctgctctttctttcacactttgatgtccacagtattattcgtaaaagaaatttaattatcaaaatattattattattattattattattattattattattattattattattgaagagtaaacgaggcagttacgagttccggtattattatataaagttacgatttacttataattctgatcctatactccacccaattattttattactttttaattttagattcttaatttttagtttacctgtaatttttatcttaacatttttttttaaatttgatctttgaattttaattatatgtaactttattcaagtttcaatttaatttattccaattgtttagagtcaagatggaatctcctttcaaatatataaaattataataggaatttcagatttcagataatatttctaatacactgattatttctaatgatattatttaaagaaattaaagttaaaatattggcaccattcattgaaataaaaacttctttcattcttgactggagaatatattctgatacacccgataggttaaactattgtgatagttgaagttaaaatataattatttatttttataaacttaaatatgatacaaatcatcgaaagagaggggtttcttttcataataactcagaatatttttaaaattaaaagttagataaagtctctgtatacataatcaaaaattagaaaactgaaattgtaaaaaaaaaattcgggacaaaagtgcttgaaattaaaataacaaactttttataaaaccaacctcataaattatcgttatcgtatactcccaagagcacgaaatgtaattatcgttatcgtacaccacagaaattgaaaaagaatagtgagaaaacgacgtcgacatgggaaaagtcgtagcatagttgtgcccccaatggaacagattgagacacgtggacgttcaagatttcacatactctcagcgactgcttataaattgggatcacgcttcgtttaaacccattcgagtgagtttagcaaacaaccaataatcttttctttgttcttcatcagtttgtgcctttcaattcctccattttgaga

SEQ ID NO: 124 - DNA, 1500 bp, Cajanus cajan
gccttcattagcttcttttttaaaaaagaaaatattgttaaaatatttgttaaaataatttacatttataaatatcgttaaaaatatataactaaaataatatatatatatatattctctatatctaaattcttattctttaaataataataattattattataattattattatactaatattatatatattgcatatgtaattaggtaaatattttttggtggattgtggatttcctaatttacatcaatttttagcttcatttccaggtgtaagatatcatctacaagattttgcaggtcagggtaatgatcctgaaaatgagaaagaattgttcaattataatgtgatcgagagaatatttggtatttttaaatcaaggttcacaatttttaagtcaacgcctccatttttatttaagacacaaatagaacttgtgttagtatgcgcaacactacataactttctacgtaaagaatttctttcccatgaattttcaattgaagcgttgtgtgaaccttcatcttcaatgttaccagttaatgaagatcatacttttgagcctattattcaaacccaggagcaagagtgaaaagatactaatgcttggaggactagtatagttatgaatatgtgggtgaatgctatttaacatgatagaatattatggagaattattattattattattattattatttatctagacattattatttttttattatttttgtctttgactctttttaaatttacatgtatgaaattttttttgaaatcttatcaaatgtaaaaaatccatgagaattctataaaatccaaaaaaatccattaaaatcctgatttaaaagttttgcagaatctattaaaatcctgattttaagtcttgtaaaatcctgcaaaatcataaaagtcgtaaaaatcctgcaaaatcttttaaaatctgcaagattttttttaactaaaaaagtcatttaaaatcctaatccaatacactccttagataaatttttagggaagtcgtataaaaaaaaaaggagtaaaatgaaagtgagaggagatagaacgattgaatgagtgtcacaatcacaatgcgacaatttccacagatattattttcacagcctaccatcacatcaaatctcacccgttgattttccctcgaccgtcatcaaatatctcatccaacggtacccaactccccaaccatacaatttcgaacataccagaaaacccctcatcactgcggcgagagacgagtgatgaaaacgacgtcaacgtgggagaagatccgcaaaaggatcagaaccgccctgagatcgcgacacgtgtacggcggatacatcacactctctcccctcaaccgtgttataaattcgggtcgtggcatagccttgaaactcgttccagtgtgtgtaataatagtttcggtgttgtaacttgttcctcctcgtcgttatcttcttttcttctgtgttcggtttttctctcacctcaaa

SEQ ID NO: 125 - DNA, 1580 bp, Glycine max
tatttaagtaataacgagtttaatttgttttaaaccaaaatttgaattgtgaatgggaaaagcgatgaaagagcattgctctcttaacttaagttcactgaactttatttttcaaaataattgaggctcaaaaataatttatgatatcaaagattttaaagataagtaagtatttgtaaaagtatcaatgatgttttatacgattcgattttcttgaaaactaggttttttaaggccttaaaatttagttgtgacaaagttaatttcatataattttacactaattaaaattgatttaaaaataatatactttgtttgaatgatataaaaaaattatataacaagtcatcagaacaataacttgaaattataacttttagaagaatagaaaaaaccaattttagtaaacaaaaccaatattttttattttttaatataaaaatatcactactagattattttttttcttagccctttaatttatcagggaaagaaacctagttaatccaaaattcagtcaaaggataaataaaatttaatcaagctaatctagaatttagtcaaaagataaataaaatttaattaaaaataatttctatcaaagattaaattcaaataatatccaaataattcaattttaatttcaacatatgtctcccatcatttaatttattactacatgatgtaaaggaagtttttaatttcctaaggataaaaaataaatttttttaatccactagatttaatcttagtttacacgagatctcaaacttaattatctatacattaaaaaaaagagagtaaaggaaacttttatatattttttatataaaaaaaaaatcttctcattgggcaatgtggcgtaatagttgaatacacgtgtccttcactcggtgacagaaagtcatgttacagctccacgtatgatcacagcatatatttccacaacaaacaaatattaacaatattttttattaatcacaagttaataaaaattgtaaaaacttgattatataaaatataagatacacaaaaatgtatactaatttctaataaattttaactgttactacaataaacatgttcatatgattgtgctgcgcatttctcaaatgaatgaagatgtataaacattatttatatgatatttgatatttgagtgagaagaataaaaagtataaatataataagtagtatgatttaatagaaaagaaatgataaagtataaataagagtgtgaattaggggtataaaaatcagaagtgtggttaaatttacaccgactggcagccattaggggccacagaagcgcgcgtagatgtaaaacccctgcacccacaaattcatggccatagaaataaaaaaaaagtaggaattttcttttaattaataagtcattaatagcaagaatgaagaaaatgtgaagagaaaagggagctactcagatctggggctgaaaacggctataaataacccgtaaaccactcctaatacctcgcactctcttctctgcttcaacttgctccctcagaagcgaagaagaagccacagagaactagtctcctactctcacccgcaag

SEQ ID NO: 126 - DNA, 1526 bp, Medicago truncatula
ttggagttcctgaattcaaattcgaacaaagattaaactataatataacaattaactactaatatctgataggtagtgtaggttttagacattgaagtgtgttcctctccgtgttgggttgccctatgtgccaacaatttatatgtttgatcggtccataattttgtttggtctaacttcaactggaccatgactagtagaatatgtatttgatccccctgattaacatgtctttgagttgaataccaagttttgtaaaaaaaaaaagaagcaattacttgttctttttattctcgacaacaaatctataatctacaactctgattaaagcagtacagatctcgatagtttcacacaatttgagatgcttgaacaatccatatacaatgacatcagagtttctaatcatttgttaagagtgcttgaattttttactaattaaaataatgattataaaaaatctattttttaagcaaataggatgcagatggatctgccacacctcaagtagattaaaaaaggaagaaaaaatatagaaaattaaagcgacataaaacctgacccaatgcaaacagaaactaacaatgagccactaatatcaaacggaacagtcaaccacacaccaattgcacaatctagcgaacccacccaaattgcaaaccaacagaggtgtgtgagttgacaagcatgtatataagttattgatataggcgaatgagtctctctaccaacacggtaacaaatccacgcaccaatagtttacatagacaaacaacaactaatccacatagaaacacatgaagttcatatcgaatccaaccatctacgaaaagacaaaaacttaacaaatgttcgcactggtggcgccataagtttttgactgtaaatcgtaactagactagtctagaaccacccaaaatcgcgtcaacatatcgtatttagttatcagcaaaaacaaagggtctacgatgacagaccaacctacacatgtgaccgcttgaaataggccaccggaaacactaattaatttgcgcgagtttataatttctctgtcgtttcatatataaaaaaaacctaactaattccaacattatttttgaatgagctaattaattccaatactattctactctctccgtttcaaaatataagtaaattttattttttaggttcattcaattaatgatgtatgtggtctataatatgaaccacatacatcattaattgaatgaatctaaaaaataaaatttgcttatattttgagacggagggagtatttgtattatttttccacattaataattaatttaattaaaagaaaataataacgtgactagaaaatgaggagaaggtgggatttgtgattgtgtgagagtcagctcagatctgagtctgaaaacggctataaataacaggaaagacacacttaacacctcgcactctgttctctgcttcaagctactccttaacaagaagcgaagaagattcacagagaacactagtctcctctacactaacaccgcaag

SEQ ID NO: 127 - DNA, 1522 bp, Cajanus cajan
agttataaaaattttggtgccaagatgtatatatatcttaattattattgacgctttagatactagctattcattcaaatatatcatataatgttgattacaatgaaatttttaataattttaatgtttaactttggtgatatatacagttaaaatgttatccatcttttaattaattttagagttaatcattttataaatggaatataactaaaaacatggaatagtgaatttgagttcttacaagatataagcaacggacagataaaatttaaaataaggcgaatataagaagttataggtgtagttattttttagcttttaatttttctttaccgtataaacttttcccttagttttttttaattaattaattaatttattattattattattacataaaaagtagatcctacgaatcacaaaactaatcgcgatgatgattggtgaagaaggaaagataaagattaaaaaaaagcaaatgatagaaatggttttttttaatcatatatatgagagtaatgttaccttttcttggtttcaaaaaagaaaatgtaactttttcagatttcttttaataaagtaccgagcatattccctttaaactatgaaaattttaacaagtatagttattaattgaaagtaattttaaaaatatttgctcaaaaaatattaaaatatatatatttgttgaaataaaatgaaattattaaaataattaaaaatattaaaatatctatattttattaaaaaattaaatactcaaattaattaaaaataatacaataattatatttaaattttcaattataatttaaatagtctttaaaaaatttaaacaaaaaaattaaaaaattggattttttttttttgaaaaaatgagtaaccttgttgagaaatcagtttttgaaaaatcgatttctcatctcttaaaaaactaaaaaagaacatctatgctgagaaatcgattcacatgtgaaataatatgaaaaatagattatttatgtaaatataaattaaaaacaatatattataaaaaaaaagtcttaaacatgacatatcataaatatttatttaaaatatgatgtattattatatattaaagtaaatgaaatatgctcatttgacaaaagataggttgaatccacgtgtctttcagtgacagaaagtctattacagctccacgtaaatctgagcagcgcatacattttgggcaacgggtcacacaagcgcgtgcatacaaaccatcacacatttctcgcccacaaattcttcatcattcaatagcaaagaaattatgcgattctatttataatttcctcttaattaattaatattaaataaatcattaataggaagagagaaaaaaaatgtgaagagaaaaggttgagtcactcagatctggagcggaaacggctataaataacccgagaaccactccttacacctcgcactctgttctcggcttcaaccaactatcttagaagaagaagccacagagaaccactcactcacccctaag

SEQ ID NO: 128 - DNA, 1590 bp, Lotus japonicus
ttataaattcagaaaaagttataaaaaatactaaatatgttataatactaaatatctagaaaaagacacaaaaaattatataaataatacaacataaatgacatatagccaaatatcatgaaaacataaaatttcattatcaaatgacatgaaacaatctaatataaatagtatctaaaaagtgaaaaacaacactaaatgtcatgtcatgacacttataacttagatgtctccaacatgccaaataactacatataaacatgtaacaaataactacgaacaaatactctaagttcaaatatgacaaataactacaaatacttaagtctcaaagtttcaaaaagtcatcactccgatactagcactccaagtatgagtaaaattataaaaattattattatatatatggattctgggttgagttggatggatttgagctgaatccgaaatccgatccgaacttggttgggttgtagtaaaatccatccgactaacccgattgcccgatctaacccgcattttttctattggatcgaattgggttgggcgggttcattggatttacccggcccatgttcacccctgtttgaaccccctcaactttttttgttgttgaagggtttcaatttaatatttaaaatttaaataaatatacatgagtttgggttcgatcccctctgaggtaaaaaataatacctttgtggcaagggacatgactaccgtatcccgagccagattagtcacgtggggcccctttccccgtggagactggtggccaaaggacaaaaaaatatacataagtttacatcattattattattattattattattattattattaaattacttggttcaattgacaaagacttattttggcacctaagttttaggtatgaattatctccactcatattttataaaatgatgaattttacattagagattaaattggccgatgtttgtcactgataaaattatgaattttacattaaggactaaattaatcatgtcaatatttaacaacaatattaccttttttagctgaccgaaattgacgtagggaccaaatttgatttacatccttctactttcgaggactataagagctattttaatccgggatcaacttatgccagttaaggaccaaaatcctaattgactatttatccaaaagtgaatgaaatgagatagaagagaaaatgaaatgttgtcgagtcattatacttaataaaaaaaaccctcaaaatatcttcaatattttttttcacatctccaaatccatgaccgtacatgaattaaaaaaaaaagcaaaaaaggtaaaaacaaaacaaaattccaatattctatttctaaaaggcaaattcattattaattaattaataaaagaaaagaaaacatggagagagagttggtgtgatctaattcattttcattctcactcactcagattcagatctagaccaaacggctataaataacccgaaaacctctccttacaactcgccttctgttctctgcttcaatctctccataaggaagaacgaccgcagaaaactggtctcatacccctctaaccaccgtaactcag

SEQ ID NO: 129 - DNA, 1401 bp, Cicer arietinum
attcgtaatttatttaaactaacaatttatgctcattaaacaagaccattatgccctaggtttttatttttattttttcttctgtttttcatggtttttttatggatatgtcggaaattaaacgtggataactctcttgatagatattcaaatttaaatttatcacatattaaaaatctaacattttccgttgatagtttttggaattaattatttgcaatgaaattatatctaaaaaatttataaaagtaaaagtgatggcagagaagagtattttatttaaatgaaaaataatgatcaaaatattatgagctaattcaaattataaaatattaaataaaatacgaatgataaaatgtgatcataggactaaggtttcgttgacatagatataggtaaaactcatactagaattaaaaaatatagttgaacttatgaatatcgttcgctcttttaaaaagatgtagtaattttaaattattaggtaaatctgagacacgatctattataaccaataaaaaaaaaaaaacagattatcattataaataattaaatactccttccttatatcaaatataaaataaaatgaataaataaaaatgcatatacttaattcaaatttgttcttttatatttaagattatatgttgattatatatatattttgtatatttaagattagatgaagtaggtgatttaaattttgtaccacatatatttaatttatttaatctatttttgtttatattatgagataagatagaaagaactttgttagtccatcataactattaatgagtacgactttaaaataattatgataaaaataaaaaatctctaataatataataattatgattgaatgacaatacaaaaaacttttatgctgataatgtacatgaattaaattatttattatttaatagaaaaataattgtttttaaaaaactcttaattatttactatcttattttatctactttttttctacaatatattactaaaataataattaatattacagtgaacttttgaatattaatttttctacaaatccgacaattaaaaaagaattgatggaacatattataaaatagaaaattaattaattttattattctatttcactttgcaaagaaaattattctatttcaatatttttccttttaataaaaaagatagttaattccaataaattatataataaaaataaaattttaagaaagaaaagcgacaaaaaatgggggagggtgggattgtgttaaatctgtcactcagtactgagagaaaacggctataaataacacgaaagccattccatattgctcgcactctgttctctgcttcaatcacctcccttaagaagaatcagagaactagtctcctacacttaaccgaaag

SEQ ID NO: 130 - DNA, 1500 bp, Cajanus cajan
ttattttatctttaatgagtttctagccatgataaatgacacataactcaggctgaaccatctagcatggacccaacttacaacagtagacaagtatgaagttgcttacatgactatgaataatacctccacacaaacaaactgcactttgatataatggtacaatccatcacaattgagcttaagacattcttttaagagtttaacctaattgatataaaaactataaaattatgacagttcaaaagtccacggtcgttcaatccatgaaattctaaaattatgagcttgtttctcaagagccatttgttaacattcacacctacgttctggaacaggaaataattttgcgtcttctgaatcgagccaggagacaaatattaagttgtccagatgactatgaataatacctcccacaaatcgcactttgatataatgatataatctcatcactgttgagtttaacacatccttccgaaagtttaacctaactgatataacaaccttaaagattatggcagtttaaaagtctgcaatcataaatttcaatccatgaaaatttcaaagtatgaccctgtttttatgagccatttgttaaacattcacacttacattctgaacacatgaaacaccacaaaataaataataaaaactcttacatgcatattatacttatattaaaattagtagaaatagacttaaaaatagtagaaaaatggataaataatctaattattacggttaacaaattttctaatgtaatttaataattttaatctaacttctcttccatgtgttttttaaataacaactacatgatttttttccataatttctcttacgtttcttgcactacaaccttaatttttatgtttctaattttaaatataataaaaaagtaatattttattttgttttatgtcataaataaaaataaaataaaataaaattcaatttgagcaaatattccatagaagtgggctatgggtgattcattcgataaatgttacaagtgtccaatacgcatgaaacatgcaatgggctttggcaaattggactggaccgaagtgatggccgaaaagagaaacgagaaagagtaagaagatcggcgatgaagtagaaagcacacagaaaacacaaaatgggaacggaggcataatccaaggaataacgcagccaacacaagagaagggcacggatcaatgacatggcaataaatttaaattcccgcctaaacccctgacctccccttgatttcatttcgtttgcaattgaatttcaatttccctaggggcttaatatctcccctcatttgtgcctagccttatgtcatagtatcatttcccttatctttctctctctccccctctccacccaaaccctaactcccacctcttcgtacgtattttccttcgctttttcatcctttttttttcgttctccacgaattcacgctcagaaatctaactctaactcgttcctctgattgcagcatcagcc

SEQ ID NO: 131 - DNA, 1376 bp, Glycine max
caaattcaaacaaacttacatcccccgaaagctttcaagtaagccgaagtgacctattagcttcgtttttcatgcttccatgattaaaaaagtaattgcagaactccttccagtatatgagctcgaattggggtgccagatatgctaattaattacttttatgatgcaggcaagtgtgttttcagatgctgttcgggatgatgttgatttttataagtatgaactgcactcctttagctttgaagtttgatcactagtggataaattatttatatcttgagctttgaaaatcttccaatgaaacttcattcacactttctgtgaaaaatatcatctattaaagaatcttgtcgtggtcaatgctatgcatgacaacatattaactatttaaaagataacttaaaattataaatagttacatattataaaatgctgtggtggacatgttaccacttgtagaatttatatagtaaaattgaatctataacaacacgacactttatgtttatggaattgtcatgtatgtgataataaataagattaatgaaattattataaaaaacaaggccttaatcgaaaattcaatatctttctatttttttattttataatagtaatataattaaggtgctttattttgttttctcttaattaccgtaattctcatttaaatacaacttttctagcttatgatattgaaagttaattaaaaaaccaataacgtcctgaattttaatctaaaacttctagtacgtttggtttcacattttagatataatttttaaaagttaatagttataaatttgtattttaaatgtgattttttttctatttttaatgactttttaaccatgtacagttattcatactgattttttaaactgtttgcttgaagagacggtttttgacaccaagttaaagaggtcattatctcttctaaaatcctaagatataatttccaaatgaaaccaaatttgtaatgtagcgaagatgagctgccacattgtgttcttgaggtcgaggcactactagccctgtgtttctgtgtagttttactagcaacacaagttttttctttttttcaaaaataaaaaagaaagaagaaaaaaaatgtgatgggtcactcacgggtttgcaatgttagtgtagcatcagcccaatccaaactatcttccataccaatttccaagttttaatttaaatttaaatttcttgactctgttggacctgaattgtgtggcttacattgaccctctcgaaaacatctgaggaagaagtctttgcatccacgtggcagaataagagccactatagcatgacaaaatatcagcatgagaatccacatccaaatccacgaccaatgaggtgttgctgag

SEQ ID NO: 132 - DNA, 1362 bp, Glycine max
cccagcatttgccacgttcgaacgtgagccaaaacgatgtcgttacaatatcttaacctagccgaaacgatgtcgtggtaactcataatatcgcagccttctgtttcgtccttgcccaatgccaactggactacgccgaacccacaaaacccgacatccgcgcgaaccaaagcgcgtgacgccaaattgctcaaaccacaaaggaaacaaaggcgcgttaataacacgcgctatcccacgcgtgttaagcacagtaacgttcaacaaagagaagcagggtataatgggaaaccaaagagaaacctaaggccattatgggaaacaaaaagtagctcactcattataaaattcaagtaaccctagttttcatcatcactctgctgcctcgctctgtttctgtctctgtgtttgcggctgaggattccgaacgagcgaccttcttcgtttctcgcaaaggtaacagcctctgctcttgtctcttccattcgatccatgcctgtctcttctttacgatgatgtttcttcggtgtatgtttatttatttatttatttatgctttatgttgtgaatgttcggttgttttgtttcgctttgcttttgtggattctattggtttttgaatcagttaatcggaagagattttcgagttgttttgtgttttggaggtgaatctttttgttgaggtcgcagatctgttgattttgtgtcataaacgtgcgactctgtttgattttttacgaggttatgacgttttggttgttttattatggatctgttaaggcagaaccatgatttatgtttatgttcgtttacacgattaaattttcttgtaacacgatgaagtttttttaaacacgttgaaggagtcttgttgatatgaatttgtcgattgtttttttgtggttttgttctcacgttatcaagcgtaatcttttactatgtacgcgaacatatctagatctagcagagctttttttttttttaattctttgtgaagcttttgaaatatgaaatttgtttttcaaatttttttttaatttattgaaaacactgtggatactgaataaggttctatgtatgatgcgaaccatgtttgatatgtttgtttttgttgttccatatggatttttctgttagatttccatatgcttttgagtttgtttttgctgttacagattgatttactttttagaatatttctcttggctttcactgttttagaaattttttgttattggtaactataaatgtgtgaatttggattatacctttaccttgttatttagttgtgtgataatttggtttatagttattttgagttctgactcgtgtttctttgaattgattccagtttaagtcatc

SEQ ID NO: 133 - DNA, 1500 bp, Glycine max
ttatttattgattattttatgtgtcaaatttatagtttgtaaagccagtggcatgaggttcaaatccaaattgatgtatctcatattattattgatatttttatttgttttttatactaattactaaatgttttagtaaagagtaatataataagtaacttttttggtatttgtgagttgcaactttgcgagcctcaagattaatcccaaatcacgagttttgctgccttaaccctaatcctaaatcgcgagccccagcctatgttagtcgtcggaccttggaatgttgttaacaaccactcctcattcctcaaggagtgctctgttttttgttttaacatttttttttcgtttcaacctctattcactaacccgtatctttttctcatattggcgactacaataggtcctttgagaatagagttactttgtgtatttgtgtgtttcaccaatagaaccgttgcttggatattccgttaagaaatcatcatggcttgatttgtcgctttgtcttggtcatacaaagtaagagtttgcttctttttttatattgttaccgtctcatgaaattgtttttctttgtgttgcttcttaaattagtaaattataatgcattgaagttttgtccataagagtttgtgctttatcaagtttactttatgtgtatttcattacttgagaaaacttttgcactcaggcgcaaaagaaatcatgattagagtgtaataacatatatgatttgctatggtacaaactgtatatatagatcaactattatgaaattattttaattttaacaacgaacttaagttcaagttttaaatgtaataacatcttttgagcgtggttaagtaattagacatgcttaaaagttggatttgaaactcgtgaaatcaaaatataaatgcaaatttcattagtggaggattttagacatcacctttagctatcggttgaaataaaccgaagtgttgtctaatgtgacaagaattggtgaggtaaacaccaacaacggagacaagattttaggccacaaccaaacgaggaaaagttaacttgaacaactgagttgattagcctaaccaataaaaggttaatgatgggtttagaaagggagttacaagagtgataaaatcctaacaaacaataaaatattttcaaagcaaacaataaaatattttcaaagcgaaaataagtaatatgaaactaaaaaaattgaattaaatggtggaaaggaaagaaagggagaatcacctagaaaatatatacaatttatttatttttatatgaattttgactaaacacgtgaaattcatcatctccttttcatttcatcattaccaaataaagtactagtgtaatggaagaagtatcttgttaatgtatgagtagtgcgtctttgattctcactacacaaaaaatgggcttattagagctggaccgggcccaagtgagtagaaaagaaaggcaacagcgaaaataaacataggcggcgacaca

SEQ ID NO: 134 - DNA, 1500 bp, Glycine max
ggaaaacattgaattggagaatgaggtttagggttagtgtgagttttttttccaaagaggtgatagtgatgttgggtggttctttttcatcattgttgttttgtttgtttgagttgacgatagtcaatgtggtgatggttgtgtaacttggtcggtggttgcgtagtgtaatcaatagtggtggcacgatggcagtggtttctattgaagaaggggagaaatggaagaaggagaatgagaaaaaggtggtgaggtggcgttgaagtgtcagtgacaaagcacgatgttgtgatatttcatctgtcacattatatttaatgaaagaaaattaatgatgagtaataaaatgaaacctaaaattttgtagatacttaattgaaaaaaataaatgttaagtaataattttaaaatgagttatattgcaagtacgaaaaacttaattaaacctttatttcttatataaattttcactaaacacatgaaattcattgtctccttttcatttcactctctggtttaaatccttaccaattattaccaaacaaagcactagttagtggaaattgaagaagtgttttgttaatgcatatgagcagtgcatctttgtttttttgttagccagaatacgcgattaagaatatagcaagaaggtggcgttttgttttgtgtggcggataaaaaaaaaaaaatcagcgaagaataagtgttccattggaatgggctttggtagagacaatagttgtccaataaacaagaaatatacttattgggcttattggagctgaaccggaccaaaatcacaatagagaacaccagcaaaaaggggcgttggtttgggcagccagcacaatgataatacacccagcaaaatagtgaaatttcatttttacccttccataaagctgatatggactggcgaatccataaacctaatacggatttaaaaatatattttatgaattaatttaaaaataatggttcatcttggaagcaaaccttattattaataggatgctctaatcaattaagttaggcaaattttgttataaaataattaatgtcgctatatataataataagattttcaatgtatatttaatatacatgtaaatttaaataataaattttgtgataattaattttgatctaataattaatttgtttacatatataaattataaataaaatcataaatttcataaaaaattatatacataaacaaattaattattagatcaaaattaatttataaaatttattatctaaatttatatgtaaacaaatatatatttaaatttttattattatacatagcaatattaattattttataacataattaatttattaattatgttgatgagaacatcatattaataatatgaaaatcacacgtgttgaataattatatgaaccattagtctaattcgtatcatgtagattgattgatatcaattttatacaaagcaaatgcccgtaaaagaggagaatgaagaccgc

SEQ ID NO: 135 - DNA, 1712 bp, Glycine max
aaatgagccgaatgttaagtgagtgtgataatatggttcatcgtgctaacttatttatttccttttttagttttcatttttattatataataaaataaaagaatttacctcttattcaattatacattattatgtacgatattgttaatttatgtaatacttattttaaatgttataattatagtgatttttaattagagagtgaattttaattgatttataatataaaaaaatatatttaattatatcacaattaaattcaaaataaaaagtaaattttaaagtctaaaaaattgaaagaactgttttatattaattataaaaaatgaaaaattaatgttttgtgcttaaatataatataaataaatcaattaaaaggataaaaattattttttaaaaactaagttgaagtttaaaatagaaaatagaaaatataacaaaatttcttttaatttaatgtaattttacaattatttatatattgaaaacttaattatattcttaatctttgaaaatatgatctttcctaaaactactctatttttttatatttattaaaattttaaatattatttaattttagtacctaaaatatcttagcataaaattaaaaacacattcttttttttttatttttatcaaaagtaaagattagaataaaaaaattaagaggaactcattctaaaaagtaccatattttcaaaactccaaaacacatttaaattttaatataatgataacattggtattttatattcaatattaacactcacggacattaagtgagctgaaccgttttttattcagtatttactttttcatttaactgcacaaataaaaaatattgataaataaataaatttgtaacatttttaatatttttatttctgtttatttattgatttaaaatttatatatataatctgaaaaataatatatatctatatatataaacctaaatttataattaaaaaaattaaataaaatatgaggatgcagagttaattagaaaattaataaataaaaatataattcataaaattaatacaatgatataaagcccatatatataaataattaattgaataagaaaacctcttgcgtcaggaacaacagttaataattcaaattaattaatgtatattacaagagtctaaaacagtggtctcagtataaaaaaattaaaaactcagattaatgtcttgtacttttgctatagagagaaaaatggattaaaatgaaatgagcaagctttataaaaaaaataataataaaagaaaaagtaaaatgaacaagaggtagagtagaatgattgaatgagtgccacattcgatagatattttcacaatccttccatcacatcaactctcacccgttgatcgtcgtttgaccatcatttaaaatctcatccaacggcaaacaaactccagaaagcgtacattttagaacataccagaaaacccctctcatcactccagtgataaaaacgacgtcgacgtgggaaaagatcctcaacgcaaacggattagacgcgtcgtgagatcgtgacacgtggacggtggatgtttcacactctctcccctcaaccgctttataaattggggtcgtggcttctccttgaaactcgttctagtgtgtgattgtttgttgttgtaactcgttcttcttcttttgttgttctgtgtttgtgtttgttttttctctcacctgac

SEQ ID NO: 136 - DNA, 1069 bp, Glycine max
caaatggtaaaagaaataaatagttcatactatttttttccaaattttgaaagactttgtgatctcttacagacacaatagcaatcaaatagagaataatcttacgataaaaaaagagaaacaaactcattaaatataataataataactaggataaaataagtttatagccaatttttgttgaatctggtttttagtctatgatatatcatgtaatttagttcctaaaatttatatgaaaaaaaatggttttagtgtatgggttcactcctcaatttttgtacttttcttaacttgagatttagaaaagacactaaaaacaagtattttataaaattctgggacaaaattgtttagtatgaaagtttggaaattagacacaagatttaataaaagtttgtagactaaaaacatattttatctactaaaagtcatcaatttttataacaattatatataattatgtttataatgtaatcttagtaattagatatataaatagataaatgagacttataataataaaaaaaacatataattaactcaagtaataacaattgagaaaaagacatagaaatgtcattcttattatatctattaattagtatacatatttctttgggttagattataattaatgaaaacaaatattagtaaaaagattaatatataataattgataacaaatattattatatttattaataggtgtattttttttactataattattatatattcattaaaaaatttaaggaagggttactaaaagaaaataaaatggttttgagaagagggggggctgatttagatgtctgatttttttaattaaactgtgaagtattcttttagctgagaaacaattctgtttttgtaattgtatttttaagggatatgaagctagctaggaagatgaataagaggtgtgagttgtaagggttgggtaagggaatatgagattgtaattggtccaaggcatatcaccttatccatatccacatccaaaatctcctactttactctctctctctctctgacaaccccaacacttgtattgtctcaagcaacc

SEQ ID NO: 137 - DNA, 644 bp, Glycine max
ctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttga

SEQ ID NO: 138 - DNA, 1369 bp, Glycine max
ccgggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaat

SEQ ID NO: 139 - DNA, 727 bp, Helianthus annuus
ccgactggattaaacgcagtagtttgaaagtaacccaagtccaacctactcgtagacacaacagtaaaattactggtaaaacctgttggtttccctgagttttctgtcattgtccactaatgcctcattcttcaccaaatagaagttgaacctgcatatacaataaggctatgggatatactttcacgccccttaactacacgtaggcatcacatcacgtacttgaccctctttcacttcacttcacaacaaggaaattgtttaacgcccattaacaccccttaaattaaataaaatacatatatatagcatgttgtgtgtgtgtaaattttgcgatgcttgattctctccctctctctcctaattaacttcttaacatataaagcttttaataactcttaacactcttcaggaacggtataataaatgttaacggataccaactaggattacgtgatacaagccttcctctctaaaacttaagaaagtcaccaaaaagaaagtcaaagacggcaacggcaacccagaagctacgttctctttctagattctatgccctacctaccgtgtggtgcacaacaacttgacccccatcattgactctccaccgtacacgtgtcacgataccatttgttggtgtcctaccacttacgtaacattaatattccatccgtgaccgcctcttcttcatcatcctataaattcaccttcttatctcttccgcttt

SEQ ID NO: 140 - DNA, 1118 bp, Lotus japonicus
cttccatactttcatggtgtggatgcaccccaactgatataaatactccctttgtcctacgataagtctatgagttttttaatatgaaaatataagtcactgcgcattttctataaagcactatttatatttttctatgtttacccaagttattattcatcattataaaaaaaattacagctttcacttattatatttttctataattatctctcttaagtggatttcacattccaatattattatttggtctttataatatatgtgaaaaagttttgcatatgcacagaatgtagagagataagaataccaatacttctttttatcaactcatgcgaattacttttagccaatccaagtgcaatgaaggatgaagggcacaatgaaaatgactttccatcggagataatcaaactgcaaattacaagtttgatttaaataaatttctacatgtaacatgatgatgatggaataagattggtttccaaatcttcaaaacaatgaagatccacgtttgaggctctgatcctcgtttgaagcttagacccacgtttgataattggatcaaaacacaccctaatatggagagaggattttgaggaaataattaattgaattacttgtattattgataaagtaatttagataagttgttagtacaacttattgcaactatggatagacaaaaatcacttattttaaggggagaagtatataacaacttattataaaatttctgagcagccggcctcctccaatcaatcatagagagtggagcccattctggaaaaccaaggaacccccaacttgcactggtgcggtggcccaattcaaaaaaaacggggcccagcaaagtaacgccgttgtaacgatttatcaatccaaactcaaaaggcggcgagatgcgtttaactccgtgaaattaacaaaccgccaacaacttgcaatttgcaactaccgtttccaagaagaactcaaccacacaacgtatcctatcccaaaccacacgcaactagtgacgcgtcataaggacacgtgtcacaatttgactggttaataatttcaccgcttttgctataaattacctccaatccccttagcttcttcacaattcagtt

SEQ ID NO: 141 - DNA, 1000 bp, Nicotiana tabacum
gcaagtaacaagacatttctaatgatcaaatttattcttttatgtacagttttttctttgggttgatatttgctagtttggacaaaccttttgtcaataaatatttttagatttgtacttttgagtactttaatttagaatattacagtctatgactacatgcactagttagtattcaaaccgaataaatcaaagttaccaaaccgaataaatcgaaaccgaaaggagaaaccgcaccataccgaatttaattaggtacgatattgatattgtattttaaaaaatcgaataccaaaaataccgaattaaaatatctaaatatcgtacagtaccgaccgataaatactctataagcatcatcatgtcaccattcctgcttggaaatagatgtaataatgtaattcaaggtgaagatcattgaaaatgagatatttggactcttagataattgtgcaactgatatttttatttactttttttctttcatccaataattgcgttacattaaaaatgagatatttggattaatattcttctccttgaccacaaagcaaggaaagcctaaggaccgatagtaaagttgtattcgtgtggttgcgtgttagttttgagcggcaaaataaattatgttaaggtaaattatttttggaacaataataaaattatttctgtataatatataaatcatatatttgaaccgtagaattatcagttaatacttgtatatgaggaggctaactacgttagagcgctaacgagaatacttcatataccgtattttttacgataataataatgtaatgtgaaattgctatccaaaaggcacctaattttgtccaccgttcaaaggaaaggacaaggaagtagtagcgtgtaggtttggtgctgtacaaaataagcaagacacgtgttgccttattataggataatccataaggcaatttcgtcttaagtcggccattgcacctttaaaaggagcctctttgttcccaaaatcttc

SEQ ID NO: 142 - DNA, 2380 bp, Prunus persica
aagtattgtattaagaaaaaaaaggaaaatgtaatataatatatttaaaaggtatagccgggcggctatactagcatcccttatcctcacttctcattccacgttcagacccacgacagccccaaattgagagagagagagagagagagagagagagagagagagagaggtagtctggtgggcttttatgtggggctgccgttgggtgtgtagggggtggggagggatgtggaacaaccataagagaagaaagagaatgtttgaaaggggaatcaatgggggaagaccaattattgcagatttgattcctcatcagtgacaggattgcagatcagggaaaaaaaactttgatgagggtttgtgtccaaagctttgatgcatatctgggttacagatttgttttcattgttgctttctttggcctagttttggttctttttttgtttttctcgaacttttggctgtttacagagagagaggagagacgaagaagaaggatcaagagatttgggttgcagggttggcatgtgccattcacgtggtgtttttaatagagatttggatggaaattgccaaaaactaaaggtgggggtaaattggccataaaatttagtacaagtgctaaatcagccgaaaaattaatttatgatgtagattttaactagcgtgataattcaggaagttattcgacagtttaccatttataatatcaaacaataaaagactattcacttaaattgaagagtcgtgttatcatcaaacatctatgcacacgtaaacaatttcctcaactgacttacaaaatgattatgatcaagtcacaacatctagtgatgagcataatcgcaactgaatcctaccgcatcataacttataagttggtgacaaaaattggttacaatgtatgctttcaaggactgagacttgatattagggttaggtaacaagtcattctcagtgaacatattgagggggcctaaagagtcaacacatagacttcaattaagttgactagtattgcttcattaatgcaacagattggcatggtccagcgtgtgccctttcgatgcatttctttctcatctttaaacctctctttttcagctttcttcacctcctctttacttcacttgattacaatttatttagtactaggatatatatatatatatatatatgatttgttcgtgcatgtcacgcccacatggagctcgtcttaatgaaaaacataatcccactaattaggcattgcctcaaatccaagcaaacctttcagggaagtcaagccaaccttggccattagttcttgacgtcaattattgcttgcatcaaagcataaatgatatagtttgggttggcacaaacctaaaccacaaaggatttttttacccaaaaaaatctaccaagggatttgggatcatttggtattgcttctccaagaagcacttgtgcttataagcgtttttattataataatgtagagattttcataagaaattcaagtatttctctaaaaaaatacttctatgattcataagaatttctaaatttttttgttaacattgttaatagcgtttttggttatcaaaaaatacttttaatccttctaaaagtgctcccaaacatgccattttattcaccttattgtgtaattttcaaaacttcaattcatattacttatgtaataagttatgcaacgtgcatcaactattaactcaattgttcactgaaaatagagataaaaggtgcttagagttattggtactgaatatgggacgatgattttatttcgtttatcatggtaattttcttttctgtgctatgaatgatatctgtgatggctattttacggccccacattagctattttattttctataatcatgctggataatatagaattgcagctgttgttataggttttaaaaaaaaaaaagcaaagatattttcaaggatttataatttactatattttccatatactcaaatactaatgtaatgcgcagataggatcacacgtatatgattgatcattgaggaggtaagccatgatggcacgtaacaggcatgcacatatatcatctccatcagaaccgttgatcacaatttgatcttacccatcagcaatctgacggtggagaaacttaaaggaagaggtacagttctagaagacacgagaaacacccaccctaattatgtaggcataaagacgggagggaaaaccgacagatacgtggaagaaaataggcataaagggattggaccgatatgcatggatagacatgacgtatgatcatgccacgtgtacggtgcacataaccccattctctctcctcccctgcactataaattgagggcatcgccttcctataactcgttc

SEQ ID NO: 143 - DNA, 1500 bp, Phaseolus vulgaris
aaaatattgttttaacgagataaaatgttaaatctagataaaagggtgtgattttagttatttatagtttcgtagaccaaaataagtgcttaaaaacttaagttttaaaggatttacacgtgataacttatatatacatgttgttgtcttcctttttttttttttctataacataatatttgactatatataactttaatttcatcctgactaagattgggttatggaatattgttataaagttagtcaactttaatgattagttggtaaatatctttgtcaaatttattcatggaataaacactgactacatacttagtaaatttggtatatataatttatatagtgaaagtttggaaatgtattaaaatcaaaataatatatttaagtgtaattaatttatataataaaatcacaaccatattctatctttacttcaaccttttccctctttttattttaacaattttgtaatcatgaccaccgtaataatgattcacaattggtttatatatgaaaatggtttcttataaaaaaaatgtaaaaaaaggtgtaaagaaagaaactattttttaattcaaaatttagtaatcaattttataattatattctcaatatgaatattttaatcgatattattatttaaaattaatttaattaatttattattatgcttttaaaattttcttataattaatatgttttttaacacataagtaacaacaatcctataattaatgttgagcaaacatccattcacaaaatttcgcaatgaaatattttactatcatattcatttgatttaagaaaattaagtaaatataaattattttttaatgaaataaagaaagttaaatttaacaaattaagttaaaatggagacagactaaataatttaacctaattattttatactaatgtttcctcttttactcattttttagatgactattttttacgatgatagaaatgtaacatacatatataaataattttagatgagtcaaaattgaaaacacaaaaaatggaatgcaaataatgattgaaaatggaagttattttattttataggtaagatttggtatgaaaatgtttaaaggtatgctctatttgttgccttagaataagaaagagtgagaaatcatataacaattatacattaatattaattttaaaaaattaattaattttttaaataattcatgtgaaatgagaaaagggtagatagtggaactgttttgataaaattagttttttttttaatataatgtatgagttatggaaagtgggtgtgtttttgtttggtggatgaaggaagaaggaatgggcttgaaagaaggcaataaaactagaaatgggtttgatagagtggcccaaatcacagtcggaaaatagaagtaaaagcaatgaagaagatcggcgattgcagaagaaagcacagagaaaacacaaaagggtaacggaggcataagcttaggaataacgcacccaacacaaaat

SEQ ID NO: 144 - DNA, 1000 bp, Vitis vinifera
gaaaattaatatgattaaaactaattaaaaacttatgtatttttaaattatttaatctttatattgataagttaaaataaataaaatgagtttgaagtaacaaaaaaaataatttatcaacttttaatctattttttttattttcctctgcttttcctttgtattttctttcccttacattttccttcaaattttttgggaaccaaacatagccaaagagaaagtgttatttaattataaagatgaaatatttcctaatttgaaaaattaattttttttttatttttattataaaaataattattttaaataagaatattctcattttttttataaaaataaattttaaatcatcacaatatcaaagaatatttatataaaaattaaaaaatggggacaattatttttacaaatctggaaattttcaccctttttaattaattaattcaaaaagaaaaagtattatatatataaattttattaagaaaagaaaaataaattaatatatggagaagtctgggtggttgcagacttgcagttaacggttctctttaaaaggcacggcggctgcgagatataacattttaaaaaacaattcactttttgaaaaagaaaaagaaacggtgacggagtcgttagccagagacaactccgttacgccgagcgtcacacctaacgtccacccacccccatttaccaaagtaatctccactaaatatcctaccccaccgcccccactttaacgaacggcccaaaatcacccccccccaaatcccacccacagaacaccaccgtgatcgtactcgccatgaaaacgtgtgggacttctcctcaccgcctagcaccctccgtcattaaagtagagccacgagacatctagatgccgtggaggataggataaaggacacgtggcatcactctattcgtttatcctaatcggtttcgtcaccgactcggcaactgcgactacaaatagacgccctctcttcacagcctctcc

SEQ ID NO: 145 - DNA, 1518 bp, Vitis vinifera
taaatatggtaaaaatgtataaatatgaaataagaattaaaagttaatatatatatattttcatagcttcgagttatgattattttatcataacaaaaatgtgcaaagtaatatatagtttcaagctatgaattattttatgtaaatttgtgcataacttcaggttgtgaactttcattatgtaaatttgtacatagcttcaggctatgaacatttatttattttatagtttttggctgtataatattgtttggtataacatccagttataccgtataattaaaaataaataattagggatcatatacataacatctggtcatgtatctgtaattctgtaatttttcttggttatatgaattgcacatatttttcataacttttggttatgaatttaccctataatttataatttatcccataacttctcgttataaggattatacatatttatgttttaaaagataataaaatacaaaatcatgatataagtttatcacatacctttttgtgagaaagaagagagaaatttttttctatttatagaatgtttttctatttttctgaatagagaaaaatatttctatttagtttattaatttattataagacttgattttaattttgattttttttaatataattttcacgatatcttaaaatattttttttttcaaaaatggatatgattttaataattaaaaatgttcaggataatggatagcaagtggctaacgtttccttaactaatacgatatctcatcttctccttaagaaaatcgatttgattttagagaagaaaatcgatttgattttgagtattaaagaagtgtcattttaagtgttattaaacaagtgccttgaatgtggacgttttagaatcttgattatttttacttgttttttaaaataataaacataaaattatttttaaaaataattcaaattattctacaaaatataaaaaacaaaataatttttaaagtttgttcctaaaaattatttttggaattgtttttctattgcgaaagcatggtgggtgcgagaaaaatgttcttctgccggagtttaacattttaaaaacgattttttaaaaggggcaaaaagaaaagctgacggagtcgttagtcatcaaccactccgtcacgtggagcgtcacagttaacgtccgccaacccccatttaccaaaaaaaactccactaaaaatcctaccccaccgcccccacttataacgaacggcccaaaatcacactccacaaatctcaccccacagaaacgcaccgtgaacgtactcgccacgaacacgtgtgggacttctccccccatctccgtgcttcaagtagagccaagggacatctagatgccgtgaaggatagtgccaagagccatctagatgccgtgcaggaaaggttagcgatacacgtggcagaaatctatttgctatttcctcatcggtttcgtcaccgagtaggcaacctcgactacaaatagacgctctcacggggacttctacatca

SEQ ID NO: 146 - DNA, 1549 bp, Arabidopsis thaliana
aatttattatgtgttttttttccgtggtcgagattgtgtattattctttagttattacaagacttttagctaaaatttgaaagaatttactttaagaaaatcttaacatctgagataatttcagcaatagattatatttttcattactctagcagtatttttgcagatcaatcgcaacatatatggttgttagaaaaaatgcactatatatatatatattattttttcaattaaaagtgcatgatatataatatatatatatatatatatatgtgtgtgtgtatatggtcaaagaaattcttatacaaatatacacgaacacatatatttgacaaaatcaaagtattacactaaacaatgagttggtgcatggccaaaacaaatatgtagattaaaaattccagcctccaaaaaaaaatccaagtgttgtaaagcattatatatatatagtagatcccaaatttttgtacaattccacactgatcgaatttttaaagttgaatatctgacgtaggatttttttaatgtcttacctgaccatttactaataacattcatacgttttcatttgaaatatcctctataattatattgaatttggcacataataagaaacctaattggtgatttattttactagtaaatttctggtgatgggctttctactagaaagctctcggaaaatcttggaccaaatccatattccatgacttcgattgttaaccctattagttttcacaaacatactatcaatatcattgcaacggaaaaggtacaagtaaaacattcaatccgatagggaagtgatgtaggaggttgggaagacaggcccagaaagagatttatctgacttgttttgtgtatagttttcaatgttcataaaggaagatggagacttgagaagttttttttggactttgtttagctttgttgggcgttttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaatt

SEQ ID NO: 147 - DNA, 569 bp, Arabidopsis thaliana
aatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaatt

SEQ ID NO: 148 - DNA, 275 bp, Arabidopsis thaliana
tcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaatt

SEQ ID NO: 149 - DNA, 1506 bp, Cicer arietinum; identical to SEQ ID NO: 10

SEQ ID NO: 150 - DNA, 421 bp, Cicer arietinum
ctagaaatcaactgtaaattttatttttaaaaaatgtaatgagcaatggggttgcactatagcaccagccaaaatgtccaaaccactcttccaagtttcagtttttgtttctcaagtttggtggacctaattttccttggctcacattcaccctctccaaaacatctaagcaagcaaaattatgtatccacatggcacaaagagagccacattattagtacaaaatatcaggttgagaatccacatccaaaatcaccaccaatgagatgttgctgactagtgcatatcctaatccacaacatcttatgatttccaccgtgtttactctctacgcttctataaaccattgcagcctttccttattcttcacaacactcactagctagtagcaacaaagggaacaaacaacacaatcggatcc

SEQ ID NO: 151 - DNA, 145 bp, Cicer arietinum
ctagtgcatatcctaatccacaacatcttatgatttccaccgtgtttactctctacgcttctataaaccattgcagcctttccttattcttcacaacactcactagctagtagcaacaaagggaacaaacaacacaatcggatcc

SEQ ID NO: 152 - DNA, 1506 bp, Cicer arietinum
attaaggtttatccaaagaaaattttgccaatatgtgaacaaattgtacaaacttcataataccatatctttgaattacgatcacaatgaaaaactaactacttgaagatgacaaaaggcaactaactacatttatattttgtatcagcaaatgccctaattcttacatcatgaatcacaggccttaaagttacactgctagaaaaactgagaacaacattactgcatcaaagttgcaaaataatgccgaagataaacagaaaaaaataagtcagaacctcatcagtaatacaacaaaatttcaaacatgtcatcaacatcaacatcatcattgagtaatacaagctaaagatgaagtttcaaacatctttcagttttttacaggccagttatcctttcataatctcaactattcaatattttagttatagatcctattgtttacatagttatccatgtaatatgctctttaagaatcagctgtctctaactttaatttttgtcttgacattttcaacatattataagggtatgtttagtacttgtgtgtctttttttgttaatcaatgtaacataaatgtctactttgcgaccgtcttcgaggggattttgaattttgtaccatgaggttacaagactaaactcttaactcgtggcgtgattttatttagtcacgcatgacgtatcatagactaaaaacaacattgtttaaaacctaaattacatgattaaaatcaaaaaatgttataatcacagatacttaaatcatatttaaatctaaattaaatattagagaacacattataaagtttttaaatatgtatcttaaaaatagattgattttatgcaattttacttggctgagaggaaaatgtagaaataaatttctcatgtaactatgaagacagatgtacattctcaactaaccaacaaaacggataagagagtgtcagctgagaagtttcattccattaattttttttcttcttattctaatttttattttcctccctaaggagccaaaagacttggacaggaacaaatgttacccattccaatccattcatgataaatttttgtgaagtaacagtcacataattccattatgaggccaatacactagagtagaatttacaaggatacaattaaattattaaaaaaaatatcaacataaccaccatcacatatttacactctactcatgaggagataagattagtggggccataatccacgtggcactatcatagtggtgcctaatgataaggctaagatacaaaaagtttcaaacttgtgtggttaatatggctgcaatgtttatcattttcacaatccaacaaattggtattagacagtagctaattaccacatattttaagatcatattggaaatattgaaaatagcaaaaataaaacattatatatagcaaggtatagtagaagctttgcaaattcagtacaagtgtttctgattaaagagttaagtaagggagaaaaacaggatcc

SEQ ID NO: 153 - DNA, 737 bp, Cicer arietinum
atttaaatctaaattaaatattagagaacacattataaagtttttaaatatgtatcttaaaaatagattgattttatgcaattttacttggctgagaggaaaatgtagaaataaatttctcatgtaactatgaagacagatgtacattctcaactaaccaacaaaacggataagagagtgtcagctgagaagtttcattccattaattttttttcttcttattctaatttttattttcctccctaaggagccaaaagacttggacaggaacaaatgttacccattccaatccattcatgataaatttttgtgaagtaacagtcacataattccattatgaggccaatacactagagtagaatttacaaggatacaattaaattattaaaaaaaatatcaacataaccaccatcacatatttacactctactcatgaggagataagattagtggggccataatccacgtggcactatcatagtggtgcctaatgataaggctaagatacaaaaagtttcaaacttgtgtggttaatatggctgcaatgtttatcattttcacaatccaacaaattggtattagacagtagctaattaccacatattttaagatcatattggaaatattgaaaatagcaaaaataaaacattatatatagcaaggtatagtagaagctttgcaaattcagtacaagtgtttctgattaaagagttaagtaagggagaaaaacaggatcc

SEQ ID NO: 154 - DNA, 496 bp, Cicer arietinum
cctaaggagccaaaagacttggacaggaacaaatgttacccattccaatccattcatgataaatttttgtgaagtaacagtcacataattccattatgaggccaatacactagagtagaatttacaaggatacaattaaattattaaaaaaaatatcaacataaccaccatcacatatttacactctactcatgaggagataagattagtggggccataatccacgtggcactatcatagtggtgcctaatgataaggctaagatacaaaaagtttcaaacttgtgtggttaatatggctgcaatgtttatcattttcacaatccaacaaattggtattagacagtagctaattaccacatattttaagatcatattggaaatattgaaaatagcaaaaataaaacattatatatagcaaggtatagtagaagctttgcaaattcagtacaagtgtttctgattaaagagttaagtaagggagaaaaacaggatcc

SEQ ID NO: 155 - DNA, 428 bp, Cicer arietinum
gtgaagtaacagtcacataattccattatgaggccaatacactagagtagaatttacaaggatacaattaaattattaaaaaaaatatcaacataaccaccatcacatatttacactctactcatgaggagataagattagtggggccataatccacgtggcactatcatagtggtgcctaatgataaggctaagatacaaaaagtttcaaacttgtgtggttaatatggctgcaatgtttatcattttcacaatccaacaaattggtattagacagtagctaattaccacatattttaagatcatattggaaatattgaaaatagcaaaaataaaacattatatatagcaaggtatagtagaagctttgcaaattcagtacaagtgtttctgattaaagagttaagtaagggagaaaaacaggatcc

SEQ ID NO: 156 - DNA, 1506 bp, Cicer arietinum
atttgtctccatcgaagagtcatatcagatatctctcaacattgttatttttatgatttaaatatttatttgattatatagatatattattttttatattagtctatataaattttattgtttaaatttaattattataaaattaaagataattaatttttagtttaaaaaaagagttaataaattgcaggataagtgaatttataaatacacataaatatattttttatgaaatattaaaaattaaaacattttataatattaattaattatttaataattagtattattttataaaaaaatacaaataaaaattaaaagaatattttaaaagtgttttaaaacttcgttattcttttatattaatttgatattaagaactaaaaccatttataaaatttaattcaaaattttttatagaaactaaaataaaaaaattatattcataaaaattaaatatatattttttaaatatattttactataaatacaaattttgtttttttactcataaaactcgaacttaataccatatcgaaccaatagtatgatagtagaaccaaaaaattaatttatttaacattgtaaaaaaaaatacaatttatttaaccattctaatattctctttgttgaatttaatataattttcatagcagtcggccttccaacggtggagtggagttcatattgtaaaacagacgacgtgggcccatacgaaataagtgacccaataaagtaacgccgtggtaacgattaatccagattccagtagaaggttgcattcgactccgtcaagattaaacgccaactaccatttcccaattgcaattaataatttccgagaaagaccgccctgaacgcattggataccaaaactaagccagatacaacaagcaacccaacaactagcaccgaatgacatataatcaagacacgtgtcacaattctagtggttacgtttttgacccttttgctataaattaaccccaatgctcccatacaaatcgcagtttcatttctattgaattactttctttgagccctatctacttctcaacaaatcctcaaggtattttccatttcttttctgattttcgattcgggttttcgtctttcctttcgtagatcgtttagaaatcgattagacagagtttatataagctatatttattagaaatgatctcagatcgttttctaatatatattctcaataaatctgtttgaattgcatgatcgtcaattaggtttgttgtatctggtttatgaacaggttttgcatgaacggatctgataacgtttaatttgaaatgcttcatattcgtttgttatacactgtttttaatatgtattagagtcaattcaatttctggtgtttgattttagtttatttgttgttttgatctcaagctgttttggtctgttgttatagattcataatccagtttctaattcgatttctggcgtttgattctgatttatttgttgctgctacttccacagggatcc

SEQ ID NO: 157 - DNA, 438 bp, Cicer arietinum
gaatttaatataattttcatagcagtcggccttccaacggtggagtggagttcatattgtaaaacagacgacgtgggcccatacgaaataagtgacccaataaagtaacgccgtggtaacgattaatccagattccagtagaaggttgcattcgactccgtcaagattaaacgccaactaccatttcccaattgcaattaataatttccgagaaagaccgccctgaacgcattggataccaaaactaagccagatacaacaagcaacccaacaactagcaccgaatgacatataatcaagacacgtgtcacaattctagtggttacgtttttgacccttttgctataaattaaccccaatgctcccatacaaatcgcagtttcatttctattgaattactttctttgagccctatctacttctcaacaaatcctcaag

SEQ ID NO: 158 - DNA, 137 bp, Cicer arietinum
cacgtgtcacaattctagtggttacgtttttgacccttttgctataaattaaccccaatgctcccatacaaatcgcagtttcatttctattgaattactttctttgagccctatctacttctcaacaaatcctcaag

SEQ ID NO: 159 - DNA, 869 bp, Cicer arietinum
gaatttaatataattttcatagcagtcggccttccaacggtggagtggagttcatattgtaaaacagacgacgtgggcccatacgaaataagtgacccaataaagtaacgccgtggtaacgattaatccagattccagtagaaggttgcattcgactccgtcaagattaaacgccaactaccatttcccaattgcaattaataatttccgagaaagaccgccctgaacgcattggataccaaaactaagccagatacaacaagcaacccaacaactagcaccgaatgacatataatcaagacacgtgtcacaattctagtggttacgtttttgacccttttgctataaattaaccccaatgctcccatacaaatcgcagtttcatttctattgaattactttctttgagccctatctacttctcaacaaatcctcaaggtattttccatttcttttctgattttcgattcgggttttcgtctttcctttcgtagatcgtttagaaatcgattagacagagtttatataagctatatttattagaaatgatctcagatcgttttctaatatatattctcaataaatctgtttgaattgcatgatcgtcaattaggtttgttgtatctggtttatgaacaggttttgcatgaacggatctgataacgtttaatttgaaatgcttcatattcgtttgttatacactgtttttaatatgtattagagtcaattcaatttctggtgtttgattttagtttatttgttgttttgatctcaagctgttttggtctgttgttatagattcataatccagtttctaattcgatttctggcgtttgattctgatttatttgttgctgctacttccacag

SEQ ID NO: 160 - DNA, 568 bp, Cicer arietinum
cacgtgtcacaattctagtggttacgtttttgacccttttgctataaattaaccccaatgctcccatacaaatcgcagtttcatttctattgaattactttctttgagccctatctacttctcaacaaatcctcaaggtattttccatttcttttctgattttcgattcgggttttcgtctttcctttcgtagatcgtttagaaatcgattagacagagtttatataagctatatttattagaaatgatctcagatcgttttctaatatatattctcaataaatctgtttgaattgcatgatcgtcaattaggtttgttgtatctggtttatgaacaggttttgcatgaacggatctgataacgtttaatttgaaatgcttcatattcgtttgttatacactgtttttaatatgtattagagtcaattcaatttctggtgtttgattttagtttatttgttgttttgatctcaagctgttttggtctgttgttatagattcataatccagtttctaattcgatttctggcgtttgattctgatttatttgttgctgctacttccacag

SEQ ID NO: 161 - DNA, 473 bp, Cicer arietinum
tactttctttgagccctatctacttctcaacaaatcctcaaggtattttccatttcttttctgattttcgattcgggttttcgtctttcctttcgtagatcgtttagaaatcgattagacagagtttatataagctatatttattagaaatgatctcagatcgttttctaatatatattctcaataaatctgtttgaattgcatgatcgtcaattaggtttgttgtatctggtttatgaacaggttttgcatgaacggatctgataacgtttaatttgaaatgcttcatattcgtttgttatacactgtttttaatatgtattagagtcaattcaatttctggtgtttgattttagtttatttgttgttttgatctcaagctgttttggtctgttgttatagattcataatccagtttctaattcgatttctggcgtttgattctgatttatttgttgctgctacttccacag

SEQ ID NO: 162 - DNA, 402 bp, Chrysanthemum x morifolium
gagtctgcccatgtacgaaataacttctattatttggtattgggcctaagcccagctcagagtacgtgggggtaccacatataggaaggtaacaaaatactgcaagatagccccataacgtaccagcctctccttaccacgaagagataagatataagacccaccctgccacgtgtcacatcgtcatggtggttaatgataagggattacatccttctatgtttgtggacatgatgcatgtaatgtcatgagccacagcatccaatggccacaggaacgtaagaatgtagatagatttgattttgtccgttagatagcaaacaacattataaaaggtgtgtatcaataggaactaattcactcattggattcatagaagtccattcctcctaagtatctaaa

SEQ ID NO: 163 - DNA, 137 bp, Chrysanthemum x morifolium
tggccacaggaacgtaagaatgtagatagatttgattttgtccgttagatagcaaacaacattataaaaggtgtgtatcaataggaactaattcactcattggattcatagaagtccattcctcctaagtatctaaa

SEQ ID NO: 164 - DNA, 235 bp, Lotus japonicus
ctagtttctgattttgtgatctattcattatgtgtgaattcgttccccaaatttgtggttttaatgtttatggtctcaataggtgcttatgattttgatggatttagggtttatattttgatttctatggtgtagttgctgtgttttttatgattttgatggatttagggtttatattttgatttctatgatgtggttgctgttttttgatgttttagtggtttgatgttatcag

SEQ ID NO: 165 - DNA, 517 bp, Lotus japonicus
atcgacattctcattcgaattgaatcagttcatcaaaacctaattcctcttctctctatttctcctaccaaggtgcgtcttcgtctattttgaattcatcaaattgcgactctggtttcattatcttgaattctgattcatcccaaatttatggtttaatatttgtagttctaataggttacaatccgattagggtttgatggattagggatttgtttgttataatctgattagggtttgataattgatcctgcttatgttttcaatgtcatgagagattcactagtttctgattttgtgatctattcattatgtgtgaattcgttccccaaatttgtggttttaatgtttatggtctcaataggtgcttatgattttgatggatttagggtttatattttgatttctatggtgtagttgctgtgttttttatgattttgatggatttagggtttatattttgatttctatgatgtggttgctgttttttgatgttttagtggtttgatgttatcag

SEQ ID NO: 166 - DNA, 569 bp, Lotus japonicus
attgggtcaaaaaataaaactataaataagctcctccttgccctctctattcatcgacattctcattcgaattgaatcagttcatcaaaacctaattcctcttctctctatttctcctaccaaggtgcgtcttcgtctattttgaattcatcaaattgcgactctggtttcattatcttgaattctgattcatcccaaatttatggtttaatatttgtagttctaataggttacaatccgattagggtttgatggattagggatttgtttgttataatctgattagggtttgataattgatcctgcttatgttttcaatgtcatgagagattcactagtttctgattttgtgatctattcattatgtgtgaattcgttccccaaatttgtggttttaatgtttatggtctcaataggtgcttatgattttgatggatttagggtttatattttgatttctatggtgtagttgctgtgttttttatgattttgatggatttagggtttatattttgatttctatgatgtggttgctgttttttgatgttttagtggtttgatgttatcag

SEQ ID NO: 167 - DNA, 704 bp, Lotus japonicus
taatatgcaagcaatgacataattattttaaacagaatataatagcatcttctagagcattcaagaagagtgacctgggtcagcttatgtacacgtggcaagtctttactcgtcaaacacaaggatagtttcgtcattgggtcaaaaaataaaactataaataagctcctccttgccctctctattcatcgacattctcattcgaattgaatcagttcatcaaaacctaattcctcttctctctatttctcctaccaaggtgcgtcttcgtctattttgaattcatcaaattgcgactctggtttcattatcttgaattctgattcatcccaaatttatggtttaatatttgtagttctaataggttacaatccgattagggtttgatggattagggatttgtttgttataatctgattagggtttgataattgatcctgcttatgttttcaatgtcatgagagattcactagtttctgattttgtgatctattcattatgtgtgaattcgttccccaaatttgtggttttaatgtttatggtctcaataggtgcttatgattttgatggatttagggtttatattttgatttctatggtgtagttgctgtgttttttatgattttgatggatttagggtttatattttgatttctatgatgtggttgctgttttttgatgttttagtggtttgatgttatcag

SEQ ID NO: 168 - DNA, 844 bp, Lotus japonicus
gcatcacaatcacttggcaagaaacttttgtagactgtttgatttaaaatttactcattagaaaaaaaaaaaattattagggctcaaatcatgattgaattgagagagatttggatcaaaattcattgagagagacggcttaatatgcaagcaatgacataattattttaaacagaatataatagcatcttctagagcattcaagaagagtgacctgggtcagcttatgtacacgtggcaagtctttactcgtcaaacacaaggatagtttcgtcattgggtcaaaaaataaaactataaataagctcctccttgccctctctattcatcgacattctcattcgaattgaatcagttcatcaaaacctaattcctcttctctctatttctcctaccaaggtgcgtcttcgtctattttgaattcatcaaattgcgactctggtttcattatcttgaattctgattcatcccaaatttatggtttaatatttgtagttctaataggttacaatccgattagggtttgatggattagggatttgtttgttataatctgattagggtttgataattgatcctgcttatgttttcaatgtcatgagagattcactagtttctgattttgtgatctattcattatgtgtgaattcgttccccaaatttgtggttttaatgtttatggtctcaataggtgcttatgattttgatggatttagggtttatattttgatttctatggtgtagttgctgtgttttttatgattttgatggatttagggtttatattttgatttctatgatgtggttgctgttttttgatgttttagtggtttgatgttatcag

SEQ ID NO: 169 - DNA, 1049 bp, Lotus japonicus
tataatttaaattttacctcatgatgtgaactaagtgaaacttttttttattcatctgaaaattaacaaacaaactaaaaaaactcaaggaactaacacaatatctctcattaccagggtctcaacctccactggaggctcctccaacacacaaaaatcgacgaatgcagaattagctccacacatggtaagaaaatctgcaggggcatcacaatcacttggcaagaaacttttgtagactgtttgatttaaaatttactcattagaaaaaaaaaaaattattagggctcaaatcatgattgaattgagagagatttggatcaaaattcattgagagagacggcttaatatgcaagcaatgacataattattttaaacagaatataatagcatcttctagagcattcaagaagagtgacctgggtcagcttatgtacacgtggcaagtctttactcgtcaaacacaaggatagtttcgtcattgggtcaaaaaataaaactataaataagctcctccttgccctctctattcatcgacattctcattcgaattgaatcagttcatcaaaacctaattcctcttctctctatttctcctaccaaggtgcgtcttcgtctattttgaattcatcaaattgcgactctggtttcattatcttgaattctgattcatcccaaatttatggtttaatatttgtagttctaataggttacaatccgattagggtttgatggattagggatttgtttgttataatctgattagggtttgataattgatcctgcttatgttttcaatgtcatgagagattcactagtttctgattttgtgatctattcattatgtgtgaattcgttccccaaatttgtggttttaatgtttatggtctcaataggtgcttatgattttgatggatttagggtttatattttgatttctatggtgtagttgctgtgttttttatgattttgatggatttagggtttatattttgatttctatgatgtggttgctgttttttgatgttttagtggtttgatgttatcag

SEQ ID NO: 170 - DNA, 949 bp, Lotus japonicus
cgtgggcgtgggcgtgtgttgtctatctccttattatccaaaaaaattcaatatagggttgatagttgagttgcttaaggtggcaaagagttcctatagttaaagaatgaaaagggctagcaacacattcttttacagtttcttttcaacactatatcattgattgattgaaatttatgtaggttcaataaaaatatgggtcacactttcaatttaatgagactcacatgaattttaatttcttaattttgaaaaaatttggatctcctcaaactaaatttctctcgatctttcttaatccatgaccatgaacggttgattttaaatctaacttattaaaaaaaatataatttaaattttacctcatgatgtgaactaagtgaaacttttttttattcatctgaaaattaacaaacaaactaaaaaaactcaaggaactaacacaatatctctcattaccagggtctcaacctccactggaggctcctccaacacacaaaaatcgacgaatgcagaattagctccacacatggtaagaaaatctgcaggggcatcacaatcacttggcaagaaacttttgtagactgtttgatttaaaatttactcattagaaaaaaaaaaaattattagggctcaaatcatgattgaattgagagagatttggatcaaaattcattgagagagacggcttaatatgcaagcaatgacataattattttaaacagaatataatagcatcttctagagcattcaagaagagtgacctgggtcagcttatgtacacgtggcaagtctttactcgtcaaacacaaggatagtttcgtcattgggtcaaaaaataaaactataaataagctcctccttgccctctctattcatcgacattctcattcgaattgaatcagttcatcaaaacctaattcctcttctctctatttctcctaccaag

SEQ ID NO: 171 - DNA, 300 bp, Cajanus cajan
cctttttttttctctagattggactgaattgtatttctcgtcccgtatctggttgatttaatgattccttttcgtaagggttttacttatgtcgatgttttatacaaatttacagtaatttgtgtatctatgactgtttcctcggtatgttgattgtttaggatgactttgataatcaacgactttgattacttactttcagtcctgttgttttgtataatattttcatcatctattttcattcactgcatagcgcgtgttgaagaattggcgattgattttttactttttgttttgtag

SEQ ID NO: 172 - DNA, 646 bp, Cajanus cajan
aaagcataatagataatcgtattgttgcagagaaagaaaccctatcaaggtatcttcttctcctcttctccttcttattaagatttcgattattagggttttatgtttagatcataggatatgtcgagccctttcattttgatctgggtcgtttcgggtttgtgaaattcttttgaatctaattgtcgtgcatgattttaccttctcaataatcatggatgaggagatctttgcaagcttttattgaataataggctagtttgtttgtaagagatcttaagatctgttgtaatatgtttaatttgtcagatcgtgtacatgtactctgtattgtgtaattgtaaaacctttttttttctctagattggactgaattgtatttctcgtcccgtatctggttgatttaatgattccttttcgtaagggttttacttatgtcgatgttttatacaaatttacagtaatttgtgtatctatgactgtttcctcggtatgttgattgtttaggatgactttgataatcaacgactttgattacttactttcagtcctgttgttttgtataatattttcatcatctattttcattcactgcatagcgcgtgttgaagaattggcgattgattttttactttttgttttgtag

SEQ ID NO: 173 - DNA, 703 bp, Cajanus cajan
aagcaacgcatcagttttgctataaaagcgaagccccggacgccaacttttcttatcaaagcataatagataatcgtattgttgcagagaaagaaaccctatcaaggtatcttcttctcctcttctccttcttattaagatttcgattattagggttttatgtttagatcataggatatgtcgagccctttcattttgatctgggtcgtttcgggtttgtgaaattcttttgaatctaattgtcgtgcatgattttaccttctcaataatcatggatgaggagatctttgcaagcttttattgaataataggctagtttgtttgtaagagatcttaagatctgttgtaatatgtttaatttgtcagatcgtgtacatgtactctgtattgtgtaattgtaaaacctttttttttctctagattggactgaattgtatttctcgtcccgtatctggttgatttaatgattccttttcgtaagggttttacttatgtcgatgttttatacaaatttacagtaatttgtgtatctatgactgtttcctcggtatgttgattgtttaggatgactttgataatcaacgactttgattacttactttcagtcctgttgttttgtataatattttcatcatctattttcattcactgcatagcgcgtgttgaagaattggcgattgattttttactttttgttttgtag

SEQ ID NO: 174 - DNA, 836 bp, Cajanus cajan
ccaaatatagttgcaacgtttatcggttcccggaaaagactcgtacaccaaactttgccgtgtcaatagcgtttacggtcctaattcttccacgcgtcacaacatgattggaagtggtaccggtgccgtgacgaagcaacgcatcagttttgctataaaagcgaagccccggacgccaacttttcttatcaaagcataatagataatcgtattgttgcagagaaagaaaccctatcaaggtatcttcttctcctcttctccttcttattaagatttcgattattagggttttatgtttagatcataggatatgtcgagccctttcattttgatctgggtcgtttcgggtttgtgaaattcttttgaatctaattgtcgtgcatgattttaccttctcaataatcatggatgaggagatctttgcaagcttttattgaataataggctagtttgtttgtaagagatcttaagatctgttgtaatatgtttaatttgtcagatcgtgtacatgtactctgtattgtgtaattgtaaaacctttttttttctctagattggactgaattgtatttctcgtcccgtatctggttgatttaatgattccttttcgtaagggttttacttatgtcgatgttttatacaaatttacagtaatttgtgtatctatgactgtttcctcggtatgttgattgtttaggatgactttgataatcaacgactttgattacttactttcagtcctgttgttttgtataatattttcatcatctattttcattcactgcatagcgcgtgttgaagaattggcgattgattttttactttttgttttgtag

SEQ ID NO: 175 - DNA, 1030 bp, Cajanus cajan
acaattaacatttaagcatcggatatttacgaaaaataagatcaaatctatcactttggatatttcctttttacaccaaggatggatattttttttaatcgttcataattccaagagtaatagagaaaaattaaatatgtaaaaaaattgggatttattaaaaatggatgaattctaaaaatggccaacagattccaaatatagttgcaacgtttatcggttcccggaaaagactcgtacaccaaactttgccgtgtcaatagcgtttacggtcctaattcttccacgcgtcacaacatgattggaagtggtaccggtgccgtgacgaagcaacgcatcagttttgctataaaagcgaagccccggacgccaacttttcttatcaaagcataatagataatcgtattgttgcagagaaagaaaccctatcaaggtatcttcttctcctcttctccttcttattaagatttcgattattagggttttatgtttagatcataggatatgtcgagccctttcattttgatctgggtcgtttcgggtttgtgaaattcttttgaatctaattgtcgtgcatgattttaccttctcaataatcatggatgaggagatctttgcaagcttttattgaataataggctagtttgtttgtaagagatcttaagatctgttgtaatatgtttaatttgtcagatcgtgtacatgtactctgtattgtgtaattgtaaaacctttttttttctctagattggactgaattgtatttctcgtcccgtatctggttgatttaatgattccttttcgtaagggttttacttatgtcgatgttttatacaaatttacagtaatttgtgtatctatgactgtttcctcggtatgttgattgtttaggatgactttgataatcaacgactttgattacttactttcagtcctgttgttttgtataatattttcatcatctattttcattcactgcatagcgcgtgttgaagaattggcgattgattttttactttttgttttgtag

SEQ ID NO: 176 - DNA, 1183 bp, Cajanus cajan
gatttatcatctaggctacaaaataggttattcaagtaaataaaaattaacccagattatttaaatattaattaaaaaaataatatatttataaataaaaaaaagtatagaattggagcactagtttaacttattgacttgtgacgacatttaacaattaacatttaagcatcggatatttacgaaaaataagatcaaatctatcactttggatatttcctttttacaccaaggatggatattttttttaatcgttcataattccaagagtaatagagaaaaattaaatatgtaaaaaaattgggatttattaaaaatggatgaattctaaaaatggccaacagattccaaatatagttgcaacgtttatcggttcccggaaaagactcgtacaccaaactttgccgtgtcaatagcgtttacggtcctaattcttccacgcgtcacaacatgattggaagtggtaccggtgccgtgacgaagcaacgcatcagttttgctataaaagcgaagccccggacgccaacttttcttatcaaagcataatagataatcgtattgttgcagagaaagaaaccctatcaaggtatcttcttctcctcttctccttcttattaagatttcgattattagggttttatgtttagatcataggatatgtcgagccctttcattttgatctgggtcgtttcgggtttgtgaaattcttttgaatctaattgtcgtgcatgattttaccttctcaataatcatggatgaggagatctttgcaagcttttattgaataataggctagtttgtttgtaagagatcttaagatctgttgtaatatgtttaatttgtcagatcgtgtacatgtactctgtattgtgtaattgtaaaacctttttttttctctagattggactgaattgtatttctcgtcccgtatctggttgatttaatgattccttttcgtaagggttttacttatgtcgatgttttatacaaatttacagtaatttgtgtatctatgactgtttcctcggtatgttgattgtttaggatgactttgataatcaacgactttgattacttactttcagtcctgttgttttgtataatattttcatcatctattttcattcactgcatagcgcgtgttgaagaattggcgattgattttttactttttgttttgtag

SEQ ID NO: 177 - DNA, 903 bp, Cajanus cajan
gcatgcccaactgaagcttaagcctcatcaccaacccataaaaggctcaacatgccaaatatatatatatataatgccaacaaaatgctactccaaggtagcctacaaaaaaaaatcatctactcaagctcctaccaccaagtggcatccaacagctcacaataacctatagaaaattaaaataaattattttttatttttaaattttgaaagaaaaaaaaaaccaagtgaaaaattagttgctccaataaccgatttctcaataaaattttaaattttttttcaaaaatcttattgaaaaatcggttatccaataagatttatcatctaggctacaaaataggttattcaagtaaataaaaattaacccagattatttaaatattaattaaaaaaataatatatttataaataaaaaaaagtatagaattggagcactagtttaacttattgacttgtgacgacatttaacaattaacatttaagcatcggatatttacgaaaaataagatcaaatctatcactttggatatttcctttttacaccaaggatggatattttttttaatcgttcataattccaagagtaatagagaaaaattaaatatgtaaaaaaattgggatttattaaaaatggatgaattctaaaaatggccaacagattccaaatatagttgcaacgtttatcggttcccggaaaagactcgtacaccaaactttgccgtgtcaatagcgtttacggtcctaattcttccacgcgtcacaacatgattggaagtggtaccggtgccgtgacgaagcaacgcatcagttttgctataaaagcgaagccccggacgccaacttttcttatcaaagcataatagataatcgtattgttgcagagaaagaaaccctatcaag

SEQ ID NO: 178 - DNA, 308 bp, Cicer arietinum
tgaaattggttaatgctgtgttatttcatttctttttgttgtgcttgctacacaaatgtgaactttttctgttttagctagaaaaaaatagtgttcgagtaaaactaatcttctaaatttgtaattggctgaattctgtgggtggtccatttgaactttttagcattgacaattttattactgcatactgtagctttgtaaaagagatctaggttagtactttgttgttttttgttttgttatgtttgtgtcttttatttgtgtttttgttactgaacaatttgttaatctataatctgtgtgcaggc

SEQ ID NO: 179 - DNA, 444 bp, Cicer arietinum
agcttaagattacaggttgaacatgtggatatgttctattttattattgctctactttctttctttgaagacctcaaattgtctaacctttttctttttgttgagctcaaattatctaatataagtacttggtaaatgaaattggttaatgctgtgttatttcatttctttttgttgtgcttgctacacaaatgtgaactttttctgttttagctagaaaaaaatagtgttcgagtaaaactaatcttctaaatttgtaattggctgaattctgtgggtggtccatttgaactttttagcattgacaattttattactgcatactgtagctttgtaaaagagatctaggttagtactttgttgttttttgttttgttatgtttgtgtcttttatttgtgtttttgttactgaacaatttgttaatctataatctgtgtgcaggc

SEQ ID NO: 180 - DNA, 604 bp, Cicer arietinum
ttttgttgatttgttgactggatttactgtctgatctgttgttttaagtgggaatctcgccgtttttttttatagaacttcatttggtgtttggaaatttgagatttgagataccctgactcatagttaattgaagattttagtgcatagcttaatgtaaagcttaagattacaggttgaacatgtggatatgttctattttattattgctctactttctttctttgaagacctcaaattgtctaacctttttctttttgttgagctcaaattatctaatataagtacttggtaaatgaaattggttaatgctgtgttatttcatttctttttgttgtgcttgctacacaaatgtgaactttttctgttttagctagaaaaaaatagtgttcgagtaaaactaatcttctaaatttgtaattggctgaattctgtgggtggtccatttgaactttttagcattgacaattttattactgcatactgtagctttgtaaaagagatctaggttagtactttgttgttttttgttttgttatgtttgtgtcttttatttgtgtttttgttactgaacaatttgttaatctataatctgtgtgcaggc

SEQ ID NO: 181 - DNA, 794 bp, Cicer arietinum
atctatgttactcggatgtagtagatcgcggataactaatgagtgatctgtgatctggtatatgcttatgatgcgtcactctatctgaatctggtcttctacactctggatctaggatcttttataactattgtgtgtttgtaccggtcatagattccattcgctagcgttagtttcaaaataattccggttttgttgatttgttgactggatttactgtctgatctgttgttttaagtgggaatctcgccgtttttttttatagaacttcatttggtgtttggaaatttgagatttgagataccctgactcatagttaattgaagattttagtgcatagcttaatgtaaagcttaagattacaggttgaacatgtggatatgttctattttattattgctctactttctttctttgaagacctcaaattgtctaacctttttctttttgttgagctcaaattatctaatataagtacttggtaaatgaaattggttaatgctgtgttatttcatttctttttgttgtgcttgctacacaaatgtgaactttttctgttttagctagaaaaaaatagtgttcgagtaaaactaatcttctaaatttgtaattggctgaattctgtgggtggtccatttgaactttttagcattgacaattttattactgcatactgtagctttgtaaaagagatctaggttagtactttgttgttttttgttttgttatgtttgtgtcttttatttgtgtttttgttactgaacaatttgttaatctataatctgtgtgcaggc

SEQ ID NO: 182 - DNA, 604 bp, Glycine max
aaatatgtaaaaattatgataaattaattctattaaatttgtgaatttattttattattaagttataatatttaatgactaatttgataatatattatatttttaagattaatttgatattaaaggataaaatttatgatcaatttttttattaaattatatgttaaaattaatttattgtattttttatatatttggagattaaatttttttttctgttcacactttgtcagcacttttgttgttttttttttcaaaaagagaaaaagagaatataaatttaaatttaaagcagaagagaacgaagcggcgtcgtttgttgcggcctgaaaaaagtccacactcgtgaaagtcattggcataatgacgagcatatccgtgagtgacctctgatccgctccactaaccctagtcaactccaaactcaaccatagttactttacttcactcacaccccgccacgtgttccaatcgaacggtcacttctgcatcacgcgccactataaatatctctctctcgtcatccgcaaccccaagcaaaaccctaatccctctttcttcctcttcctcagtagtgcgattttcgattctcttctctgcaact

SEQ ID NO: 183 - DNA, 406 bp, Glycine max
agattaaatttttttttctgttcacactttgtcagcacttttgttgttttttttttcaaaaagagaaaaagagaatataaatttaaatttaaagcagaagagaacgaagcggcgtcgtttgttgcggcctgaaaaaagtccacactcgtgaaagtcattggcataatgacgagcatatccgtgagtgacctctgatccgctccactaaccctagtcaactccaaactcaaccatagttactttacttcactcacaccccgccacgtgttccaatcgaacggtcacttctgcatcacgcgccactataaatatctctctctcgtcatccgcaaccccaagcaaaaccctaatccctctttcttcctcttcctcagtagtgcgattttcgattctcttctctgcaact

SEQ ID NO: 184 - DNA, 266 bp, Glycine max
cacactcgtgaaagtcattggcataatgacgagcatatccgtgagtgacctctgatccgctccactaaccctagtcaactccaaactcaaccatagttactttacttcactcacaccccgccacgtgttccaatcgaacggtcacttctgcatcacgcgccactataaatatctctctctcgtcatccgcaaccccaagcaaaaccctaatccctctttcttcctcttcctcagtagtgcgattttcgattctcttctctgcaact

SEQ ID NO: 185 - DNA, 123 bp, Glycine max
acttctgcatcacgcgccactataaatatctctctctcgtcatccgcaaccccaagcaaaaccctaatccctctttcttcctcttcctcagtagtgcgattttcgattctcttctctgcaact

SEQ ID NO: 186 - DNA, 583 bp, Lotus japonicus
ctagatgaacatggggttatgcttttttttttttgtcaataatggatttcattgataaaaaaggcaagaagcccaatacacggcctgctaaaagtgaggggccacacatacaacaggggaaaaggctgatgatgatcagcaccagaggaataaccttgttaggagtaaatttgtgataaccccacatttcatcgagaattgatcatcgatatattaatcctcgtcgacattcatttggcccacatgaacccccaaaaaagtgattccaaaatacaaaatttttaaattaaaaaaaaaaacgaaaaatacacagtgaattaaagcacgaagcggcgtcgtttgtttgtgcatgaaaagtccacacccataagcatatccgcctccttcgatccgctccactaaccctagttaattcctaactcaaccatagttacatcactccatcacaacacaccacgtgtcctgatccaacggtgaattcacaacgtcttcagatgcacgcgccactataaataaccttcgtcctcacgttcattcgccaaaccctaatcccttttcactcttcatcactctctctgcatcc

SEQ ID NO: 187 - DNA, 386 bp, Lotus japonicus
attgatcatcgatatattaatcctcgtcgacattcatttggcccacatgaacccccaaaaaagtgattccaaaatacaaaatttttaaattaaaaaaaaaaacgaaaaatacacagtgaattaaagcacgaagcggcgtcgtttgtttgtgcatgaaaagtccacacccataagcatatccgcctccttcgatccgctccactaaccctagttaattcctaactcaaccatagttacatcactccatcacaacacaccacgtgtcctgatccaacggtgaattcacaacgtcttcagatgcacgcgccactataaataaccttcgtcctcacgttcattcgccaaaccctaatcccttttcactcttcatcactctctctgcatcc

SEQ ID NO: 188 - DNA, 242 bp, Lotus japonicus
gtttgtgcatgaaaagtccacacccataagcatatccgcctccttcgatccgctccactaaccctagttaattcctaactcaaccatagttacatcactccatcacaacacaccacgtgtcctgatccaacggtgaattcacaacgtcttcagatgcacgcgccactataaataaccttcgtcctcacgttcattcgccaaaccctaatcccttttcactcttcatcactctctctgcatcc

SEQ ID NO: 189 - DNA, 96 bp, Lotus japonicus
tcttcagatgcacgcgccactataaataaccttcgtcctcacgttcattcgccaaaccctaatcccttttcactcttcatcactctctctgcatcc

SEQ ID NO: 190 - DNA, 630 bp, Cicer arietinum
ttttattgtaaaagttaataattttaatatattttagatttttgaattaaaaaaataatgatttgacatattattatgagatacaaataaatgacatagaattatttatatgcatcatcgcatttatagaatcatctatatgcattaattatttatatatatacttttatttaaagtatcttacaacattattttttatctaaaaaatcatagaaatgatgaaaactattgatttttaaaataacatataaattttgtaaaatagtacatatagtacaaaccctttaattagacaaattcaaaactaaaaatacttttttttacaaaaaataaaataaaaaattagaacttagaaatgaataacattaacaggctgtcacgaaatacgatttgtttattttctgggttggtattttataaaattttgattcctcacaaatttggaagtattctaaacgaatttgtaatattgaagtattttaaccggtaaccggttacagcccgtgaaagtggaggccggtcgttctccatataagaagacacaagcgaaggaagcgaaaacaagaagaagcgaaagaaagaaagaaagcgctttcagtgactgagagaggtataaaacgctattccaac

SEQ ID NO: 191 - DNA, 404 bp, Cicer arietinum
tattgatttttaaaataacatataaattttgtaaaatagtacatatagtacaaaccctttaattagacaaattcaaaactaaaaatacttttttttacaaaaaataaaataaaaaattagaacttagaaatgaataacattaacaggctgtcacgaaatacgatttgtttattttctgggttggtattttataaaattttgattcctcacaaatttggaagtattctaaacgaatttgtaatattgaagtattttaaccggtaaccggttacagcccgtgaaagtggaggccggtcgttctccatataagaagacacaagcgaaggaagcgaaaacaagaagaagcgaaagaaagaaagaaagcgctttcagtgactgagagaggtataaaacgctattccaac

SEQ ID NO: 192 - DNA, 260 bp, Cicer arietinum
aggctgtcacgaaatacgatttgtttattttctgggttggtattttataaaattttgattcctcacaaatttggaagtattctaaacgaatttgtaatattgaagtattttaaccggtaaccggttacagcccgtgaaagtggaggccggtcgttctccatataagaagacacaagcgaaggaagcgaaaacaagaagaagcgaaagaaagaaagaaagcgctttcagtgactgagagaggtataaaacgctattccaac

SEQ ID NO: 193 - DNA, 120 bp, Cicer arietinum
tggaggccggtcgttctccatataagaagacacaagcgaaggaagcgaaaacaagaagaagcgaaagaaagaaagaaagcgctttcagtgactgagagaggtataaaacgctattccaac

SEQ ID NO: 194 - DNA, 620 bp, Cicer arietinum
attatcaattttaatttttagtttataattgaaaacccaatctaaagttttgaccgttcaattttaatctaactgtgatcgatgtctaaatgcctgattgcatgattttcttaaccgcaggaaactcaaatgagtataatatgaggtataagaaagatttttcttacttcaattactgaaataaaaaataattgggctctttgggccaaacacgatatgaaccaagatccaacccaagttcctagctaaataacctaattgttgttaccacttgtcttaaagcaagtgtgaatatgatgcgggagtaaagtcgtccaataaacacgcgtaaccaaccacgcaaacaatacgaacagaaaaaagaaaaactccaatttgtccaccaacggcaccttctacttagttattatattattatttattatttatttgttaggataaaaattaaaagtggaccccagtcatgaaatagaacattctagacactctcacattctaacctatataacacacatctcttccttagaccactcattctcaaatcacctttcagatccaaaatctgaaaccctaattttctttctctctgcttttttgattttctcttagttttcagtaaa

SEQ ID NO: 195 - DNA, 455 bp, Cicer arietinum
acttcaattactgaaataaaaaataattgggctctttgggccaaacacgatatgaaccaagatccaacccaagttcctagctaaataacctaattgttgttaccacttgtcttaaagcaagtgtgaatatgatgcgggagtaaagtcgtccaataaacacgcgtaaccaaccacgcaaacaatacgaacagaaaaaagaaaaactccaatttgtccaccaacggcaccttctacttagttattatattattatttattatttatttgttaggataaaaattaaaagtggaccccagtcatgaaatagaacattctagacactctcacattctaacctatataacacacatctcttccttagaccactcattctcaaatcacctttcagatccaaaatctgaaaccctaattttctttctctctgcttttttgattttctcttagttttcagtaaa

SEQ ID NO: 196 - DNA, 280 bp, Cicer arietinum
caaacaatacgaacagaaaaaagaaaaactccaatttgtccaccaacggcaccttctacttagttattatattattatttattatttatttgttaggataaaaattaaaagtggaccccagtcatgaaatagaacattctagacactctcacattctaacctatataacacacatctcttccttagaccactcattctcaaatcacctttcagatccaaaatctgaaaccctaattttctttctctctgcttttttgattttctcttagttttcagtaaa

SEQ ID NO: 197 - DNA, 139 bp, Cicer arietinum
gacactctcacattctaacctatataacacacatctcttccttagaccactcattctcaaatcacctttcagatccaaaatctgaaaccctaattttctttctctctgcttttttgattttctcttagttttcagtaaa

SEQ ID NO: 198 - DNA, 1274 bp, Cicer arietinum
tattaaatttaaaagtaaatattaataattaattttaaaaaatatgtatgatttaatgacaagggtagttttatcattttatataatattataaattattatccaaaattaaaattaaaaatataattattaattttaatgtaattttttaacaagggtagtttttcgatttacatataatatatattatatattcatactttaaaaagtttgaaagtgtcgatgaatatttttaatataaaattattttattatttatttaatattttttgttaatttataatatttaaaaatattttaagttaatttgaaaaaatattatattattgttacacaatagtgattttgtcatttaaatatgatatattttatatcatgtcatgatgaaatatgtactaaatgcatgacatgaaaaaataatgttatataaatataaatataaatagaaaaagaatttgaaattaataaaaataaacatatcaataattaattttataaatatatataatttttattacaaggtaattttttaatttacatattctaaaaaattaaaccaattggtattgaatatttaaaaataaaattattattaaaatttacaaaaatatgaatatgaattaaatttttttaacaataaatataattctttttgtagatatagtttcattatttacttataatatatatcatatctatatttaatttgtctaacatatataatagaattattttaattagtcattactttttaaattgaaattatttgtttatgagagttaatggattttgtcaaactaattaattttctctgtatttatttatttataaaaattaaagatatttccaattaattaatttgaataaaatatatgtaattatatcacaaaagtgattttgttatatatttttaatcttaaaagtgatagtaaatttcaccttaataaacttaaatataataatatccacatttattatttaaatatgatatatataccaaaatataatatatattaataaaaaaatacaatataataaataatgttctacactattattttttctttcttaaaaccacaatttttttggagctaggatgttttttattctatctcttaactctataaattacgtaatttgggttggactttattatattcaatttgtcatagttcttgcttcggctttccctctcttcactgttctctcatctcttccctccgttttttctccttcacttttcttcattcattctcctctaccttcctccaacc

SEQ ID NO: 199 - DNA, 1542 bp, Arabidopsis thaliana
aatttattatgtgttttttttccgtggtcgagattgtgtattattctttagttattacaagacttttagctaaaatttgaaagaatttactttaagaaaatcttaacatctgagataatttcagcaatagattatatttttcattactctagcagtatttttgcagatcaatcgcaacatatatggttgttagaaaaaatgcactatatatatatatattattttttcaattaaaagtgcatgatatataatatatatatatatatatatatgtgtgtgtgtatatggtcaaagaaattcttatacaaatatacacgaacacatatatttgacaaaatcaaagtattacactaaacaatgagttggtgcatggccaaaacaaatatgtagattaaaaattccagcctccaaaaaaaaatccaagtgttgtaaagcattatatatatatagtagatcccaaatttttgtacaattccacactgatcgaatttttaaagttgaatatctgacgtaggatttttttaatgtcttacctgaccatttactaataacattcatacgttttcatttgaaatatcctctataattatattgaatttggcacataataagaaacctaattggtgatttattttactagtaaatttctggtgatgggctttctactagaaagctctcggaaaatcttggaccaaatccatattccatgacttcgattgttaaccctattagttttcacaaacatactatcaatatcattgcaacggaaaaggtacaagtaaaacattcaatccgatagggaagtgatgtaggaggttgggaagacaggcccagaaagagatttatctgacttgttttgtgtatagttttcaatgttcataaaggaagatggagacttgagaagttttttttggactttgtttagctttgttgggcgttttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaag

SEQ ID NO: 200 - DNA, 1004 bp, Chrysanthemum x morifolium
aagcttagacaaacaccccttgttatacaaagaatttcgctttacaaaatcaaattcgagaaaataatatatgcactaaataagatcattcgcatccaatctaaccaattacgatacgctttgggtacacttgatttttgtttcagtggttacatatatcttgttttatatgctatctttaaggatctgcacaaagattatttgttgatgttcttgatggggctcagaagatttgatatgatacactctaatctttaggagataccagccaggattatattcagtaagacaatcaaattttacgtgttcaaactcgttatcttttcattcaaaggatgagccagaatctttatagaatgattgcaatcgagaatatgttcggccgatatgcctttgttggcttcaatattctacatatcacacaagaatcgaccgtattgtaccctctttccataaaggaaaacacaatatgcagatgcttttttcccacatgcagtaacatataggtattcaaaaatggctaaaagaagttggataacaaattgacaactatttccatttctgttatataaatttcacaacacacaaaagcccgtaatcaagagtctgcccatgtacgaaataacttctattatttggtattgggcctaagcccagctcagagtacgtgggggtaccacatataggaaggtaacaaaatactgcaagatagccccataacgtaccagcctctccttaccacgaagagataagatataagacccaccctgccacgtgtcacatcgtcatggtggttaatgataagggattacatccttctatgtttgtggacatgatgcatgtaatgtcatgagccacagcatccaatggccacaggaacgtaagaatgtagatagatttgattttgtccgttagatagcaaacaacattataaaaggtgtgtatcaataggaactaattcactcattggattcatagaagtccattcctcctaagtatctaaa

SEQ ID NO: 201 - DNA, 1305 bp, Arabidopsis thaliana
atcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaaca

SEQ ID NO: 202 - DNA, 1442 bp, Cicer arietinum
gcaatagggaaatcaaaactcacatagttcttgagtagaaaatatttatgtgttaatcgattttattattaattttactttatatgtatatcaaaataaattaacaacataatggtgataataatagtaataatggtaataatgaaaataataataggagtgacgtgttgtatattgatttgtgtatcgttttattttaaagaaacatttttacttccttttttatttttataaactgtaaaggataaaaattcaagagcagatccgcgagtcggcgacgcatatgtggtgcgaaccgacggcggtaataataagagaatttaagtactaacgcacaagaaataaaacacaataaagatgagagattgcgaatttgataaagaaataaacgattatatacaacaatcaaattgatttttattagtagattccaaaacttggtcgtgaaaattaaaaagaaaaaattattgtttatatcatacgtgcatagaattgtaaaaaaaaatctataacaataataatgtaaaaaaaaatagacaagaagaaaaaagaaaaataagtgtttaccacactagaaaggttctttactagctcgttccaatctttcttctttcttttctctctattattctctcttctattttttttttttgtgctatcaattgtgctcccaatttctctcaattgtgctccgaatctcccccacttgtcttcttgtggtagactatttatagactttttgggtgtagattacaaaagacaagagaataattgttaatgaattaccattaatctgtttctttcagtttattttcatttaattggccacaattatactcattcttattattattgattcatttcttcgatttcttttctattgaattgaccaccattatactgatttttatataccgaaattccgttatggtccaattctgacctataatagataccagggctttgtaatatttgattgattactattctgtatattccatttactatagaagttcccagggaatctattaggggaatgtttccaataaaaataatttgttcttggatctacctactgtttttccaaattaatcccgcggatatatataattcagaggaatatgtaagtgattcatatacagcatctttttcttttatcgcgggttctaccaattgatatgtttccacaaataactgaaattcaatttcttgatcggtatcttctatttttggaaactttaaaagttcttctgttaagccccgatcaatgaatctagaaaacccttcaaattgtatttgattcaatccgggtagtgtagacattccttcattcccaacgccaaccattttcaatttccccatttcttttatagaaaatattccacgatttgctatgattcttcatcaactcacatgaatcgatttgctttgttatcgaacac

SEQ ID NO: 203 - DNA, 1145 bp, Cicer arietinum
atgggatctaattcttagggatcacttatcattatcattatcattatcatacgtctgtaatgctatatttttccaaagggtctcggaaaaataatatttaaatggtagtttaaaatatttaaaatttattgtttttttttagaaagaaaataaaagtaagactcttcgatttcgacaaaatacccatacctaagtttcatagctctcgttccactaggccggtcatgattgattttcctacccccaagggccttccccttttagccagttgtgggcgaggagggattcgaacccccgacaccgtggttcgtagccacgtgctctaatcctctgagctataggccccaccctgtctccatttgatctattcccagagtaccaccttccctttccatttttgcttaagaaaatgttaaaacgcatgcctctctctctataagaataagaacggtacgtttcgaggtgtgaagcgggagagattggattttataattggggtttttaataagacaactttttcattcttattccctttttgaatagggataggaaataagaatgaaaagtgttaagtcttttttttatcatcctggcgccgagctatttttccgcagggcctcccctacagtatcgtcaccgcagtagagtttaaccaccaagttcgggatggattggtgtggttcctctacgcctaggacaccagaatatcgaacatgaacgaagtaaaggcctgagaaaaaaagcatattgacactgtaggggacaccaaagtcctccgcccgtccatccctatctattcaagggatgggggacagagcttttggttttttcatgttgtcaaagagttgaacaataaaaatagatggcgagtacctgatcgaattgatcaggtcatgtaggaacaaggttcaagtcatccggtctgttaggatgcctcagctgcatacattactgcacttccacttgacacctatcgttaatgaaaaacggctcgtctcgccgtgaccttctcttgaatttccaaaacttcttttgctccatccccgcaggggcagagaacccatcgctgtttcggctgtgataccgaaggctctggggaagtcggaataggagagcactcatcttggggtgggcttactacttag

SEQ ID NO: 204 - DNA, 1634 bp, Cicer arietinum
tattttaaaatttaaagttgttacatatatttgaagcacaaatactcactttcattaaaccaaaaactctaacacgatcatatagaattataagtttactgcatataataaaactatcacattcaaatatcataatagaactataagtctattcatatttaaataacatgtttgtatctaaaaagtgtttataaaatgacaacataaaatagtggtttatccttgaaactagtcctcctattccacataatttgtaatagattgaaataactgaatgaattgctaattaatccaaattcatagaactataagaagaaacaaaacagtgcaatttggagtccaacttgtgagcaaacatcgaacccaacttggaactccaatgaagagtggccataacatgactctttgtccgttagtatctcctatcacatattaatttgtttgtcatttttcttaatcttattctaataaagtaaacaatttattttaaaaaataaatagaatattatcaattattgattaataaattaaagatagatattatagttacatatgagcaaacaatgaatattgaaaaataatttaaattttttcttcattttatactctatattttctagtcactttagacaaatttgtttttttttgttatccatgttgtccacatatttaatctatattaatttttgttttgtaatttttatactttctacctatgaatctgtaaaagttgaataatttctagaagttattattctacatccaaattatcttatattataaataacattaccagttaaacagatttttcctaataaaaaagaaggttaaaataaatatatcaaactaagaaaaaagtagtcctattcttattaaaaaccaattaaggaaagtgtagaatttcatagaattttattgtccattgatataatttgaaaacccgaagacttaatccatttaagatttcacaaaattaaagtccaacactcaaattacaaaagaaaaaaaaaataaaccttaatttgagttctgaaattgtaacttcgatcaatttagaattttaaatttataaaatattaagttactcttttacattttttaacccattagcaaaaagttccataagatagtttgatagaaaaactattatgagtgattttaaacaatttaagaaaacaacttataatttgaagaacaaaaatagtagtttacttggaaaaatatgagtgagtgagagtgatatcatgattggacttggaagcacaaaaacaaagcatgaattacagccattcattgaatgaaaatgctatccaaaatctaatccttacttggttgggctcatgagtcacaaccacacatccacgttgacacctcttactcgatcacttcactttatagtacataataataacattacatgtaactaagaatttgtttcccttgcaaagctcaatactacaacttcaaaaaaatcactttttctttcttgtagacttaatcgttcaacatatattactcattattttttctaatgtttcattaatgtagtgcatctttataagttcatattaactagtagcacaattttgtggtaaaacttataatcattttttttcttcttttgatgtattcag

SEQ ID NO: 205 - DNA, 1500 bp, Cicer arietinum
taacacgatatattaaaaaaacaaacccattagtaaaaataacaaactaaaattaaagatcacccaactactcacctgttaccaccggaaccaagtcttttccttcttctcctcttgttactctattgtcacaacttttctgtttgtccttctttttcttgttactgtgtggctctattttcattttttttttcttcacacgtgtcaagcccaatgaaacaatattccttttttctttctttcttttcgttctttaataataataacaataataatactactaattataataatactaataaaattgataggttccatttgtagagtaagctcacctaacacatgttcaaacttttaacaagatcattaaagggttgtcatgcattgatagttaaaatattttgtggactttacaatcatgagttatttgatattttaaattatcatttatttctaggacgtttaagttgcaagattgaaacaaggacagtcttactattctcatacaaatgtataagaagttatagagcatgtaaacatcaactttgagaggtcttaaaacaaagatgcaaaatctaacaatatcaatagaaaaggaaaagtatgtgtgtggaaataaaacacataaaatctcaaatataatgagttatatattttgatgcttccatattcgatcaagttattgaacatgttttctatcatattaaacattgacgtccgacacaaaattaatttattgaaatgaaagataactaagatatttcatatgattcgttggaataggggtatactttgaatttggattcgacactataaacgaaaatcacacaattacacattcacatcgatgaccactcatcaaaataagacaatctaaaaaattatcaattttaatttttagtttataattgaaaacccaatctaaagttttgaccgttcaattttaatctaactgtgatcgatgtctaaatgcctgattgcatgattttcttaaccgcaggaaactcaaatgagtataatatgaggtataagaaagatttttcttacttcaattactgaaataaaaaataattgggctctttgggccaaacacgatatgaaccaagatccaacccaagttcctagctaaataacctaattgttgttaccacttgtcttaaagcaagtgtgaatatgatgcgggagtaaagtcgtccaataaacacgcgtaaccaaccacgcaaacaatacgaacagaaaaaagaaaaactccaatttgtccaccaacggcaccttctacttagttattatattattatttattatttatttgttaggataaaaattaaaagtggaccccagtcatgaaatagaacattctagacactctcacattctaacctatataacacacatctcttccttagaccactcattctcaaatcacctttcagatccaaaatctgaaaccctaattttctttctctctgcttttttgattttctcttagttttcagtaaa

SEQ ID NO: 206 - DNA, 1500 bp, Cicer arietinum
ggtacttatttcttttgctcaaatttacgaaaatgttttgacatgtctttcacacaattggttatactatacattaaaagagatgtattgtctattcaaattcttgaggtgaatataaagtcgatagggttagaagattgcaaggtcaggtttggttcactttaaggtgatatacattcctttataaactgtggaattgaatatcaagttgtcttatatttaaaatttgagtagtcattgatttttaatctcttggaaaataatattaagagttttatttctctttaaaagtacataatataatgtttaagataacattacgtagtataatattaaataaaatataatcacttagtttcagtactcaaatttaattgatttgatttaatttgaacatgaatataaaaagaagaaaaaaattaaactaatttagtttatttgaattggtttggtttaagttaagttagtcaaggtattggtttggtgtttacatcatactttatacaccctctgtcctttattatgaaatctatttttctttgccccttaatacaacagtatatgatatattcaatattaaatatacataatggcttttttttaataaaaaacctctatttaattttcattaaaaatattaattttttaaatttatgatatccataactatataaataaatgtagaaaaataagacactttcaacaaaattattgctccaattatttttaatattcgtgaatatttgtcagtagaattttataataaaagacagatagagtatcaagtcaaataaagttaaaaagatttaatttgtaaaaaagaaaataaagaatctaaataaaaaatttgaatataaaatttaatgtattaaattcatttaaaaattatctatttggtcttaaatatatgaaaaaaattatgttaataaatgttaatgtatttatttatttttttgtctttttttaagtgtcatttttattttttatttggctttttcaacttttaaataaaaattgtatttattgcaatgagggaaataaatgtgaataataaaaaatagttaaaaataatatagaaaaaaattaaattgtttataaaaatatcaaaatttaatagttttattttgtttggtttaacaaacttaaaatgagaattaaaaaggtatattaaacgatcttaagaaatgtgttccattgatttttgatcataatcgaatctatagtaaaaaacatacaccctcgaacacacaacaaaaacccttgaacaaatctgtagatacgtgtttacgaaaacgacgtagacgtggtaatagatccgcaaatagattagaaccaccttaagatttgaacacgtgtccattgatatttattattctctctcctcaaatcacgctataaatcagacttcaaggcactcttcattcattcaacgttgtttgtttgttttgtgtaaaaaatcatcttttgttgtttgttgtctagtttttattagaactgaaa